SEQUENCE LISTING (20 sequences)

SEQ ID NO: 1 - protein, 254 aa, Homo sapiens
  source 1..254: note = Human 4-1BBL (HUMAN Tumor necrosis factor ligand superfamily member 9/h4-1BBL)
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - DNA, 765 bp, Homo sapiens
  source 1..765: mol_type = genomic DNA; note = Nucleotide sequence of Human 4-1BBL (HUMAN Tumor necrosis factor ligand superfamily member 9/h4-1BBL)
atggagtatgccagtgatgcatccctggacccagaggccccctggcccccagccccaagggccagggcttgcagagttctgccctgggctttggtggcaggcctgctcctcctgctcctcctggcagcagcctgtgctgtgttcctggcctgcccctgggcagtcagtggggccagggccagcccagggtctgctgccagccccagactcagagagggccctgagttgagcccagatgaccctgcaggcctgctggacctgagacaagggatgtttgcccagctggtggcccagaatgtgctcctgatcgatggccccttgagctggtacagtgacccaggcctggctggggtcagcctgacagggggcctgagctacaaggaggacaccaaggagctggtggtggccaaggcaggagtctactatgtgttcttccagctggagctgagaagggtggtggcaggagagggcagtggctcagtgtcactggccctccacctgcagcccctcagatcagctgctggggctgcggccctggccctcactgtggacttgccccctgccagcagtgaggccagaaacagtgcctttggcttccaaggcaggctgctgcacctgagtgccgggcagagactgggggtccacctccacactgaggcgagagccaggcatgcctggcagctcacccaaggggccactgtcctgggcctgttcagagtcaccccagagatccctgctggcctccccagccccaggagtgagtga

SEQ ID NO: 3 - protein, 153 aa, Homo sapiens
  source 1..153: note = Amino acid sequence of Human Interleukin-2 (hIL-2)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - DNA, 462 bp, Homo sapiens
  source 1..462: mol_type = genomic DNA; note = Nucleotide sequence of Human Interleukin-2 (hIL-2)
atgtacagaatgcagctgctgagctgcattgccctgagcctggccctggtgaccaacagtgcccccaccagcagcagcaccaagaagacacagctgcagctggagcacctgctgctggacctgcagatgatcctgaatggcatcaacaactacaagaaccccaagctgaccagaatgctgaccttcaagttctacatgcccaagaaggccactgagctgaagcacctgcagtgcctggaggaggagctgaagcccctggaagaggtgctgaacctggcccagagcaagaacttccacctgagacccagagacctgatcagcaacatcaatgtgattgtgctggagctgaagggcagtgagaccaccttcatgtgtgagtatgcagacgagacagccaccattgtggagttcctgaacagatggatcaccttctgccagagcatcatcagcaccctgacctga

SEQ ID NO: 5 - protein, 410 aa, synthetic construct
  DOMAIN 1..153: note = Human Interleukin 2 (IL2)
  DOMAIN 154..177: note = Rigid Linker
  DOMAIN 178..410: note = CD16b/FCGR3B - GPI anchor
  source 1..410: note = Amino acid sequence of Human Interleukin 2-Fusion Protein (hIL2-RG)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTAEAAAKEAAAKEAAAKEAAAKAAAMWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 6 - DNA, 1233 bp, synthetic construct
  source 1..1233: mol_type = other DNA; note = Nucleotide sequence of Human Interleukin 2-Fusion Protein (hIL2-RG)
atgtacagaatgcagctgctgagctgcattgccctgagcctggccctggtgaccaacagtgcccccaccagcagcagcaccaagaagacccagctgcagctggagcacctgctgctggacctgcagatgatcctgaatggcatcaacaactacaagaaccccaagctgaccagaatgctgacgttcaagttctacatgcccaagaaggccacagagctgaagcacctgcagtgcctggaggaggagctgaagcccctggaagaggtgctgaacctggcccagagcaagaacttccacctgagacccagagacctgatcagcaacatcaatgtgattgtgctggagctgaaggggagtgagaccaccttcatgtgtgaatatgcagatgagacagccaccattgtggagttcctgaacagatggatcaccttctgccagagcatcatcagcaccctgacagctgaggctgctgccaaagaggcagcagcgaaagaagctgcagccaaggaagcggcagccaaggctgcagccatgtggcagctcctgttgcccacagccctgctgctgctggtgagtgcaggcatgagaacagaggacctgcccaaggcagtggtgttcctggagccccagtggtacagtgtgctggagaaggacagtgtgaccctgaagtgccaaggtgcctacagccctgaggacaacagcacccagtggttccacaatgagagcctgatcagcagccaggccagcagctacttcattgatgctgccaccgtgaatgacagtggtgagtacagatgccagaccaacctgagcaccctgagtgaccctgtgcagctggaggtgcacatcggctggctgctgctgcaggcccccagatgggtgttcaaggaggaggaccccatccacctgagatgccacagctggaagaacacagccctgcacaaggtgacctacctgcagaatggcaaggacagaaagtacttccaccacaacagtgacttccacatcccaaaggccaccctgaaggacagtggcagctacttctgcagaggcctggtgggcagcaagaatgtgagcagtgagacagtgaacatcaccatcacccaaggcctggctgtgagcaccatcagcagcttcagcccccctggctaccaagtgagcttctgcctggtgatggtgctgctgtttgcagtggacacaggcctgtacttcagtgtgaagaccaacatctga

SEQ ID NO: 7 - protein, 177 aa, Homo sapiens
  source 1..177: note = Amino acid sequence of Human Interleukin 7 (hIL-7)
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 8 - DNA, 534 bp, Homo sapiens
  source 1..534: mol_type = genomic DNA; note = Nucleotide sequence of Human Interleukin 7 (hIL-7)
atgttccatgtgagcttcagatacatctttggcctgccccccctgatcctggtcctgctgcctgtggccagcagtgactgtgacatagagggcaaggatggcaagcagtatgagagtgtgctgatggtgagcattgaccagctgctggacagcatgaaggagattggcagcaactgcctgaacaatgagttcaacttcttcaagagacacatctgtgatgccaacaaggagggcatgttcctgttcagggctgccagaaagctgagacagttcctgaagatgaacagcactggggactttgacctgcacctcctgaaggtgagtgagggcaccaccatcctgctgaactgcactggccaagtgaaaggcagaaagccagcggcccttggggaggcccagcccaccaagagcctggaggagaacaagagcctgaaggagcagaagaagctcaatgacctgtgcttcctgaagagactgctccaagagatcaagacctgctggaacaagatcctgatgggcaccaaggagcactga

SEQ ID NO: 9 - protein, 266 aa, Mus musculus
  source 1..266: note = Amino acid sequence of Mouse Interleukin-33 (mIL-33)
MRPRMKYSNSKISPAKFSSTAGEALVPPCKIRRSQQKTKEFCHVYCMRLRSGLTIRKETSYFRKEPTKRYSLKSGTKHEENFSAYPRDSRKRSLLGSIQAFAASVDTLSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 10 - DNA, 801 bp, Mus musculus
  source 1..801: mol_type = genomic DNA; note = Nucleotide sequence of Mouse Interleukin-33 (mIL-33)
atgagacccagaatgaagtacagcaacagcaagatcagccctgccaagttcagcagcacggcaggggaggccctggtgcccccctgcaagatcagaagatcccagcagaagaccaaggagttctgccatgtgtactgcatgagactgagaagtggcctgaccatcagaaaggagaccagctacttcagaaaggagcccaccaagagatacagcctgaagagtggcaccaagcatgaggagaacttcagtgcctaccccagagacagcagaaagagaagcctgctgggcagcatccaagcctttgctgccagtgtggacaccctgagcatccaaggcaccagcctgctgacccagtccccagccagcctgagcacctacaatgaccagagtgtgagctttgtgctggagaatggctgctatgtcatcaatgtggatgacagtggcaaggaccaagagcaagaccaagtgctgctgagatactatgagagcccctgccccgcctcccagagtggtgatggtgtggatggcaagaagctcatggtgaacatgagccccatcaaggacacagacatctggctgcatgccaatgacaaggactacagtgtggagctgcagagaggagatgtgagccccccagagcaagccttctttgtgctgcacaagaagagcagtgactttgtgagctttgagtgcaagaacctgccaggcacctacattggtgtgaaggacaaccagctggccctggtggaggagaaggatgagagctgcaacaacatcatgttcaagctgagcaagatctga

SEQ ID NO: 11 - protein, 543 aa, synthetic construct
  DOMAIN 1..335: note = Mouse IL-12p70 heterodimer (mIL12) and fusion protein of p40 (mIl12 p40)
  DOMAIN 336..350: note = GS linker V1
  DOMAIN 351..543: note = mIl12 p35
  source 1..543: note = Amino acid sequence of Mouse IL-12p70 heterodimer (mIL12). Reference: bioactive mouse IL-12p70 heterodimer, fusion protein of p40, GS linker and p35 (without signal sequence) IL-12.p40.L.Lamda-p35
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 12 - DNA, 1632 bp, Mus musculus
  source 1..1632: mol_type = genomic DNA; note = Nucleotide sequence of Mouse IL-12p70 heterodimer (mIL12)
atgtgcccccagaagctcaccatcagctggtttgccattgtgctgctggtcagccccctgatggctatgtgggaactggagaaggatgtgtatgtggtggaggtggattggaccccagatgccccaggggagactgtcaacctgacttgtgacacccctgaggaagatgacatcacctggacctcagaccagaggcatggagtgattggcagtggcaagactctcaccatcacagtcaaggagtttctggatgcaggccagtacacttgtcacaaagggggggagactctgagccacagccacctcctcctccacaagaaggagaatggcatctggtccactgagatcctgaagaacttcaagaacaagacctttctgaagtgtgaggcccccaattacagtggcaggttcacttgcagctggctggtccagagaaacatggacctcaagttcaacatcaagtccagcagcagcagccctgacagcagagcagtgacttgtggcatggcctctctctctgcagagaaggtcaccctggatcagagggactatgagaagtactctgtgagctgccaagaggatgtcacatgccccactgctgaggaaactctgcccatagagctggcccttgaggcaaggcagcagaacaagtatgagaattacagcaccagtttcttcatcagggacatcatcaagccagatcctcccaagaatctgcagatgaagcccctgaagaacagccaggtggaagtcagctgggagtacccagacagctggagcaccccccattcctacttctccctcaagttctttgtcagaatccagaggaagaaggagaagatgaaggagacagaggagggctgcaatcagaagggagccttcctggttgaaaagaccagcactgaagtccagtgcaaggggggcaatgtctgtgttcaagcccaagacaggtactacaactccagttgcagcaagtgggcttgtgtcccttgcagagtcaggagtggaggaggaggcagtggaggaggaggcagtggtgggggaggcagcagagtcattccagtcagtggcccagccaggtgtctttcccagtccaggaacctcctcaagacaacagatgacatggtcaagactgctagggagaaactgaagcactacagctgcacagctgaagacattgaccatgaggacatcaccagggaccagaccagcactctgaagacttgcctgcctctggaactccacaagaatgagagttgtctggccacaagagagaccagcagcacaaccagaggcagctgcctccccccccagaaaaccagcctcatgatgaccctctgcctgggcagcatctatgaggacctgaagatgtatcagactgagttccaagccatcaatgctgctctgcagaatcacaaccatcaacagatcatcctagacaagggcatgctggtggctattgatgagctcatgcagtccctcaatcacaatggggagaccctgaggcagaagcccccagttggtgaggctgacccctacagagtcaagatgaagctgtgcattctgctccatgccttctcaaccagggtggtcaccatcaacagggtcatgggctacctgagcagtgcctga

SEQ ID NO: 13 - protein, 260 aa, Mus musculus
  source 1..260: note = Amino acid sequence of Mouse CD40L (mCD40L)
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 14 - DNA, 783 bp, Mus musculus
  source 1..783: mol_type = genomic DNA; note = Nucleotide sequence of Mouse CD40L (mCD40L)
atgattgaaacatacagccagccttcccccagatcagttgccactggacttccagcaagcatgaagattttcatgtatttgctcactgtcttcctcatcacacaaatgattggatctgtactttttgctgtgtatcttcacagaagattggacaaagttgaagaggaagtgaaccttcatgaagattttgtcttcatcaaaaagctcaagagatgcaacaaaggagaaggatctttgtctttgctgaactgtgaggagatgaggaggcaatttgaagatcttgtgaaggacatcactttgaacaaagaagagaaaaaagaaaacagctttgaaatgcaaagaggagatgaggatccccaaattgcagcacatgttgtcagtgaagccaacagcaatgcagcatcagttcttcagtgggccaagaagggatattacacaatgaaaagcaacttggtgatgcttgaaaatgggaaacaactgacagtcaaaagagaaggactctattatgtttacactcaagtcaccttctgttcaaacagggagccttccagtcaaagaccattcattgttggcctctggctcaagccaagcagtggatcagagagaatcttgctcaaggcagcaaacacccacagttcctcccaactttgtgagcagcagtcagttcatttggggggtgtctttgaattgcaagctggtgcatctgtttttgtcaatgtgactgaagcaagccaagtgatccacagagttggcttctcatcttttggcttgctcaaactctga

SEQ ID NO: 15 - protein, 309 aa, Mus musculus
  source 1..309: note = Amino acid sequence of Mouse 4-1BBL (m4-1BBL)
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 16 - DNA, 930 bp, Mus musculus
  source 1..930: mol_type = genomic DNA; note = Nucleotide sequence of Mouse 4-1BBL (m4-1BBL)
atggaccagcacacccttgatgtggaggacacagctgatgccagacaccctgctggcacttcctgcccttcagatgctgccctcctcagagacactgggctcctggcagatgctgctctcctctcagacactgtgagacccacaaatgctgcactccccactgatgctgcctaccctgctgtcaatgtgagggacagagaggcagcctggccccctgccctgaacttctgttccagacaccccaagctctatggcctggttgccttggttttgctgctgctgatagcagcctgtgttcctatcttcaccagaactgagccaaggcctgccctcaccatcaccacctcacccaacctgggaaccagagagaacaatgcagaccaggtcacccctgtttctcacattggctgccccaacaccacacaacagggctctcctgtgtttgccaagctccttgctaaaaaccaagcctccttgtgcaacacaaccctgaactggcacagccaggatggggctgggagctcatacctctctcaagggctgaggtatgaggaagacaagaaggagttggtggttgacagtcctggtctctactatgtgttcttggagctgaagctcagtcccacattcaccaacacaggacacaaggtgcagggctgggtctctctggtcttgcaagcaaagcctcaggttgatgactttgacaacttggccctgacagtggaactgttcccttgctccatggagaacaagttggtggacaggtcctggagccaactgttgctcctgaaggctggccacaggctgagtgtgggtctgagggcttatctgcatggagcccaggatgcctacagagactgggagctgtcctatcccaacaccaccagctttgggctctttcttgtgaaaccagacaacccctgggaatga

SEQ ID NO: 17 - protein, 183 aa, Homo sapiens
  source 1..183: note = Amino acid sequence of Human Tumor necrosis factor ligand superfamily member 4 (hOX40L)
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 18 - DNA, 552 bp, Homo sapiens
  source 1..552: mol_type = genomic DNA; note = Nucleotide sequence of Human Tumor necrosis factor ligand superfamily member 4 (hOX40L)
atggagagagtgcagcccctggaggagaatgtgggcaatgcagccagacccagatttgagagaaacaagctgctgctggtggccagtgtgatccaaggcctgggcctgctgctgtgcttcacctacatctgcctgcacttcagtgccctgcaagtgagccacagataccccagaatccagagcatcaaggtgcagttcactgagtacaagaaggagaagggcttcatcctgacctcccagaaggaggatgagatcatgaaggtgcagaacaacagtgtgatcatcaactgtgatggcttctacctgatcagcctgaagggctacttcagccaagaggtgaacatcagcctgcactaccagaaggatgaggagcccctgttccagctgaagaaggtgagaagtgtgaacagcctgatggtggccagcctgacctacaaggacaaggtgtacctgaatgtgaccacagacaacaccagcctggatgacttccatgtgaatggtggtgagctgatcctgatccaccagaaccctggcgagttctgtgtgctgtga

SEQ ID NO: 19 - protein, 450 aa, synthetic construct
  DOMAIN 1..134: note = IL15-N72D
  DOMAIN 135..218: note = IL15RaSu
  DOMAIN 219..450: note = IgG1 CH2-CH3 (Fc Domain)
  source 1..450: note = Amino acid sequence of ALT-803 (IL15/IL15Ra)
METDTLLLWVLLLWVPGSTGNWVNVISDLKKIEDLIQSMHIDATLYTESDVHPSCKVTAMKCELLELQVISLESGDASIHDTVENLIILANDSLSSNGNVTESGCKECEELEEKNIKEFLQSFVHIVQMFINTSMDRLTSSFLLLIVPAYVLSITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1353 bp, synthetic construct
  source 1..1353: mol_type = other DNA; note = Nucleotide sequence of ALT-803 (IL15/IL15Ra)
atggagacggacaccctcctgctgtgggtgctgttgctgtgggtgccaggcagcacaggcaactgggtgaatgtgatcagtgacctgaagaagatcgaggacctgatccagagcatgcacattgatgccaccctgtacacagagagtgatgtgcaccccagctgcaaggtgacagccatgaagtgtgagctgctggagctccaagtgatcagcctggagagtggtgatgccagcatccatgacacagtggagaacctgatcatcctggccaatgacagcctgagcagcaatggcaatgtgactgagagtggctgcaaggagtgtgaggagctggaggagaagaacatcaaggagttcctgcagagctttgtgcacattgtgcagatgttcatcaacaccagcatggacagactgaccagcagcttcctgctgctgattgtgcctgcctatgtgctgagcatcacctgccccccccccatgagtgtggagcatgcagacatctgggtgaagagctacagcctgtacagcagagagagatacatctgcaacagtggcttcaagagaaaggcaggcaccagcagcctgacagagtgtgtgctgaacaaggccaccaatgtggcccactggaccacccccagcctgaagtgcatcagagagccgaagagctgtgacaagacccacacctgcccaccctgcccagccccagaactcctgggaggccccagtgtgttcctgttcccccccaagcccaaggacaccctgatgatcagcagaaccccagaagtgacctgtgtggtggtggatgtgagccatgaggacccagaggtgaagttcaactggtatgtggacggggtggaggtgcacaatgccaagaccaagcccagagaggagcagtacaacagcacctacagagtggtgagtgtgctgacagtgctgcaccaagactggctgaatggcaaggagtacaagtgcaaggtgagcaacaaggccctgccagcccccatcgagaagaccatcagcaaggccaaggggcagcccagagagccccaagtgtacaccctgccccccagcagggatgagctgaccaagaaccaagtgagcctgacctgcctggtgaagggcttctaccccagtgacattgcagtggagtgggagagcaatgggcagccagagaacaactacaagaccaccccccctgtgctggacagtgatggcagcttcttcctgtacagcaagctgacagtggacaagagcagatggcagcaaggcaatgtgttcagctgcagtgtgatgcatgaggccctgcacaaccactacacccagaagagcctgagcctgagccctggcaagtga

(19) United States
(12) Patent Application Publication
(10) Pub. No.: US 2025/0057931 A1
(43) Pub. Date: Feb. 20, 2025

(54) COMBINATION THERAPY WITH ARENAVIRUS PARTICLES AND IMMUNE CHECKPOINT MODULATORS OR CYTOKINES

(71) Applicant: Hookipa Biotech GmbH, Vienna (AT)

(72) Inventors: Henning LAUTERBACH, Herrsching am Ammersee (DE); Josipa RAGUZ, Vienna (AT); Timo SCHIPPERS, Vienna (AT); Sarah SCHMIDT, Vienna (AT); Sarah AHMADI-ERBER, Sallingberg (AT); Klaus ORLINGER, Vienna (AT); Judith STRAUSS, Vienna (AT)

(21) Appl. No.: 18/836,679

(22) PCT Filed: Feb. 7, 2023
(86) PCT No.: PCT/EP2023/052952

§ 371 Date: Aug. 7, 2024

Related U.S. Application Data
(60) Provisional application No. 63/307,992, filed on Feb. 8, 2022.
(60) Provisional application No. 63/385,792, filed on Dec. 2, 2022.

(51) Int. Cl. A61K 39/12; A61K 9/00; A61K 38/20; A61K 39/00; A61K 45/06; A61P 35/00; C07K 16/24; C12N 7/00; C12N 15/86

(52) U.S. Cl. CPC A61K 39/12; A61K 9/0019; A61K 38/2013; A61K 45/06; A61P 35/00; C07K 16/246; C12N 7/00; C12N 15/86; A61K 2039/5254; A61K 2039/5256; A61K 2039/54; C12N 2760/10034; C12N 2760/10043

(57) ABSTRACT

Provided herein are methods and compositions for the treatment of neoplastic and infectious diseases. Specifically, provided herein are combination treatments that combine arenavirus-vectored antigens, such as tumor antigens or antigens of pathogens, with various immune checkpoint modulators or cytokines, which may in turn themselves be expressed using the arenavirus-based expression system.

### CROSS-REFERENCE TO RELATED APPLICATIONS

This application claims priority to U.S. Provisional Application No. 63/307,992 filed Feb. 8, 2022, and U.S. Provisional Application No. 63/385,792 filed Dec. 2, 2022, the content of each of which is incorporated by reference in its entirety herein, and to which priority is claimed.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “13194-087-228_SEQ_LISTING.xml”, was created on Jan. 24, 2023, and is 38,079 bytes in size.

### 1. INTRODUCTION

Provided herein are methods and compositions for the treatment of neoplastic and infectious diseases. Specifically, provided herein are combination treatments that combine arenavirus-vectored antigens, such as tumor antigens or antigens of pathogens, with various immune checkpoint modulators or cytokines, which may in turn themselves be expressed using the arenavirus-based expression system.

### 2. BACKGROUND

There is an ongoing medical need for the treatment of neoplastic diseases, such as cancer. The emerging field of immunotherapies holds promise for the treatment of these life-threatening diseases. In addition, combination therapies are being explored. However, as more therapies become available, the possible combinations are complex. Similarly, there is an ongoing need for the treatment and prevention of infectious diseases.

One strategy for immunotherapies involves arenavirus-based expression of tumor antigens. See for example, International Patent Application Publication Nos. WO2009/083210; WO/2016/075250; WO2017/198726; and WO2021/089853. Intratumoral administration of these immunotherapies has been described. See for example, International Patent Application Publication No. WO2018/185307. One main reason for the low efficiency and efficacy of immunotherapy with immune checkpoint inhibitors in non-inflamed (cold) tumors is the lack of anti-tumoral T cell responses (Chen & Mellman, Nature (2017); 18;541(7637):321-330). Arenavirus-based cancer vaccines are suited to induce tumor specific T cells but are hampered by the presence of the immunosuppressive factors within the tumor (Bonilla et al., Cell Rep Med. (2021); 2:100209; Kallert et al., Nat Commun (2017); 8:15327; Schmidt et al., Oncoimmunology. 2020; 9:1809960; Lauterbach et al., Front Oncol. 2021; 11:732166). 4-1BB can promote the activation, expansion, and effector function of activated T cells (Etxeberria et al., ESMO Open. 2020 July; 4(Suppl 3):e000733; Hashimoto. Cancers. 2021; 13:2288; Claus et al., Sci Transl Med. 2019;11(496)).

Citation of a reference herein shall not be construed as an admission that such is prior art to the present disclosure.

### 3. SUMMARY OF THE INVENTION

Provided herein are methods for treating or preventing a neoplastic disease in a subject in need thereof, using a combination of (1) a tumor antigen, tumor associated antigen, or antigenic fragment thereof, encoded by an arenavirus particle, and (2) at least one immune checkpoint modulator and/or at least one cytokine. The at least one immune checkpoint modulator and/or at least one cytokine can each be administered in combination with the arenavirus particle encoding the tumor antigen, tumor associated antigen, or antigenic fragment thereof, or be encoded by the same arenavirus particle or a different arenavirus particle.

Provided herein are methods for treating or preventing an infectious disease in a subject in need thereof, using a combination of (1) an antigen of a pathogen that causes the infectious disease, or antigenic fragment thereof, encoded by an arenavirus particle, and (2) at least one immune checkpoint modulator and/or at least one cytokine. The at least one immune checkpoint modulator and/or at least one cytokine can each be administered in combination with the arenavirus particle encoding the antigen of a pathogen that causes the infectious disease, or antigenic fragment thereof, or be encoded by the same arenavirus particle or a different arenavirus particle.

Illustrative Embodiments of the present disclosure are provided in the paragraphs below.
1. A method for treating or preventing a neoplastic disease in a subject in need thereof, wherein the method comprises administering to the subject (i) an arenavirus particle; and (ii) two different immune checkpoint modulators; wherein
   a. the arenavirus particle comprises an arenavirus genome comprising a heterologous ORF encoding a tumor antigen, tumor associated antigen, or antigenic fragment thereof; and
   b. (i) at least one arenavirus open reading frame (ORF) of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
2. The method of paragraph 1, wherein at least one of the two different immune checkpoint modulators targets a member of the tumor necrosis factor receptor superfamily (“TNFRSF”).
3. The method of paragraph 1 or 2, wherein the two different immune checkpoint modulators comprise an immune checkpoint modulator that is an agonist of the 4-1BB costimulatory pathway and another immune checkpoint modulator other than an agonist of the 4-1BB costimulatory pathway.
4. The method of paragraph 3, wherein the immune checkpoint modulator other than an agonist of the 4-1BB costimulatory pathway is an agonist of the OX40 costimulatory pathway.
5. The method of paragraph 4, wherein the agonist of the OX40 costimulatory pathway is an agonistic antibody of OX40.
6. The method of paragraph 5, wherein the agonistic antibody of OX40 is an antigen-binding fragment (Fab) or single-chain variable fragment (scFv).
7. The method of paragraph 4, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
8. The method of paragraph 1 or 2, wherein the two different immune checkpoint modulators comprise an immune checkpoint modulator that is an agonist of the OX40 costimulatory pathway and another immune checkpoint modulator other than an agonist of the OX40 costimulatory pathway.
9. The method of paragraph 8, wherein the agonist of the OX40 costimulatory pathway is an agonistic antibody of OX40.
10. The method of paragraph 9, wherein the agonistic antibody of OX40 is an antigen-binding fragment (Fab) or single-chain variable fragment (scFv).
11. The method of paragraph 8, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
12. The method of any one of paragraphs 8-11, wherein the immune checkpoint modulator other than an agonist of the OX40 costimulatory pathway is an agonist of the 4-1BB costimulatory pathway.
13. The method of any one of paragraphs 3-7 and 12, wherein the agonist of the 4-1BB costimulatory pathway is a bispecific antibody that binds to 4-1BB and to a molecule other than 4-1BB.
14. The method of paragraph 13, wherein the molecule other than 4-1BB is a costimulatory molecule, a tumor antigen, a tumor associated antigen, or a molecule expressed on the surface of cells in the tumor or in proximity to the tumor, optionally wherein the cells are cells of the tumor stroma.
15. The method of any one of paragraphs 3-7 and 12, wherein the agonist of the 4-1BB costimulatory pathway is an agonistic antibody of 4-1BB.
16. The method of paragraph 15, wherein the agonistic antibody of 4-1BB simultaneously targets and activates 4-1BB and another costimulatory molecule.
17. The method of paragraph 15, wherein the agonistic antibody of 4-1BB is an antigen-binding fragment (Fab) or single-chain variable fragment (scFv).
18. The method of any one of paragraphs 3-7 and 12, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
19. The method of any one of paragraphs 3-7 and 12, wherein the agonist of the 4-1BB costimulatory pathway is 4-1BBL.
20. The method of any one of paragraphs 3-7 and 12-19, wherein the arenavirus particle and the agonist of the 4-1BB costimulatory pathway are administered at the same time.
21. The method of any one of paragraphs 1-20, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
22. A method for treating or preventing a neoplastic disease in a subject in need thereof, wherein the method comprises administering to the subject (i) an arenavirus particle; and (ii) an immune checkpoint modulator other than an agonist of the 4-1BB costimulatory pathway; wherein
   a. the arenavirus particle comprises an arenavirus genome comprising:
      i. a first heterologous ORF encoding a tumor antigen, tumor associated antigen, or antigenic fragment thereof; and
      ii. a second heterologous ORF encoding an immune checkpoint modulator that is an agonist of the 4-1BB costimulatory pathway; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
23. The method of paragraph 22, wherein the immune checkpoint modulator other than an agonist of the 4-1BB costimulatory pathway targets a member of the TNFRSF.
24. The method of paragraph 22 or 23, wherein the immune checkpoint modulator other than an agonist of the 4-1BB costimulatory pathway is an agonist of the OX40 costimulatory pathway.
25. The method of paragraph 24, wherein the agonist of the OX40 costimulatory pathway is an agonistic antibody of OX40.
26. The method of paragraph 25, wherein the agonistic antibody of OX40 is an antigen-binding fragment (Fab) or single-chain variable fragment (scFv).
27. The method of paragraph 24, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
28. The method of any one of paragraphs 22-27, wherein the agonist of the 4-1BB costimulatory pathway is a bispecific antibody that binds to 4-1BB and to a molecule other than 4-1BB.
29. The method of paragraph 28, wherein the molecule other than 4-1BB is a costimulatory molecule, a tumor antigen, a tumor associated antigen, or a molecule expressed on the surface of cells in the tumor or in proximity to the tumor, optionally wherein the cells are cells of the tumor stroma.
30. The method of any one of paragraphs 22-27, wherein the agonist of the 4-1BB costimulatory pathway is an agonistic antibody of 4-1BB.
31. The method of paragraph 30, wherein the agonistic antibody of 4-1BB simultaneously targets and activates 4-1BB and another costimulatory molecule.
32. The method of paragraph 30, wherein the agonistic antibody of 4-1BB is an antigen-binding fragment (Fab) or single-chain variable fragment (scFv).
33. The method of any one of paragraphs 22-27, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
34. The method of any one of paragraphs 22-27, wherein the agonist of the 4-1BB costimulatory pathway is 4-1BBL.
35. The method of any one of paragraphs 22-34, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
36. The method of paragraph 35, wherein (i) a first S segment is engineered to carry an arenaviral ORF encoding GP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the immune checkpoint modulator that is an agonist of the 4-1BB costimulatory pathway in a position under control of an arenavirus genomic 5′ UTR; (ii) a first S segment is engineered to carry an arenaviral ORF encoding NP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the immune checkpoint modulator that is an agonist of the 4-1BB costimulatory pathway in a position under control of an arenavirus genomic 5′ UTR; or (iii) an S segment is engineered to carry both the first and the second heterologous ORFs.
37. The method of any one of paragraphs 22-36, wherein the arenavirus particle and the immune checkpoint modulator other than an agonist of the 4-1BB costimulatory pathway are administered at the same time.
38. A method for treating or preventing a neoplastic disease in a subject in need thereof, wherein the method comprises administering to the subject (i) a first arenavirus particle; and (ii) a second arenavirus particle; and (iii) an immune checkpoint modulator other than an agonist of the 4-1BB costimulatory pathway; wherein
   a. the first arenavirus particle comprises a first arenavirus genome comprising: a first heterologous ORF encoding a tumor antigen, tumor associated antigen, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
   b. the second arenavirus particle comprises a second arenavirus genome comprising: a second heterologous ORF encoding an immune checkpoint modulator that is an agonist of the 4-1BB costimulatory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
39. The method of paragraph 38, wherein the immune checkpoint modulator other than an agonist of the 4-1BB costimulatory pathway targets a member of the TNFRSF.
40. The method of paragraph 38 or 39, wherein the immune checkpoint modulator other than an agonist of the 4-1BB costimulatory pathway is an agonist of the OX40 costimulatory pathway.
41. The method of paragraph 40, wherein the agonist of the OX40 costimulatory pathway is an agonistic antibody of OX40.
42. The method of paragraph 41, wherein the agonistic antibody of OX40 is an antigen-binding fragment (Fab) or single-chain variable fragment (scFv).
43. The method of paragraph 40, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
44. The method of any one of paragraphs 38-43, wherein the agonist of the 4-1BB costimulatory pathway is a bispecific antibody that binds to 4-1BB and to a molecule other than 4-1BB.
45. The method of paragraph 44, wherein the molecule other than 4-1BB is a costimulatory molecule, a tumor antigen, a tumor associated antigen, or a molecule expressed on the surface of cells in the tumor or in proximity to the tumor, optionally wherein the cells are cells of the tumor stroma.
46. The method of any one of paragraphs 38-43, wherein the agonist of the 4-1BB costimulatory pathway is an agonistic antibody of 4-1BB.
47. The method of paragraph 46, wherein the agonistic antibody of 4-1BB simultaneously targets and activates 4-1BB and another costimulatory molecule.
48. The method of paragraph 46, wherein the agonistic antibody of 4-1BB is an antigen-binding fragment (Fab) or single-chain variable fragment (scFv).
49. The method of any one of paragraphs 38-43, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
50. The method of any one of paragraphs 38-43, wherein the agonist of the 4-1BB costimulatory pathway is 4-1BBL.
51. The method of any one of paragraphs 38-50, wherein the first arenavirus particle, the second arenavirus particle, or both the first and second arenavirus particles are tri-segmented and replication-competent and comprise one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
52. A method for treating or preventing a neoplastic disease in a subject in need thereof, wherein the method comprises administering to the subject (i) an arenavirus particle; and (ii) an immune checkpoint modulator other than an agonist of the OX40 costimulatory pathway; wherein
   a. the arenavirus particle comprises an arenavirus genome comprising:
      i. a first heterologous ORF encoding a tumor antigen, tumor associated antigen, or antigenic fragment thereof, and
      ii. a second heterologous ORF encoding an immune checkpoint modulator that is an agonist of the OX40 costimulatory pathway; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
53. The method of paragraph 52, wherein the immune checkpoint modulator other than an agonist of the OX40 costimulatory pathway targets a member of the TNFRSF.
54. The method of paragraph 52 or 53, wherein the immune checkpoint modulator other than an agonist of the OX40 costimulatory pathway is an agonist of the 4-1BB costimulatory pathway.
55. The method of paragraph 54, wherein the agonist of the 4-1BB costimulatory pathway is a bispecific antibody that binds to 4-1BB and to a molecule other than 4-1BB.
56. The method of paragraph 55, wherein the molecule other than 4-1BB is a costimulatory molecule, a tumor antigen, a tumor associated antigen, or a molecule expressed on the surface of cells in the tumor or in proximity to the tumor, optionally wherein the cells are cells of the tumor stroma.
57. The method of paragraph 54, wherein the agonist of the 4-1BB costimulatory pathway is an agonistic antibody of 4-1BB.
58. The method of paragraph 57, wherein the agonistic antibody of 4-1BB simultaneously targets and activates 4-1BB and another costimulatory molecule.
59. The method of paragraph 57, wherein the agonistic antibody of 4-1BB is an antigen-binding fragment (Fab) or single-chain variable fragment (scFv).
60. The method of paragraph 54, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
61. The method of paragraph 54, wherein the agonist of the 4-1BB costimulatory pathway is 4-1BBL.
62. The method of any one of paragraphs 54-61, wherein the arenavirus particle and the agonist of the 4-1BB costimulatory pathway are administered at the same time.
63. The method of any one of paragraphs 52-62, wherein the agonist of the OX40 costimulatory pathway is an agonistic antibody of OX40.
64. The method of paragraph 63, wherein the agonistic antibody of OX40 is an antigen-binding fragment (Fab) or single-chain variable fragment (scFv).
65. The method of any one of paragraphs 52-62, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
66. The method of any one of paragraphs 52-65, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
67. The method of paragraph 66, wherein (i) a first S segment is engineered to carry an arenaviral ORF encoding GP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the immune checkpoint modulator that is an agonist of the OX40 costimulatory pathway in a position under control of an arenavirus genomic 5′ UTR; or (ii) a first S segment is engineered to carry an arenaviral ORF encoding NP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the immune checkpoint modulator that is an agonist of the OX40 costimulatory pathway in a position under control of an arenavirus genomic 5′ UTR; or (iii) an S segment is engineered to carry both the first and the second heterologous ORFs.
68. The method of any one of paragraphs 52-67, wherein the arenavirus particle and the immune checkpoint modulator other than an agonist of the OX40 costimulatory pathway are administered at the same time.
69. A method for treating or preventing a neoplastic disease in a subject in need thereof, wherein the method comprises administering to the subject (i) a first arenavirus particle; and (ii) a second arenavirus particle; and (iii) an immune checkpoint modulator other than an agonist of the OX40 costimulatory pathway; wherein
   a. the first arenavirus particle comprises a first arenavirus genome comprising: a first heterologous ORF encoding a tumor antigen, tumor associated antigen, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
   b. the second arenavirus particle comprises a second arenavirus genome comprising: a second heterologous ORF encoding an immune checkpoint modulator that is an agonist of the OX40 costimulatory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
70. The method of paragraph 69, wherein the immune checkpoint modulator other than an agonist of the OX40 costimulatory pathway targets a member of the TNFRSF.
71. The method of paragraph 69 or 70, wherein the immune checkpoint modulator other than an agonist of the OX40 costimulatory pathway is an agonist of the 4-1BB costimulatory pathway.
72. The method of paragraph 71, wherein the agonist of the 4-1BB costimulatory pathway is a bispecific antibody that binds to 4-1BB and to a molecule other than 4-1BB.
73. The method of paragraph 72, wherein the molecule other than 4-1BB is a costimulatory molecule, a tumor antigen, a tumor associated antigen, or a molecule expressed on the surface of cells in the tumor or in proximity to the tumor, optionally wherein the cells are cells of the tumor stroma.
74. The method of paragraph 71, wherein the agonist of the 4-1BB costimulatory pathway is an agonistic antibody of 4-1BB.
75. The method of paragraph 74, wherein the agonistic antibody of 4-1BB simultaneously targets and activates 4-1BB and another costimulatory molecule.
76. The method of paragraph 74, wherein the agonistic antibody of 4-1BB is an antigen-binding fragment (Fab) or single-chain variable fragment (scFv).
77. The method of paragraph 71, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
78. The method of paragraph 71, wherein the agonist of the 4-1BB costimulatory pathway is 4-1BBL.
79. The method of any one of paragraphs 71-78, wherein (1) the first arenavirus particle, the second arenavirus particle, or both the first and second arenavirus particles and (2) the agonist of the 4-1BB costimulatory pathway are administered at the same time.
80. The method of any one of paragraphs 69-79, wherein the agonist of the OX40 costimulatory pathway is an agonistic antibody of OX40.
81. The method of paragraph 80, wherein the agonistic antibody of OX40 is an antigen-binding fragment (Fab) or single-chain variable fragment (scFv).
82. The method of any one of paragraphs 69-79, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
83. The method of any one of paragraphs 69-82, wherein the first arenavirus particle, the second arenavirus particle, or both the first and second arenavirus particles are tri-segmented and replication-competent and comprise one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
84. A method for treating or preventing a neoplastic disease in a subject in need thereof, wherein the method comprises administering to the subject an arenavirus particle; wherein
   a. the arenavirus particle comprises an arenavirus genome comprising:
      i. a first heterologous ORF encoding a tumor antigen, tumor associated antigen, or antigenic fragment thereof; and
      ii. a second heterologous ORF encoding an immune checkpoint modulator that is an agonist of the 4-1BB costimulatory pathway; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
85. The method of paragraph 84, wherein the agonist of the 4-1BB costimulatory pathway is a bispecific antibody that binds to 4-1BB and to a molecule other than 4-1BB.
86. The method of paragraph 85, wherein the molecule other than 4-1BB is a costimulatory molecule, a tumor antigen, a tumor associated antigen, or a molecule expressed on the surface of cells in the tumor or in proximity to the tumor, optionally wherein the cells are cells of the tumor stroma.
87. The method of paragraph 84, wherein the agonist of the 4-1BB costimulatory pathway is an agonistic antibody of 4-1BB.
88. The method of paragraph 87, wherein the agonistic antibody of 4-1BB simultaneously targets and activates 4-1BB and another costimulatory molecule.
89. The method of paragraph 87, wherein the agonistic antibody of 4-1BB is an antigen-binding fragment (Fab) or single-chain variable fragment (scFv).
90. The method of paragraph 84, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
91. The method of paragraph 84, wherein the agonist of the 4-1BB costimulatory pathway is 4-1BBL.
92. The method of any one of paragraphs 84-91, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
93. The method of paragraph 92, wherein (i) a first S segment is engineered to carry an arenaviral ORF encoding GP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the immune checkpoint modulator that is an agonist of the 4-1BB costimulatory pathway in a position under control of an arenavirus genomic 5′ UTR; or (ii) a first S segment is engineered to carry an arenaviral ORF encoding NP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the immune checkpoint modulator that is an agonist of the 4-1BB costimulatory pathway in a position under control of an arenavirus genomic 5′ UTR; or (iii) an S segment is engineered to carry both the first and the second heterologous ORFs.
94. A method for treating or preventing a neoplastic disease in a subject in need thereof, wherein the method comprises administering to the subject (i) an arenavirus particle; and (ii) an immune checkpoint modulator that is an agonist of the 4-1BB costimulatory pathway; wherein:
   a. the arenavirus particle comprises an arenavirus genome comprising a heterologous ORF encoding a tumor antigen, tumor associated antigen, or antigenic fragment thereof; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
95. The method of paragraph 94, wherein the agonist of the 4-1BB costimulatory pathway is a bispecific antibody that binds to 4-1BB and to a molecule other than 4-1BB.
96. The method of paragraph 95, wherein the molecule other than 4-1BB is a costimulatory molecule, a tumor antigen, a tumor associated antigen, or a molecule expressed on the surface of cells in the tumor or in proximity to the tumor, optionally wherein the cells are cells of the tumor stroma.
97. The method of paragraph 94, wherein the agonist of the 4-1BB costimulatory pathway is an agonistic antibody of 4-1BB.
98. The method of paragraph 97, wherein the agonistic antibody of 4-1BB simultaneously targets and activates 4-1BB and another costimulatory molecule.
99. The method of paragraph 97, wherein the agonistic antibody of 4-1BB is an antigen-binding fragment (Fab) or single-chain variable fragment (scFv).
100. The method of paragraph 94, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
101. The method of paragraph 94, wherein the agonist of the 4-1BB costimulatory pathway is 4-1BBL.
102. The method of any one of paragraphs 94-101, wherein the arenavirus particle and the agonist of the 4-1BB costimulatory pathway are administered at the same time.
103. The method of any one of paragraphs 94-102, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
104. A method for treating or preventing a neoplastic disease in a subject in need thereof, wherein the method comprises administering to the subject an arenavirus particle; wherein
   a. the arenavirus particle comprises an arenavirus genome comprising:
      i. a first heterologous ORF encoding a tumor antigen, tumor associated antigen, or antigenic fragment thereof; and
      ii. a second heterologous ORF encoding an immune checkpoint modulator that is a ligand of 4-1BB; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
105. The method of paragraph 104, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
106. The method of paragraph 105, wherein (i) a first S segment is engineered to carry an arenaviral ORF encoding GP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the immune checkpoint modulator that is a ligand of 4-1BB under control of an arenavirus genomic 5′ UTR; (ii) a first S segment is engineered to carry an arenaviral ORF encoding NP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the immune checkpoint modulator that is a ligand of 4-1BB under control of an arenavirus genomic 5′ UTR; or (iii) an S segment is engineered to carry both the first and the second heterologous ORFs.
107. A method for treating or preventing a neoplastic disease in a subject in need thereof, wherein the method comprises administering to the subject (i) a first arenavirus particle; and (ii) a second arenavirus particle; wherein
   a. the first arenavirus particle comprises a first arenavirus genome comprising: a first heterologous ORF encoding a tumor antigen, tumor associated antigen, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
   b. the second arenavirus particle comprises a second arenavirus genome comprising: a second heterologous ORF encoding an immune checkpoint modulator that is a ligand of 4-1BB; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
108. The method of paragraph 107, wherein the first arenavirus particle, the second arenavirus particle, or both the first and second arenavirus particles are tri-segmented and replication-competent and comprise one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
109. A method for treating or preventing a neoplastic disease in a subject in need thereof, wherein the method comprises administering to the subject an arenavirus particle; wherein
   a. the arenavirus particle comprises an arenavirus genome comprising:
      i. a first heterologous ORF encoding a tumor antigen, tumor associated antigen, or antigenic fragment thereof; and
      ii. a second heterologous ORF encoding an immune checkpoint modulator that is an antagonist of the NKG2A coinhibitory pathway; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
110. The method of paragraph 109, wherein the antagonist of the NKG2A coinhibitory pathway is an antagonistic antibody of NKG2A.
111. The method of paragraph 109 or 110, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
112. The method of paragraph 111, wherein (i) a first S segment is engineered to carry an arenaviral ORF encoding GP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the immune checkpoint modulator that is an antagonist of the NKG2A coinhibitory pathway in a position under control of an arenavirus genomic 5′ UTR; or (ii) a first S segment is engineered to carry an arenaviral ORF encoding NP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the immune checkpoint modulator that is an antagonist of the NKG2A coinhibitory pathway in a position under control of an arenavirus genomic 5′ UTR; or (iii) an S segment is engineered to carry both the first and the second heterologous ORFs.
113. A method for treating or preventing a neoplastic disease in a subject in need thereof, wherein the method comprises administering to the subject (i) an arenavirus particle; and (ii) an immune checkpoint modulator that is an antagonist of the NKG2A coinhibitory pathway; wherein:
   a. the arenavirus particle comprises an arenavirus genome comprising a heterologous ORF encoding a tumor antigen, tumor associated antigen, or antigenic fragment thereof; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
114. The method of paragraph 113, wherein the antagonist of the NKG2A coinhibitory pathway is an antagonistic antibody of NKG2A.
115. The method of paragraph 113 or 114, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
116. A method for treating or preventing a neoplastic disease in a subject in need thereof, wherein the method comprises administering to the subject (i) a first arenavirus particle; and (ii) a second arenavirus particle; wherein
   a. the first arenavirus particle comprises a first arenavirus genome comprising: a first heterologous ORF encoding a tumor antigen, tumor associated antigen, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
   b. the second arenavirus particle comprises a second arenavirus genome comprising: a second heterologous ORF encoding an antagonist of the NKG2A coinhibitory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
117. The method of paragraph 116, wherein the antagonist of the NKG2A coinhibitory pathway is an antagonistic antibody or antigen-binding fragment thereof of NKG2A.
118. The method of paragraph 116 or 117, wherein the first arenavirus particle, the second arenavirus particle, or both the first and second arenavirus particles are tri-segmented and replication-competent and comprise one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
119. A method for treating or preventing a neoplastic disease in a subject in need thereof, wherein the method comprises administering to the subject an arenavirus particle; wherein
   a. the arenavirus particle comprises an arenavirus genome comprising:
      i. a first heterologous ORF encoding a tumor antigen, tumor associated antigen, or antigenic fragment thereof; and
      ii. a second heterologous ORF encoding a cytokine, optionally the cytokine is IL-12; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
120. The method of paragraph 119, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
121. The method of paragraph 120, wherein (i) a first S segment is engineered to carry an arenaviral ORF encoding GP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the cytokine under control of an arenavirus genomic 5′ UTR; or (ii) a first S segment is engineered to carry an arenaviral ORF encoding NP in a position under control of an arenavirus genomic 3′ UTR and the first heterologous ORF encoding the tumor antigen, tumor associated antigen, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the cytokine under control of an arenavirus genomic 5′ UTR; or (iii) an S segment is engineered to carry both the first and the second heterologous ORFs.
122. A method for treating or preventing a neoplastic disease in a subject in need thereof, wherein the method comprises administering to the subject (i) a first arenavirus particle; and (ii) a second arenavirus particle; wherein
   a. the first arenavirus particle comprises a first arenavirus genome comprising: a first heterologous ORF encoding a tumor antigen, tumor associated antigen, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
   b. the second arenavirus particle comprises a second arenavirus genome comprising: a second heterologous ORF encoding a cytokine, optionally the cytokine is IL-12; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
123. The method of paragraph 122, wherein the first arenavirus particle, the second arenavirus particle, or both the first and second arenavirus particles are tri-segmented and replication-competent and comprise one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
124. The method of any one of paragraphs 1-37, 52-68, 84-106, 109-115 and 119-121, wherein the arenavirus particle is administered via intravenous injection.
125. The method of any one of paragraphs 15-17, 57-59 and 97-99, wherein the arenavirus particle is administered via intravenous injection.
126. The method of any one of paragraphs 1-37, 52-68, 84-106, 109-115 and 119-121, wherein the neoplastic disease is a solid tumor and wherein the arenavirus particle is administered via intratumoral injection.
127. The method of any one of paragraphs 22-37, 52-68, 84-93 104-106, 109-112 and 119-121, wherein the neoplastic disease is a solid tumor and wherein the arenavirus particle is administered via intratumoral injection.
128. The method of any one of paragraphs 1-37, 52-68, 84-106, 109-115, 119-121 and 124-127, wherein the at least one arenavirus ORF encodes the glycoprotein (“GP”), the nucleoprotein (“NP”), the matrix protein Z (“Z protein”) or the RNA dependent RNA polymerase L (“L protein”) of the arenavirus particle.
129. The method of any one of paragraphs 1-37, 52-68, 84-106, 109-115, 119-121 and 124-128, wherein the at least one arenavirus ORF is either functionally inactivated or deleted and wherein the arenavirus particle has the ability to amplify and express its genetic information in cells infected with the arenavirus particle but is unable to produce further infectious progeny particles in normal, non-complementing cells.
130. The method of any one of paragraphs 1-37, 52-68, 84-106, 109-115, 119-121 and 124-129, wherein the arenavirus particle is derived from lymphocytic choriomeningitis virus (LCMV) or Pichinde virus.
131. The method of any one of paragraphs 38-51, 69-83, 107-108, 116-118 and 122-123, wherein the first arenavirus particle, the second arenavirus particle, or both the first and second arenavirus particles are administered via intravenous injection.
132. The method of any one of paragraphs 74-76, wherein the first arenavirus particle, the second arenavirus particle, or both the first and second arenavirus particles are administered via intravenous injection.
133. The method of any one of paragraphs 38-51, 69-83, 107-108, 116-118 and 122-123, wherein the neoplastic disease is a solid tumor and wherein the first arenavirus particle is administered via intravenous injection and the second arenavirus particle is administered via intratumoral injection.
134. The method of any one of paragraphs 38-51, 69-83, 107-108, 116-118 and 122-123, wherein the neoplastic disease is a solid tumor and wherein both the first and second arenavirus particles are administered via intratumoral injection.
135. The method of any one of paragraphs 38-51, 69-83, 107-108, 116-118 and 122-123, wherein the neoplastic disease is a solid tumor and wherein the first arenavirus particle is administered via intratumoral injection and the second arenavirus particle is administered via intravenous injection.
136. The method of any one of paragraphs 38-51, 69-83, 107-108, 116-118 and 122-123, wherein the neoplastic disease is a solid tumor and wherein both the first and second arenavirus particles are administered via intravenous injection.
137. The method of any one of paragraphs 38-51, 69-83, 107-108, 116-118, 122-123 and 131-136, wherein the at least one first arenavirus ORF encodes the glycoprotein (“GP”), the nucleoprotein (“NP”), the matrix protein Z (“Z protein”) or the RNA dependent RNA polymerase L (“L protein”) of the first arenavirus particle; and/or the at least one second arenavirus ORF encodes the glycoprotein (“GP”), the nucleoprotein (“NP”), the matrix protein Z (“Z protein”) or the RNA dependent RNA polymerase L (“L protein”) of the second arenavirus particle.
138. The method of any one of paragraphs 38-51, 69-83, 107-108, 116-118, 122-123 and 131-137, wherein the at least one first arenavirus ORF is either functionally inactivated or deleted and wherein the first arenavirus particle has the ability to amplify and express its genetic information in cells infected with the first arenavirus particle but is unable to produce further infectious progeny particles in normal, non-complementing cells; and/or wherein the at least one second arenavirus ORF is either functionally inactivated or deleted and wherein the second arenavirus particle has the ability to amplify and express its genetic information in cells infected with the second arenavirus particle but is unable to produce further infectious progeny particles in normal, non-complementing cells.
139. The method of any one of paragraphs 38-51, 69-83, 107-108, 116-118, 122-123 and 131-138, wherein the first arenavirus particle, the second arenavirus particle, or both the first and second arenavirus particles are derived from lymphocytic choriomeningitis virus (LCMV) or Pichinde virus.
140. The method of any one of the preceding paragraphs, wherein the neoplastic disease is a solid tumor and wherein the method results in an increase of the concentration of T cells within the solid tumor.
141. The method of paragraph 140, wherein the method results in an increase of the concentration of CD8+ T cells, the concentration of CD4+ T cells, the concentration of tumor antigen specific T cells, the concentration of T cells producing IFN-gamma, and/or the concentration of T cells producing granzyme B, within the solid tumor.
142. The method of any one of the preceding paragraphs, wherein the neoplastic disease is a solid tumor and wherein the method results in an increase of the ratio of effector T cells/regulatory T cells within the solid tumor.
143. The method of any one of the preceding paragraphs, wherein the method has a higher anti-tumor efficacy as compared to administration of a control arenavirus particle expressing the tumor antigen, tumor associated antigen, or antigenic fragment thereof, alone.
144. The method of any one of the preceding paragraphs, wherein the method results in an increase in the survival rate of subjects treated with the method, compared to subjects having the same neoplastic disease in the absence of such treatment.
145. The method of any one of the preceding paragraphs, wherein the neoplastic disease is acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; Hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, nonsmall cell lung cancer; non-Hodgkin lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; childhood thyroid cancer; transitional cell cancer of the renal pelvis and ureter; urethral cancer; uterine cancer, endometrial; uterine sarcoma; vaginal cancer; vulvar cancer; or Wilms tumor.
146. The method of any one of the preceding paragraphs, wherein the tumor antigen, tumor associated antigen, or antigenic fragment thereof is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCl, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68KP1, CO-029, HTgp-175, M344, MG7-Ag, MOV18, NB70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, an endogenous retroviral protein, mutated KRAS, mutated p53, mutated PI3K, mutated Braf, and ROR1.
147. A method for preventing or treating an infectious disease in a subject in need thereof, wherein the method comprises administering to the subject (i) an arenavirus particle; and (ii) two different immune checkpoint modulators; wherein
   a. the arenavirus particle comprises an arenavirus genome comprising a heterologous ORF encoding an antigen of a pathogen that causes the infectious disease, or antigenic fragment thereof, and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
148. The method of paragraph 147, wherein at least one of the two different immune checkpoint modulators targets a member of the tumor necrosis factor receptor superfamily (“TNFRSF”).
149. The method of paragraph 147 or 148, wherein the two different immune checkpoint modulators comprise an immune checkpoint modulator that is an agonist of the 4-1BB costimulatory pathway and another immune checkpoint modulator other than an agonist of the 4-1BB costimulatory pathway.
150. The method of paragraph 149, wherein the immune checkpoint modulator other than an agonist of the 4-1BB costimulatory pathway is an agonist of the OX40 costimulatory pathway.
151. The method of paragraph 150, wherein the agonist of the OX40 costimulatory pathway is an agonistic antibody of OX40.
152. The method of paragraph 151, wherein the agonistic antibody of OX40 is an antigen-binding fragment (Fab) or single-chain variable fragment (scFv).
153. The method of paragraph 150, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
154. The method of paragraph 147 or 148, wherein the two different immune checkpoint modulators comprise an immune checkpoint modulator that is an agonist of the OX40 costimulatory pathway and another immune checkpoint modulator other than an agonist of the OX40 costimulatory pathway.
155. The method of paragraph 154, wherein the agonist of the OX40 costimulatory pathway is an agonistic antibody of OX40.
156. The method of paragraph 155, wherein the agonistic antibody of OX40 is an antigen-binding fragment (Fab) or single-chain variable fragment (scFv).
157. The method of paragraph 154, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
158. The method of any one of paragraphs 154-157, wherein the immune checkpoint modulator other than an agonist of the OX40 costimulatory pathway is an agonist of the 4-1BB costimulatory pathway.
159. The method of any one of paragraphs 149-153 and 158, wherein the agonist of the 4-1BB costimulatory pathway is a bispecific antibody that binds to 4-1BB and to a molecule other than 4-1BB.
160. The method of paragraph 159, wherein the molecule other than 4-1BB is a costimulatory molecule or an antigen of a pathogen that causes the infectious disease.
161. The method of any one of paragraphs 149-153 and 158, wherein the agonist of the 4-1BB costimulatory pathway is an agonistic antibody of 4-1BB.
162. The method of paragraph 161, wherein the agonistic antibody of 4-1BB simultaneously targets and activates 4-1BB and another costimulatory molecule.
163. The method of paragraph 161, wherein the agonistic antibody of 4-1BB is an antigen-binding fragment (Fab) or single-chain variable fragment (scFv).
164. The method of any one of paragraphs 149-153 and 158, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
165. The method of any one of paragraphs 149-153 and 158, wherein the agonist of the 4-1BB costimulatory pathway is 4-1BBL.
166. The method of any one of paragraphs 149-153 and 158-165, wherein the arenavirus particle and the agonist of the 4-1BB costimulatory pathway are administered at the same time.
167. The method of any one of paragraphs 147-166, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
168. A method for preventing or treating an infectious disease in a subject in need thereof, wherein the method comprises administering to the subject (i) an arenavirus particle; and (ii) an immune checkpoint modulator other than an agonist of the 4-1BB costimulatory pathway; wherein
   a. the arenavirus particle comprises an arenavirus genome comprising:
      i. a first heterologous ORF encoding an antigen of a pathogen that causes the infectious disease, or antigenic fragment thereof; and
      ii. a second heterologous ORF encoding an immune checkpoint modulator that is an agonist of the 4-1BB costimulatory pathway; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
169. The method of paragraph 168, wherein the immune checkpoint modulator other than an agonist of the 4-1BB costimulatory pathway targets a member of the TNFRSF.
170. The method of paragraph 168 or 169, wherein the immune checkpoint modulator other than an agonist of the 4-1BB costimulatory pathway is an agonist of the OX40 costimulatory pathway.
171. The method of paragraph 170, wherein the agonist of the OX40 costimulatory pathway is an agonistic antibody of OX40.
172. The method of paragraph 171, wherein the agonistic antibody of OX40 is an antigen-binding fragment (Fab) or single-chain variable fragment (scFv).
173. The method of paragraph 170, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
174. The method of any one of paragraphs 168-173, wherein the agonist of the 4-1BB costimulatory pathway is a bispecific antibody that binds to 4-1BB and to a molecule other than 4-1BB.
175. The method of paragraph 174, wherein the molecule other than 4-1BB is a costimulatory molecule or an antigen of a pathogen that causes the infectious disease.
176. The method of any one of paragraphs 168-173, wherein the agonist of the 4-1BB costimulatory pathway is an agonistic antibody of 4-1BB.
177. The method of paragraph 176, wherein the agonistic antibody of 4-1BB simultaneously targets and activates 4-1BB and another costimulatory molecule.
178. The method of paragraph 176, wherein the agonistic antibody of 4-1BB is an antigen-binding fragment (Fab) or single-chain variable fragment (scFv).
179. The method of any one of paragraphs 168-173, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
180. The method of any one of paragraphs 168-173, wherein the agonist of the 4-1BB costimulatory pathway is 4-1BBL.
181. The method of any one of paragraphs 168-180, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
182. The method of paragraph 181, wherein (i) a first S segment is engineered to carry an arenaviral ORF encoding GP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the immune checkpoint modulator that is an agonist of the 4-1BB costimulatory pathway in a position under control of an arenavirus genomic 5′ UTR; or (ii) a first S segment is engineered to carry an arenaviral ORF encoding NP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the immune checkpoint modulator that is an agonist of the 4-1BB costimulatory pathway in a position under control of an arenavirus genomic 5′ UTR; or (iii) an S segment is engineered to carry both the first and the second heterologous ORFs.
183. The method of any one of paragraphs 168-182, wherein the arenavirus particle and the immune checkpoint modulator other than an agonist of the 4-1BB costimulatory pathway are administered at the same time.
184. A method for preventing or treating an infectious disease in a subject in need thereof, wherein the method comprises administering to the subject (i) a first arenavirus particle; and (ii) a second arenavirus particle; and (iii) an immune checkpoint modulator other than an agonist of the 4-1BB costimulatory pathway; wherein
   a. the first arenavirus particle comprises a first arenavirus genome comprising: a first heterologous ORF encoding an antigen of a pathogen that causes the infectious disease, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
   b. the second arenavirus particle comprises a second arenavirus genome comprising: a second heterologous ORF encoding an immune checkpoint modulator that is an agonist of the 4-1BB costimulatory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
185. The method of paragraph 184, wherein the immune checkpoint modulator other than an agonist of the 4-1BB costimulatory pathway targets a member of the TNFRSF.
186. The method of paragraph 184 or 185, wherein the immune checkpoint modulator other than an agonist of the 4-1BB costimulatory pathway is an agonist of the OX40 costimulatory pathway.
187. The method of paragraph 186, wherein the agonist of the OX40 costimulatory pathway is an agonistic antibody of OX40.
188. The method of paragraph 187, wherein the agonistic antibody of OX40 is an antigen-binding fragment (Fab) or single-chain variable fragment (scFv).
189. The method of paragraph 186, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
190. The method of any one of paragraphs 184-189, wherein the agonist of the 4-1BB costimulatory pathway is a bispecific antibody that binds to 4-1BB and to a molecule other than 4-1BB.
191. The method of paragraph 190, wherein the molecule other than 4-1BB is a costimulatory molecule or an antigen of a pathogen that causes the infectious disease.
192. The method of any one of paragraphs 184-189, wherein the agonist of the 4-1BB costimulatory pathway is an agonistic antibody of 4-1BB.
193. The method of paragraph 192, wherein the agonistic antibody of 4-1BB simultaneously targets and activates 4-1BB and another costimulatory molecule.
194. The method of paragraph 192, wherein the agonistic antibody of 4-1BB is an antigen-binding fragment (Fab) or single-chain variable fragment (scFv).
195. The method of any one of paragraphs 184-189, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
196. The method of any one of paragraphs 184-189, wherein the agonist of the 4-1BB costimulatory pathway is 4-1BBL.
197. The method of any one of paragraphs 184-196, wherein the first arenavirus particle, the second arenavirus particle, or both the first and second arenavirus particles are tri-segmented and replication-competent and comprise one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
198. A method for preventing or treating an infectious disease in a subject in need thereof, wherein the method comprises administering to the subject (i) an arenavirus particle; and (ii) an immune checkpoint modulator other than an agonist of the OX40 costimulatory pathway; wherein
   a. the arenavirus particle comprises an arenavirus genome comprising:
      i. a first heterologous ORF encoding an antigen of a pathogen that causes the infectious disease, or antigenic fragment thereof, and
      ii. a second heterologous ORF encoding an immune checkpoint modulator that is an agonist of the OX40 costimulatory pathway; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
199. The method of paragraph 198, wherein the immune checkpoint modulator other than an agonist of the OX40 costimulatory pathway targets a member of the TNFRSF.
200. The method of paragraph 198 or 199, wherein the immune checkpoint modulator other than an agonist of the OX40 costimulatory pathway is an agonist of the 4-1BB costimulatory pathway.
201. The method of paragraph 200, wherein the agonist of the 4-1BB costimulatory pathway is a bispecific antibody that binds to 4-1BB and to a molecule other than 4-1BB.
202. The method of paragraph 201, wherein the molecule other than 4-1BB is a costimulatory molecule or an antigen of a pathogen that causes the infectious disease.
203. The method of paragraph 200, wherein the agonist of the 4-1BB costimulatory pathway is an agonistic antibody of 4-1BB.
204. The method of paragraph 203, wherein the agonistic antibody of 4-1BB simultaneously targets and activates 4-1BB and another costimulatory molecule.
205. The method of paragraph 203, wherein the agonistic antibody of 4-1BB is an antigen-binding fragment (Fab) or single-chain variable fragment (scFv).
206. The method of paragraph 200, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
207. The method of paragraph 200, wherein the agonist of the 4-1BB costimulatory pathway is 4-1BBL.
208. The method of any one of paragraphs 200-207, wherein the arenavirus particle and the agonist of the 4-1BB costimulatory pathway are administered at the same time.
209. The method of any one of paragraphs 198-208, wherein the agonist of the OX40 costimulatory pathway is an agonistic antibody of OX40.
210. The method of paragraph 209, wherein the agonistic antibody of OX40 is an antigen-binding fragment (Fab) or single-chain variable fragment (scFv).
211. The method of any one of paragraphs 198-208, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
212. The method of any one of paragraphs 198-211, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
213. The method of paragraph 212, wherein (i) a first S segment is engineered to carry an arenaviral ORF encoding GP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the immune checkpoint modulator that is an agonist of the OX40 costimulatory pathway in a position under control of an arenavirus genomic 5′ UTR; or (ii) a first S segment is engineered to carry an arenaviral ORF encoding NP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the immune checkpoint modulator that is an agonist of the OX40 costimulatory pathway in a position under control of an arenavirus genomic 5′ UTR; or (iii) an S segment is engineered to carry both the first and the second heterologous ORFs.
214. The method of any one of paragraphs 198-213, wherein the arenavirus particle and the immune checkpoint modulator other than an agonist of the OX40 costimulatory pathway are administered at the same time.
215. A method for preventing or treating an infectious disease in a subject in need thereof, wherein the method comprises administering to the subject (i) a first arenavirus particle; and (ii) a second arenavirus particle; and (iii) an immune checkpoint modulator other than an agonist of the OX40 costimulatory pathway; wherein
   a. the first arenavirus particle comprises a first arenavirus genome comprising: a first heterologous ORF encoding an antigen of a pathogen that causes the infectious disease, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
   b. the second arenavirus particle comprises a second arenavirus genome comprising: a second heterologous ORF encoding an immune checkpoint modulator that is an agonist of the OX40 costimulatory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
216. The method of paragraph 215, wherein the immune checkpoint modulator other than an agonist of the OX40 costimulatory pathway targets a member of the TNFRSF.
217. The method of paragraph 215 or 216, wherein the immune checkpoint modulator other than an agonist of the OX40 costimulatory pathway is an agonist of the 4-1BB costimulatory pathway.
218. The method of paragraph 217, wherein the agonist of the 4-1BB costimulatory pathway is a bispecific antibody that binds to 4-1BB and to a molecule other than 4-1BB.
219. The method of paragraph 218, wherein the molecule other than 4-1BB is a costimulatory molecule or an antigen of a pathogen that causes the infectious disease.
220. The method of paragraph 217, wherein the agonist of the 4-1BB costimulatory pathway is an agonistic antibody of 4-1BB.
221. The method of paragraph 220, wherein the agonistic antibody of 4-1BB simultaneously targets and activates 4-1BB and another costimulatory molecule.
222. The method of paragraph 220, wherein the agonistic antibody of 4-1BB is an antigen-binding fragment (Fab) or single-chain variable fragment (scFv).
223. The method of paragraph 217, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
224. The method of paragraph 217, wherein the agonist of the 4-1BB costimulatory pathway is 4-1BBL.
225. The method of any one of paragraphs 217-224, wherein (1) the first arenavirus particle, the second arenavirus particle, or both the first and second arenavirus particles and (2) the agonist of the 4-1BB costimulatory pathway are administered at the same time.
226. The method of any one of paragraphs 215-225, wherein the agonist of the OX40 costimulatory pathway is an agonistic antibody of OX40.
227. The method of paragraph 226, wherein the agonistic antibody of OX40 is an antigen-binding fragment (Fab) or single-chain variable fragment (scFv).
228. The method of any one of paragraphs 215-225, wherein the agonist of the OX40 costimulatory pathway is INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, or SL-279252 (PD1-Fc-OX40L).
229. The method of any one of paragraphs 215-228, wherein the first arenavirus particle, the second arenavirus particle, or both the first and second arenavirus particles are tri-segmented and replication-competent and comprise one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
230. A method for preventing or treating an infectious disease in a subject in need thereof, wherein the method comprises administering to the subject an arenavirus particle; wherein
   a. the arenavirus particle comprises an arenavirus genome comprising:
      i. a first heterologous ORF encoding an antigen of a pathogen that causes the infectious disease, or antigenic fragment thereof; and
      ii. a second heterologous ORF encoding an immune checkpoint modulator that is an agonist of the 4-1BB costimulatory pathway; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
231. The method of paragraph 230, wherein the agonist of the 4-1BB costimulatory pathway is a bispecific antibody that binds to 4-1BB and to a molecule other than 4-1BB.
232. The method of paragraph 231, wherein the molecule other than 4-1BB is a costimulatory molecule or an antigen of a pathogen that causes the infectious disease.
233. The method of paragraph 230, wherein the agonist of the 4-1BB costimulatory pathway is an agonistic antibody of 4-1BB.
234. The method of paragraph 233, wherein the agonistic antibody of 4-1BB simultaneously targets and activates 4-1BB and another costimulatory molecule.
235. The method of paragraph 233, wherein the agonistic antibody of 4-1BB is an antigen-binding fragment (Fab) or single-chain variable fragment (scFv).
236. The method of paragraph 230, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
237. The method of paragraph 230, wherein the agonist of the 4-1BB costimulatory pathway is 4-1BBL.
238. The method of any one of paragraphs 230-237, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
239. The method of paragraph 238, wherein (i) a first S segment is engineered to carry an arenaviral ORF encoding GP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the immune checkpoint modulator that is an agonist of the 4-1BB costimulatory pathway in a position under control of an arenavirus genomic 5′ UTR; or (ii) a first S segment is engineered to carry an arenaviral ORF encoding NP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the immune checkpoint modulator that is an agonist of the 4-1BB costimulatory pathway in a position under control of an arenavirus genomic 5′ UTR; or (iii) an S segment is engineered to carry both the first and the second heterologous ORFs.
240. A method for preventing or treating an infectious disease in a subject in need thereof, wherein the method comprises administering to the subject (i) an arenavirus particle; and (ii) an immune checkpoint modulator that is an agonist of the 4-1BB costimulatory pathway; wherein:
   a. the arenavirus particle comprises an arenavirus genome comprising a heterologous ORF encoding an antigen of a pathogen that causes the infectious disease, or antigenic fragment thereof; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
241. The method of paragraph 240, wherein the agonist of the 4-1BB costimulatory pathway is a bispecific antibody that binds to 4-1BB and to a molecule other than 4-1BB.
242. The method of paragraph 241, wherein the molecule other than 4-1BB is a costimulatory molecule or an antigen of a pathogen that causes the infectious disease.
243. The method of paragraph 240, wherein the agonist of the 4-1BB costimulatory pathway is an agonistic antibody of 4-1BB.
244. The method of paragraph 243, wherein the agonistic antibody of 4-1BB simultaneously targets and activates 4-1BB and another costimulatory molecule.
245. The method of paragraph 243, wherein the agonistic antibody of 4-1BB is an antigen-binding fragment (Fab) or single-chain variable fragment (scFv).
246. The method of paragraph 240, wherein the agonist of the 4-1BB costimulatory pathway is utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, or SL-279137 (PD-1-Fc-4-1BBL).
247. The method of paragraph 240, wherein the agonist of the 4-1BB costimulatory pathway is 4-1BBL.
248. The method of any one of paragraphs 240-247, wherein the arenavirus particle and the agonist of the 4-1BB costimulatory pathway are administered at the same time.
249. The method of any one of paragraphs 240-248, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
250. A method for preventing or treating an infectious disease in a subject in need thereof, wherein the method comprises administering to the subject an arenavirus particle; wherein
   a. the arenavirus particle comprises an arenavirus genome comprising:
      i. a first heterologous ORF encoding an antigen of a pathogen that causes the infectious disease, or antigenic fragment thereof; and
      ii. a second heterologous ORF encoding an immune checkpoint modulator that is a ligand of 4-1BB; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
251. The method of paragraph 250, wherein the arenavirus particle is tri-segmented and replication-competent and comprises one L segment and two S segments, wherein one of the two S segments is selected from the group consisting of.
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
252. The method of paragraph 251, wherein (i) a first S segment is engineered to carry an arenaviral ORF encoding GP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the immune checkpoint modulator that is a ligand of 4-1BB under control of an arenavirus genomic 5′ UTR; or (ii) a first S segment is engineered to carry an arenaviral ORF encoding NP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the immune checkpoint modulator that is a ligand of 4-1BB under control of an arenavirus genomic 5′ UTR; or (iii) an S segment is engineered to carry both the first and the second heterologous ORFs.
253. A method for preventing or treating an infectious disease in a subject in need thereof, wherein the method comprises administering to the subject (i) a first arenavirus particle; and (ii) a second arenavirus particle; wherein
   a. the first arenavirus particle comprises a first arenavirus genome comprising: a first heterologous ORF encoding an antigen of a pathogen that causes the infectious disease, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
   b. the second arenavirus particle comprises a second arenavirus genome comprising: a second heterologous ORF encoding an immune checkpoint modulator that is a ligand of 4-1BB; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
254. The method of paragraph 253, wherein the first arenavirus particle, the second arenavirus particle, or both the first and second arenavirus particles are tri-segmented and replication-competent and comprise one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
255. A method for preventing or treating an infectious disease in a subject in need thereof, wherein the method comprises administering to the subject an arenavirus particle; wherein
   a. the arenavirus particle comprises an arenavirus genome comprising:
      i. a first heterologous ORF encoding an antigen of a pathogen that causes the infectious disease, or antigenic fragment thereof; and
      ii. a second heterologous ORF encoding an immune checkpoint modulator that is an antagonist of the NKG2A coinhibitory pathway; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
256. The method of paragraph 255, wherein the antagonist of the NKG2A coinhibitory pathway is an antagonistic antibody of NKG2A.
257. The method of paragraph 255 or 256, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
258. The method of paragraph 257, wherein (i) a first S segment is engineered to carry an arenaviral ORF encoding GP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the immune checkpoint modulator that is an antagonist of the NKG2A coinhibitory pathway in a position under control of an arenavirus genomic 5′ UTR; or (ii) a first S segment is engineered to carry an arenaviral ORF encoding NP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the immune checkpoint modulator that is an antagonist of the NKG2A coinhibitory pathway in a position under control of an arenavirus genomic 5′ UTR; or (iii) an S segment is engineered to carry both the first and the second heterologous ORFs.
259. A method for preventing or treating an infectious disease in a subject in need thereof, wherein the method comprises administering to the subject (i) an arenavirus particle; and (ii) an immune checkpoint modulator that is an antagonist of the NKG2A coinhibitory pathway; wherein:
   a. the arenavirus particle comprises an arenavirus genome comprising a heterologous ORF encoding an antigen of a pathogen that causes the infectious disease, or antigenic fragment thereof; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
260. The method of paragraph 259, wherein the antagonist of the NKG2A coinhibitory pathway is an antagonistic antibody of NKG2A.
261. The method of paragraph 259 or 260, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
262. A method for preventing or treating an infectious disease in a subject in need thereof, wherein the method comprises administering to the subject (i) a first arenavirus particle; and (ii) a second arenavirus particle; wherein
   a. the first arenavirus particle comprises a first arenavirus genome comprising: a first heterologous ORF encoding an antigen of a pathogen that causes the infectious disease, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
   b. the second arenavirus particle comprises a second arenavirus genome comprising: a second heterologous ORF encoding an antagonist of the NKG2A coinhibitory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
263. The method of paragraph 262, wherein the antagonist of the NKG2A coinhibitory pathway is an antagonistic antibody or antigen-binding fragment thereof of NKG2A.
264. The method of paragraph 262 or 263, wherein the first arenavirus particle, the second arenavirus particle, or both the first and second arenavirus particles are tri-segmented and replication-competent and comprise one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
265. A method for preventing or treating an infectious disease in a subject in need thereof, wherein the method comprises administering to the subject an arenavirus particle; wherein
   a. the arenavirus particle comprises an arenavirus genome comprising:
      i. a first heterologous ORF encoding an antigen of a pathogen that causes the infectious disease, or antigenic fragment thereof; and
      ii. a second heterologous ORF encoding a cytokine, optionally the cytokine is IL-12; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
266. The method of paragraph 265, wherein the arenavirus particle is tri-segmented and replication-competent and comprises one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
267. The method of paragraph 266, wherein (i) a first S segment is engineered to carry an arenaviral ORF encoding GP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding NP in a position under control of an arenavirus genomic 3′ UTR and the second heterologous ORF encoding the cytokine under control of an arenavirus genomic 5′ UTR; or (ii) a first S segment is engineered to carry an arenaviral ORF encoding NP in a position under control of an arenavirus genomic 3′ UTR and the first heterologous ORF encoding the antigen of a pathogen that causes the infectious disease, or antigenic fragment thereof in a position under control of an arenavirus genomic 5′ UTR, and a second S segment is engineered to carry an arenaviral ORF encoding GP in a position under control of an arenavirus genomic 3′ UTR and the second heterologous ORF encoding the cytokine under control of an arenavirus genomic 5′ UTR; or (iii) an S segment is engineered to carry both the first and the second heterologous ORFs.
268. A method for preventing or treating an infectious disease in a subject in need thereof, wherein the method comprises administering to the subject (i) a first arenavirus particle; and (ii) a second arenavirus particle; wherein
   a. the first arenavirus particle comprises a first arenavirus genome comprising: a first heterologous ORF encoding an antigen of a pathogen that causes the infectious disease, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
   b. the second arenavirus particle comprises a second arenavirus genome comprising: a second heterologous ORF encoding a cytokine, optionally the cytokine is IL-12; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
269. The method of paragraph 268, wherein the first arenavirus particle, the second arenavirus particle, or both the first and second arenavirus particles are tri-segmented and replication-competent and comprise one L segment and two S segments, wherein one of the two S segments is selected from the group consisting of:
   a. an S segment, wherein the ORF encoding the NP is under control of an arenavirus genomic 5′ UTR;
   b. an S segment, wherein the ORF encoding the Z protein is under control of an arenavirus genomic 5′ UTR;
   c. an S segment, wherein the ORF encoding the L protein is under control of an arenavirus genomic 5′ UTR;
   d. an S segment, wherein the ORF encoding the GP is under control of an arenavirus genomic 3′ UTR;
   e. an S segment, wherein the ORF encoding the L protein is under control of an arenavirus genomic 3′ UTR; and
   f. an S segment, wherein the ORF encoding the Z protein is under control of an arenavirus genomic 3′ UTR.
270. The method of any one of paragraphs 147-183, 198-214, 230-252, 255-261 and 265-267, wherein the arenavirus particle is administered via intravenous injection.
271. The method of any one of paragraphs 161-163, 203-205 and 243-245, wherein the arenavirus particle is administered via intravenous injection.
272. The method of any one of paragraphs 147-183, 198-214, 230-252, 255-261, 265-267 and 270-271, wherein the at least one arenavirus ORF encodes the glycoprotein (“GP”), the nucleoprotein (“NP”), the matrix protein Z (“Z protein”) or the RNA dependent RNA polymerase L (“L protein”) of the arenavirus particle.
273. The method of any one of paragraphs 147-183, 198-214, 230-252, 255-261, 265-267 and 270-272, wherein the at least one arenavirus ORF is either functionally inactivated or deleted and wherein the arenavirus particle has the ability to amplify and express its genetic information in cells infected with the arenavirus particle but is unable to produce further infectious progeny particles in normal, non-complementing cells.
274. The method of any one of paragraphs 147-183, 198-214, 230-252, 255-261, 265-267 and 270-273, wherein the arenavirus particle is derived from lymphocytic choriomeningitis virus (LCMV) or Pichinde virus.
275. The method of any one of paragraphs 184-197, 215-229, 253-254, 262-264 and 268-269, wherein the first arenavirus particle, the second arenavirus particle, or both the first and second arenavirus particles are administered via intravenous injection.
276. The method of any one of paragraphs 220-222, wherein the first arenavirus particle, the second arenavirus particle, or both the first and second arenavirus particles are administered via intravenous injection.
277. The method of any one of paragraphs 184-197, 215-229, 253-254, 262-264, 268-269 and 275-276, wherein the at least one first arenavirus ORF encodes the glycoprotein (“GP”), the nucleoprotein (“NP”), the matrix protein Z (“Z protein”) or the RNA dependent RNA polymerase L (“L protein”) of the first arenavirus particle; and/or the at least one second arenavirus ORF encodes the glycoprotein (“GP”), the nucleoprotein (“NP”), the matrix protein Z (“Z protein”) or the RNA dependent RNA polymerase L (“L protein”) of the second arenavirus particle.
278. The method of any one of paragraphs 184-197, 215-229, 253-254, 262-264, 268-269 and 275-277, wherein the at least one first arenavirus ORF is either functionally inactivated or deleted and wherein the first arenavirus particle has the ability to amplify and express its genetic information in cells infected with the first arenavirus particle but is unable to produce further infectious progeny particles in normal, non-complementing cells; and/or wherein the at least one second arenavirus ORF is either functionally inactivated or deleted and wherein the second arenavirus particle has the ability to amplify and express its genetic information in cells infected with the second arenavirus particle but is unable to produce further infectious progeny particles in normal, non-complementing cells.
279. The method of any one of paragraphs 184-197, 215-229, 253-254, 262-264, 268-269 and 275-278, wherein the first arenavirus particle, the second arenavirus particle, or both the first and second arenavirus particles are derived from lymphocytic choriomeningitis virus (LCMV) or Pichinde virus.
280. The method of any one of paragraphs 147-279, wherein the method results in an increase of the concentration of T cells near cells infected with the pathogen.
281. The method of paragraph 280, wherein the method results in an increase of the concentration of CD8+ T cells, the concentration of CD4+ T cells, the concentration of T cells specific for the antigen of the pathogen, the concentration of T cells producing IFN-gamma, and/or the concentration of T cells producing granzyme B, near cells infected with the pathogen.
282. The method of any one of paragraphs 147-281, wherein the method results in an increase of the ratio of effector T cells/regulatory T cells near cells infected with the pathogen.
283. The method of any one of paragraphs 147-282, wherein the method has a higher anti-infection efficacy as compared to administration of a control arenavirus particle expressing the antigen of a pathogen that causes the infectious disease, or antigenic fragment thereof, alone.
284. The method of any one of paragraphs 147-283, wherein the method results in an increase in the survival rate of subjects treated with the method, compared to subjects having the same infectious disease in the absence of such treatment.
285. The method of any one of paragraphs 147-284, wherein the method is a method for treating the infectious disease.
286. The method of any one of paragraphs 147-285, wherein the infectious disease is a chronic infectious disease.
287. The method of any one of paragraphs 147-285, wherein the infectious disease is an acute infectious disease.
288. The method of any one of paragraphs 147-287, wherein the pathogen is a bacterium, virus, fungus, parasite, helminth or protist.
289. The method of any one of paragraphs 147-288, wherein the pathogen is HIV-1, HIV-2, HBV, HCV, HPV, CMV, HSV-1, HSV-2, EBV, Plasmodium falciparum, Mycobacterium tuberculosis, JC virus, HHV-6, HHV-7, HTLV-1, HTLV-2, VZV, Measles virus, or coronavirus.
290. The method of any one of paragraphs 147-288, wherein the pathogen is enterovirus, poliovirus, West Nile virus, Anaplasma phagocytophilum, Bacillus anthracis, Babesia microti, Brucella, Campylobacter, Enterobacterale, Haemophilus ducreyi, chikungunya virus, Chlamydia trachomatis, Clostridium difficile, coccidioides, SARS-CoV-2, Cryptosporidium, Cyclospora, Dengue virus, Corynebacterium diphtheriae, E. coli, Eastern equine encephalitis virus, Ebola virus, Ehrlichia chaffeensis, E. ewingii, E. muris eauclairensis, arbovirus, enterovirus, Giardia duodenalis, Burkholderia mallei, Neisseria gonorrhoeae, Klebsiella granulomatis, Type B Haemophilus influenzae, hantavirus, Escherichia coli O157:H7, hepatitis A virus, hepatitis B virus, hepatitis C virus, hepatitis D virus, hepatitis E virus, herpes simplex virus, varicella-zoster virus, Histoplasma, human immunodeficiency virus, human papillomavirus, influenza virus, Legionella, Mycobacterium leprae, Leptospira, Listeria monocytogenes, Borrelia burgdorferi, Borrelia mayonii, Chlamydia trachomatis, Plasmodium falciparum, P. vivax, P. ovale, P. malariae, P. knowlesi, measles virus, Burkholderiapseudomallei, mumps virus, rubella virus, MERS-CoV, norovirus, louse, Bordetella pertussis, Yersinia pestis, Streptococcus pneumoniae, polio virus, powassan virus, Chlamydiap sittaci, variola virus, monkeypox virus, cowpox virus, Coxiella burnetii, rabies virus, R. parkeri, Salmonella, Sarcoptes scabiei var. hominis, SARS-CoV, Shigella, Staphylococcus, Streptococcus, Treponema pallidum, Clostridium tetani, Trichomonas vaginalis, Trichinella, Mycobacterium tuberculosis, Francisella tularensis, Salmonella typhi, Rickettsia prowazekii, varicella-zoster virus, Vibrio cholerae, vibriosis, Marburg virus, Lassa virus, West Nile virus, coronavirus, yeast, yellow fever virus, Yersinia enterocolitica, or zika virus.
291. The method of any one of paragraphs 147-290, wherein the infectious disease is acute flaccid myelitis, anaplasmosis, anthrax, babesiosis, brucellosis, campylobacteriosis, carbapenem-resistant infection, chancroid, chikungunya virus infection, chlamydia, Clostridium difficile infection, coccidioidomycosis fungal infection, Covid-19, cryptosporidiosis, cyclosporiasis, dengue fever, diphtheria, E. coli infection, eastern equine encephalitis, Ebola hemorrhagic fever, ehrlichiosis, arboviral encephalitis, parainfectious encephalitis, enterovirus infection, giardiasis, glanders, gonococcal infection, granuloma inguinale, type b haemophilus influenza disease, hantavirus pulmonary syndrome, hemolytic uremic syndrome, hepatitis A, hepatitis B, hepatitis C, hepatitis D, hepatitis E, herpes, herpes zoster, histoplasmosis infection, acquired immunodeficiency syndrome, human papillomavirus infection, influenza, legionellosis, leprosy, leptospirosis, listeriosis, lyme disease, lymphogranuloma venereum infection, malaria, measles, melioidosis, viral meningitis, viral meningitis, middle east respiratory syndrome, multisystem inflammatory syndrome, mumps, norovirus infection, pediculosis, pelvic inflammatory disease, pertussis, plague, pneumococcal disease, poliomyelitis, powassan virus infection, psittacosis, pthiriasis, pustular rash disease, Q-fever, rabies, rickettsiosis, rubella, salmonellosis gastroenteritis, scabies infestation, sepsis, severe acute respiratory syndrome, shigellosis gastroenteritis, smallpox, staphyloccal infection, staphylococcal infection, streptococcal disease, streptococcal toxic-shock syndrome, syphilis, tetanus infection, trichomoniasis, trichonosis infection, tuberculosis, tularemia, typhoid fever, typhus, varicella, Vibrio cholera, vibriosis, viral hemorrhagic fever, west nile virus infection, coronavirus infection, yeast infection, yellow fever, yersenia, or zika virus infection.
292. A method for treating or preventing a neoplastic disease or preventing or treating an infectious disease in a subject in need thereof, wherein the method comprises administering to the subject an arenavirus particle; wherein
   a. the arenavirus particle comprises an arenavirus genome comprising a heterologous ORF encoding a cytokine, optionally the cytokine is IL-12; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
293. A method for treating or preventing a neoplastic disease or preventing or treating an infectious disease in a subject in need thereof, wherein the method comprises administering to the subject an arenavirus particle; wherein
   a. the arenavirus particle comprises an arenavirus genome comprising a heterologous ORF encoding an immune checkpoint modulator, optionally the immune checkpoint modulator is a ligand of 4-1BB; and
   b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
294. A method for treating or preventing a neoplastic disease in a subject in need thereof, wherein the method comprises administering to the subject (i) an arenavirus particle; and (ii) a cytokine; wherein
   a. the arenavirus particle comprises an arenavirus genome comprising a heterologous ORF encoding a tumor antigen, tumor associated antigen, or antigenic fragment thereof, and
   b. (i) at least one arenavirus open reading frame (ORF) of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
295. A method for treating or preventing an infectious disease in a subject in need thereof, wherein the method comprises administering to the subject (i) an arenavirus particle; and (ii) a cytokine, optionally the cytokine is IL-2; wherein
   a. the arenavirus particle comprises an arenavirus genome comprising a heterologous ORF encoding an antigen of a pathogen that causes the infectious disease, or antigenic fragment thereof; and
   b. (i) at least one arenavirus open reading frame (ORF) of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
296. The method of paragraph 294 or 295, further comprising administering to the subject an antibody that specifically binds to the cytokine, wherein the antibody is administered with the cytokine in the same composition.
297. The method of paragraph 296, wherein the cytokine is IL-2 and the antibody is an anti-IL-2 antibody.
298. The method of paragraph 295, wherein the cytokine is a fusion protein comprising IL-2 linked to an immunoglobulin, optionally wherein the immunoglobulin is an anti-IL-2 antibody.
299. The method of any one of paragraphs 295-298, wherein, the IL-2 is a modified IL-2 that has abrogated binding to CD25.
300. The method of any one of paragraphs 295-299, wherein the IL-2 is selected from the group consisting of ANV419, XTX202, AB248, MDNA11, STK-012, and combinations thereof.
A1. A method for treating or preventing a neoplastic disease or an infectious disease in a subject in need thereof, wherein the method comprises administering to the subject (i) an arenavirus particle, and (ii) an immune checkpoint modulator and/or a cytokine, optionally wherein the cytokine is IL-12; wherein
a. the arenavirus particle comprises an arenavirus genome comprising a heterologous ORF encoding an antigen or an antigenic fragment thereof, and
b. (i) at least one arenavirus open reading frame (ORF) of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
A2. The method of paragraph A1, wherein the method further comprises administering to the subject an antibody that specifically binds to the cytokine, wherein the antibody is administered with the cytokine in the same composition, optionally wherein the cytokine is TL-2 and the antibody is an anti-IL-2 antibody.
A3. The method of paragraph A1 or A2, wherein the cytokine is selected from the group consisting of an IL-2-immunoglobulin fusion protein, a modified TL-2 molecule having abrogated binding to CD25, ANV419, XTX202, AB248, MDNA11, STK-012, and combinations thereof.
A4. The method of any one of paragraphs A1-A3, wherein the arenavirus particle is tri-segmented and replication-competent and comprises one L segment and two S segments, wherein one of the two S segments is selected from the group consisting of:
a. an S segment, wherein the ORF encoding the NP is under control of an arenavirus genomic 5′ UTR;
b. an S segment, wherein the ORF encoding the Z protein is under control of an arenavirus genomic 5′ UTR;
c. an S segment, wherein the ORF encoding the L protein is under control of an arenavirus genomic 5′ UTR;
d. an S segment, wherein the ORF encoding the GP is under control of an arenavirus genomic 3′ UTR;
e. an S segment, wherein the ORF encoding the L protein is under control of an arenavirus genomic 3′ UTR; and
f. an S segment, wherein the ORF encoding the Z protein is under control of an arenavirus genomic 3′ UTR.
A5. A method for treating or preventing a neoplastic disease or an infectious disease in a subject in need thereof, wherein the method comprises administering to the subject an arenavirus particle, wherein
a. the arenavirus particle comprises an arenavirus genome comprising:
   i. a first heterologous ORF encoding an antigen; and
   ii. a second heterologous ORF encoding an immune checkpoint modulator or a cytokine, optionally wherein the cytokine is IL-12; and
b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
A6. The method of paragraph A5, wherein the arenavirus particle is tri-segmented and replication-competent and comprises one L segment and two S segments, wherein one of the two S segments is selected from the group consisting of:
a. an S segment, wherein the ORF encoding the NP is under control of an arenavirus genomic 5′ UTR;
b. an S segment, wherein the ORF encoding the Z protein is under control of an arenavirus genomic 5′ UTR;
c. an S segment, wherein the ORF encoding the L protein is under control of an arenavirus genomic 5′ UTR;
d. an S segment, wherein the ORF encoding the GP is under control of an arenavirus genomic 3′ UTR;
e. an S segment, wherein the ORF encoding the L protein is under control of an arenavirus genomic 3′ UTR; and
f. an S segment, wherein the ORF encoding the Z protein is under control of an arenavirus genomic 3′ UTR.
A7. The method of paragraph A6, wherein (i) a first S segment is engineered to carry an arenaviral ORF encoding GP in a position under control of an arenavirus genomic 3′ UTR and the first heterologous ORF in a position under control of an arenavirus genomic 5′ UTR, and a second S segment is engineered to carry an arenaviral ORF encoding NP in a position under control of an arenavirus genomic 3′ UTR and the second heterologous ORF in a position under control of an arenavirus genomic 5′ UTR; or (ii) a first S segment is engineered to carry an arenaviral ORF encoding NP in a position under control of an arenavirus genomic 3′ UTR and the first heterologous ORF in a position under control of an arenavirus genomic 5′ UTR, and a second S segment is engineered to carry an arenaviral ORF encoding GP in a position under control of an arenavirus genomic 3′ UTR and the second heterologous ORF in a position under control of an arenavirus genomic 5′ UTR; or (iii) an S segment is engineered to carry both the first and the second heterologous ORFs.
A8. The method of any one of paragraphs A1-A7, wherein the arenavirus particle is administered via intravenous injection or via intratumoral injection.
A9. The method of any one of paragraphs A1-A8, wherein the at least one arenavirus ORF encodes the glycoprotein (“GP”), the nucleoprotein (“NP”), the matrix protein Z (“Z protein”) or the RNA dependent RNA polymerase L (“L protein”) of the arenavirus particle.
A10. The method of any one of paragraphs A1-A3, A5, A8, and A9, wherein the at least one arenavirus ORF is either functionally inactivated or deleted and wherein the arenavirus particle has the ability to amplify and express its genetic information in cells infected with the arenavirus particle but is unable to produce further infectious progeny particles in normal, non-complementing cells.
A11. The method of any one of paragraphs A1-A10, wherein the arenavirus particle is derived from lymphocytic choriomeningitis virus (LCMV) or Pichinde virus.
A12. A method for treating or preventing a neoplastic disease or an infectious disease in a subject in need thereof, wherein the method comprises administering to the subject a first and a second arenavirus particles, wherein
(a) the first arenavirus particle comprises a first arenavirus genome comprising: a first heterologous ORF encoding an antigen; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
(b) the second arenavirus particle comprises a second arenavirus genome comprising: a second heterologous ORF encoding an immune checkpoint modulator or a cytokine, optionally wherein the cytokine is IL-12; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.
A13. The method of paragraph A12, wherein the first arenavirus particle, the second arenavirus particle, or both the first and second arenavirus particles are tri-segmented and replication-competent and comprise one L segment and two S segments, wherein one of the two S segments is selected from the group consisting of:
a. an S segment, wherein the ORF encoding the NP is under control of an arenavirus genomic 5′ UTR;
b. an S segment, wherein the ORF encoding the Z protein is under control of an arenavirus genomic 5′ UTR;
c. an S segment, wherein the ORF encoding the L protein is under control of an arenavirus genomic 5′ UTR;
d. an S segment, wherein the ORF encoding the GP is under control of an arenavirus genomic 3′ UTR;
e. an S segment, wherein the ORF encoding the L protein is under control of an arenavirus genomic 3′ UTR; and
f. an S segment, wherein the ORF encoding the Z protein is under control of an arenavirus genomic 3′ UTR.
A14. The method of paragraph A12 or A13, wherein the first and/or the second arenavirus particles are administered via intravenous injection or via intratumoral injection.
A15. The method of any one of paragraphs A12-A14, wherein the at least one first arenavirus ORF encodes the glycoprotein (“GP”), the nucleoprotein (“NP”), the matrix protein Z (“Z protein”) or the RNA dependent RNA polymerase L (“L protein”) of the first arenavirus particle; and/or the at least one second arenavirus ORF encodes the glycoprotein (“GP”), the nucleoprotein (“NP”), the matrix protein Z (“Z protein”) or the RNA dependent RNA polymerase L (“L protein”) of the second arenavirus particle.
A16. The method of any one of paragraphs A12, A14, and A15, wherein the at least one first arenavirus ORF is either functionally inactivated or deleted and wherein the first arenavirus particle has the ability to amplify and express its genetic information in cells infected with the first arenavirus particle but is unable to produce further infectious progeny particles in normal, non-complementing cells; and/or wherein the at least one second arenavirus ORF is either functionally inactivated or deleted and wherein the second arenavirus particle has the ability to amplify and express its genetic information in cells infected with the second arenavirus particle but is unable to produce further infectious progeny particles in normal, non-complementing cells.
A17. The method of any one of paragraphs A12-A16, wherein the first arenavirus particle, the second arenavirus particle, or both the first and second arenavirus particles are derived from lymphocytic choriomeningitis virus (LCMV) or Pichinde virus.
A18. A method for treating or preventing a neoplastic disease or preventing or treating an infectious disease in a subject in need thereof, wherein the method comprises administering to the subject an arenavirus particle; wherein
a. the arenavirus particle comprises an arenavirus genome comprising a heterologous ORF encoding (i) a cytokine, optionally the cytokine is IL-12, or (ii) an immune checkpoint modulator, optionally the immune checkpoint modulator is a ligand of 4-1BB; and
b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.
A19. The method of any one of paragraphs A1-A18, further comprising administering an additional immune checkpoint modulator that is different from the immune checkpoint modulator.
A20. The method of any one of paragraphs A1-A19, wherein the immune checkpoint modulator of any one of paragraphs 1-18 and/or the additional immune checkpoint modulator of paragraph 19 is an agonist of 4-1BB costimulatory pathway, an agonist of OX40 costimulatory pathway, an antagonist of NKG2A coinhibitory pathway, or a combination thereof,
optionally wherein
(i) the agonist of 4-1BB costimulatory pathway is an agonistic antibody of 4-1BB or 4-1BBL, optionally wherein the agonist of the 4-1BB costimulatory pathway is selected from the group consisting of utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, SL-279137 (PD-1-Fc-4-1BBL), and combinations thereof;
(ii) the agonist of OX40 costimulatory pathway is an agonistic antibody of OX40, optionally wherein the agonist of the OX40 costimulatory pathway is selected from the group consisting of INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, SL-279252 (PD1-Fc-OX40L), and combinations thereof, and/or
(iii) the antagonist of the NKG2A coinhibitory pathway is an antagonistic antibody of NKG2A.
A21. The method of any one of paragraphs 1-17, 19, and 20, wherein the method is for treating or preventing a neoplastic disease, and the antigen is a tumor antigen, tumor associated antigen, or antigenic fragment thereof.
A22. The method of paragraph 21, wherein the neoplastic disease is a solid tumor and wherein the method results in an increase of the concentration of T cells within the solid tumor, optionally wherein the method results in an increased concentration of CD8+ T cells, an increased concentration of CD4+ T cells, an increased concentration of tumor antigen specific T cells, an increased concentration of T cells producing IFN-gamma, an increased concentration of T cells producing granzyme B, and/or an increased ratio of effector T cells/regulatory T cells within the solid tumor.
A23. The method paragraph A21 or A22, wherein the method has a higher anti-tumor efficacy as compared to administration of a control arenavirus particle expressing the tumor antigen, tumor associated antigen, or antigenic fragment thereof, alone; and/or the method results in an increase in the survival rate of subjects treated with the method, compared to subjects having the same neoplastic disease in the absence of such treatment.
A24. The method of any one of paragraphs A1-A17, A19, and A20, wherein the method is for preventing or treating an infectious disease, and the antigen is an antigen of a pathogen that causes the infectious disease, or antigenic fragment thereof.
A25. The method of paragraph A24, wherein the method results in an increase of the concentration of T cells near cells infected with the pathogen, optionally wherein the method results in an increased concentration of CD8+ T cells, an increased concentration of CD4+ T cells, an increased concentration of tumor antigen specific T cells, an increased concentration of T cells producing IFN-gamma, an increased concentration of T cells producing granzyme B, and/or an increased ratio of effector T cells/regulatory T cells, near cells infected with the pathogen.
A26. The method of paragraph A24 or A25, wherein the method has a higher anti-infection efficacy as compared to administration of a control arenavirus particle expressing the antigen of a pathogen that causes the infectious disease, or antigenic fragment thereof, alone; and/or the method results in an increase in the survival rate of subjects treated with the method, compared to subjects having the same infectious disease in the absence of such treatment.

### 4. BRIEF DESCRIPTION OF THE FIGURES

FIGS. 1A and 1B: GP70-specific CD8 T cell responses in mice 7 days after single administration of indicated vector or vector/antibody combination (FIG. 1A) as determined by dextramer staining and flow cytometry analysis. CD127 geometric mean fluorescence intensity (GMFI) of GP70-specific CD8 T cells in mice 7 days after single administration of indicated vector or vector/antibody combination (FIG. 1).

FIGS. 2A-2C: FIG. 2A: Tumor growth development over time after single administration of the indicated treatment on day 8 after tumor challenge. FIG. 2B: Survival of tumor-bearing mice after single administration of the indicated treatment on day 8 after tumor challenge. FIG. 2C: GP70-specific (left panel) and LCMV NP-specific (right panel) CD8 T cell responses in mice 7 days after single administration of indicated vector or vector/antibody combination as determined by dextramer analysis.

FIGS. 3A-3C: FIG. 3A: Tumor growth development over time after single administration of the indicated treatment on day 6 after tumor challenge. FIG. 3B: Survival of tumor-bearing mice after single administration of the indicated treatment on day 6 after tumor challenge. CR=complete responders, MST=median survival time. FIG. 3C: GP70-specific (left panel) and LCMV NP-specific (right panel) CD8 T cell responses in mice 7 days after single administration of indicated vector or vector/antibody combination as determined by dextramer analysis.

FIGS. 4A and 4B: FIG. 4A: Tumor growth development over time after administration of artLCMV-GP70 vector on day 7 after tumor challenge and anti-4-1BB antibody treatment on the indicated days following tumor challenge. FIG. 4B: Survival of tumor-bearing mice after administration of artLCMV-GP70 vector on day 7 and anti-4-1BB antibody treatment on the indicated days following tumor challenge. CR=complete responders, MST=median survival time.

FIGS. 5A and 5B: FIG. 5A: Tumor growth development over time after administration of artLCMV-GP70 vector on day 8 after tumor challenge and agonistic antibody treatment on day 10 following tumor challenge. FIG. 5B: GP70-specific CD8 T cell responses in mice 7 days after single administration of indicated vector or vector/antibody combination as determined by dextramer analysis.

FIGS. 6A and 6B: FIG. 6A: Tumor growth development over time after tumor challenge on day 0 and intratumoral administration of the indicated artLCMV vectors on day 7. FIG. 6B: Survival of tumor-bearing mice after intratumoral administration of the indicated artLCMV-vectors seven days after tumor challenge.

FIGS. 7A and 7B: FIG. 7A: Tumor growth development over time after tumor challenge on day 0 and intratumoral administration of the indicated artLCMV vectors on day 7. FIG. 7B: Survival of tumor-bearing mice after intratumoral administration of the indicated artLCMV-vectors seven days after tumor challenge. CR=complete responders, MST=median survival time.

FIGS. 8A and 8B: FIG. 8A: Tumor growth development over time after tumor challenge on day 0 and intratumoral (i.t.) or intravenous (i.v.) administration of the indicated artLCMV vectors on day 7. FIG. 8B: Survival of tumor-bearing mice after intratumoral or intravenous administration of the indicated artLCMV-vectors seven days after tumor challenge. CR=complete responders, MST=median survival time.

FIGS. 9A and 9B: FIG. 9A: Tumor growth development over time after tumor challenge on day 0 and intravenous administration of the indicated artLCMV and artPICV vectors on day 11 in combination with either anti-NKG2A antibody or isotype control antibody. FIG. 9B: Survival of tumor bearing mice after intravenous administration of the indicated artLCMV and artPICV vectors in combination with either anti-NKG2A antibody or isotype control antibody.

FIGS. 10A-10C: schematic illustration of the genetic composition of wildtype and exemplary artArena particle constructs disclosed herein. FIG. 10A: schematic view of a wildtype particle (left side) and its genomic organization (right side). FIG. 10B: schematic view of an artArena particle (left side) and its genomic organization (right side) of an artArena particle encoding a tumor-associated antigen and α4-1BB Ligand. The ambisense RNA genome encodes for 4 viral proteins: GP (glycoprotein) and NP (nucleoprotein) on the S-segment; L (RNA-directed RNA polymerase) and Z (RING finger protein Z) on the L-segment. For artArena vector generation, viral NP and GP were segregated onto artificially duplicated S-segments. Each artLCMV vector therefore contains three genome segments, including one L-segment and two S-segments, where each S-segment contains a gene of interest and either NP or GP. All three genetic segments are required for artLCMV proliferation. artLCMV-GP70/4-1BBL and artLCMV-TRP2/4-1BBL vectors used in the disclosed Examples are engineered artArena particles based on LCMV, engineered to encode the tumor-associated antigens GP70 or TRP2, respectively, on the NP-S-Segment (1. S-Segment), and α4-1BB Ligand on the GP-S segment (2. S-Segment). FIG. 10C: schematic view of an artArena particle (left side) and its genomic organization (right side) of an artArena particle encoding a tumor-associated antigen and a cytokine. artLCMV-GP70-IL12 and artLCMV-GFP-IL12 used in the disclosed Examples are engineered artArena particles based on LCMV, engineered to encode the tumor-associated antigen GP70 or the irrelevant GFP, respectively, on the NP-S-Segment (1. S-Segment), and Interleukin 12 on the GP-S segment (2. S-Segment). All artLCMV vectors used were replication competent but stably attenuated by means of artificial genome organization.

FIG. 11: Enhancement of anti-tumor effects of artLCMV-TRP2 by single administration of agonistic α4-1BB antibodies in the B16.F10 model. C57BL/6 mice were subcutaneously injected with 2×10⁵ B16.F10 cells. Animals of groups 3 and 4 were treated intravenously with 1×10⁵ RCV FFU of artLCMV-TRP2 vector when tumors reached about 100 mm³ (day 7). On the day of vector application (day 7), animals of groups 2 and 4 were treated intraperitoneally with α4-1BB (100 μg). Tumor growth (left panel) and survival (middle panel) were monitored over time, and the frequency of antigen specific CD8 T cells was analyzed in blood on day 14 (right panel). Data shown are mean±SEM, n=7/group. CR=complete responder, MST=Median survival time.

FIGS. 12A-12F: Effect of α4-1BB treatment on number, cytotoxicity, proliferation and survival of GP70-specific CD8+ T cells in the tumor and draining lymph nodes. C57BL/6 mice were subcutaneously injected with 2x 105 B16.F10 cells. Animals of groups 2 and 3 were immunized intravenously with 1x 105 RCV FFU of artLCMV-GP70 when tumors reached about 100 mm³ (day 8). Agonistic α4-1BB (100 μg) was administered intraperitoneally to animals of group 3 on the day of vector administration (day 8). On day 8 and day 12 post vector application (day 16 and day 20 post tumor cell engraftment) tumor, spleen and tumor-draining lymph nodes were isolated and GP70-specific lymphocytes were quantified. The number of GP70-specific CD8+ T cells in tumor (FIG. 12A), spleen (FIG. 12C) and draining lymph nodes (dLNs) (FIG. 12E) were measured. Percentages of granzyme B, Ki-67, PD-1 and Bcl-XL expressing cells among GP70-specific CD8+ T cells in tumor (FIG. 12B), spleen (FIG. 12D) and dLNs (FIG. 12F) were analyzed by flow cytometry on day 8 after vector administration. Data shown are mean±SEM, n=3-5/group.

FIGS. 13A-13C: Increase of tumor efficacy by vector-encoded 4-1BBL, particularly after intratumoral administration, in the B16.F10 model but not in the MC-38 model. C57BL/6 mice were subcutaneously injected with 1×10⁶ MC-38 cells (FIG. 13A) or 2×10⁵ B16.F10 cells (FIGS. 13B and 13C). Animals were immunized intratumorally (G1-G3) or intravenously (G4-G6) with 1×10⁵ RCV FFU of artLCMV-GP70 or artLCMV-GP70/4-1BBL vectors as indicated (FIGS. 13A and 13B) or artLCMV-TRP2 or artLCMV-TRP2/4-1BBL vectors as indicated (FIG. 13C) when tumors reached around 100 mm³ (day 8). Tumor growth (left panel), animal survival (middle panel) were monitored and the frequency of antigen specific CD8 T cells (right panel) was analyzed in blood 7 days after vector administration. Data shown are mean±SEM, n=5-8/group. TAA=Tumor associated antigen (GP70 or TRP2), CR=complete responder, MST=Median survival time.

FIGS. 14A-14F: Anti-tumor efficacy of intravenous and intratumoral administration of artLCMV vectors expressing interleukin 12 (IL-12). C57BL/6 mice were subcutaneously injected with 2×10⁵ B16.F10 cells into the right flank. Animals were immunized intravenously (G1-G3) or intratumorally (G4 and G5) with 1x 105 RCV FFU of artLCMV vector encoding IL-12 and GP70 (G2 and G4) or artLCMV vector encoding IL-12 and GFP (G3 and G5) when tumors reached around 100 mm³ (day 8). (FIG. 14A) Tumor growth, (FIG. 14B) survival and (FIG. 14C) body weight were monitored and (FIG. 14D) the frequency of antigen specific CD8 T cells was analyzed in blood 7 days after vector administration. (FIGS. 14E and 14F) On day 107 after tumor cell engraftment, tumor-free animals were re-challenged by subcutaneous injection of 2×10⁵ B16.F10 cells into the left flank. As a control, 5 naïve animals were also engrafted with B16.F10 cells (G6). Data shown are mean±SEM, n=7/group. CR=complete responder, MST=Median survival time.

FIGS. 15A-15D: Anti-tumor efficacy of intravenously administered artLCMV vectors encoding GP70 and interleukin 12 (IL-12). C57BL/6 mice were subcutaneously injected with 2×10⁵ B16.F10 cells. Animals were immunized intravenously with 1x105 RCV FFU of artLCMV-GP70 vector (G2) or different doses of GP70 and IL-12 encoding vectors (G3-G5: artLCMV-GP70-IL12, 1×10³, 1×10⁴ and 1×10⁵ RCV FFU) when tumors reached around 100 mm³ (day 7). (FIG. 15A) Tumor growth (FIG. 15B) survival and (FIG. 15C) body weight were monitored and (FIG. 15D) the frequency of antigen specific CD8 T cells was analyzed in blood 7 days after vector administration. Data shown are mean±SEM, n=5/group. CR=complete responder, MST=Median survival time.

FIGS. 16A-16D: C57BL/6 mice were subcutaneously injected on the right flank with 1×10⁶ TC-1 cells. Animals in groups 3 and 4 (G3 and G4) were immunized intravenously with 1×10⁵ RCV FFU of artLCMV-E7E6 on day 10. In addition, animals in groups 2 and 4 (G2 and G4) were treated intraperitoneally with an IL-2 complex every 2-3 days starting on day 4 after vector administration (5 treatments in total on day 14, 16, 18, 21 and 23) (FIG. 16A) Tumor growth, (FIG. 16B) body weight, and (FIG. 16C) survival were monitored and (FIG. 16D) the number of antigen-specific CD8 T cells was analyzed in blood on day 8, day 15 and day 22 after vector administration. Data shown are mean±SEM, n=8/group. CR=complete responder, MST=Median survival time.

### 5. DETAILED DESCRIPTION OF THE INVENTION

Provided herein are methods for treating or preventing a neoplastic disease in a subject in need thereof, using a combination of (1) a tumor antigen, tumor associated antigen, or antigenic fragment thereof, encoded by an arenavirus particle, and (2) at least one immune checkpoint modulator and/or at least one cytokine. The at least one immune checkpoint modulator and/or at least one cytokine can each be administered in combination with the arenavirus particle encoding the tumor antigen, tumor associated antigen, or antigenic fragment thereof, or be encoded by the same arenavirus particle or a different arenavirus particle.

Provided herein are methods for treating or preventing an infectious disease in a subject in need thereof, using a combination of (1) an antigen of a pathogen that causes the infectious disease, or antigenic fragment thereof, encoded by an arenavirus particle, and (2) at least one immune checkpoint modulator and/or at least one cytokine. The at least one immune checkpoint modulator and/or at least one cytokine can each be administered in combination with the arenavirus particle encoding the antigen of a pathogen that causes the infectious disease, or antigenic fragment thereof, or be encoded by the same arenavirus particle or a different arenavirus particle.

Provided herein are also methods for treating or preventing a neoplastic disease, or treating or preventing an infectious disease in a subject in need thereof, using a presently disclosed arenavirus particle encoding at least one immune checkpoint modulator and/or at least one cytokine.

The term “immune checkpoint modulator” as used in this disclosure refers to an agonist of a costimulatory pathway or an antagonist of a coinhibitory pathway. An agonist of a costimulatory pathway activates the costimulatory pathway, and can be, for example, an agonistic antibody that binds to and activates the corresponding costimulatory immune checkpoint receptor, an agonist aptamer that binds to and activates the corresponding costimulatory immune checkpoint receptor, or a ligand that binds to and activates the corresponding costimulatory immune checkpoint receptor. An antagonist of a coinhibitory pathway inhibits the coinhibitory pathway, and can be, for example, an antagonistic antibody that binds to and inhibits the corresponding coinhibitory immune checkpoint receptor, an antagonistic aptamer that binds to and inhibits the corresponding coinhibitory immune checkpoint receptor, or a ligand that binds to and inhibits the corresponding coinhibitory immune checkpoint receptor.

Provided herein are methods for treating or preventing a neoplastic disease (see Section 5.1) in a subject in need thereof, wherein the methods comprise delivering to the subject an arenavirus particle (see arenavirus particles specified in Section 5.4-5.6) and at least two different immune checkpoint modulators (see Sections 5.8), wherein the arenavirus particle is engineered to contain an arenavirus genomic segment comprising a nucleotide sequence encoding an antigen, such as a tumor antigen, tumor associated antigen or antigenic fragment thereof (see Section 5.1). Optionally, one or two of the immune checkpoint modulators can be encoded by a nucleotide sequence comprised in the arenavirus genome of the same arenavirus particle that contains the arenavirus genomic segment comprising the nucleotide sequence encoding the tumor antigen, tumor associated antigen or antigenic fragment thereof or in the arenavirus genome of a separate arenavirus particle from the arenavirus particle that encodes the tumor antigen, tumor associated antigen or antigenic fragment thereof.

In one aspect, provided herein is a method for treating or preventing a neoplastic disease in a subject in need thereof, wherein the method comprises administering to the subject (i) an arenavirus particle; and (ii) two different immune checkpoint modulators; wherein (a) the arenavirus particle comprises an arenavirus genome comprising a heterologous ORF encoding a tumor antigen, tumor associated antigen, or antigenic fragment thereof; and (b) (i) at least one arenavirus open reading frame (ORF) of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

The two different immune checkpoint modulators can be (1) two different agonists of one or more costimulatory pathways (e.g., two different agonists of the same costimulatory pathway, or two different agonists of two different costimulatory pathways, respectively), (2) two different antagonists of one or more coinhibitory pathways (e.g., two different antagonists of the same coinhibitory pathway, or two different antagonists of two different coinhibitory pathways, respectively), or (3) an agonist of one or more costimulatory pathways (e.g., an agonist of one costimulatory pathway) and an antagonist of one or more coinhibitory pathways (e.g., an antagonist of one or more coinhibitory pathway).

In certain embodiments, at least one of the two different immune checkpoint modulators targets a member of the tumor necrosis factor receptor superfamily (“TNFRSF”). In certain embodiments, the two different immune checkpoint modulators comprise an immune checkpoint modulator that is an agonist of the 4-1BB costimulatory pathway and another immune checkpoint modulator other than an agonist of the 4-1BB costimulatory pathway. In specific embodiments, the immune checkpoint modulator other than an agonist of the 4-1BB costimulatory pathway is an agonist of the OX40 costimulatory pathway. In certain embodiments, the two different immune checkpoint modulators comprise an immune checkpoint modulator that is an agonist of the OX40 costimulatory pathway and another immune checkpoint modulator other than an agonist of the OX40 costimulatory pathway. In specific embodiments, the immune checkpoint modulator other than an agonist of the OX40 costimulatory pathway is an agonist of the 4-1BB costimulatory pathway.

Also provided herein are methods for treating or preventing an infectious disease (see Section 5.2) in a subject in need thereof, wherein the methods comprise delivering to the subject an arenavirus particle (see arenavirus particles specified in Section 5.4-5.6) and at least two different immune checkpoint modulators (see Sections 5.8), wherein the arenavirus particle is engineered to contain an arenavirus genomic segment comprising a nucleotide sequence encoding an antigen, such as an antigen of a pathogen that causes the infectious disease, or antigenic fragment thereof (see Section 5.2). Optionally, one or two of the immune checkpoint modulators can be encoded by a nucleotide sequence comprised in the arenavirus genome of the same arenavirus particle that contains the arenavirus genomic segment comprising the nucleotide sequence encoding the antigen of a pathogen that causes the infectious disease or antigenic fragment thereof, or in the arenavirus genome of a separate arenavirus particle from the arenavirus particle that encodes the antigen of a pathogen that causes the infectious disease or antigenic fragment thereof.

In one aspect, provided herein is a method for treating or preventing an infectious disease in a subject in need thereof, wherein the method comprises administering to the subject (i) an arenavirus particle; and (ii) two different immune checkpoint modulators; wherein (a) the arenavirus particle comprises an arenavirus genome comprising a heterologous ORF encoding an antigen of a pathogen that causes the infectious disease, or antigenic fragment thereof, and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

The two different immune checkpoint modulators can be (1) two different agonists of one or more costimulatory pathways (e.g., two different agonists of the same costimulatory pathway, or two different agonists of two different costimulatory pathways, respectively), (2) two different antagonists of one or more coinhibitory pathways (e.g., two different antagonists of the same coinhibitory pathway, or two different antagonists of two different coinhibitory pathways, respectively), or (3) an agonist of one or more costimulatory pathways (e.g., an agonist of one costimulatory pathway) and an antagonist of one or more coinhibitory pathways (e.g., an antagonist of one or more coinhibitory pathway).

In certain embodiments, at least one of the two different immune checkpoint modulators targets a member of the tumor necrosis factor receptor superfamily (“TNFRSF”). In certain embodiments, the two different immune checkpoint modulators comprise an immune checkpoint modulator that is an agonist of the 4-1BB costimulatory pathway and another immune checkpoint modulator other than an agonist of the 4-1BB costimulatory pathway. In specific embodiments, the immune checkpoint modulator other than an agonist of the 4-1BB costimulatory pathway is an agonist of the OX40 costimulatory pathway. In certain embodiments, the two different immune checkpoint modulators comprise an immune checkpoint modulator that is an agonist of the OX40 costimulatory pathway and another immune checkpoint modulator other than an agonist of the OX40 costimulatory pathway. In specific embodiments, the immune checkpoint modulator other than an agonist of the OX40 costimulatory pathway is an agonist of the 4-1BB costimulatory pathway.

Also provided herein are methods for treating or preventing a neoplastic disease in a subject in need thereof, wherein the methods comprise administering to the subject an arenavirus particle, wherein the arenavirus particle comprises a genome that is engineered to comprise (i) a nucleotide sequence encoding a tumor antigen, tumor associated antigen, or antigenic fragment thereof, and (ii) a nucleotide sequence encoding an immune checkpoint modulator that is a ligand of 4-1BB.

In one aspect, provided herein is a method for treating or preventing a neoplastic disease in a subject in need thereof, wherein the method comprises administering to the subject an arenavirus particle; wherein (a) the arenavirus particle comprises an arenavirus genome comprising: (i) a first heterologous ORF encoding a tumor antigen, tumor associated antigen, or antigenic fragment thereof, and (ii) a second heterologous ORF encoding an immune checkpoint modulator that is a ligand of 4-1BB; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing an infectious disease in a subject in need thereof, wherein the methods comprise administering to the subject an arenavirus particle, wherein the arenavirus particle comprises a genome that is engineered to comprise (i) a nucleotide sequence encoding an antigen of a pathogen that causes the infectious disease, or antigenic fragment thereof, and (ii) a nucleotide sequence encoding an immune checkpoint modulator that is a ligand of 4-1BB.

In one aspect, provided herein is a method for treating or preventing an infectious disease in a subject in need thereof, wherein the method comprises administering to the subject an arenavirus particle; wherein (a) the arenavirus particle comprises an arenavirus genome comprising: (i) a first heterologous ORF encoding an antigen of a pathogen that causes the infectious disease, or antigenic fragment thereof; and (ii) a second heterologous ORF encoding an immune checkpoint modulator that is a ligand of 4-1BB; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF

Also provided herein are methods for treating or preventing a neoplastic disease in a subject in need thereof, wherein the methods comprise administering to the subject (i) a first arenavirus particle, wherein the arenavirus particle comprises a genome that is engineered to comprise a nucleotide sequence encoding a tumor antigen, tumor associated antigen, or antigenic fragment thereof, and (ii) a second arenavirus particle, wherein the arenavirus particle comprises a genome that is engineered to comprise a nucleotide sequence encoding an immune checkpoint modulator that is a ligand of 4-1BB.

In one aspect, provided herein is a method for treating or preventing a neoplastic disease in a subject in need thereof, wherein the method comprises administering to the subject (i) a first arenavirus particle; and (ii) a second arenavirus particle; wherein (a) the first arenavirus particle comprises a first arenavirus genome comprising: a first heterologous ORF encoding a tumor antigen, tumor associated antigen, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and (b) the second arenavirus particle comprises a second arenavirus genome comprising: a second heterologous ORF encoding an immune checkpoint modulator that is a ligand of 4-1BB; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

Also provided herein are methods for treating or preventing an infectious disease in a subject in need thereof, wherein the methods comprise administering to the subject (i) a first arenavirus particle, wherein the arenavirus particle comprises a genome that is engineered to comprise a nucleotide sequence encoding an antigen of a pathogen that causes the infectious disease, or antigenic fragment thereof, and (ii) a second arenavirus particle, wherein the arenavirus particle comprises a genome that is engineered to comprise a nucleotide sequence encoding an immune checkpoint modulator that is a ligand of 4-1BB.

In one aspect, provided herein is a method for treating or preventing an infectious disease in a subject in need thereof, wherein the method comprises administering to the subject (i) a first arenavirus particle; and (ii) a second arenavirus particle; wherein (a) the first arenavirus particle comprises a first arenavirus genome comprising: a first heterologous ORF encoding an antigen of a pathogen that causes the infectious disease, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and (b) the second arenavirus particle comprises a second arenavirus genome comprising: a second heterologous ORF encoding an immune checkpoint modulator that is a ligand of 4-1BB; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

Also provided herein are methods for treating or preventing a neoplastic disease in a subject in need thereof, wherein the methods comprise administering to the subject (i) an arenavirus particle, wherein the arenavirus particle comprises a genome that is engineered to comprise a nucleotide sequence encoding a tumor antigen, tumor associated antigen, or antigenic fragment thereof, and (ii) an immune checkpoint modulator that is an agonist of the 4-1BB costimulatory pathway.

In one aspect, provided herein is a method for treating or preventing a neoplastic disease in a subject in need thereof, wherein the method comprises administering to the subject (i) an arenavirus particle; and (ii) an immune checkpoint modulator that is an agonist of the 4-1BB costimulatory pathway; wherein: (a) the arenavirus particle comprises an arenavirus genome comprising a heterologous ORF encoding a tumor antigen, tumor associated antigen, or antigenic fragment thereof; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing an infectious disease in a subject in need thereof, wherein the methods comprise administering to the subject (i) an arenavirus particle, wherein the arenavirus particle comprises a genome that is engineered to comprise a nucleotide sequence encoding an antigen of a pathogen that causes the infectious disease, or antigenic fragment thereof, and (ii) an immune checkpoint modulator that is an agonist of the 4-1BB costimulatory pathway.

In one aspect, provided herein is a method for treating or preventing an infectious disease in a subject in need thereof, wherein the method comprises administering to the subject (i) an arenavirus particle; and (ii) an immune checkpoint modulator that is an agonist of the 4-1BB costimulatory pathway; wherein: (a) the arenavirus particle comprises an arenavirus genome comprising a heterologous ORF encoding an antigen of a pathogen that causes the infectious disease, or antigenic fragment thereof, and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing a neoplastic disease in a subject in need thereof, wherein the methods comprise administering to the subject an arenavirus particle, wherein the arenavirus particle comprises a genome that is engineered to comprise (i) a nucleotide sequence encoding a tumor antigen, tumor associated antigen, or antigenic fragment thereof, and (ii) a nucleotide sequence encoding an immune checkpoint modulator that is an agonist of the 4-1BB costimulatory pathway.

In one aspect, provided herein is a method for treating or preventing a neoplastic disease in a subject in need thereof, wherein the method comprises administering to the subject an arenavirus particle; wherein (a) the arenavirus particle comprises an arenavirus genome comprising: (i) a first heterologous ORF encoding a tumor antigen, tumor associated antigen, or antigenic fragment thereof, and (ii) a second heterologous ORF encoding an immune checkpoint modulator that is an agonist of the 4-1BB costimulatory pathway; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing an infectious disease in a subject in need thereof, wherein the methods comprise administering to the subject an arenavirus particle, wherein the arenavirus particle comprises a genome that is engineered to comprise (i) a nucleotide sequence encoding an antigen of a pathogen that causes the infectious disease, or antigenic fragment thereof, and (ii) a nucleotide sequence encoding an immune checkpoint modulator that is an agonist of the 4-1BB costimulatory pathway.

In one aspect, provided herein is a method for treating or preventing an infectious disease in a subject in need thereof, wherein the method comprises administering to the subject an arenavirus particle; wherein (a) the arenavirus particle comprises an arenavirus genome comprising: (i) a first heterologous ORF encoding an antigen of a pathogen that causes the infectious disease, or antigenic fragment thereof; and (ii) a second heterologous ORF encoding an immune checkpoint modulator that is an agonist of the 4-1BB costimulatory pathway; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing a neoplastic disease in a subject in need thereof, wherein the methods comprise administering to the subject (i) an arenavirus particle, wherein the arenavirus particle comprises a genome that is engineered to comprise (a) a nucleotide sequence encoding a tumor antigen, tumor associated antigen, or antigenic fragment thereof, and (b) a nucleotide sequence encoding an immune checkpoint modulator that is an agonist of the 4-1BB costimulatory pathway; and (ii) an immune checkpoint modulator other than an agonist of the 4-1BB costimulatory pathway.

In one aspect, provided herein is a method for treating or preventing a neoplastic disease in a subject in need thereof, wherein the method comprises administering to the subject (i) an arenavirus particle; and (ii) an immune checkpoint modulator other than an agonist of the 4-1BB costimulatory pathway; wherein (a) the arenavirus particle comprises an arenavirus genome comprising: (i) a first heterologous ORF encoding a tumor antigen, tumor associated antigen, or antigenic fragment thereof, and (ii) a second heterologous ORF encoding an immune checkpoint modulator that is an agonist of the 4-1BB costimulatory pathway; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

In certain embodiments, the immune checkpoint modulator other than an agonist of the 4-1BB costimulatory pathway targets a member of the TNFRSF. In specific embodiments, the immune checkpoint modulator other than an agonist of the 4-1BB costimulatory pathway is an agonist of the OX40 costimulatory pathway.

Also provided herein are methods for treating or preventing an infectious disease in a subject in need thereof, wherein the methods comprise administering to the subject (i) an arenavirus particle, wherein the arenavirus particle comprises a genome that is engineered to comprise (a) a nucleotide sequence encoding an antigen of a pathogen that causes the infectious disease, or antigenic fragment thereof, and (b) a nucleotide sequence encoding an immune checkpoint modulator that is an agonist of the 4-1BB costimulatory pathway; and (ii) an immune checkpoint modulator other than an agonist of the 4-1BB costimulatory pathway.

In one aspect, provided herein is a method for treating or preventing an infectious disease in a subject in need thereof, wherein the method comprises administering to the subject (i) an arenavirus particle; and (ii) an immune checkpoint modulator other than an agonist of the 4-1BB costimulatory pathway; wherein (a) the arenavirus particle comprises an arenavirus genome comprising: (i) a first heterologous ORF encoding an antigen of a pathogen that causes the infectious disease, or antigenic fragment thereof, and (ii) a second heterologous ORF encoding an immune checkpoint modulator that is an agonist of the 4-1BB costimulatory pathway; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

In certain embodiments, the immune checkpoint modulator other than an agonist of the 4-1BB costimulatory pathway targets a member of the TNFRSF. In specific embodiments, the immune checkpoint modulator other than an agonist of the 4-1BB costimulatory pathway is an agonist of the OX40 costimulatory pathway.

Also provided herein are methods for treating or preventing a neoplastic disease in a subject in need thereof, wherein the methods comprise administering to the subject (i) a first arenavirus particle, wherein the first arenavirus particle comprises a genome that is engineered to comprise a nucleotide sequence encoding a tumor antigen, tumor associated antigen, or antigenic fragment thereof, and (ii) a second arenavirus particle, wherein the second arenavirus particle comprises a genome that is engineered to comprise a nucleotide sequence encoding an immune checkpoint modulator that is an agonist of the 4-1BB costimulatory pathway; and (iii) an immune checkpoint modulator other than an agonist of the 4-1BB costimulatory pathway.

In one aspect, provided herein is a method for treating or preventing a neoplastic disease in a subject in need thereof, wherein the method comprises administering to the subject (i) a first arenavirus particle; and (ii) a second arenavirus particle; and (iii) an immune checkpoint modulator other than an agonist of the 4-1BB costimulatory pathway; wherein (a) the first arenavirus particle comprises a first arenavirus genome comprising: a first heterologous ORF encoding a tumor antigen, tumor associated antigen, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and (b) the second arenavirus particle comprises a second arenavirus genome comprising: a second heterologous ORF encoding an immune checkpoint modulator that is an agonist of the 4-1BB costimulatory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

In certain embodiments, the immune checkpoint modulator other than an agonist of the 4-1BB costimulatory pathway targets a member of the TNFRSF. In specific embodiments, the immune checkpoint modulator other than an agonist of the 4-1BB costimulatory pathway is an agonist of the OX40 costimulatory pathway.

Also provided herein are methods for treating or preventing an infectious disease in a subject in need thereof, wherein the methods comprise administering to the subject (i) a first arenavirus particle, wherein the first arenavirus particle comprises a genome that is engineered to comprise a nucleotide sequence encoding an antigen of a pathogen that causes the infectious disease, or antigenic fragment thereof, and (ii) a second arenavirus particle, wherein the second arenavirus particle comprises a genome that is engineered to comprise a nucleotide sequence encoding an immune checkpoint modulator that is an agonist of the 4-1BB costimulatory pathway; and (iii) an immune checkpoint modulator other than an agonist of the 4-1BB costimulatory pathway.

In one aspect, provided herein is a method for treating or preventing an infectious disease in a subject in need thereof, wherein the method comprises administering to the subject (i) a first arenavirus particle; and (ii) a second arenavirus particle; and (iii) an immune checkpoint modulator other than an agonist of the 4-1BB costimulatory pathway; wherein (a) the first arenavirus particle comprises a first arenavirus genome comprising: a first heterologous ORF encoding an antigen of a pathogen that causes the infectious disease, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and (b) the second arenavirus particle comprises a second arenavirus genome comprising: a second heterologous ORF encoding an immune checkpoint modulator that is an agonist of the 4-1BB costimulatory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

In certain embodiments, the immune checkpoint modulator other than an agonist of the 4-1BB costimulatory pathway targets a member of the TNFRSF. In specific embodiments, the immune checkpoint modulator other than an agonist of the 4-1BB costimulatory pathway is an agonist of the OX40 costimulatory pathway.

Also provided herein are methods for treating or preventing a neoplastic disease in a subject in need thereof, wherein the methods comprise administering to the subject (i) an arenavirus particle, wherein the arenavirus particle comprises a genome that is engineered to comprise (a) a nucleotide sequence encoding a tumor antigen, tumor associated antigen, or antigenic fragment thereof, and (b) a nucleotide sequence encoding an immune checkpoint modulator that is an agonist of the OX40 costimulatory pathway; and (ii) an immune checkpoint modulator other than an agonist of the OX40 costimulatory pathway.

In one aspect, provided herein is a method for treating or preventing a neoplastic disease in a subject in need thereof, wherein the method comprises administering to the subject (i) an arenavirus particle; and (ii) an immune checkpoint modulator other than an agonist of the OX40 costimulatory pathway; wherein (a) the arenavirus particle comprises an arenavirus genome comprising: (i) a first heterologous ORF encoding a tumor antigen, tumor associated antigen, or antigenic fragment thereof, and (ii) a second heterologous ORF encoding an immune checkpoint modulator that is an agonist of the OX40 costimulatory pathway; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

In certain embodiments, the immune checkpoint modulator other than an agonist of the OX40 costimulatory pathway targets a member of the TNFRSF. In specific embodiments, the immune checkpoint modulator other than an agonist of the OX40 costimulatory pathway is an agonist of the 4-1BB costimulatory pathway.

Also provided herein are methods for treating or preventing an infectious disease in a subject in need thereof, wherein the methods comprise administering to the subject (i) an arenavirus particle, wherein the arenavirus particle comprises a genome that is engineered to comprise (a) a nucleotide sequence encoding an antigen of a pathogen that causes the infectious disease, or antigenic fragment thereof, and (b) a nucleotide sequence encoding an immune checkpoint modulator that is an agonist of the OX40 costimulatory pathway; and (ii) an immune checkpoint modulator other than an agonist of the OX40 costimulatory pathway.

In one aspect, provided herein is a method for treating or preventing an infectious disease in a subject in need thereof, wherein the method comprises administering to the subject (i) an arenavirus particle; and (ii) an immune checkpoint modulator other than an agonist of the OX40 costimulatory pathway; wherein (a) the arenavirus particle comprises an arenavirus genome comprising: (i) a first heterologous ORF encoding an antigen of a pathogen that causes the infectious disease, or antigenic fragment thereof, and (ii) a second heterologous ORF encoding an immune checkpoint modulator that is an agonist of the OX40 costimulatory pathway; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

In certain embodiments, the immune checkpoint modulator other than an agonist of the OX40 costimulatory pathway targets a member of the TNFRSF. In specific embodiments, the immune checkpoint modulator other than an agonist of the OX40 costimulatory pathway is an agonist of the 4-1BB costimulatory pathway.

Also provided herein are methods for treating or preventing a neoplastic disease in a subject in need thereof, wherein the methods comprise administering to the subject (i) a first arenavirus particle, wherein the first arenavirus particle comprises a genome that is engineered to comprise a nucleotide sequence encoding a tumor antigen, tumor associated antigen, or antigenic fragment thereof, and (ii) a second arenavirus particle, wherein the second arenavirus particle comprises a genome that is engineered to comprise a nucleotide sequence encoding an immune checkpoint modulator that is an agonist of the OX40 costimulatory pathway; and (iii) an immune checkpoint modulator other than an agonist of the OX40 costimulatory pathway.

In one aspect, provided herein is a method for treating or preventing a neoplastic disease in a subject in need thereof, wherein the method comprises administering to the subject (i) a first arenavirus particle; and (ii) a second arenavirus particle; and (iii) an immune checkpoint modulator other than an agonist of the OX40 costimulatory pathway; wherein (a) the first arenavirus particle comprises a first arenavirus genome comprising: a first heterologous ORF encoding a tumor antigen, tumor associated antigen, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and (b) the second arenavirus particle comprises a second arenavirus genome comprising: a second heterologous ORF encoding an immune checkpoint modulator that is an agonist of the OX40 costimulatory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

In certain embodiments, the immune checkpoint modulator other than an agonist of the OX40 costimulatory pathway targets a member of the TNFRSF. In specific embodiments, the immune checkpoint modulator other than an agonist of the OX40 costimulatory pathway is an agonist of the 4-1BB costimulatory pathway.

Also provided herein are methods for treating or preventing an infectious disease in a subject in need thereof, wherein the methods comprise administering to the subject (i) a first arenavirus particle, wherein the first arenavirus particle comprises a genome that is engineered to comprise a nucleotide sequence encoding an antigen of a pathogen that causes the infectious disease, or antigenic fragment thereof, and (ii) a second arenavirus particle, wherein the second arenavirus particle comprises a genome that is engineered to comprise a nucleotide sequence encoding an immune checkpoint modulator that is an agonist of the OX40 costimulatory pathway; and (iii) an immune checkpoint modulator other than an agonist of the OX40 costimulatory pathway.

In one aspect, provided herein is a method for treating or preventing an infectious disease in a subject in need thereof, wherein the method comprises administering to the subject (i) a first arenavirus particle; and (ii) a second arenavirus particle; and (iii) an immune checkpoint modulator other than an agonist of the OX40 costimulatory pathway; wherein (a) the first arenavirus particle comprises a first arenavirus genome comprising: a first heterologous ORF encoding an antigen of a pathogen that causes the infectious disease, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and (b) the second arenavirus particle comprises a second arenavirus genome comprising: a second heterologous ORF encoding an immune checkpoint modulator that is an agonist of the OX40 costimulatory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

In certain embodiments, the immune checkpoint modulator other than an agonist of the OX40 costimulatory pathway targets a member of the TNFRSF. In specific embodiments, the immune checkpoint modulator other than an agonist of the OX40 costimulatory pathway is an agonist of the 4-1BB costimulatory pathway.

Also provided herein are methods for treating or preventing a neoplastic disease in a subject in need thereof, wherein the methods comprise administering to the subject an arenavirus particle, wherein the arenavirus particle comprises a genome that is engineered to comprise (i) a nucleotide sequence encoding a tumor antigen, tumor associated antigen, or antigenic fragment thereof, and (ii) a nucleotide sequence encoding an immune checkpoint modulator that is an antagonist of the NKG2A coinhibitory pathway.

In one aspect, provided herein is a method for treating or preventing a neoplastic disease in a subject in need thereof, wherein the method comprises administering to the subject an arenavirus particle; wherein (a) the arenavirus particle comprises an arenavirus genome comprising: (i) a first heterologous ORF encoding a tumor antigen, tumor associated antigen, or antigenic fragment thereof, and (ii) a second heterologous ORF encoding an immune checkpoint modulator that is an antagonist of the NKG2A coinhibitory pathway; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing an infectious disease in a subject in need thereof, wherein the methods comprise administering to the subject an arenavirus particle, wherein the arenavirus particle comprises a genome that is engineered to comprise (i) a nucleotide sequence encoding an antigen of a pathogen that causes the infectious disease, or antigenic fragment thereof, and (ii) a nucleotide sequence encoding an immune checkpoint modulator that is an antagonist of the NKG2A coinhibitory pathway.

In one aspect, provided herein is a method for treating or preventing an infectious disease in a subject in need thereof, wherein the method comprises administering to the subject an arenavirus particle; wherein (a) the arenavirus particle comprises an arenavirus genome comprising: (i) a first heterologous ORF encoding an antigen of a pathogen that causes the infectious disease, or antigenic fragment thereof; and (ii) a second heterologous ORF encoding an immune checkpoint modulator that is an antagonist of the NKG2A coinhibitory pathway; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing a neoplastic disease in a subject in need thereof, wherein the methods comprise administering to the subject (i) an arenavirus particle, wherein the arenavirus particle comprises a genome that is engineered to comprise a nucleotide sequence encoding a tumor antigen, tumor associated antigen, or antigenic fragment thereof, and (ii) an immune checkpoint modulator that is an antagonist of the NKG2A coinhibitory pathway.

In one aspect, provided herein is a method for treating or preventing a neoplastic disease in a subject in need thereof, wherein the method comprises administering to the subject (i) an arenavirus particle; and (ii) an immune checkpoint modulator that is an antagonist of the NKG2A coinhibitory pathway; wherein: (a) the arenavirus particle comprises an arenavirus genome comprising a heterologous ORF encoding a tumor antigen, tumor associated antigen, or antigenic fragment thereof; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing an infectious disease in a subject in need thereof, wherein the methods comprise administering to the subject (i) an arenavirus particle, wherein the arenavirus particle comprises a genome that is engineered to comprise a nucleotide sequence encoding an antigen of a pathogen that causes the infectious disease, or antigenic fragment thereof, and (ii) an immune checkpoint modulator that is an antagonist of the NKG2A coinhibitory pathway.

In one aspect, provided herein is a method for treating or preventing an infectious disease in a subject in need thereof, wherein the method comprises administering to the subject (i) an arenavirus particle; and (ii) an immune checkpoint modulator that is an antagonist of the NKG2A coinhibitory pathway; wherein: (a) the arenavirus particle comprises an arenavirus genome comprising a heterologous ORF encoding an antigen of a pathogen that causes the infectious disease, or antigenic fragment thereof, and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing a neoplastic disease in a subject in need thereof, wherein the methods comprise administering to the subject (i) a first arenavirus particle, wherein the arenavirus particle comprises a genome that is engineered to comprise a nucleotide sequence encoding a tumor antigen, tumor associated antigen, or antigenic fragment thereof, and (ii) a second arenavirus particle, wherein the arenavirus particle comprises a genome that is engineered to comprise a nucleotide sequence encoding an antagonist of the NKG2A coinhibitory pathway.

In one aspect, provided herein is a method for treating or preventing a neoplastic disease in a subject in need thereof, wherein the method comprises administering to the subject (i) a first arenavirus particle; and (ii) a second arenavirus particle; wherein (a) the first arenavirus particle comprises a first arenavirus genome comprising: a first heterologous ORF encoding a tumor antigen, tumor associated antigen, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and (b) the second arenavirus particle comprises a second arenavirus genome comprising: a second heterologous ORF encoding an antagonist of the NKG2A coinhibitory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

Also provided herein are methods for treating or preventing an infectious disease in a subject in need thereof, wherein the methods comprise administering to the subject (i) a first arenavirus particle, wherein the arenavirus particle comprises a genome that is engineered to comprise a nucleotide sequence encoding a tumor antigen, tumor associated antigen, or antigenic fragment thereof, and (ii) a second arenavirus particle, wherein the arenavirus particle comprises a genome that is engineered to comprise a nucleotide sequence encoding an antagonist of the NKG2A coinhibitory pathway.

In a specific embodiment, provided herein is a method for treating or preventing an infectious disease in a subject in need thereof, wherein the method comprises administering to the subject (i) a first arenavirus particle; and (ii) a second arenavirus particle; wherein (a) the first arenavirus particle comprises a first arenavirus genome comprising: a first heterologous ORF encoding an antigen of a pathogen that causes the infectious disease, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and (b) the second arenavirus particle comprises a second arenavirus genome comprising: a second heterologous ORF encoding an antagonist of the NKG2A coinhibitory pathway; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

Also provided herein are methods for treating or preventing a neoplastic disease in a subject in need thereof, wherein the methods comprise administering to the subject an arenavirus particle, wherein the arenavirus particle comprises a genome that is engineered to comprise (i) a nucleotide sequence encoding a tumor antigen, tumor associated antigen, or antigenic fragment thereof, and (ii) a nucleotide sequence encoding a cytokine, such as IL-12.

In one aspect, provided herein is a method for treating or preventing a neoplastic disease in a subject in need thereof, wherein the method comprises administering to the subject an arenavirus particle; wherein (a) the arenavirus particle comprises an arenavirus genome comprising: (i) a first heterologous ORF encoding a tumor antigen, tumor associated antigen, or antigenic fragment thereof; and (ii) a second heterologous ORF encoding a cytokine, such as IL-12; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing an infectious disease in a subject in need thereof, wherein the methods comprise administering to the subject an arenavirus particle, wherein the arenavirus particle comprises a genome that is engineered to comprise (i) a nucleotide sequence encoding an antigen of a pathogen that causes the infectious disease, or antigenic fragment thereof, and (ii) a nucleotide sequence encoding a cytokine, such as IL-12.

In one aspect, provided herein is a method for treating or preventing an infectious disease in a subject in need thereof, wherein the method comprises administering to the subject an arenavirus particle; wherein (a) the arenavirus particle comprises an arenavirus genome comprising: (i) a first heterologous ORF encoding an antigen of a pathogen that causes the infectious disease, or antigenic fragment thereof; and (ii) a second heterologous ORF encoding a cytokine, such as IL-12; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

Also provided herein are methods for treating or preventing a neoplastic disease in a subject in need thereof, wherein the methods comprise administering to the subject (i) a first arenavirus particle, wherein the arenavirus particle comprises a genome that is engineered to comprise a nucleotide sequence encoding a tumor antigen, tumor associated antigen, or antigenic fragment thereof, and (ii) a second arenavirus particle, wherein the arenavirus particle comprises a genome that is engineered to comprise a nucleotide sequence encoding a cytokine, such as IL-12.

In one aspect, provided herein is a method for treating or preventing a neoplastic disease in a subject in need thereof, wherein the method comprises administering to the subject (i) a first arenavirus particle; and (ii) a second arenavirus particle; wherein (a) the first arenavirus particle comprises a first arenavirus genome comprising a first heterologous ORF encoding a tumor antigen, tumor associated antigen, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and (b) the second arenavirus particle comprises a second arenavirus genome comprising a second heterologous ORF encoding a cytokine, such as IL-12; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

Also provided herein are methods for treating or preventing an infectious disease in a subject in need thereof, wherein the methods comprise administering to the subject (i) a first arenavirus particle, wherein the arenavirus particle comprises a genome that is engineered to comprise a nucleotide sequence encoding an antigen of a pathogen that causes the infectious disease, or antigenic fragment thereof, and (ii) a second arenavirus particle, wherein the arenavirus particle comprises a genome that is engineered to comprise a nucleotide sequence encoding a cytokine, such as IL-12.

In one aspect, provided herein is a method for treating or preventing an infectious disease in a subject in need thereof, wherein the method comprises administering to the subject (i) a first arenavirus particle; and (ii) a second arenavirus particle; wherein (a) the first arenavirus particle comprises a first arenavirus genome comprising a first heterologous ORF encoding an antigen of a pathogen that causes the infectious disease, or antigenic fragment thereof; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and (b) the second arenavirus particle comprises a second arenavirus genome comprising a second heterologous ORF encoding a cytokine, such as IL-12; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

In one aspect, provided herein is a method for treating or preventing a neoplastic disease in a subject in need thereof, wherein the method comprises administering to the subject (i) an arenavirus particle; and (ii) a cytokine (e.g., cytokines disclosed in Section 5.9); wherein (a) the arenavirus particle comprises an arenavirus genome comprising a heterologous ORF encoding a tumor antigen, tumor associated antigen, or antigenic fragment thereof; and (b) (i) at least one arenavirus open reading frame (ORF) of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF. In certain embodiments, the cytokine is directly administered to a subject (preferably in the form of a pharmaceutical composition), and is not encoded by any arenavirus genome. In certain embodiments, the composition comprising the cytokine further comprises an antibody that specifically binds to the cytokine. In certain embodiments, the cytokine is IL-2. In certain embodiments, the composition comprising IL-2 further comprises an anti-IL-2 antibody. In certain embodiments, the cytokine is a fusion protein comprising IL-2 linked to an immunoglobulin, optionally the immunoglobulin is an anti-IL-2 antibody. In certain embodiments, the cytokine is a modified IL-2 that has abrogated binding to CD25. In certain embodiments, the IL-2 is selected from the group consisting of ANV419, XTX202, AB248, MDNA11, STK-012, and combinations thereof.

In one aspect, provided herein is a method for treating or preventing an infectious disease in a subject in need thereof, wherein the method comprises administering to the subject (i) an arenavirus particle; and (ii) a cytokine (e.g., cytokines disclosed in Section 5.9); wherein (a) the arenavirus particle comprises an arenavirus genome comprising a heterologous ORF encoding an antigen of a pathogen that causes the infectious disease, or antigenic fragment thereof; and (b) (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF. In certain embodiments, the cytokine is directly administered to a subject (preferably in the form of a pharmaceutical composition), and is not encoded by any arenavirus genome. In certain embodiments, the composition comprising the cytokine further comprises an antibody that specifically binds to the cytokine. In certain embodiments, the cytokine is IL-2. In certain embodiments, the composition comprising IL-2 further comprises an anti-IL-2 antibody. In certain embodiments, the cytokine is a fusion protein comprising IL-2 linked to an immunoglobulin, optionally the immunoglobulin is an anti-IL-2 antibody. In certain embodiments, the cytokine is a modified IL-2 that has abrogated binding to CD25. In certain embodiments, the IL-2 is selected from the group consisting of ANV419, XTX202, AB248, MDNA11, STK-012, and combinations thereof.

Also provided herein are methods for treating or preventing a neoplastic disease or treating or preventing an infectious disease in a subject in need thereof, wherein the methods comprise administering to the subject an arenavirus particle, wherein the arenavirus particle comprises a genome that is engineered to comprise a nucleotide sequence encoding an immune checkpoint modulator (e.g., an immune checkpoint modulator disclosed in Section 5.8, an agonist of the 4-1BB costimulatory pathway, an agonist of the OX40 costimulatory pathway, a ligand of 4-1BB, a ligand of OX40, or an antagonist of the NKG2A coinhibitory pathway). In certain embodiments, the arenavirus particle does not encode a tumor antigen, tumor associated antigen, an antigen of a pathogen that causes an infectious disease, or an antigenic fragment of any of the foregoing.

Also provided herein are methods for treating or preventing a neoplastic disease or treating or preventing an infectious disease in a subject in need thereof, wherein the methods comprise administering to the subject an arenavirus particle, wherein the arenavirus particle comprises a genome that is engineered to comprise a nucleotide sequence encoding a cytokine (e.g., a cytokine disclosed in Section 5.9, IL-12). In certain embodiments, the arenavirus particle does not encode a tumor antigen, tumor associated antigen, an antigen of a pathogen that causes an infectious disease, or an antigenic fragment of any of the foregoing.

In various embodiments, the agonist of the 4-1BB costimulatory pathway is an agonistic antibody or antigen-binding fragment thereof of 4-1BB. In various embodiments, the agonist of the 4-1BB costimulatory pathway is a ligand of 4-1BB.

In various embodiments, the agonist of the OX40 costimulatory pathway is an agonistic antibody or antigen-binding fragment thereof of OX40. In various embodiments, the agonist of the OX40 costimulatory pathway is a ligand of OX40.

In various embodiments, the antagonist of the NKG2A coinhibitory pathway is an antagonist antibody or antigen-binding fragment thereof of NKG2A. In various embodiments, the antagonist of the NKG2A coinhibitory pathway is a ligand of NKG2A.

In certain embodiments, the at least one arenavirus ORF described herein encodes the glycoprotein (“GP”), the nucleoprotein (“NP”), the matrix protein Z (“Z protein”) or the RNA dependent RNA polymerase L (“L protein”) of the arenavirus particle.

In certain embodiments, the at least one arenavirus ORF is either functionally inactivated or deleted and the arenavirus particle has the ability to amplify and express its genetic information in cells infected with the arenavirus particle but is unable to produce further infectious progeny particles in normal, non-complementing cells.

In certain embodiments wherein a method described herein comprises administering a first arenavirus particle and a second arenavirus particle as described herein, the at least one first arenavirus ORF encodes the glycoprotein (“GP”), the nucleoprotein (“NP”), the matrix protein Z (“Z protein”) or the RNA dependent RNA polymerase L (“L protein”) of the first arenavirus particle; and/or the at least one second arenavirus ORF encodes the glycoprotein (“GP”), the nucleoprotein (“NP”), the matrix protein Z (“Z protein”) or the RNA dependent RNA polymerase L (“L protein”) of the second arenavirus particle.

In certain embodiments wherein a method described herein comprises administering a first arenavirus particle and a second arenavirus particle as described herein, the at least one first arenavirus ORF is either functionally inactivated or deleted and the first arenavirus particle has the ability to amplify and express its genetic information in cells infected with the first arenavirus particle but is unable to produce further infectious progeny particles in normal, non-complementing cells; and/or the at least one second arenavirus ORF is either functionally inactivated or deleted and the second arenavirus particle has the ability to amplify and express its genetic information in cells infected with the second arenavirus particle but is unable to produce further infectious progeny particles in normal, non-complementing cells.

In specific embodiments, a method for treating or preventing a neoplastic disease described herein is a method for treating a neoplastic disease. In specific embodiments, a method for treating or preventing a neoplastic disease described herein is a method for preventing a neoplastic disease.

In specific embodiments, a method for treating or preventing an infectious disease described herein is a method for treating an infectious disease. In specific embodiments, a method for treating or preventing an infectious disease described herein is a method for preventing an infectious disease.

Neoplastic diseases and infectious diseases and their associated antigens are further described in Sections 5.1 and 5.2, respectively. Arenavirus particles that can be used in accordance with a method described herein are further described in Sections 5.4-5.6. Methods that can be used to generate arenavirus particles described herein are further described in Section 5.7. Immune checkpoint modulators are further described in Section 5.8. Cytokines are further described in Section 5.9. Additional non-limiting embodiments and disclosure regarding the use of an arenavirus particle involved in a combination therapy method described herein are provided in Section 5.10. The associated compositions, administration routes and dosages that can be used in accordance with a method described herein are further described in Section 5.11. Non-limiting exemplary assays that may be used to demonstrate efficacy of a combination therapy method described herein or activity of an ingredient used in the combination therapy are provided in Section 5.12.

Also provided herein is an arenavirus particle whose genome is constructed as described in Sections 5.4, 5.5, or 5.6 and comprises a nucleotide sequence encoding an immune checkpoint modulator (as described in Section 5.8) or a cytokine (as described in Section 5.9). Specifically, the immune checkpoint modulator can be an agonist of the 4-1BB costimulatory pathway (such as a 4-1BB ligand) or the OX40 costimulatory pathway. In specific embodiments, the genome of the arenavirus comprises two nucleotide sequences encoding two immune checkpoint modulators. The two immune checkpoint modulators can be the same or different. In a specific embodiment, the immune checkpoint modulator is a bispecific antibody. In another embodiment, the genome of the arenavirus particle comprises a nucleotide sequence encoding a cytokine, such as IL-12.

As used in this disclosure including the claims, the singular forms “a,” “an” and “the” include plural referents unless the context clearly dictates otherwise. The terms “a” (or “an”), as well as the terms “one or more,” and “at least one” can be used interchangeably herein unless the context clearly dictates otherwise.

### 5.1 Neoplastic Diseases, Tumor Antigens, Tumor Associated Antigens, and Antigenic Fragments Thereof

Neoplastic diseases that can be treated or prevented with the methods and compositions described herein include acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; Hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor. In a specific embodiment, the neoplastic diseases that can be treated or prevented with the methods and compositions described herein is a solid tumor. As such, the tumor antigen, tumor associated antigen or antigenic fragment thereof that is encoded by the genome of an arenaviral particle described herein is associated with or specific to one of these neoplastic diseases disclosed herein.

In certain embodiments, arenavirus particles with a nucleotide sequence encoding a tumor antigen, tumor associated antigen or an antigenic fragment thereof provided herein can be used with the methods and compositions provided herein. In certain embodiments, arenavirus particles with a nucleotide sequence encoding a tumor antigen, tumor associated antigen or an antigenic fragment thereof provided herein can be used with the methods and compositions provided herein in combination with arenavirus particles not encoding a foreign antigen. In certain embodiments, a tumor antigen or tumor associated antigen for use with the methods and compositions described herein is an immunogenic protein expressed in or on a neoplastic cell or tumor, such as a cancer cell or malignant tumor. In certain embodiments, a tumor antigen or tumor associated antigen for use with the methods and compositions described herein is a non-specific, mutant, overexpressed or abnormally expressed protein, which can be present on both a neoplastic cell or tumor and a normal cell or tissue. In certain embodiments, a tumor antigen or tumor associated antigen for use with the methods and compositions described herein is a tumor-specific antigen which is restricted to tumor cells. In certain embodiments, a tumor antigen for use with the methods and compositions described herein is a cancer-specific antigen which is restricted to cancer cells.

In certain embodiments, a tumor antigen or tumor associated antigen can exhibit one, two, three, or more, including all, of the following characteristics: overexpressed/accumulated (i.e., expressed by both normal and neoplastic tissue, but highly expressed in neoplasia), oncofetal (i.e., usually only expressed in fetal tissues and in cancerous somatic cells), oncoviral or oncogenic viral (i.e., encoded by tumorigenic transforming viruses), cancer-testis (i.e., expressed only by cancer cells and adult reproductive tissues, e.g., the testis), lineage-restricted (i.e., expressed largely by a single cancer histotype), mutated (i.e., only expressed in neoplastic tissue as a result of genetic mutation or alteration in transcription), post-translationally altered (e.g., tumor-associated alterations in glycosylation), or idiotypic (i.e., developed from malignant clonal expansions of B or T lymphocytes).

In certain embodiments, the fragment of the tumor antigen or tumor associated antigen is antigenic when it is capable of (i) eliciting an antibody immune response in a host (e.g., mouse, rabbit, goat, donkey or human) wherein the resulting antibodies bind specifically to an immunogenic protein expressed in or on a neoplastic cell (e.g., a cancer cell); and/or (ii) eliciting a specific T cell immune response.

In certain embodiments, the nucleotide sequence encoding an antigenic fragment of a tumor antigen or tumor associated antigen is 8 to 100 nucleotides, 15 to 100 nucleotides, 25 to 100 nucleotides, 50 to 200 nucleotides, 50 to 400 nucleotides, 200 to 500 nucleotides, or 400 to 600 nucleotides, or 500 to 800 nucleotides in length. In other embodiments, the nucleotide sequence encoding an antigenic fragment of a tumor antigen or tumor associated antigen is 750 to 900 nucleotides, 800 to 1000 nucleotides, 850 to 1000 nucleotides, 900 to 1200 nucleotides, 1000 to 1200 nucleotides, 1000 to 1500 nucleotides, 1500 to 2000 nucleotides, 1700 to 2000 nucleotides, 2000 to 2300 nucleotides, 2200 to 2500 nucleotides, 2500 to 3000 nucleotides, 3000 to 3200 nucleotides, 3000 to 3500 nucleotides, 3200 to 3600 nucleotides, 3300 to 3800 nucleotides, 4000 nucleotides to 4400 nucleotides, 4200 to 4700 nucleotides, 4800 to 5000 nucleotides, 5000 to 5200 nucleotides, 5200 to 5500 nucleotides, 5500 to 5800 nucleotides, 5800 to 6000 nucleotides, 6000 to 6400 nucleotides, 6200 to 6800 nucleotides, 6600 to 7000 nucleotides, 7000 to 7200 nucleotides, 7200 to 7500 nucleotides, or more than 7500 nucleotides in length. In some embodiments, the nucleotide sequence encoding an antigenic fragment of a tumor antigen or tumor associated antigen encodes a peptide or polypeptide that is 5 to 10 amino acids, 10 to 25 amino acids, 25 to 50 amino acids, 50 to 100 amino acids, 100 to 150 amino acids, 150 to 200 amino acids, 200 to 250 amino acids, 250 to 300 amino acids, 300 to 400 amino acids, 400 to 500 amino acids, 500 to 750 amino acids, 750 to 1000 amino acids, 1000 to 1250 amino acids, 1250 to 1500 amino acids, 1500 to 1750 amino acids, 1750 to 2000 amino acids, 2000 to 2500 amino acids, or more than 2500 amino acids in length. In some embodiments, the nucleotide sequence encodes a polypeptide that does not exceed 2500 amino acids in length. In specific embodiments the nucleotide sequence does not contain a stop codon. In certain embodiments, the nucleotide sequence is codon-optimized. In certain embodiments the nucleotide composition, nucleotide pair composition or both can be optimized. Techniques for such optimizations are known in the art and can be applied to optimize a nucleotide sequence of a tumor antigen, tumor associated antigen, or an antigenic fragment thereof.

In certain embodiments, the tumor antigen or tumor associated antigen for use with the methods and compositions disclosed herein is selected from the group consisting of oncogenic viral antigens, cancer-testis antigens, oncofetal antigens, tissue differentiation antigens, mutant protein antigens, Adipophilin, AIM-2, ALDH1AI, BCLX (L), BING-4, CALCA, CD45, CPSF, cyclin D1, DKKI, ENAH (hMcna), Ga733 (EpCAM), EphA3, EZH2, FGF5, glypican-3, G250/MN/CAIX, HER-2/neu, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-1, RGS5, RhoC, RNF43, RU2AS, secernin 1, SOX1O, STEAP1 (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferaseAS fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSX1 or -SSX2 fusion protein, TGF-betaRII, Triosephosphate isomerase, ormdm-2, LMP2, HPV E6, HPV E7, HPV E7/E6 fusion protein, EGFRvIII (epidermal growth factor variant III), Idiotype, GD2, ganglioside G2), Ras-mutant, p53 (mutant), Proteinase3 (PR1), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NA17, PAX3, ALK, Androgen Receptor, Cyclin B1, Polysialic acid, MYCN, TRP2, TRP2-Int2, GD3, Fucosyl GM1, Mesothelin, PSCA, sLe(a), cyp1B1, PLAC1, GM3, BORIS, Tn, GLoboH, NY-BR-1, SART3, STn, Carbonic Anhydrase IX, OY-TES1, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-1, FAP, PDGFR-beta, MAD-CT-2, For-related antigen 1, TRP-1, GP100, CA-125, CA19-9, Calretinin, Epithelial membrane antigen (EMA), Epithelial tumor antigen (ETA), CD19, CD34, CD99, CD117, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuron-specific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-1, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXB1, SPA17, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-A11, HSP70-2, KIAAO205, MUM-1, MUM-2, MUM-3, Myosin class I, GnTV, Herv-K-mel, LAGE-1, LAGE-2, (sperm protein) SP17, SCP-1, P15(58), Hom/Mel-40, E2A-PRL, H4-RET, IGH-IGK, MYL-RAR, TSP-180, P185erbB2, pi80erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68KP1, CO-029, HTgp-175, M344, MG7-Ag, MOV18, NB70K, NY-CO-1, RCAS1, SDCCAGi6, TA-90, TAAL6, TLP, TPS, CD22, CD27, CD30, CD70, prostein, TARP (T cell receptor gamma alternate reading frame protein), Trp-p8, integrin avP3 (CD61), galactin, or Ral-B, CD123, CLL-1, CD38, CS-1, CD138, and ROR1.

In certain embodiments, the tumor antigen or tumor associated antigen is a neoantigen. A “neoantigen,” as used herein, means an antigen that arises by mutation in a tumor cell and such an antigen is not generally expressed in normal cells or tissue. Without being bound by theory, because healthy tissues generally do not possess these antigens, neoantigens represent a preferred target. Additionally, without being bound by theory, in the context of the present invention, since the T cells that recognize the neoantigen may not have undergone negative thymic selection, such cells can have high avidity to the antigen and mount a strong immune response against tumors, while lacking the risk to induce destruction of normal tissue and autoimmune damage. In certain embodiments, the neoantigen is an MHC class I-restricted neoantigen. In certain embodiments, the neoantigen is an MHC class II-restricted neoantigen. In certain embodiments, a mutation in a tumor cell of the patient results in a novel protein that produces the neoantigen.

In certain embodiments, the tumor antigen or tumor associated antigen can be an antigen ortholog, e.g., a mammalian (i.e., non-human primate, pig, dog, cat, or horse) tumor antigen or tumor associated antigen.

In certain embodiments, an antigenic fragment of a tumor antigen or tumor associated antigen described herein is encoded by the nucleotide sequence included within the arenavirus genome. In certain embodiments, a fragment is antigenic when it is capable of (i) eliciting an antibody immune response in a host (e.g., mouse, rabbit, goat, donkey or human) wherein the resulting antibodies bind specifically to an immunogenic protein expressed in or on a neoplastic cell (e.g., a cancer cell); and/or (ii) eliciting a specific T cell immune response.

In certain embodiments, the arenavirus genomic segment, the arenavirus particle or the tri-segmented arenavirus particle can comprise one or more nucleotide sequences encoding tumor antigens, tumor associated antigens, or antigenic fragments thereof. In other embodiments, the arenavirus genomic segment, the arenavirus particle or the tri-segmented arenavirus particle can comprise at least one nucleotide sequence encoding a tumor antigen, tumor associated antigen, or antigenic fragment thereof, at least two nucleotide sequences encoding tumor antigens, tumor associated antigens, or antigenic fragments thereof, at least three nucleotide sequences encoding tumor antigens, tumor associated antigens, or antigenic fragments thereof, or more nucleotide sequences encoding tumor antigens, tumor associated antigens, or antigenic fragments thereof.

In certain embodiments, an arenavirus particle comprising a nucleotide sequence encoding a tumor antigen, tumor associated antigen or antigenic fragment thereof as provided herein, which either is administered in combination with an immune checkpoint modulator or a cytokine, or comprises a nucleotide sequence encoding an immune checkpoint modulator or a cytokine, further comprises at least one nucleotide sequence encoding at least one polypeptide or protein. In preferred embodiments, the at least one polypeptide or protein is not antigenic but is capable of enhancing antigenicity of the tumor antigen, tumor associated antigen or antigenic fragment thereof. In certain embodiments, the polypeptide or protein is Calreticulin (CRT), or a fragment thereof, Ubiquitin or a fragment thereof, Granulocyte-Macrophage Colony-Stimulating Factor (GM-CSF), or a fragment thereof, Invariant chain (CD74) or a fragment thereof; Mycobacterium tuberculosis Heat shock protein 70 or a fragment thereof, Herpes simplex virus 1 protein VP22 or a fragment thereof, CD40 ligand or a fragment thereof, or Fms-related tyrosine kinase 3 (Flt3) ligand or a fragment thereof. In certain embodiments wherein the arenavirus particle comprising a nucleotide sequence encoding a tumor antigen, tumor associated antigen or antigenic fragment thereof as provided herein is administered in combination with an immune checkpoint modulator, or comprises a nucleotide sequence encoding an immune checkpoint modulator, the at least one polypeptide or protein is a cytokine or a different immune checkpoint modulator. In certain embodiments wherein the arenavirus particle comprising a nucleotide sequence encoding a tumor antigen, tumor associated antigen or antigenic fragment thereof as provided herein is administered in combination with a cytokine, or comprises a nucleotide sequence encoding a cytokine, the at least one polypeptide or protein is an immune checkpoint modulator or a different cytokine. An immune checkpoint modulator can be an agonist of a costimulatory pathway or an antagonist of a coinhibitory pathway, and can be one as described in Section 5.8. A cytokine can be one described in Section 5.9, for example, IL-2, IL-7, IL-12, IL-15, IL-15/IL-15Ra, IL-15/IL-15Ra sushi domain (e.g., ALT-803, which is an IL-15/IL-15Ra sushi domain fusion protein with an additional mutation (N72D)), IL-21, or IL-33, or a variant (e.g., an engineered/modified form) of any of the forgoing.

In certain embodiments, an arenavirus particle provided herein comprises a genomic segment that a) has at least one arenavirus ORF located in a position other than the wild-type position of said at least one arenavirus ORF; and b) encodes (either in sense or antisense): (i) one or more tumor antigens, tumor associated antigens or antigenic fragments thereof provided herein, and (ii) one or more immune checkpoint modulators and/or cytokines provided herein.

In certain embodiments, the nucleotide sequence encoding the tumor antigen, tumor associated antigen or an antigenic fragment thereof provided herein, and the nucleotide sequence encoding the immune checkpoint modulator and/or cytokine provided herein, are on the same segment of the viral genome. In certain embodiments, the nucleotide sequence encoding the tumor antigen, tumor associated antigen or an antigenic fragment thereof provided herein, and the nucleotide sequence encoding the immune checkpoint modulator and/or cytokine provided herein, are on different segments of the viral genome.

In certain embodiments, the nucleotide sequence encoding the tumor antigen, tumor associated antigen or an antigenic fragment thereof provided herein, and the nucleotide sequence encoding the immune checkpoint modulator and/or cytokine provided herein, are separated via a spacer sequence. In certain embodiments, the sequence encoding the tumor antigen, tumor associated antigen or an antigenic fragment thereof provided herein, and the nucleotide sequence encoding the immune checkpoint modulator and/or cytokine provided herein, are separated by an internal ribosome entry site, or a sequence encoding a protease cleavage site. In certain embodiments, the nucleotide sequence encoding the tumor antigen, tumor associated antigen or an antigenic fragment thereof provided herein, and the nucleotide sequence encoding the immune checkpoint modulator and/or cytokine provided herein, are separated by a nucleotide sequence encoding a linker or a self-cleaving peptide. Any linker peptide or self-cleaving peptide known to the skilled artisan can be used with the compositions and methods provided herein. A non-limiting example of a peptide linker is GSG. Non-limiting examples of a self-cleaving peptide are Porcine teschovirus-1 2A peptide, Thoseaasignavirus 2A peptide, or Foot-and-mouth disease virus 2A peptide.

In certain embodiments, the tumor antigen, tumor associated antigen or an antigenic fragment thereof provided herein, and the immune checkpoint modulator and/or cytokine provided herein, are directly fused together. In certain embodiments, the tumor antigen, tumor associated antigen or an antigenic fragment thereof provided herein, and the immune checkpoint modulator and/or cytokine provided herein, are fused together via a peptide linker. In certain embodiments, the tumor antigen, tumor associated antigen or an antigenic fragment thereof provided herein, and the immune checkpoint modulator and/or cytokine provided herein are separated from each other via a self-cleaving peptide. A non-limiting example of a peptide linker is GSG. Non-limiting examples of a self-cleaving peptide are Porcine teschovirus-1 2A peptide, Thoseaasignavirus 2A peptide, or Foot-and-mouth disease virus 2A peptide.

In certain embodiments, the tumor antigen, tumor associated antigen or an antigenic fragment thereof provided herein, and the immune checkpoint modulator and/or cytokine provided herein are expressed on the same arenavirus particle. In certain embodiments, the tumor antigen, tumor associated antigen or an antigenic fragment thereof provided herein, and the immune checkpoint modulator and/or cytokine provided herein are expressed on different arenavirus particles. In certain embodiments, the tumor antigen, tumor associated antigen or an antigenic fragment thereof provided herein, and the immune checkpoint modulator and/or cytokine provided herein are expressed on different particles derived from the same arenavirus strain. In certain embodiments, the tumor antigen, tumor associated antigen or an antigenic fragment thereof provided herein, and the immune checkpoint modulator and/or cytokine provided herein are expressed on different particles derived from different arenavirus strains.

In certain embodiments, an arenavirus particle engineered to encode one or more tumor antigens, tumor associated antigens or antigenic fragments thereof comprises one or more nucleotide sequences encoding tumor antigens, tumor associated antigens or antigenic fragments thereof provided herein. In specific embodiments the tumor antigens, tumor associated antigens or antigenic fragments thereof provided herein are separated by various one or more linkers, spacers, or cleavage sites as described herein.

### 5.2 Infectious Diseases And Antigens of Pathogens

In certain embodiments, the infectious disease that can be treated or prevented with the methods and compositions described herein is a chronic infectious disease. In certain embodiments, the infectious disease that can be treated or prevented with the methods and compositions described herein is an acute infectious disease.

In certain embodiments, the pathogen is a bacterium, virus, fungus, parasite, helminth or protist. In specific embodiments, the pathogen is a bacterium. In specific embodiments, the pathogen is a virus. In specific embodiments, the pathogen is HIV-1, HIV-2, HBV, HCV, HPV, CMV, HSV-1, HSV-2, EBV, Plasmodium falciparum, Mycobacterium tuberculosis, JC virus, HHV-6, HHV-7, HTLV-1, HTLV-2, VZV, Measles virus, or coronavirus. In specific embodiments, the pathogen is enterovirus, poliovirus, West Nile virus, Anaplasma phagocytophilum, Bacillus anthracis, Babesia microti, Brucella, Campylobacter, Enterobacterale, Haemophilus ducreyi, chikungunya virus, Chlamydia trachomatis, Clostridium difficile, coccidioides, SARS-CoV-2, Cryptosporidium, Cyclospora, Dengue virus, Corynebacterium diphtheriae, E. coli, Eastern equine encephalitis virus, Ebola virus, Ehrlichia chaffeensis, E. ewingii, E. muris eauclairensis, arbovirus, enterovirus, Giardia duodenalis, Burkholderia mallei, Neisseria gonorrhoeae, Klebsiella granulomatis, Type B Haemophilus influenzae, hantavirus, Escherichia coli O157:H7, hepatitis A virus, hepatitis B virus, hepatitis C virus, hepatitis D virus, hepatitis E virus, herpes simplex virus, varicella-zoster virus, Histoplasma, human immunodeficiency virus, human papillomavirus, influenza virus, Legionella, Mycobacterium leprae, Leptospira, Listeria monocytogenes, Borrelia burgdorferi, Borrelia mayonii, Chlamydia trachomatis, Plasmodium falciparum, P. vivax, P. ovale, P. malariae, P. knowlesi, measles virus, Burkholderia pseudomallei, mumps virus, rubella virus, MERS-CoV, norovirus, louse, Bordetella pertussis, Yersinia pestis, Streptococcus pneumoniae, polio virus, powassan virus, Chlamydia psittaci, variola virus, monkeypox virus, cowpox virus, Coxiella burnetii, rabies virus, R. parkeri, Salmonella, Sarcoptes scabiei var. hominis, SARS-CoV, Shigella, Staphylococcus, Streptococcus, Treponema pallidum, Clostridium tetani, Trichomonas vaginalis, Trichinella, Mycobacterium tuberculosis, Francisella tularensis, Salmonella typhi, Rickettsia prowazekii, varicella-zoster virus, Vibrio cholerae, vibriosis, Marburg virus, Lassa virus, West Nile virus, coronavirus, yeast, yellow fever virus, Yersinia enterocolitica, or zika virus.

In certain embodiments, the infectious diseases that can be treated or prevented with the methods and compositions described herein include acute flaccid myelitis, anaplasmosis, anthrax, babesiosis, brucellosis, campylobacteriosis, carbapenem-resistant infection, chancroid, chikungunya virus infection, chlamydia, Clostridium difficile infection, coccidioidomycosis fungal infection, Covid-19, cryptosporidiosis, cyclosporiasis, dengue fever, diphtheria, E. coli infection, eastern equine encephalitis, Ebola hemorrhagic fever, ehrlichiosis, arboviral encephalitis, parainfectious encephalitis, enterovirus infection, giardiasis, glanders, gonococcal infection, granuloma inguinale, type b haemophilus influenza disease, hantavirus pulmonary syndrome, hemolytic uremic syndrome, hepatitis A, hepatitis B, hepatitis C, hepatitis D, hepatitis E, herpes, herpes zoster, histoplasmosis infection, acquired immunodeficiency syndrome, human papillomavirus infection, influenza, legionellosis, leprosy, leptospirosis, listeriosis, lyme disease, lymphogranuloma venereum infection, malaria, measles, melioidosis, viral meningitis, viral meningitis, middle east respiratory syndrome, multisystem inflammatory syndrome, mumps, norovirus infection, pediculosis, pelvic inflammatory disease, pertussis, plague, pneumococcal disease, poliomyelitis, powassan virus infection, psittacosis, pthiriasis, pustular rash disease, Q-fever, rabies, rickettsiosis, rubella, salmonellosis gastroenteritis, scabies infestation, sepsis, severe acute respiratory syndrome, shigellosis gastroenteritis, smallpox, staphyloccal infection, staphylococcal infection, streptococcal disease, streptococcal toxic-shock syndrome, syphilis, tetanus infection, trichomoniasis, trichonosis infection, tuberculosis, tularemia, typhoid fever, typhus, varicella, Vibrio cholera, vibriosis, viral hemorrhagic fever, west nile virus infection, coronavirus infection, yeast infection, yellow fever, yersenia, and zika virus infection. As such, the antigen of a pathogen that causes an infectious disease or antigenic fragment thereof that is encoded by the genome of an arenaviral particle described herein is associated with or can be specific to one of the infectious diseases disclosed herein.

In certain embodiments, arenavirus particles with nucleotide sequence encoding an antigen of a pathogen that causes an infectious disease or an antigenic fragment thereof provided herein can be used with the methods and compositions provided herein. In certain embodiments, arenavirus particles with a nucleotide sequence encoding an antigen of a pathogen that causes an infectious disease or an antigenic fragment thereof provided herein can be used with the methods and compositions provided herein in combination with arenavirus particles not encoding a foreign antigen. In certain embodiments, an antigen of a pathogen that causes an infectious disease for use with the methods and compositions described herein is an immunogenic protein expressed in, on, or by the pathogen.

In certain embodiments, the fragment of the antigen of a pathogen that causes an infectious disease is antigenic when it is capable of (i) eliciting an antibody immune response in a host (e.g., mouse, rabbit, goat, donkey or human) wherein the resulting antibodies bind specifically to an immunogenic protein expressed in, on or by the pathogen; and/or (ii) eliciting a specific T cell immune response.

In certain embodiments, the nucleotide sequence encoding an antigenic fragment of an antigen of a pathogen that causes an infectious disease is 8 to 100 nucleotides, 15 to 100 nucleotides, 25 to 100 nucleotides, 50 to 200 nucleotides, 50 to 400 nucleotides, 200 to 500 nucleotides, 400 to 600 nucleotides, or 500 to 800 nucleotides in length. In other embodiments, the nucleotide sequence encoding an antigenic fragment of an antigen of a pathogen that causes an infectious disease is 750 to 900 nucleotides, 800 to 1000 nucleotides, 850 to 1000 nucleotides, 900 to 1200 nucleotides, 1000 to 1200 nucleotides, 1000 to 1500 nucleotides, 1500 to 2000 nucleotides, 1700 to 2000 nucleotides, 2000 to 2300 nucleotides, 2200 to 2500 nucleotides, 2500 to 3000 nucleotides, 3000 to 3200 nucleotides, 3000 to 3500 nucleotides, 3200 to 3600 nucleotides, 3300 to 3800 nucleotides, 4000 nucleotides to 4400 nucleotides, 4200 to 4700 nucleotides, 4800 to 5000 nucleotides, 5000 to 5200 nucleotides, 5200 to 5500 nucleotides, 5500 to 5800 nucleotides, 5800 to 6000 nucleotides, 6000 to 6400 nucleotides, 6200 to 6800 nucleotides, 6600 to 7000 nucleotides, 7000 to 7200 nucleotides, 7200 to 7500 nucleotides, or more than 7500 nucleotides in length. In some embodiments, the nucleotide sequence encoding an antigenic fragment of an antigen of a pathogen that causes an infectious disease encodes a peptide or polypeptide that is 5 to 10 amino acids, 10 to 25 amino acids, 25 to 50 amino acids, 50 to 100 amino acids, 100 to 150 amino acids, 150 to 200 amino acids, 200 to 250 amino acids, 250 to 300 amino acids, 300 to 400 amino acids, 400 to 500 amino acids, 500 to 750 amino acids, 750 to 1000 amino acids, 1000 to 1250 amino acids, 1250 to 1500 amino acids, 1500 to 1750 amino acids, 1750 to 2000 amino acids, 2000 to 2500 amino acids, or more than 2500 amino acids in length. In some embodiments, the nucleotide sequence encodes a polypeptide that does not exceed 2500 amino acids in length. In specific embodiments the nucleotide sequence does not contain a stop codon. In certain embodiments, the nucleotide sequence is codon-optimized. In certain embodiments the nucleotide composition, nucleotide pair composition or both can be optimized. Techniques for such optimizations are known in the art and can be applied to optimize a nucleotide sequence of an antigen of a pathogen that causes an infectious disease, or an antigenic fragment thereof.

In certain embodiments, an antigenic fragment of an antigen of a pathogen that causes an infectious disease antigen described herein is encoded by the nucleotide sequence included within the arenavirus genome. In certain embodiments, a fragment is antigenic when it is capable of (i) eliciting an antibody immune response in a host (e.g., mouse, rabbit, goat, donkey or human) wherein the resulting antibodies bind specifically to an immunogenic protein expressed in, on or by the pathogen; and/or (ii) eliciting a specific T cell immune response.

In certain embodiments, the arenavirus genomic segment, the arenavirus particle or the tri-segmented arenavirus particle can comprise one or more nucleotide sequences encoding antigens of a pathogen that causes an infectious disease, or antigenic fragments thereof. In other embodiments, the arenavirus genomic segment, the arenavirus particle or the tri-segmented arenavirus particle can comprise at least one nucleotide sequence encoding an antigen of a pathogen that causes an infectious disease, or antigenic fragment thereof, at least two nucleotide sequences encoding antigens of a pathogen that causes an infectious disease, or antigenic fragments thereof, at least three nucleotide sequences encoding antigens of a pathogen that causes an infectious disease, or antigenic fragments thereof, or more nucleotide sequences encoding antigens of a pathogen that causes an infectious disease, or antigenic fragments thereof.

In certain embodiments, an arenavirus particle comprising a nucleotide sequence encoding an antigen of a pathogen that causes an infectious disease or antigenic fragment thereof as provided herein, which either is administered in combination with an immune checkpoint modulator or a cytokine, or comprises a nucleotide sequence encoding an immune checkpoint modulator or a cytokine, further comprises at least one nucleotide sequence encoding at least one polypeptide or protein. In preferred embodiments, the at least one polypeptide or protein is not antigenic but is capable of enhancing antigenicity of the antigen of a pathogen that causes an infectious disease or antigenic fragment thereof. In certain embodiments, the polypeptide or protein is Calreticulin (CRT), or a fragment thereof, Ubiquitin or a fragment thereof; Granulocyte-Macrophage Colony-Stimulating Factor (GM-CSF), or a fragment thereof; Invariant chain (CD74) or a fragment thereof; Mycobacterium tuberculosis Heat shock protein 70 or an fragment thereof, Herpes simplex virus 1 protein VP22 or fragment thereof, or Fms-related tyrosine kinase 3 (Flt3) ligand or a fragment thereof. In certain embodiments wherein the arenavirus particle comprising a nucleotide sequence encoding an antigen of a pathogen that causes an infectious disease or antigenic fragment thereof as provided herein is administered in combination with an immune checkpoint modulator, or comprises a nucleotide sequence encoding an immune checkpoint modulator, the at least one polypeptide or protein is a cytokine or a different immune checkpoint modulator. In certain embodiments wherein the arenavirus particle comprising a nucleotide sequence encoding an antigen of a pathogen that causes an infectious disease or antigenic fragment thereof as provided herein is administered in combination with a cytokine, or comprises a nucleotide sequence encoding a cytokine, the at least one polypeptide or protein is an immune checkpoint modulator or a different cytokine. An immune checkpoint modulator can be an agonist of a costimulatory pathway or an antagonist of a coinhibitory pathway, and can be one as described in Section 5.8. A cytokine can be one as described in Section 5.9, for example, IL-2, IL-7, IL-12, IL-15, IL-15/IL-15Ra, IL-15/IL-15Ra sushi domain (e.g., ALT-803, which is an IL-15/IL-15Ra sushi domain fusion protein with an additional mutation (N72D)), IL-21, or IL-33, or a variant (e.g., an engineered/modified form) of any of the forgoing.

In certain embodiments, an arenavirus particle provided herein comprises a genomic segment that a) has at least one arenavirus ORF located in a position other than the wild-type position of said at least one arenavirus ORF; and b) encodes (either in sense or antisense): (i) one or more antigens of a pathogen that causes an infectious disease or an antigenic fragment thereof provided herein, and (ii) one or more immune checkpoint modulators and/or cytokines provided herein.

In certain embodiments, the nucleotide sequence encoding the antigen of a pathogen that causes an infectious disease or an antigenic fragment thereof provided herein, and the nucleotide sequence encoding the immune checkpoint modulator and/or cytokine provided herein, are on the same segment of the viral genome. In certain embodiments, the nucleotide sequence encoding the antigen of a pathogen that causes an infectious disease or an antigenic fragment thereof provided herein, and the nucleotide sequence encoding the immune checkpoint modulator and/or cytokine provided herein, are on different segments of the viral genome.

In certain embodiments, the nucleotide sequence encoding the antigen of a pathogen that causes an infectious disease or an antigenic fragment thereof provided herein, and the nucleotide sequence encoding the immune checkpoint modulator and/or cytokine provided herein, are separated via a spacer sequence. In certain embodiments, the sequence encoding the antigen of a pathogen that causes an infectious disease or an antigenic fragment thereof provided herein, and the nucleotide sequence encoding the immune checkpoint modulator and/or cytokine provided herein, are separated by an internal ribosome entry site, or a sequence encoding a protease cleavage site. In certain embodiments, the nucleotide sequence encoding the antigen of a pathogen that causes an infectious disease or an antigenic fragment thereof provided herein, and the nucleotide sequence encoding the immune checkpoint modulator and/or cytokine provided herein, are separated by a nucleotide sequence encoding a linker or a self-cleaving peptide. Any linker peptide or self-cleaving peptide known to the skilled artisan can be used with the compositions and methods provided herein. A non-limiting example of a peptide linker is GSG. Non-limiting examples of a self-cleaving peptide are Porcine teschovirus-1 2A peptide, Thoseaasignavirus 2A peptide, or Foot-and-mouth disease virus 2A peptide.

In certain embodiments, the antigen of a pathogen that causes an infectious disease or an antigenic fragment thereof provided herein, and the immune checkpoint modulator and/or cytokine provided herein, are directly fused together. In certain embodiments, the antigen of a pathogen that causes an infectious disease or an antigenic fragment thereof provided herein, and the immune checkpoint modulator and/or cytokine provided herein, are fused together via a peptide linker. In certain embodiments, the antigen of a pathogen that causes an infectious disease or an antigenic fragment thereof provided herein, and the immune checkpoint modulator and/or cytokine provided herein are separated from each other via a self-cleaving peptide. A non-limiting example of a peptide linker is GSG. Non-limiting examples of a self-cleaving peptide are Porcine teschovirus-1 2A peptide, Thoseaasignavirus 2A peptide, or Foot-and-mouth disease virus 2A peptide.

In certain embodiments, the antigen of a pathogen that causes an infectious disease or an antigenic fragment thereof provided herein, and the immune checkpoint modulator and/or cytokine provided herein are expressed on the same arenavirus particle. In certain embodiments, the antigen of a pathogen that causes an infectious disease or an antigenic fragment thereof provided herein, and the immune checkpoint modulator and/or cytokine provided herein are expressed on different arenavirus particles. In certain embodiments, the antigen of a pathogen that causes an infectious disease or an antigenic fragment thereof provided herein, and the immune checkpoint modulator and/or cytokine provided herein are expressed on different particles derived from the same arenavirus strain. In certain embodiments, the antigen of a pathogen that causes an infectious disease or an antigenic fragment thereof provided herein, and the immune checkpoint modulator and/or cytokine provided herein are expressed on different particles derived from different arenavirus strains.

In certain embodiments, an arenavirus particle engineered to encode one or more antigens of a pathogen that causes an infectious disease or antigenic fragments thereof comprises one or more nucleotide sequences encoding antigens of a pathogen that causes an infectious disease or antigenic fragments thereof provided herein. In specific embodiments the antigens of a pathogen that causes an infectious disease or antigenic fragments thereof provided herein are separated by various one or more linkers, spacers, or cleavage sites as described herein.

### 5.3 Arenavirus Constructs

In certain embodiments, the arenavirus particles that can be engineered for the methods and compositions described herein include the constructs listed below. In certain embodiments, the arenavirus construct is a non-replicating arenavirus construct as described in International Patent Application Publication No. WO2009/083210 (which is incorporated herein in its entirety). In certain embodiments, the arenavirus construct is a replicating or a non-replicating tri-segmented arenavirus construct as described in International Patent Application Publication Nos. WO2016/075250 and WO2021/089853 (both of which are incorporated herein in their entireties).

Arenaviruses for use with the methods and compositions provided herein can be Old World viruses such as, for example, Lassa virus, Lymphocytic choriomeningitis virus (LCMV), Mobala virus, Mopeia virus, or Ippy virus, or New World viruses such as, for example, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, Allpahuayo virus (ALLV), or Whitewater Arroyo virus. Arenaviruses for use with the methods and compositions provided herein can be, for example, arenaviruses, mammarenaviruses, Old World mammarenaviruses, New World mammarenaviruses, New World mammarenaviruses of Clade A, New World mammarenaviruses of Clade B, New World mammarenaviruses of Clade C, or New World mammarenaviruses of Clade D. Arenaviruses for use with the methods and compositions provided herein can be a mammarenavirus including, but not limited to, Allpahuayo virus, Alxa virus, Junin virus, Bear Canyon virus, Sabia virus, Pichinde virus, Chapare virus, Lijiang virus, Cupixi virus, Flexal virus, Gairo virus, Guanarito virus, Ippy virus, Lassa virus, Latino virus, Loei River virus, Lujo virus, Luna virus, Luli virus, Lunk virus, lymphocytic choriomeningitis virus, Machupo virus, Mariental virus, Merino Walk virus, Mobala virus, Mopeia virus, Morogoro virus, Okahandja virus, Oliveros virus, Parana virus, Pirital virus, Apore virus, Ryukyu virus, Amapari virus, Solwezi virus, souris virus, Tacaribe virus, Tamiami virus, Wenzhou virus, Whitewater Arroyo virus, Big Brushy Tank virus, Catarina virus, Skinner Tank virus, Tonto Creek virus, or Xapuri virus. In certain embodiments, the arenavirus for use with the methods and compositions provided herein is lymphocytic choriomeningitis virus (LCMV). In certain embodiments, the arenavirus for use with the methods and compositions provided herein is Pichinde virus.

In certain embodiments, an arenavirus particle for use with a method described herein is derived from lymphocytic choriomeningitis virus (LCMV). In certain embodiments, an arenavirus particle for use with a method described herein is derived from Pichinde virus. In certain embodiments wherein a method described herein comprises administering a first arenavirus particle and a second arenavirus particle as described herein, the first arenavirus particle, the second arenavirus particle, or both the first and second arenavirus particles are derived from lymphocytic choriomeningitis virus (LCMV) or Pichinde virus. In certain embodiments wherein a method described herein comprises administering a first arenavirus particle and a second arenavirus particle as described herein, both the first and second arenavirus particles are derived from lymphocytic choriomeningitis virus (LCMV). In certain embodiments wherein a method described herein comprises administering a first arenavirus particle and a second arenavirus particle as described herein, both the first and second arenavirus particles are derived from Pichinde virus. In certain embodiments wherein a method described herein comprises administering a first arenavirus particle and a second arenavirus particle as described herein, the first arenavirus particle is derived from lymphocytic choriomeningitis virus (LCMV), and the second arenavirus particle is derived from Pichinde virus. In certain embodiments wherein a method described herein comprises administering a first arenavirus particle and a second arenavirus particle as described herein, the first arenavirus particle is derived from Pichinde virus, and the second arenavirus particle is derived from lymphocytic choriomeningitis virus (LCMV).

### 5.4 Replication-Defective Arenavirus Particles

In certain embodiments, an arenavirus particle for use with the methods and compositions provided herein is a replication-defective arenavirus particle. Exemplary replication-defective arenavirus particles are described, for example, in International Patent Application Publication No. WO 2009/083210, the content of which is incorporated herein in its entirety. In certain embodiments, the replication-defective arenavirus particle comprises an arenavirus genome wherein at least one arenavirus ORF (e.g., an ORF encoding GP, NP, Z protein, or L protein) is either functionally inactivated or deleted and the arenavirus particle has the ability to amplify and express its genetic information in cells infected with the arenavirus particle but is unable to produce further infectious progeny particles in normal, non-complementing cells.

In certain embodiments, the replication-defective (e.g., replication-deficient) arenavirus particle comprising a nucleotide sequence encoding a tumor antigen, tumor associated antigen, an antigen of a pathogen that causes an infectious disease, or an antigenic fragment of any of the foregoing (see Sections 5.1 and 5.2), encoding an immune checkpoint modulator (see Section 5.8), encoding a ligand of 4-1BB or another agonist of the 4-1BB costimulatory pathway, or a ligand of OX40 or another agonist of the OX40 costimulatory pathway, or encoding a cytokine (see Section 5.9) can be used with the methods and compositions provided herein. In specific embodiments, replication-defective arenavirus particles described herein are used with the methods and compositions provided herein in combination with replication-competent arenavirus particles described herein. In more specific embodiments, said replication-competent arenavirus particles are injected directly into a tumor in a subject.

In certain embodiments, the arenavirus particle as described herein is suitable for use as a vaccine, immunotherapy, or pharmaceutical composition and methods of using such arenavirus particle in the treatment of a neoplastic disease, for example, cancer, is provided. More detailed non-limiting description of the methods of using the arenavirus particle described herein is provided in Section 5.10.

### 5.5 Tri-Segmented Arenavirus Particles with Rearrangements of their ORFs

In certain embodiments, an arenavirus particle for use with the methods and compositions provided herein is a tri-segmented arenavirus particle. Exemplary tri-segmented arenavirus particles are described, for example, in International Patent Application Publication Nos. WO 2016/075250 and WO 2017/198726, which are incorporated by reference herein in their entireties.

In certain embodiments, tri-segmented arenavirus particles with rearrangements of their ORFs comprising a nucleotide sequence encoding a tumor antigen, tumor associated antigen, an antigen of a pathogen that causes an infectious disease, or an antigenic fragment of any of the foregoing (see Sections 5.1 and 5.2) as provided herein can be used with the methods and compositions provided herein. In certain embodiments, tri-segmented arenavirus particles with rearrangements of their ORFs comprising a nucleotide sequence encoding an immune checkpoint modulator can be used with the methods and compositions provided herein (see Section 5.8). In specific embodiments, the nucleotide sequence encodes a ligand of 4-1BB or another agonist of the 4-1BB costimulatory pathway, or a ligand of OX40 or another agonist of the OX40 costimulatory pathway. In certain embodiments, tri-segmented arenavirus particles with rearrangements of their ORFs comprising a nucleotide sequence encoding a cytokine can be used with the methods and compositions provided herein (see Section 5.9). In one aspect, provided herein is a tri-segmented arenavirus particle comprising one L segment and two S segments or two L segments and one S segment. In certain embodiments, propagation of the tri-segmented arenavirus particle does not result in a replication competent bi-segmented arenavirus particle. More specifically, in certain embodiments, two of the genomic segments (e.g., the two S segments or the two L segments, respectively) cannot recombine in a way to yield a single viral segment that could replace the two parent segments. In certain embodiments, inter-segmental recombination of two of the genomic segments (e.g., the two S segments or the two L segments, respectively), uniting two arenavirus ORFs on only one instead of two separate segments, abrogates viral promoter activity. In specific embodiments, the genome of the tri-segmented arenavirus particle comprises an arenaviral ORF in a position other than the wild-type position of the ORF and a nucleotide sequence encoding a tumor antigen, tumor associated antigen, an antigen of a pathogen that causes an infectious disease, or an antigenic fragment of any of the foregoing provided herein. In yet another specific embodiment, the genome of the tri-segmented arenavirus particle comprises all four arenavirus ORFs. Thus, in certain embodiments, the tri-segmented arenavirus particle is replication competent and infectious.

In certain embodiments, the genome of such a tri-segmented arenavirus that is replication competent and infectious has two available positions for insertion of heterologous nucleotide sequences. These positions can be used for heterologous nucleotide sequences, e.g., as set forth in Table 1 below. In certain embodiments, each such heterologous nucleotide sequence can be transcribed into a single transcript. In certain embodiments, each such heterologous nucleotide sequence encodes a polypeptide. In certain embodiments, such a heterologous nucleotide sequence can be polycistronic such that multiple polypeptides are ultimately produced from a single heterologous nucleotide sequence/transcript. This can be accomplished, e.g., using an internal ribosome entry site. In certain embodiments one such polypeptide can be a tumor antigen, tumor associated antigen, an antigen of a pathogen that causes an infectious disease, or an antigenic fragment of any of the foregoing (see Sections 5.1 and 5.2). In certain embodiments one such polypeptide can be an immune checkpoint modulator (see Section 5.8). In certain embodiments one such polypeptide can be a cytokine (see Section 5.9). In certain embodiments, the heterologous nucleotide sequence at one of the two available positions encodes both a tumor antigen, tumor associated antigen, an antigen of a pathogen that causes an infectious disease, or an antigenic fragment of any of the foregoing and an immune checkpoint modulator. In other embodiments, the heterologous nucleotide sequence at one of the two available positions encodes a tumor antigen, tumor associated antigen, an antigen of a pathogen that causes an infectious disease, or an antigenic fragment of any of the foregoing and the heterologous nucleotide sequence at the other of the two available positions encodes an immune checkpoint modulator. In certain embodiments, the heterologous nucleotide sequence at one of the two available positions encodes both a tumor antigen, tumor associated antigen, an antigen of a pathogen that causes an infectious disease, or an antigenic fragment of any of the foregoing and a cytokine. In other embodiments, the heterologous nucleotide sequence at one of the two available positions encodes a tumor antigen, tumor associated antigen, an antigen of a pathogen that causes an infectious disease, or an antigenic fragment of any of the foregoing and the heterologous nucleotide sequence at the other of the two available positions encodes a cytokine.

In other embodiments, the tri-segmented arenavirus particle lacks one of the four arenavirus ORFs. Thus, in certain embodiments, the tri-segmented arenavirus particle is infectious but unable to produce further infectious progeny in non-complementing cells.

In certain embodiments, tri-segmented arenavirus particles with rearrangements of their ORFs comprising a nucleotide sequence that does not encode a foreign antigen can be used with the methods and compositions provided herein. In specific embodiments, the tri-segmented arenavirus particle comprises an ORF in a position other than the wild-type position of the ORF and a nucleotide sequence comprising a deleted or inactivated arenaviral ORF. In specific embodiments, the tri-segmented arenavirus particle comprises an ORF in a position other than the wild-type position of the ORF and a nucleotide sequence wherein the untranslated region (UTR) is fused directly to the intergenic region (IGR). In specific embodiments, the tri-segmented arenavirus particle comprises an ORF in a position other than the wild-type position of the ORF and a nucleotide sequence comprising an ORF for a marker, such as GFP. In specific embodiments, the tri-segmented arenavirus particle comprises an ORF in a position other than the wild-type position of the ORF and a nucleotide sequence comprising a heterologous non-coding sequence. In yet another specific embodiment, the tri-segmented arenavirus particle comprises all four arenavirus ORFs. Thus, in certain embodiments, the tri-segmented arenavirus particle is replication competent and infectious. In other embodiments, the tri-segmented arenavirus particle lacks one of the four arenavirus ORFs. Thus, in certain embodiments, the tri-segmented arenavirus particle is infectious but unable to produce further infectious progeny in non-complementing cells.

In certain embodiments, the ORF encoding GP, NP, Z protein, or L protein of the tri-segmented arenavirus particle described herein can be under the control of an arenavirus genomic 3′ UTR or an arenavirus genomic 5′ UTR. In more specific embodiments, the arenavirus genomic 3′ UTR is the 3′ UTR of an arenavirus S segment. In another specific embodiment, the arenavirus genomic 3′ UTR is the 3′ UTR of an arenavirus L segment. In more specific embodiments, the arenavirus genomic 5′ UTR is the 5′ UTR of an arenavirus S segment. In other specific embodiments, the arenavirus genomic 5′ UTR is the 5′ UTR of an arenavirus L segment.

In other embodiments, the ORF encoding GP, NP, Z protein, or L protein of the tri-segmented arenavirus particle described herein can be under the control of the arenavirus conserved terminal sequence element (the 5′- and 3-terminal 19-20-nt regions) (see e.g., Perez & de la Torre, 2003, J Virol. 77(2): 1184-1194).

In certain embodiments, the ORF encoding GP, NP, Z protein or L protein of the tri-segmented arenavirus particle can be under the control of the promoter element of the 5′ UTR (see e.g., Albarino et al., 2011, J Virol., 85(8):4020-4). In another embodiment, the ORF encoding GP, NP, Z protein, or L protein of the tri-segmented arenavirus particle can be under the control of the promoter element of the 3′ UTR (see e.g., Albarino et al., 2011, J Virol., 85(8):4020-4). In more specific embodiments, the promoter element of the 5′ UTR is the 5′ UTR promoter element of the S segment or the L segment. In another specific embodiment, the promoter element of the 3′ UTR is the 3′ UTR promoter element of the S segment or the L segment.

In certain embodiments, the ORF encoding GP, NP, Z protein or L protein of the tri-segmented arenavirus particle can be under the control of a truncated arenavirus 3′ UTR or a truncated arenavirus 5′ UTR (see e.g., Perez & de la Torre, 2003, J Virol. 77(2): 1184-1194; Albarino et al., 2011, J Virol., 85(8):4020-4). In more specific embodiments, the truncated 3′ UTR is derived from the 3′ UTR of the arenavirus S segment or L segment. In more specific embodiments, the truncated 5′ UTR is derived from the 5′ UTR of the arenavirus S segment or L segment.

Also provided herein, is a cDNA of the genome of the tri-segmented arenavirus particle comprising a nucleotide sequence encoding a tumor antigen, tumor associated antigen, an antigen of a pathogen that causes an infectious disease, or an antigenic fragment of any of the foregoing provided herein. In more specific embodiments, provided herein is a DNA nucleotide sequence or a set of DNA nucleotide sequences encoding the genome of a tri-segmented arenavirus particle as set forth in Table 1.

In certain embodiments, the nucleotide sequences encoding the genome of the tri-segmented arenavirus particle are part of or incorporated into one or more DNA expression vectors. In a specific embodiment, nucleotide sequences encoding the genome of the tri-segmented arenavirus particle are part of or incorporated into one or more DNA expression vectors that facilitate production of a tri-segmented arenavirus particle as described herein. In another embodiment, a cDNA described herein can be incorporated into a plasmid. Techniques for the production of a cDNA and routine and conventional techniques of molecular biology and DNA manipulation and production, including any cloning technique known to the skilled artisan can be used. Such techniques are well known and are available to the skilled artesian in laboratory manuals such as, Sambrook and Russell, Molecular Cloning: A laboratory Manual, 3^{rd} edition, Cold Spring Harbor Laboratory N.Y. (2001).

In certain embodiments, the cDNA of the genome of the tri-segmented arenavirus particle comprising a nucleotide sequence encoding a tumor antigen, tumor associated antigen, an antigen of a pathogen that causes an infectious disease, or an antigenic fragment of any of the foregoing provided herein is introduced (e.g., transfected) into a host cell. Thus, in some embodiments provided herein, is a host cell comprising a cDNA of the tri-segmented arenavirus particle (i.e., a cDNA of the genomic segments of the tri-segmented arenavirus particle comprising a nucleotide sequence encoding a tumor antigen, tumor associated antigen, an antigen of a pathogen that causes an infectious disease, or an antigenic fragment of any of the foregoing provided herein). In other embodiments, the cDNA described herein is part of or can be incorporated into a DNA expression vector introduced into a host cell. Thus, in some embodiments provided herein is a host cell comprising a cDNA described herein that is incorporated into a vector. In other embodiments, the tri-segmented arenavirus genomic segments (i.e., the L segment and/or S segment or segments) described herein are introduced into a host cell.

In certain embodiments, described herein is a method of producing the tri-segmented arenavirus particle, wherein the method comprises transcribing the cDNA of the tri-segmented arenavirus particle comprising a nucleotide sequence encoding a tumor antigen, tumor associated antigen, an antigen of a pathogen that causes an infectious disease, or an antigenic fragment of any of the foregoing provided herein. In certain embodiments, a viral polymerase protein can be present during transcription of the tri-segmented arenavirus particle in vitro or in vivo. In certain embodiments, transcription of the arenavirus genomic segment is performed using a bi-directional promoter.

In other embodiments, transcription of the arenavirus genomic segment is performed using a bi-directional expression cassette (see e.g., Ortiz-Riano et al., 2013, J Gen Virol., 94(Pt 6): 1175-1188). In more specific embodiments the bi-directional expression cassette comprises both a polymerase I and a polymerase II promoter reading from opposite sides into the two termini of the inserted arenavirus genomic segment, respectively.

In other embodiments, transcription of the cDNA of the arenavirus genomic segment described herein comprises a promoter. Specific examples of promoters include an RNA polymerase I promoter, an RNA polymerase II promoter, an RNA polymerase III promoter, a T7 promoter, an SP6 promoter or a T3 promoter.

In certain embodiments, the method of producing the tri-segmented arenavirus particle can further comprise introducing into a host cell the cDNA of the genome of the tri-segmented arenavirus particle comprising a nucleotide sequence encoding a tumor antigen, tumor associated antigen, an antigen of a pathogen that causes an infectious disease, or an antigenic fragment of any of the foregoing provided herein. In certain embodiments, the method of producing the tri-segmented arenavirus particle can further comprise introducing into a host cell the cDNA of the genome of the tri-segmented arenavirus particle that comprises a nucleotide sequence encoding a tumor antigen, tumor associated antigen, an antigen of a pathogen that causes an infectious disease, or an antigenic fragment of any of the foregoing provided herein, wherein the host cell expresses all other components for production of the tri-segmented arenavirus particle; and purifying the tri-segmented arenavirus particle from the supernatant of the host cell. Such methods are well-known to those skilled in the art.

Provided herein are cell lines, cultures and methods of culturing cells transfected with nucleic acids, vectors, and compositions provided herein.

In certain embodiments, the tri-segmented arenavirus particle as described herein is an infectious and replication competent arenavirus particle. In specific embodiments, the arenavirus particle described herein is attenuated. In a particular embodiment, the tri-segmented arenavirus particle is attenuated such that the virus remains, at least partially, replication-competent and can replicate in vivo, but can only generate low viral loads resulting in subclinical levels of infection that are non-pathogenic. Such attenuated viruses can be used as an immunogenic composition.

In certain embodiments, the tri-segmented arenavirus particle has the same tropism as the bi-segmented arenavirus particle from which the tri-segmented arenavirus particle was derived.

Also provided herein, are compositions that comprise the tri-segmented arenavirus particle as described herein.

### 5.5.1 Tri-Segmented Arenavirus Particles Comprising One L Segment and Two S Segments

In one aspect, provided herein is a tri-segmented arenavirus particle comprising one L segment and two S segments. In certain embodiments, propagation of the tri-segmented arenavirus particle comprising one L segment and two S segments does not result in a replication-competent bi-segmented viral particle. In specific embodiments, propagation of the tri-segmented arenavirus particle comprising one L segment and two S segments does not result in a replication-competent bi-segmented viral particle after at least 10 days, at least 20 days, at least 30 days, at least 40 days, at least 50 days, at least 60 days, at least 70 days, at least 80 days, at least 90 days, or at least 100 days of persistent infection in mice lacking type I interferon receptor, type II interferon receptor and recombination activating gene (RAG1), and having been infected with 10⁴ PFU of the tri-segmented arenavirus particle (see Section 5.12(p)). In other embodiments, propagation of the tri-segmented arenavirus particle comprising one L segment and two S segments does not result in a replication-competent bi-segmented viral particle after at least 10 passages, at least 20 passages, at least 30 passages, at least 40 passages, or at least 50 passages.

The tri-segmented arenavirus particle with all viral genes in their respective wild-type position is known in the art (e.g., Emonet et al., 2011 J. Virol., 85(4):1473; Popkin et al., 2011, J. Virol, 85(15):7928). In a particular embodiment, the tri-segmented arenavirus genome consists of one L segment and two S segments, in which a nucleotide sequence encoding a tumor antigen, tumor associated antigen, an antigen of a pathogen that causes an infectious disease, or an antigenic fragment of any of the foregoing provided herein is inserted into one position on each S segment, more specifically, with one S segment encoding GP and a tumor antigen, tumor associated antigen, an antigen of a pathogen that causes an infectious disease, or an antigenic fragment of any of the foregoing, respectively, the other S segment encoding a tumor antigen, a tumor associated antigen, an antigen of a pathogen that causes an infectious disease, or an antigenic fragment of any of the foregoing and NP, respectively, and the L segment encoding the L protein and Z protein, wherein all segments are flanked by the respective 5′ and 3′ UTRs.

In certain embodiments, inter-segmental recombination of the two S segments of the tri-segmented arenavirus particle, provided herein, that unities the two arenaviral ORFs on one instead of two separate segments results in a non-functional promoter (i.e., a genomic segment of the structure: 5′ UTR-----------5′ UTR or a 3′ UTR------------3′ UTR), wherein each UTR forming one end of the genome is an inverted repeat sequence of the other end of the same genome.

In certain embodiments, the tri-segmented arenavirus particle comprising one L segment and two S segments has been engineered to carry an arenavirus ORF in a position other than the wild-type position of the ORF and a nucleotide sequence encoding a tumor antigen, tumor associated antigen, an antigen of a pathogen that causes an infectious disease, or an antigenic fragment of any of the foregoing provided herein. In other embodiments, the tri-segmented arenavirus particle comprising one L segment and two S segments has been engineered to carry two arenavirus ORFs, or three arenavirus ORFs, or four arenavirus ORFs, or five arenavirus ORFs, or six arenavirus ORFs in a position other than the wild-type position. In specific embodiments, the tri-segmented arenavirus particle comprising one L segment and two S segments comprises a full complement of all four arenavirus ORFs. Thus, in some embodiments, the tri-segmented arenavirus particle is an infectious and replication competent tri-segmented arenavirus particle. In specific embodiments, the two S segments of the tri-segmented arenavirus particle have been engineered to carry one of their ORFs in a position other than the wild-type position. In more specific embodiments, the two S segments comprise a full complement of the S segment ORFs. In certain specific embodiments, the L segment has been engineered to carry an ORF in a position other than the wild-type position or the L segment can be the wild-type genomic segment.

In certain embodiments, one of the two S segments can be selected from the group consisting of:
(i) an arenavirus S segment, wherein the ORF encoding the Z protein is under control of an arenavirus 5′ UTR;
(ii) an arenavirus S segment, wherein the ORF encoding the L protein is under control of an arenavirus 5′ UTR;
(iii) an arenavirus S segment, wherein the ORF encoding the NP is under control of an arenavirus 5′ UTR;
(iv) an arenavirus S segment, wherein the ORF encoding the GP is under control of an arenavirus 3′ UTR;
(v) an arenavirus S segment, wherein the ORF encoding the L protein is under control of an arenavirus 3′ UTR; and
(vi) an arenavirus S segment, wherein the ORF encoding the Z protein is under control of an arenavirus 3′ UTR.

In specific embodiments, an arenavirus particle described herein is tri-segmented and replication-competent and comprises one L segment and two S segments, wherein one of the two S segments is selected from the group consisting of:
a. an S segment, wherein the ORF encoding the NP is under control of an arenavirus genomic 5′ UTR;
b. an S segment, wherein the ORF encoding the Z protein is under control of an arenavirus genomic 5′ UTR;
c. an S segment, wherein the ORF encoding the L protein is under control of an arenavirus genomic 5′ UTR;
d. an S segment, wherein the ORF encoding the GP is under control of an arenavirus genomic 3′ UTR;
e. an S segment, wherein the ORF encoding the L protein is under control of an arenavirus genomic 3′ UTR; and
f. an S segment, wherein the ORF encoding the Z protein is under control of an arenavirus genomic 3′ UTR.

In a specific embodiment, a first S segment is engineered to carry an arenaviral ORF encoding GP in a position under control of an arenavirus genomic 3′ UTR and a first heterologous ORF encoding a tumor antigen, tumor associated antigen, an antigen of a pathogen that causes an infectious disease, or an antigenic fragment of any of the foregoing in a position under control of an arenavirus genomic 5′ UTR, and a second S segment is engineered to carry an arenaviral ORF encoding NP in a position under control of an arenavirus genomic 3′ UTR and a second heterologous ORF encoding an immune checkpoint modulator (e.g., an agonist of the 4-1BB costimulatory pathway, an agonist of the OX40 costimulatory pathway, a ligand of 4-1BB, a ligand of OX40, or an antagonist of the NKG2A coinhibitory pathway) in a position under control of an arenavirus genomic 5′ UTR.

In a specific embodiment, a first S segment is engineered to carry an arenaviral ORF encoding GP in a position under control of an arenavirus genomic 3′ UTR and a first heterologous ORF encoding an immune checkpoint modulator (e.g., an agonist of the 4-1BB costimulatory pathway, an agonist of the OX40 costimulatory pathway, a ligand of 4-1BB, a ligand of OX40, or an antagonist of the NKG2A coinhibitory pathway) in a position under control of an arenavirus genomic 5′ UTR, and a second S segment is engineered to carry an arenaviral ORF encoding NP in a position under control of an arenavirus genomic 3′ UTR and a second heterologous ORF encoding a tumor antigen, tumor associated antigen, an antigen of a pathogen that causes an infectious disease, or an antigenic fragment of any of the foregoing in a position under control of an arenavirus genomic 5′ UTR.

In a specific embodiment, an S segment is engineered to carry a first heterologous ORF encoding an immune checkpoint modulator (e.g., an agonist of the 4-1BB costimulatory pathway, an agonist of the OX40 costimulatory pathway, a ligand of 4-1BB, a ligand of OX40, or an antagonist of the NKG2A coinhibitory pathway) and a second heterologous ORF encoding a tumor antigen, tumor associated antigen, an antigen of a pathogen that causes an infectious disease, or an antigenic fragment of any of the foregoing.

In a specific embodiment, a first S segment is engineered to carry an arenaviral ORF encoding GP in a position under control of an arenavirus genomic 3′ UTR and a first heterologous ORF encoding a tumor antigen, tumor associated antigen, an antigen of a pathogen that causes an infectious disease, or an antigenic fragment of any of the foregoing in a position under control of an arenavirus genomic 5′ UTR, and a second S segment is engineered to carry an arenaviral ORF encoding NP in a position under control of an arenavirus genomic 3′ UTR and a second heterologous ORF encoding a cytokine (e.g., IL-12) in a position under control of an arenavirus genomic 5′ UTR.

In a specific embodiment, a first S segment is engineered to carry an arenaviral ORF encoding GP in a position under control of an arenavirus genomic 3′ UTR and a first heterologous ORF encoding a cytokine (e.g., IL-12) in a position under control of an arenavirus genomic 5′ UTR, and a second S segment is engineered to carry an arenaviral ORF encoding NP in a position under control of an arenavirus genomic 3′ UTR and a second heterologous ORF encoding a tumor antigen, tumor associated antigen, an antigen of a pathogen that causes an infectious disease, or an antigenic fragment of any of the foregoing in a position under control of an arenavirus genomic 5′ UTR.

In a specific embodiment, an S segment is engineered to carry a first heterologous ORF encoding a cytokine (e.g., IL-12) and a second heterologous ORF encoding a tumor antigen, tumor associated antigen, an antigen of a pathogen that causes an infectious disease, or an antigenic fragment of any of the foregoing.

In certain embodiments, the tri-segmented arenavirus particle comprising one L segment and two S segments can comprise a duplicate arenaviral ORF (i.e., two ORFs encoding e.g., GP or NP). In specific embodiments, the tri-segmented arenavirus particle comprising one L segment and two S segments can comprise one duplicate ORF (e.g., (GP, GP)) or two duplicate ORFs (e.g., (GP, GP) and (NP, NP)).

Table 1, below, is an illustration of non-limiting examples of the genome organization of a tri-segmented arenavirus particle comprising one L segment and two S segments, wherein intersegmental recombination of the two S segments in the tri-segmented arenavirus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 1
Tri-segmented arenavirus particle comprising one L segment and
two S segments.

| Position 1 | Position 2 | Position 3 | Position 4 | Position 5 | Position 6 |
|---|---|---|---|---|---|
| *ORF | GP | *ORF | NP | Z | L |
| *ORF | GP | *ORF | NP | L | Z |
| *ORF | NP | *ORF | GP | Z | L |
| *ORF | NP | *ORF | GP | L | Z |
| *ORF | NP | *ORF | Z | L | GP |
| *ORF | NP | Z | GP | *ORF | Z |
| *ORF | NP | Z | GP | Z | *ORF |
| *ORF | NP | *ORF | L | Z | GP |
| *ORF | L | *ORF | NP | Z | GP |
| *ORF | L | Z | NP | *ORF | GP |
| *ORF | L | *ORF | GP | Z | NP |
| *ORF | L | Z | GP | *ORF | NP |
| *ORF | Z | L | NP | *ORF | GP |
| *ORF | Z | *ORF | GP | L | NP |
| *ORF | Z | L | GP | *ORF | NP |
| L | GP | *ORF | NP | *ORF | Z |
| L | GP | *ORF | NP | Z | *ORF |
| L | GP | *ORF | Z | *ORF | NP |
| L | GP | Z | *ORF | *ORF | NP |
| L | *ORF | Z | GP | *ORF | NP |
| L | GP | Z | NP | *ORF | *ORF |
| L | *ORF | Z | NP | *ORF | GP |
| L | NP | *ORF | GP | Z | *ORF |
| L | NP | *ORF | Z | *ORF | GP |
| L | NP | Z | *ORF | GP | *ORF |
| L | *ORF | Z | *ORF | GP | NP |
| L | NP | Z | GP | *ORF | *ORF |
| L | NP | Z | *ORF | *ORF | GP |
| L | Z | *ORF | GP | *ORF | NP |
| L | Z | *ORF | NP | *ORF | GP |
| Z | GP | *ORF | NP | *ORF | L |
| Z | GP | *ORF | NP | L | *ORF |
| Z | GP | *ORF | L | *ORF | NP |
| Z | GP | L | *ORF | *ORF | NP |
| Z | *ORF | L | GP | *ORF | NP |
| Z | GP | L | NP | *ORF | *ORF |
| Z | *ORF | L | NP | *ORF | GP |
| Z | NP | *ORF | GP | *ORF | L |
| Z | NP | *ORF | L | *ORF | GP |
| Z | NP | L | GP | *ORF | *ORF |
| Z | NP | L | *ORF | *ORF | GP |
| Z | NP | *ORF | GP | L | *ORF |
| Z | L | *ORF | GP | *ORF | NP |

Position 1 is under the control of an arenavirus S segment 5′ UTR; Position 2 is under the control of an arenavirus S segment 3′ UTR; Position 3 is under the control of an arenavirus S segment 5′ UTR; Position 4 is under the control of an arenavirus S segment 3′ UTR; Position 5 is under the control of an arenavirus L segment 5′ UTR; Position 6 is under the control of an arenavirus L segment 3′ UTR.
*ORF indicates an inserted nucleotide sequence encoding (i) a tumor antigen, tumor associated antigen or an antigenic fragment thereof (see Section 5.1); (ii) an antigen of a pathogen that causes an infectious disease, or an antigenic fragment thereof (see Section 5.2); (iii) an immune checkpoint modulator (see Section 5.8); and/or (iv) a cytokine (see Section 5.9).

In certain embodiments, the IGR between position one and position two can be an arenavirus S segment or L segment IGR; the IGR between position three and position four can be an arenavirus S segment or L segment IGR; and the IGR between position five and position six can be an arenavirus L segment IGR. In a specific embodiment, the IGR between position one and position two can be an arenavirus S segment IGR; the IGR between position three and position four can be an arenavirus S segment IGR; and the IGR between position five and position six can be an arenavirus L segment IGR. In certain embodiments, other combinations are also possible. For example, a tri-segmented arenavirus particle comprising one L segment and two S segments, is genetically engineered such that intersegmental recombination of the two S segments in the tri-segmented arenavirus genome does not result in a replication-competent bi-segmented viral particle and abrogates arenaviral promoter activity (i.e., the resulting recombined S segment is made up of two 5′UTRs instead of a 3′ UTR and a 5′ UTR).

In certain embodiments, intersegmental recombination of an S segment and an L segment in the tri-segmented arenavirus particle comprising one L segment and two S segments, restores a functional segment with two viral genes on only one segment instead of two separate segments. In other embodiments, intersegmental recombination of an S segment and an L segment in the tri-segmented arenavirus particle comprising one L segment and two S segments does not result in a replication-competent bi-segmented viral particle.

In certain embodiments, one of skill in the art could construct an arenavirus genome with an organization as illustrated in Table 1 and as described herein, and then use an assay as described in Section 5.12 to determine whether the tri-segmented arenavirus particle is genetically stable, i.e., does not result in a replication-competent bi-segmented viral particle as discussed herein.

### 5.6 Split Arenavirus Vector Particles

Arenaviruses can also be engineered in the way described in International Patent Application Publication No. WO 2021/089853 and U.S. Provisional Application No. 63/188,317 filed May 13, 2021 (both of which are incorporated herein in their entireties). This technology is also called “split” vector technology. Similar to the trisegmented viruses described above, the technology described in WO 2021/089853 can be used to generate tri-segmented viruses with two open positions for insertion of heterologous nucleotide sequences. Such a heterologous nucleotide sequence can encode a polypeptide. In certain embodiments one such polypeptide can be a tumor antigen, tumor associated antigen, an antigen of a pathogen that causes an infectious disease, or an antigenic fragment of any of the foregoing (see Sections 5.1 and 5.2). In certain embodiments one such polypeptide can be an immune checkpoint modulator (see Section 5.8). In certain embodiments, one such polypeptide can be a ligand of 4-1BB or another agonist of the 4-1BB costimulatory pathway, or a ligand of OX40 or another agonist of the OX40 costimulatory pathway. In certain embodiments one such polypeptide can be a cytokine (see Section 5.9).

Briefly, such a “split” arenavirus particle is engineered such that an arenaviral ORF is separated over two or more mRNA transcripts. In certain embodiments, provided herein is an arenavirus genomic or antigenomic segment engineered such that the transcription thereof results in one or more mRNA transcripts comprising a nucleotide sequence encoding a functional fragment of arenavirus GP, NP, L protein, or Z protein.

In certain embodiments, the ORF encoding the arenavirus GP is separated (or split) over two mRNA transcripts and over two positions of the arenavirus genome, respectively. For example, the arenavirus GP signal peptide or a functional fragment thereof can be expressed from a first mRNA transcript (e.g., viral mRNA transcript) and arenavirus GP1 and GP2 subunits are expressed from a second mRNA transcript (e.g., viral mRNA transcript). In certain embodiments, the first mRNA transcript is under control of an arenavirus genomic 3′ UTR. In certain embodiments, the second mRNA transcript further encodes a heterologous non-arenaviral signal peptide (such as the signal peptide of the vesicular stomatitis virus serotype Indiana glycoprotein). In certain embodiments, the first mRNA transcript further comprises a nucleotide sequence encoding a heterologous non-arenaviral polypeptide, namely a tumor antigen, tumor associated antigen, an antigen of a pathogen that causes an infectious disease, or antigenic fragment of any of the foregoing (see, e.g., Sections 5.1 and 5.2) and/or a an immune checkpoint modulator (see Section 5.8) and/or a cytokine (see Section 5.9).

In certain embodiments, the genomic organization of such a “split” arenavirus vector is as follows:
First S segment: arenavirus GP1 and GP2 subunits fused to a heterologous signal peptide under control of an arenavirus genomic 5′ UTR; fusion of arenavirus GP signal peptide and a nucleotide sequence encoding the tumor antigen, tumor associated antigen, antigen of a pathogen that causes an infectious disease, or an antigenic fragment of any of the foregoing or the immune checkpoint modulator or the cytokine under control of an arenavirus genomic 3′ UTR.
Second S segment: a nucleotide sequence encoding the tumor antigen, tumor associated antigen, antigen of a pathogen that causes an infectious disease, or an antigenic fragment of any of the foregoing or the immune checkpoint modulator or the cytokine under control of an arenavirus genomic 5′ UTR and an ORF encoding the nucleoprotein (“NP”) under control of an arenavirus genomic 3′ UTR.
L segment: the arenavirus Z protein under control of an arenavirus genomic 5′ UTR; the arenavirus L protein under control of an arenavirus genomic 3′ UTR.

In certain embodiments, the nucleotide sequence encoding the tumor antigen, tumor associated antigen, an antigen of a pathogen that causes an infectious disease, or an antigenic fragment of any of the foregoing or the immune checkpoint modulator or the cytokine on the first S-Segment is different from the nucleotide sequence encoding the tumor antigen, tumor associated antigen, an antigen of a pathogen that causes an infectious disease, or an antigenic fragment of any of the foregoing or the immune checkpoint modulator or the cytokine on the second S-Segment. In certain embodiments, the nucleotide sequence encoding the tumor antigen, tumor associated antigen, an antigen of a pathogen that causes an infectious disease, or an antigenic fragment of any of the foregoing or the immune checkpoint modulator or the cytokine on the first S-Segment is the same as the nucleotide sequence encoding the tumor antigen, tumor associated antigen, an antigen of a pathogen that causes an infectious disease, or an antigenic fragment of any of the foregoing or the immune checkpoint modulator or the cytokine on the second S-Segment.

In certain embodiments, the tumor antigen, tumor associated antigen, an antigen of a pathogen that causes an infectious disease, or an antigenic fragment of any of the foregoing or the immune checkpoint modulator or the cytokine encoded on the first S-Segment is/are different from the tumor antigen, tumor associated antigen, an antigen of a pathogen that causes an infectious disease, or an antigenic fragment of any of the foregoing or the immune checkpoint modulator or the cytokine encoded on the second S-Segment. In certain embodiments, the tumor antigen, tumor associated antigen, an antigen of a pathogen that causes an infectious disease, or an antigenic fragment of any of the foregoing or the immune checkpoint modulator or the cytokine encoded on the first S-Segment is/are the same as the tumor antigen, tumor associated antigen, an antigen of a pathogen that causes an infectious disease, or an antigenic fragment of any of the foregoing or the immune checkpoint modulator or the cytokine encoded on the second S-Segment.

### 5.7 Generation of Arenavirus Particles

Generally, arenavirus particles for use in the methods and compositions provided herein can be recombinantly produced by standard reverse genetic techniques as described for LCMV (see Flatz et al., 2006, Proc Natl Acad Sci USA 103:4663-4668; Sanchez et al., 2006, Virology 350:370; Ortiz-Riano et al., 2013, J Gen Virol. 94:1175-88, which are incorporated by reference herein). To generate the arenavirus particles provided herein, these techniques can be applied as described below. The genome of the viruses can be modified as described herein. (a) Generation of Replication-Deficient Arenavirus Particles

The viruses as described in Section 5.4 can be produced as described herein. An arenavirus particle engineered to comprise a genome with the ability to amplify and express its genetic information in cells infected with the arenavirus particle but unable to produce further infectious progeny particles in normal, non-complementing cells, and wherein one arenavirus open reading frame is removed and replaced by a nucleotide sequence encoding a tumor antigen, tumor associated antigen, an antigen of a pathogen that causes an infectious disease, or an antigenic fragment of any of the foregoing and/or an immune checkpoint modulator and/or a cytokine, can be recombinantly produced by any reverse genetic techniques known to one skilled in the art. In certain embodiments, the nucleotide sequence encodes a ligand of 4-1BB or another agonist of the 4-1BB costimulatory pathway, or a ligand of OX40 or another agonist of the OX40 costimulatory pathway.

In certain embodiments, the method of generating the infectious, replication-deficient arenavirus particle comprises (i) transfecting into a host cell the cDNA of the first arenavirus genomic segment; (ii) transfecting into a host cell the cDNA of the second arenavirus genomic segment; (iii) transfecting into a host cell plasmids expressing the arenavirus' minimal trans-acting factors NP and L; (iv) maintaining the host cell under conditions suitable for virus formation; and (v) harvesting the arenavirus particle. In certain more specific embodiments, the cDNA is comprised in a plasmid.

Once generated from cDNA, the infectious, replication-deficient arenaviruses can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Owing to the removal or functional inactivation of one or more of the arenaviral ORFs in arenavirus vectors (here deletion of the glycoprotein, GP, will be taken as an example), arenavirus vectors can be generated and expanded in cells providing in trans the deleted viral gene(s), e.g., the GP in the present example. Such a complementing cell line, henceforth referred to as C-cells, is generated by transfecting a cell line such as BHK-21, HEK 293, VERO or other with one or more plasmid(s) for expression of the viral gene(s) of interest (complementation plasmid, referred to as C-plasmid). The C-plasmid(s) express the viral gene(s) deleted in the arenavirus vector to be generated under control of one or more expression cassettes suitable for expression in mammalian cells, e.g., a mammalian polymerase II promoter such as the EF1alpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, VERO, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells, transient transfection of normal cells can complement the missing viral gene(s) in each of the steps where C-cells will be used below. In addition, a helper virus can be used to provide the missing functionality in trans.

In certain embodiments, the complementing host cells are kept in culture and are transfected with one or more plasmid(s). The plasmid(s) encode the arenavirus genomic segment(s) of the arenavirus particle to be generated under control of a polymerase I promoter and terminator.

Plasmids that can be used for the generation of the arenavirus particle can include: i) a plasmid encoding the S genomic segment e.g., pol-I S, ii) a plasmid encoding the L genomic segment e.g., pol-I L. In certain embodiments, the plasmid encoding an arenavirus polymerase that direct intracellular synthesis of the viral L and S segments can be incorporated into the transfection mixture. For example, a plasmid encoding the L protein and/or a plasmid encoding NP (pC-L and pC-NP, respectively) can be present. The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

Typically, RNA polymerase I-driven expression cassettes, RNA polymerase II-driven cassettes or T7 bacteriophage RNA polymerase driven cassettes can be used, the latter preferentially with a 3′-terminal ribozyme for processing of the primary transcript to yield the correct end. In certain embodiments, the plasmids encoding the arenavirus genomic segments can be the same, i.e., the genome sequence and transacting factors can be transcribed by T7, poll and polII promoters from one plasmid.

In other embodiments, transcription of the arenavirus genomic segment is performed using a bi-directional expression cassette (see e.g., Ortiz-Riano et al., 2013, J Gen Virol., 94(Pt 6): 1175-1188). In more specific embodiments the bi-directional expression cassette comprises both a polymerase I and a polymerase II promoter reading from opposite sides into the two termini of the inserted arenavirus genomic segment, respectively.

In other embodiments, transcription of the cDNA of the arenavirus genomic segment described herein comprises a promoter. Specific examples of promoters include an RNA polymerase I promoter, an RNA polymerase II promoter, an RNA polymerase III promoter, a T7 promoter, an SP6 promoter or a T3 promoter.

For recovering the arenavirus particle described herein, the following procedures are envisaged. First day: complementing cells, are transfected with a mixture of the plasmids, as described above. For this one can exploit any commonly used strategies such as calcium-phosphate, liposome-based protocols or electroporation.

3-5 days later: The cell suspension (i.e., cells and medium) is harvested. Arenavirus particles present in the medium are cleared from cells and debris by centrifugation and the supernatant (i.e., the arenavirus vector preparation) is aliquoted and stored at 4° C., −20° C., or −80° C. The arenavirus vector preparation's infectious titer is assessed by an immunofocus assay. Alternatively, the transfected cells and supernatant may be passaged to a larger vessel on day 3-5 after transfection, and vectors are harvested up to five days after passage as described before.

### (b) Generation of a Tri-Segmented, Replication-Competent Arenavirus Particle

The viruses described in Section 5.5 and Section 5.6 can be produced as described herein and, for example, in International Patent Application Publication No. WO 2016/075250 and International Patent Application Publication No. WO 2021/089853, respectively, which are incorporated here in their entireties. A tri-segmented arenavirus particle comprising a genomic segment that has been engineered to carry a viral ORF in a position other than the wild-type position of the ORF and further comprising a nucleotide sequence encoding a tumor antigen, tumor associated antigen, an antigen of a pathogen that causes an infectious disease, or an antigenic fragment of any of the foregoing and/or an immune checkpoint modulator and/or a cytokine can be recombinantly produced by reverse genetic techniques known in the art, for example as described by Emonet et al., 2008, PNAS, 106(9):3473-3478; Popkin et al., 2011, J. Virol., 85 (15):7928-7932, which are incorporated by reference herein.

In certain embodiments, the method of generating the tri-segmented arenavirus particle comprises (i) transfecting into a host cell the cDNAs of the one arenavirus L segment and two arenavirus S segments or two arenavirus L segments and one arenavirus S segment; (ii) transfecting into a host cell plasmids expressing the arenavirus' minimal trans-acting factors NP and L; (iii) maintaining the host cell under conditions suitable for virus formation; and (iv) harvesting the arenavirus particle. In certain more specific embodiments, the cDNA of the arenavirus S and L segments is comprised in a plasmid.

Once generated from cDNA, the tri-segmented arenavirus particle (i.e., infectious and replication competent) can be propagated. In certain embodiments, the tri-segmented arenavirus particle can be propagated in any host cell that allows the virus to grow to titers that permit the uses of the virus as described herein. In one embodiment, the host cell allows the tri-segmented arenavirus particle to grow to titers comparable to those determined for the corresponding wild-type arenavirus.

In certain embodiments, the tri-segmented arenavirus particle may be propagated in host cells. Specific examples of host cells that can be used include BHK-21, HEK 293, VERO or other. In a specific embodiment, the tri-segmented arenavirus particle may be propagated in a cell line.

In certain embodiments, the host cells are kept in culture and are transfected with one or more plasmid(s). The plasmid(s) encode the arenavirus genomic segment(s) of the arenavirus particle to be generated under control of a polymerase I promoter and terminator.

In specific embodiments, the host cells are kept in culture and are transfected with one or more plasmid(s). The plasmid(s) express the viral protein(s) to be generated under control of one or more expression cassettes suitable for expression in mammalian cells, e.g., consisting of a polymerase II promoter and terminator.

Plasmids that can be used for generating the tri-segmented arenavirus particle comprising one L segment and two S segments can include: i) two plasmids each encoding the S genome segments e.g., pol-I S, ii) a plasmid encoding the L genome segment e.g., pol-I L. Plasmids needed for the tri-segmented arenavirus comprising two L segments and one S segments are: i) two plasmids each encoding the L genome segments e.g., pol-L, ii) a plasmid encoding the S genome segment e.g., pol-I S.

In certain embodiments, a plasmid encoding an arenavirus polymerase that directs intracellular synthesis of the viral L and S segments can be incorporated into the transfection mixture. For example, a plasmid encoding the L protein and a plasmid encoding NP (pC-L and pC-NP, respectively) can be used. The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In addition, the plasmid(s) can feature a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Transfection of host cells with a plasmid(s) can be performed using any of the commonly used strategies such as calcium-phosphate, liposome-based protocols or electroporation.

Typically, RNA polymerase I-driven expression cassettes, RNA polymerase II-driven cassettes or T7 bacteriophage RNA polymerase driven cassettes can be used, the latter preferentially with a 3′-terminal ribozyme for processing of the primary transcript to yield the correct end. In certain embodiments, the plasmids encoding the arenavirus genomic segments can be the same, i.e., the genome sequence and transacting factors can be transcribed by T7, poll and polII promoters from one plasmid.

In other embodiments, transcription of the arenavirus genomic segment is performed using a bi-directional expression cassette (see e.g., Ortiz-Riano et al., 2013, J Gen Virol., 94(Pt 6): 1175-1188). In more specific embodiments the bi-directional expression cassette comprises both a polymerase I and a polymerase II promoter reading from opposite sides into the two termini of the inserted arenavirus genomic segment, respectively.

In other embodiments, transcription of the cDNA of the arenavirus genomic segment described herein comprises a promoter. Specific examples of promoters include an RNA polymerase I promoter, an RNA polymerase II promoter, an RNA polymerase III promoter, a T7 promoter, an SP6 promoter or a T3 promoter.

For recovering the tri-segmented arenavirus vector, the following procedures are envisaged. First day: cells, are transfected with a mixture of the plasmids, as described above. For this one can exploit any commonly used strategies such as calcium-phosphate, liposome-based protocols or electroporation.

3-5 days later: The cell suspension (i.e., cells and medium) is harvested. Arenavirus particles present in the medium are cleared from cells and debris by centrifugation and the supernatant (i.e., the arenavirus vector preparation) is aliquoted and stored at 4° C., −20° C., or −80° C. The arenavirus vector preparation's infectious titer is assessed by an immunofocus assay. Alternatively, the transfected cells and supernatant may be passaged to a larger vessel on day 3-5 after transfection, and vectors are harvested up to five days after passage as described before.

The split arenavirus particles described in Section 5.6 can be generated with procedures similar to those described in this Section 5.7(b).

### 5.8 Immune Checkpoint Modulators

The term “immune checkpoint modulator” as used in this disclosure refers to an agonist of a costimulatory pathway or an antagonist of a coinhibitory pathway.

In certain embodiments, an immune checkpoint modulator described herein is an agonist (i.e., activator) of a costimulatory pathway. In specific embodiments, an immune checkpoint modulator described herein is an agonist of a costimulatory immune checkpoint molecule (also called a costimulatory molecule). In a specific embodiment, the costimulatory immune checkpoint molecule is a costimulatory immune checkpoint receptor. In a specific embodiment, the costimulatory immune checkpoint molecule is an agonistic ligand of a costimulatory immune checkpoint receptor. In a specific embodiment, the costimulatory immune checkpoint molecule is a member of the tumor necrosis factor receptor superfamily (“TNFRSF”) (e.g., 4-1BB, OX40, CD40, CD27, or GITR). In a specific embodiment, the costimulatory immune checkpoint molecule is a member of the B7-CD28 superfamily (e.g., CD28 or ICOS). In a specific embodiment, the costimulatory immune checkpoint molecule is 4-1BB (i.e., CD137), OX40 (i.e., CD134), CD40, CD27, GITR (i.e., CD357), CD28, ICOS (i.e., CD278), HVEM, TNFR2, CD30, or DR3. In a specific embodiment, the costimulatory immune checkpoint molecule is CD80 or CD86. In a specific embodiment, an immune checkpoint modulator described herein is an agonist of the 4-1BB costimulatory pathway. In a specific embodiment, an immune checkpoint modulator described herein is an agonist of the OX40 costimulatory pathway. In specific embodiments, an immune checkpoint modulator described herein is an agonistic ligand of a costimulatory immune checkpoint receptor. In a specific embodiment, an immune checkpoint modulator described herein is a ligand of 4-1BB. In a specific embodiment, an immune checkpoint modulator described herein is a ligand of OX40. In a specific embodiment, an immune checkpoint modulator described herein is a ligand of CD40. In specific embodiments, an immune checkpoint modulator described herein is an agonistic antibody or antigen-binding fragment thereof of a costimulatory immune checkpoint receptor (i.e., an agonistic antibody or antigen-binding fragment thereof that binds to and activates a costimulatory immune checkpoint receptor). In a specific embodiment, an immune checkpoint modulator described herein is an agonistic antibody or antigen-binding fragment thereof of 4-1BB. In a specific embodiment, an immune checkpoint modulator described herein is an agonistic antibody or antigen-binding fragment thereof of OX40. In a specific embodiment, an immune checkpoint modulator described herein is an agonistic antibody or antigen-binding fragment thereof of CD40. In specific embodiments, an immune checkpoint modulator described herein is an antagonistic antibody or antigen-binding fragment thereof of an antagonistic ligand of a costimulatory immune checkpoint receptor (i.e., an antagonistic antibody or antigen-binding fragment thereof that binds to an antagonistic ligand of a costimulatory immune checkpoint receptor and interferes with the inhibition of the costimulatory immune checkpoint receptor by the antagonistic ligand). In specific embodiments, an immune checkpoint modulator described herein is an agonistic aptamer of a costimulatory immune checkpoint receptor (i.e., an agonistic aptamer that binds to and activates a costimulatory immune checkpoint receptor). In specific embodiments, an immune checkpoint modulator described herein is an antagonistic aptamer of an antagonistic ligand of a costimulatory immune checkpoint receptor (i.e., an antagonistic aptamer that binds to an antagonistic ligand of a costimulatory immune checkpoint receptor and interferes with the inhibition of the costimulatory immune checkpoint receptor by the antagonistic ligand).

In other embodiments, an immune checkpoint modulator described herein is an antagonist (i.e., inhibitor) of a coinhibitory pathway. In specific embodiments, an immune checkpoint modulator described herein is an antagonist of a coinhibitory immune checkpoint molecule (also called a coinhibitory molecule). In a specific embodiment, the coinhibitory immune checkpoint molecule is a coinhibitory immune checkpoint receptor. In a specific embodiment, the coinhibitory immune checkpoint molecule is an agonistic ligand of a coinhibitory immune checkpoint receptor. In a specific embodiment, the coinhibitory immune checkpoint molecule is PD-1, PD-L1, PD-L2, CTLA-4, LAG-3 (CD223), Galectin-3, BTLA, TIM3, VISTA, B7-H3, B7-H4, GAL9, TIGIT (Vstm3/WUCAM/VSIG9), CGEN-15001T, CGEN-15022, CGEN-15027, CGEN-15049, CGEN-15052, CGEN-15092, CD244 (2B4), CD47, CD96 (TACTILE), NKG2A, HLA-E, or HLA-G. In a specific embodiment, an immune checkpoint modulator described herein is an antagonist of the NKG2A coinhibitory pathway. In specific embodiments, an immune checkpoint modulator described herein is an antagonistic ligand of a coinhibitory immune checkpoint receptor. In specific embodiments, an immune checkpoint modulator described herein is an antagonistic antibody or antigen-binding fragment thereof of a coinhibitory immune checkpoint receptor (i.e., an antagonistic antibody or antigen-binding fragment thereof that binds to and inhibits a coinhibitory immune checkpoint receptor). In a specific embodiment, an immune checkpoint modulator described herein is an antagonistic antibody or antigen-binding fragment thereof of NKG2A. In a specific embodiment, an immune checkpoint modulator described herein is an antagonistic antibody or antigen-binding fragment thereof of NKG2A (e.g., monalizumab (Innate Pharma)). In a specific embodiment, an immune checkpoint modulator described herein is an antagonistic antibody or antigen-binding fragment thereof of PD-1. In specific embodiments, an immune checkpoint modulator described herein is an antagonistic antibody or antigen-binding fragment thereof of an agonistic ligand of a coinhibitory immune checkpoint receptor (i.e., an antagonistic antibody or antigen-binding fragment thereof that binds to an agonistic ligand of a coinhibitory immune checkpoint receptor and inhibits the activation of the coinhibitory immune checkpoint receptor by the agonistic ligand). In specific embodiments, an immune checkpoint modulator described herein is an antagonistic aptamer of a coinhibitory immune checkpoint receptor (i.e., an antagonistic aptamer that binds to and inhibits a coinhibitory immune checkpoint receptor). In specific embodiments, an immune checkpoint modulator described herein is an antagonistic aptamer of an agonistic ligand of a coinhibitory immune checkpoint receptor (i.e., an antagonistic aptamer that binds to an agonistic ligand of a coinhibitory immune checkpoint receptor and inhibits the activation of the coinhibitory immune checkpoint receptor by the agonistic ligand). In specific embodiments, an immune checkpoint modulator described herein is a soluble form of a coinhibitory immune checkpoint receptor that can bind to an agonistic ligand of the coinhibitory immune checkpoint receptor and block the interaction between the agonistic ligand and the coinhibitory immune checkpoint receptor, thereby inhibiting the activation of the coinhibitory immune checkpoint receptor. In a specific embodiment, an immune checkpoint modulator described herein is soluble PD-1. Soluble PD-1 can bind to PD-L1 and block the interaction between PD-1 and PD-L1.

Antibodies and antigen-binding fragments thereof described herein include, but are not limited to, monoclonal antibodies, human antibodies, humanized antibodies, chimeric antibodies, synthetic antibodies, recombinantly produced antibodies, tetrameric antibodies comprising two heavy chain and two light chain molecules, multispecific antibodies (including bispecific antibodies), antibody light chain-antibody heavy chain pairs, heteroconjugate antibodies, single domain antibodies, monovalent antibodies, single chain antibodies, single-chain Fvs (scFvs), Fab fragments, F(ab′) fragments, disulfide-linked Fvs (sdFvs), and epitope-binding fragments of any of the above. In certain embodiments, antibodies to be administered with arenavirus particles described herein refer to polyclonal antibody populations. Antibodies described herein can be of any type (e.g., IgG, IgE, IgM, IgD, IgA or IgY), any class (e.g., IgG₁, IgG₂, IgG₃, IgG₄, IgA₁ or IgA₂), or any subclass (e.g., IgG2a or IgG2b) of immunoglobulin molecule. In certain embodiments, antibodies described herein are IgG antibodies, or a class (e.g., human IgG₁, IgG₂, IgG₃, or IgG₄) or subclass thereof.

In certain embodiments, an immune checkpoint modulator described herein is identical to a naturally occurring biological molecule (e.g., protein or polypeptide). In certain embodiments, an immune checkpoint modulator described herein is a variant (e.g., an engineered/modified form) of a naturally occurring biological molecule (e.g., protein or polypeptide).

In certain embodiments, an immune checkpoint modulator can be encoded by a heterologous nucleotide sequence inserted in the arenavirus genome. In certain embodiments, the same arenavirus genome carries two heterologous nucleotide sequences-one encoding an immune checkpoint modulator and one encoding a tumor antigen, tumor-associated antigen, an antigen of a pathogen that causes an infectious disease, or an antigenic fragment of any of the foregoing. In certain embodiments, the nucleotide sequence encoding the tumor antigen, tumor associated antigen, antigen of a pathogen that causes an infectious disease, or the antigenic fragment of any of the foregoing, and the nucleotide sequence encoding the immune checkpoint modulator, are at the same position of the same arenaviral genome segment for insertion of heterologous nucleotide sequence(s) (see Sections 5.5 and 5.6), and therefore are under the control of the same UTR. In certain embodiments, the nucleotide sequence encoding the tumor antigen, tumor associated antigen, antigen of a pathogen that causes an infectious disease, or the antigenic fragment of any of the foregoing, and the nucleotide sequence encoding the immune checkpoint modulator, are on the same segment of the arenaviral genome. In certain embodiments, the nucleotide sequence encoding the tumor antigen, tumor associated antigen, antigen of a pathogen that causes an infectious disease, or the antigenic fragment of any of the foregoing, and the nucleotide sequence encoding the immune checkpoint modulator, are at different positions of the arenaviral genome. In certain embodiments, the nucleotide sequence encoding the tumor antigen, tumor associated antigen, antigen of a pathogen that causes an infectious disease, or the antigenic fragment of any of the foregoing, and the nucleotide sequence encoding the immune checkpoint modulator, are on different segments of the arenaviral genome. In certain embodiments, the nucleotide sequence encoding the tumor antigen, tumor associated antigen, antigen of a pathogen that causes an infectious disease, or the antigenic fragment of any of the foregoing, and the nucleotide sequence encoding the immune checkpoint modulator, are separated via a spacer sequence. In certain embodiments, the sequence encoding the tumor antigen, tumor associated antigen, antigen of a pathogen that causes an infectious disease, or the antigenic fragment of any of the foregoing, and the nucleotide sequence encoding the immune checkpoint modulator, are separated by an internal ribosome entry site, or a sequence encoding a protease cleavage site. In certain embodiments, the nucleotide sequence encoding the tumor antigen, tumor associated antigen, antigen of a pathogen that causes an infectious disease, or the antigenic fragment of any of the foregoing, and the nucleotide sequence encoding the immune checkpoint modulator, are separated by a nucleotide sequence encoding a linker or a self-cleaving peptide. Any linker peptide or self-cleaving peptide known to the skilled artisan can be used with the compositions and methods provided herein. In certain embodiments, the tumor antigen, tumor associated antigen, antigen of a pathogen that causes an infectious disease, or the antigenic fragment of any of the foregoing, and the immune checkpoint modulator, are directly fused together. In certain embodiments, the tumor antigen, tumor associated antigen, antigen of a pathogen that causes an infectious disease, or the antigenic fragment of any of the foregoing, and the immune checkpoint modulator, are fused together via a peptide linker. In certain embodiments, the tumor antigen, tumor associated antigen, antigen of a pathogen that causes an infectious disease, or the antigenic fragment of any of the foregoing, and the immune checkpoint modulator are separated from each other via a self-cleaving peptide. In certain embodiments, the tumor antigen, tumor associated antigen, antigen of a pathogen that causes an infectious disease, or the antigenic fragment of any of the foregoing, and the immune checkpoint modulator are expressed on the same arenavirus particle. In certain embodiments, the tumor antigen, tumor associated antigen, antigen of a pathogen that causes an infectious disease, or the antigenic fragment of any of the foregoing, and the immune checkpoint modulator are expressed on different arenavirus particles. In certain embodiments, the tumor antigen, tumor associated antigen, antigen of a pathogen that causes an infectious disease, or the antigenic fragment of any of the foregoing, and the immune checkpoint modulator are expressed on different arenavirus particles derived from the same arenavirus strain. In certain embodiments, the tumor antigen, tumor associated antigen, antigen of a pathogen that causes an infectious disease, or the antigenic fragment of any of the foregoing, and the immune checkpoint modulator are expressed on different arenavirus particles derived from different arenavirus strains. In certain embodiments, an immune checkpoint modulator is administered separately from the arenavirus particle and is not encoded by the arenavirus genome. In certain embodiments, one immune checkpoint modulator is encoded by the arenavirus genome (either in the genome of the same arenavirus particle which also encodes the tumor antigen, tumor-associated antigen, antigen of a pathogen that causes an infectious disease, or the antigenic fragment of any of the foregoing or in the genome of a separate arenavirus particle) and one immune checkpoint modulator is administered separately (i.e., it is not encoded by the arenavirus genome). In certain embodiments, the arenavirus genome encodes a ligand of 4-1BB or another agonist of the 4-1BB costimulatory pathway, or a ligand of OX40 or another agonist of the OX40 costimulatory pathway. In certain embodiments, the ligand of 4-1BB or another agonist of the 4-1BB costimulatory pathway, or ligand of OX40 or another agonist of the OX40 costimulatory pathway is administered separately from the arenavirus particle and is not encoded by the arenavirus genome.

In certain embodiments, an immune checkpoint modulator can be encoded by mRNA, DNA or a non-arenavirus viral vector, and the mRNA, DNA or non-arenavirus viral vector is administered to a subject described herein to deliver the immune checkpoint modulator.

In certain embodiments, an immune checkpoint modulator can be directly administered to a subject described herein (preferably in the form of a pharmaceutical composition).

In certain embodiments, the immune checkpoint modulator is a bispecific antibody. Such a bispecific antibody can bind to a costimulatory immune checkpoint molecule or coinhibitory immune checkpoint molecule and to another molecule. In a specific embodiment, such a bispecific antibody can simultaneously bind to 4-1BB and another molecule. In a specific embodiment, such a bispecific antibody can simultaneously bind to OX40 and another molecule. In a specific embodiment, such a bispecific antibody can simultaneously bind to NKG2A and another molecule. The other molecule can be a costimulatory immune checkpoint molecule, a coinhibitory immune checkpoint molecule, a tumor antigen, a tumor associated antigen, a molecule expressed on the surface of cells in the tumor or in proximity to the tumor, optionally wherein the cells are cells of the tumor stroma, or an antigen of a pathogen that causes an infectious disease (which can be the same antigen encoded by the arenavirus particle or a different antigen not encoded by the arenavirus particle). In specific embodiments, the other molecule is a molecule expressed on the surface of cells (e.g., tumor cells, tumor stroma cells, or cells infected with the pathogen). For example, such a bispecific antibody can simultaneously target and activate two costimulatory immune checkpoint molecules. In a specific embodiment, such a bispecific antibody can simultaneously target and activate 4-1BB and another costimulatory immune checkpoint molecule. In a specific embodiment, such a bispecific antibody can simultaneously target and activate OX40 and another costimulatory immune checkpoint molecule. Such a bispecific antibody can also simultaneously target and inhibit two coinhibitory immune checkpoint molecules. In a specific embodiment, such a bispecific antibody can simultaneously target and inhibit NKG2A and another coinhibitory immune checkpoint molecule. Such a bispecific antibody can also target and activate one costimulatory immune checkpoint molecule and at the same time target and inhibit one coinhibitory immune checkpoint molecule.

In certain embodiments, the immune checkpoint modulator targets/activates a member of the tumor necrosis factor receptor superfamily (“TNFRSF”). In specific embodiments, the immune checkpoint modulator activates the 4-1BB costimulatory pathway. In specific embodiments, the immune checkpoint modulator targets/activates 4-1BB. In specific embodiments, the immune checkpoint modulator activates the OX40 costimulatory pathway. In specific embodiments, the immune checkpoint modulator targets/activates OX40.

In certain embodiments wherein an immune checkpoint modulator (e.g., an agonist of the 4-1BB costimulatory pathway, an agonist of the OX40 costimulatory pathway, an immune checkpoint modulator other than an agonist of the 4-1BB costimulatory pathway, or an immune checkpoint modulator other than an agonist of the OX40 costimulatory pathway) is administered in combination with an arenavirus particle expressing a tumor antigen, tumor associated antigen, antigen of a pathogen that causes an infectious disease, or antigenic fragment of any of the foregoing, as described herein, the immune checkpoint modulator and the arenavirus particle are administered at the same time.

In certain embodiments, the immune checkpoint modulator comprises an amino acid sequence as shown in Table 2 (see Section 6) or is encoded by a nucleotide comprising a nucleotide sequence as shown in Table 2.

The manufacturer information is provided for certain immune checkpoint modulators described in this disclosure; however, such manufacturer information shall not be construed as limiting the source of the corresponding immune checkpoint modulator. It is contemplated that the equivalent immune checkpoint modulator (for example, a generic version) produced by another manufacturer can also be used in a method described herein.

### (a) 4-1BB And Agonists Thereof

In certain embodiments, the immune checkpoint modulator is an agonist of 4-1BB. In certain embodiments, the agonist of 4-1BB is any agonist of the 4-1BB costimulatory pathway. In certain embodiments, the agonist of the 4-1BB costimulatory pathway is an agonistic antibody of 4-1BB. In specific embodiments, the agonist of the 4-1BB costimulatory pathway is a bispecific antibody that binds to 4-1BB and to a molecule other than 4-1BB. In particular embodiments, the molecule other than 4-1BB is a costimulatory molecule, a tumor antigen, a tumor associated antigen, or a molecule expressed on the surface of cells in the tumor or in proximity to the tumor, optionally wherein the cells are cells of the tumor stroma. In one embodiment, the bispecific antibody binds to 4-1BB and to another costimulatory molecule, wherein the bispecific antibody activates both 4-1BB and the other costimulatory molecule. In another embodiment, the bispecific antibody binds to 4-1BB and to a coinhibitory molecule, wherein the bispecific antibody activates 4-1BB but inhibits the coinhibitory molecule. In specific embodiments, the agonist of the 4-1BB costimulatory pathway is an agonistic antibody of 4-1BB. In particular embodiments, the agonistic antibody of 4-1BB simultaneously targets and activates 4-1BB and another costimulatory molecule. In particular embodiments, the agonistic antibody of 4-1BB targets and activates 4-1BB and at the same time targets and inhibits a coinhibitory molecule. In particular embodiments, the agonistic antibody of 4-1BB is an antigen-binding fragment (Fab) or single-chain variable fragment (scFv). In certain embodiments, the agonist of the 4-1BB costimulatory pathway is utomilumab (PF-05082566; Pfizer, Inc.), INBRX-105 (Inhibrx, Inc.), ABL503 (ABL Bio), ATOR-1017 (Alligator Bioscience), FS222 (F-Star Therapeutics), RG7827 (FAP 4-1BBL FP; Roche), RG6076 (CD19-4-1BBL; Roche), urelumab (BMS-663513; Bristol-Myers Squibb), CHU CD137 agonist switch antibody (Chugai Pharmaceutical), AGEN-2373 (Agenus), CTX-471 (Compass Therapeutics), FS-120 (F-star Therapeutics), LVGN-6051 (Lyvgen Biopharma), MCLA-145 (Merus), AMG-506 (Amgen/Molecular Partners), PRS-343 (Pieris Pharmaceuticals), STA-551 (Chugai Pharmaceutical/Roche), ADG-106 (Adagene), DSP-107 (KAHR Medical), DuoBody-CD40x4-1BB (BNT-312, GEN1042; BioNTech/Genmab), DuoBody-PD-L1x 4-1BB (GEN-1046, BNT-311; Ligand Pharmaceuticals/BioNTech/Genmab), ALG.APV-527 (Aptevo Therapeutics), CB307 (Crescendo Biologics), ABP-300 (Abpro/Mabwell Bioscience), NM21-1480 (Numab Therapeutics AG), EU101 (Eutilex), RO7227166 (Roche), ABL111 (ABL Bio), HERA-4-1BBL (Apogenix), and SL-279137 (PD-1-Fc-4-1BBL) (Shattuck Labs). In certain embodiments, the agonist of the 4-1BB costimulatory pathway is 4-1BBL. In certain embodiments, the agonist of the 4-1BB costimulatory pathway is a variant (e.g., an engineered/modified form) of 4-1BBL.

In certain embodiments wherein an agonist of the 4-1BB costimulatory pathway is administered in combination with an arenavirus particle expressing a tumor antigen, tumor associated antigen, antigen of a pathogen that causes an infectious disease, or antigenic fragment of any of the foregoing, as described herein, the agonist of the 4-1BB costimulatory pathway and the arenavirus particle are administered at the same time.

### (b) OX40 And Agonists Thereof

In certain embodiments, the immune checkpoint modulator is an agonist of OX40. In certain embodiments, the agonist of OX40 is any agonist of the OX40 costimulatory pathway. In certain embodiments, the agonist of the OX40 costimulatory pathway is an agonistic antibody of OX40. In specific embodiments, the agonistic antibody of OX40 is an antigen-binding fragment (Fab) or single-chain variable fragment (scFv). In certain embodiments, the agonist of the OX40 costimulatory pathway is INBRX-106 (Inhibrx, Inc.), PF-04518600 (Pfizer, Inc.), BMS-986178 (Bristol Myers Squibb), BGB-A445 (BeiGene), MEDI0562 (MedImmune, LLC), MEDI6469 (AstraZeneca), GSK3174998 (GlaxoSmithKline), MOXR-0916 (Pogalizumab, RG 7888; Genentech/Roche), anti-FAP/anti-OX40 bispecific agonistic antibody, anti-FAP/OX40L agonist fusion protein, INCAGN01949 (Incyte Biosciences International Sarl), HERA-OX40L, or SL-279252 (PD1-Fc-OX40L) (Shattuck Labs). In certain embodiments, the agonist of the OX40 costimulatory pathway is mRNA-2416 (Moderna). In certain embodiments, the agonist of the OX40 costimulatory pathway is an OX40 agonist described in Cebada et al., 2020, Expert Opinion on Therapeutic Patents, 31(1): 81-90, which is incorporated herein in its entirety. In certain embodiments, the agonist of the OX40 costimulatory pathway is an OX40 agonist described in U.S. Patent No. U.S. Pat. No. 9,006,399B2, U.S. Pat. No. 9,163,085B2, U.S. Pat. No. 9,695,246B2, U.S. Pat. No. 9,644,032B2, U.S. Pat. No. 9,475,880B2, U.S. Ser. No. 10/259,882B2, or U.S. Pat. No. 9,738,723B2, or U.S. Patent Application Publication No. US2018237534, US2018273632, or US2019161555, or International Patent Application Publication No. WO2019223733 or WO2019086497 (all of which are incorporated herein in their entireties). In certain embodiments, the agonist of the OX40 costimulatory pathway is MEDI6383 (AstraZeneca) or ATOR-1015 (Alligator Bioscience). In certain embodiments, the agonist of the OX40 costimulatory pathway is OX40L. In certain embodiments, the agonist of the OX40 costimulatory pathway is a variant (e.g., an engineered/modified form) of OX40L.

In certain embodiments wherein an agonist of the OX40 costimulatory pathway is administered in combination with an arenavirus particle expressing a tumor antigen, tumor associated antigen, antigen of a pathogen that causes an infectious disease, or antigenic fragment of any of the foregoing, as described herein, the agonist of the OX40 costimulatory pathway and the arenavirus particle are administered at the same time.

### 5.9 Cytokines

Cytokines described herein include, but are not limited to, interleukins, interferons, tumor necrosis factors, lymphokines, and monokines. In certain embodiments, a cytokine described herein is a T cell-stimulating factor. In certain embodiments, a cytokine described herein is an interleukin. In specific embodiments, a cytokine described herein is IL-2, IL-7, IL-12, IL-15, IL-15/IL-15Ra, IL-15/IL-15Ra sushi domain (e.g., ALT-803, which is an IL-15/IL-15Ra sushi domain fusion protein with an additional mutation (N72D)), IL-21, or IL-33, or a variant (e.g., an engineered/modified form) of any of the forgoing. In a specific embodiment, a cytokine described herein is IL-12 (e.g., IL-12p70, which can be a single chain IL-12p70), or a variant (e.g., an engineered/modified form) thereof. In certain embodiments, a cytokine described herein is identical to a naturally occurring cytokine. In certain embodiments, a cytokine described herein is a variant (e.g., an engineered/modified form) of a naturally occurring cytokine.

In certain embodiments, a cytokine can be encoded by a heterologous nucleotide sequence inserted in the arenavirus genome. In certain embodiments, the same arenavirus genome carries two heterologous nucleotide sequences-one encoding a cytokine and one encoding a tumor antigen, tumor-associated antigen, an antigen of a pathogen that causes an infectious disease, or an antigenic fragment of any of the foregoing. In certain embodiments, the nucleotide sequence encoding the tumor antigen, tumor associated antigen, antigen of a pathogen that causes an infectious disease, or the antigenic fragment of any of the foregoing, and the nucleotide sequence encoding the cytokine, are at the same position of the same arenaviral genome segment for insertion of heterologous nucleotide sequence(s) (see Sections 5.5 and 5.6), and therefore are under the control of the same UTR. In certain embodiments, the nucleotide sequence encoding the tumor antigen, tumor associated antigen, antigen of a pathogen that causes an infectious disease, or the antigenic fragment of any of the foregoing, and the nucleotide sequence encoding the cytokine, are on the same segment of the arenaviral genome. In certain embodiments, the nucleotide sequence encoding the tumor antigen, tumor associated antigen, antigen of a pathogen that causes an infectious disease, or the antigenic fragment of any of the foregoing, and the nucleotide sequence encoding the cytokine, are at different positions of the arenaviral genome. In certain embodiments, the nucleotide sequence encoding the tumor antigen, tumor associated antigen, antigen of a pathogen that causes an infectious disease, or the antigenic fragment of any of the foregoing, and the nucleotide sequence encoding the cytokine, are on different segments of the arenaviral genome. In certain embodiments, the nucleotide sequence encoding the tumor antigen, tumor associated antigen, antigen of a pathogen that causes an infectious disease, or the antigenic fragment of any of the foregoing, and the nucleotide sequence encoding the cytokine, are separated via a spacer sequence. In certain embodiments, the sequence encoding the tumor antigen, tumor associated antigen, antigen of a pathogen that causes an infectious disease, or the antigenic fragment of any of the foregoing, and the nucleotide sequence encoding the cytokine, are separated by an internal ribosome entry site, or a sequence encoding a protease cleavage site. In certain embodiments, the nucleotide sequence encoding the tumor antigen, tumor associated antigen, antigen of a pathogen that causes an infectious disease, or the antigenic fragment of any of the foregoing, and the nucleotide sequence encoding the cytokine, are separated by a nucleotide sequence encoding a linker or a self-cleaving peptide. Any linker peptide or self-cleaving peptide known to the skilled artisan can be used with the compositions and methods provided herein. In certain embodiments, the tumor antigen, tumor associated antigen, antigen of a pathogen that causes an infectious disease, or the antigenic fragment of any of the foregoing, and the cytokine, are directly fused together. In certain embodiments, the tumor antigen, tumor associated antigen, antigen of a pathogen that causes an infectious disease, or the antigenic fragment of any of the foregoing, and the cytokine, are fused together via a peptide linker. In certain embodiments, the tumor antigen, tumor associated antigen, antigen of a pathogen that causes an infectious disease, or the antigenic fragment of any of the foregoing, and the cytokine are separated from each other via a self-cleaving peptide. In certain embodiments, the tumor antigen, tumor associated antigen, antigen of a pathogen that causes an infectious disease, or the antigenic fragment of any of the foregoing, and the cytokine are expressed on the same arenavirus particle. In certain embodiments, the tumor antigen, tumor associated antigen, antigen of a pathogen that causes an infectious disease, or the antigenic fragment of any of the foregoing, and the cytokine are expressed on different arenavirus particles. In certain embodiments, the tumor antigen, tumor associated antigen, antigen of a pathogen that causes an infectious disease, or the antigenic fragment of any of the foregoing, and the cytokine are expressed on different arenavirus particles derived from the same arenavirus strain. In certain embodiments, the tumor antigen, tumor associated antigen, antigen of a pathogen that causes an infectious disease, or the antigenic fragment of any of the foregoing, and the cytokine are expressed on different arenavirus particles derived from different arenavirus strains.

In certain embodiments, a cytokine is administered separately from the arenavirus particle and is not encoded by the arenavirus genome. In certain embodiments, one cytokine is encoded by the arenavirus genome (either in the genome of the same arenavirus particle which also encodes the tumor antigen, tumor-associated antigen, antigen of a pathogen that causes an infectious disease, or the antigenic fragment of any of the foregoing or in the genome of a separate arenavirus particle) and one cytokine is administered separately (i.e., it is not encoded by the arenavirus genome). In certain embodiments, the arenavirus genome encodes IL-12. In certain embodiments, IL-12 is administered separately from the arenavirus particle and is not encoded by the arenavirus genome. In certain embodiments, a cytokine can be encoded by mRNA, DNA or a non-arenavirus viral vector, and the mRNA, DNA or non-arenavirus viral vector is administered to a subject described herein to deliver the cytokine.

In certain embodiments, the cytokine is directly administered to a subject described herein (preferably in the form of a pharmaceutical composition), and is not encoded by any arenavirus genome. In certain embodiments, the cytokine is directly administered to the subject in a composition, and an arenavirus encoding a tumor antigen, tumor-associated antigen, an antigen of a pathogen that causes an infectious disease, or an antigenic fragment of any of the foregoing is also administered to the subject. In certain embodiments, the composition comprising the cytokine further comprises an antibody that specifically binds to the cytokine. In certain embodiments, the cytokine is IL-2. In certain embodiments, the composition comprising IL-2 further comprises an anti-IL-2 antibody. In certain embodiments, the IL-2 is a recombinant human IL-2 and the anti-IL-2 antibody is an anti-huIL-2 antibody. In certain embodiments, the molar ratio of the IL-2 and anti-IL-2 antibody in the composition is between about 1:10 and about 10:1. In certain embodiments, the molar ratio of the IL-2 and anti-IL-2 antibody in the composition is about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, about 10:1. In certain embodiments, the molar ratio of the IL-2 and anti-IL-2 antibody in the composition is between about 1:1 and about 3:1. In certain embodiments, the molar ratio of the IL-2 and anti-IL-2 antibody in the composition is about 2:1. In certain embodiments, the molar ratio of the IL-2 and anti-IL-2 antibody in the composition is 2:1.

In certain embodiments, the cytokine is a fusion protein comprising IL-2 linked to an immunoglobulin. In certain embodiments, the immunoglobulin is an antibody. In certain embodiments, the immunoglobulin is an anti-IL-2 antibody. In certain embodiments, the anti-IL-2 antibody specifically binds to the IL-2Ra-binding domain of IL-2. In certain embodiments, the cytokine is a fusion protein comprising hIL-2 linked to an antibody specific for the IL-2Ra-binding domain of IL-2. In certain embodiments, the cytokine is ANV419 (see anaveon.com).

In certain embodiments, the cytokine is a modified IL-2 that has abrogated binding to CD25. In certain embodiments, the IL-2 is selected from the group consisting of ANV419 (see anaveon.com), XTX202 (see xiliotx.com), AB248 (see asherbio.com), MDNA11 (see www.medicenna.com), STK-012 (see www.synthekine.com), and combinations thereof.

In certain embodiments, the cytokine comprises an amino acid sequence as shown in Table 2 (see Section 6) or is encoded by a nucleotide comprising a nucleotide sequence as shown in Table 2.

### 5.10 Methods of Use

Provided herein are methods for treating or preventing a neoplastic disease, such as cancer, and methods for treating or preventing an infection disease. In certain embodiments, the methods comprise delivering an immune checkpoint modulator (Section 5.8) to a subject described herein. A protein can be delivered to a subject by, for example, administering the protein directly to the subject, or administering an arenavirus-vectored protein to the subject. In certain embodiments, a first and a second immune checkpoint modulator are delivered to a subject described herein, wherein the first immune checkpoint modulator is arenavirus-vectored and the second one is not, or wherein both immune checkpoint modulators are arenavirus-vectored, or wherein both immune checkpoint modulators are not arenavirus-vectored (e.g., both immune checkpoint modulators are directly administered to the subject). In certain embodiments, the methods comprise delivering a cytokine (Section 5.9) to a subject described herein (directly administered to the subject, or arenavirus-vectored). In certain embodiments, a first and a second cytokine are delivered to a subject described herein, wherein the first cytokine is arenavirus-vectored and the second one is not, or wherein both cytokines are arenavirus-vectored, or wherein both cytokines are not arenavirus-vectored (e.g., both cytokines are directly administered to the subject).

In certain embodiments, the methods comprise delivering a combination of an immune checkpoint modulator (Section 5.8) and an antigen (Sections 5.1 and 5.2) to a subject described herein. The antigen can also be expressed from an arenavirus vector as described herein or the antigen can be administered directly as a protein. In certain embodiments, a first and a second immune checkpoint modulator are delivered in addition to the antigen, wherein the first immune checkpoint modulator is arenavirus-vectored and the second one is not, or wherein both immune checkpoint modulators are arenavirus-vectored, or wherein both immune checkpoint modulators are not arenavirus-vectored (e.g., both immune checkpoint modulators are directly administered to the subject).

In certain embodiments, the methods comprise delivering a cytokine, such as IL-12 (e.g., IL-12p70, which can be a single chain IL-12p70) or IL-2 (e.g., delivered with anti-IL-2 antibody in the same composition), to a subject described herein. In certain embodiments, the method further comprises delivering an antigen (Sections 5.1 and 5.2) to the subject, wherein the antigen can also be expressed from an arenavirus vector as described herein or the antigen can be administered directly as a protein. In certain embodiments, the method even further comprises delivering an immune checkpoint modulator (Section 5.8) to the subject, wherein the immune checkpoint modulator can be arenavirus-vectored or can be administered directly.

In certain embodiments, the composition comprising the cytokine further comprises an antibody that specifically binds to the cytokine. In certain embodiments, the cytokine is IL-2. In certain embodiments, the composition comprising IL-2 further comprises an anti-IL-2 antibody. In certain embodiments, the IL-2 is a recombinant human IL-2 and the anti-IL-2 antibody is an anti-huIL-2 antibody. In certain embodiments, the cytokine is a fusion protein comprising IL-2 linked to an immunoglobulin. In certain embodiments, the immunoglobulin is an antibody. In certain embodiments, the immunoglobulin is an anti-IL-2 antibody. In certain embodiments, the anti-IL-2 antibody specifically binds to the IL-2Ra-binding domain of IL-2. In certain embodiments, the cytokine is a fusion protein comprising hIL-2 linked to an antibody specific for the IL-2Ra-binding domain of IL-2. In certain embodiments, the cytokine is ANV419 (see anaveon.com). In certain embodiments, the cytokine is a modified IL-2 that has abrogated binding to CD25. In certain embodiments, the IL-2 is selected from the group consisting of ANV419 (see anaveon.com), XTX202 (see xiliotx.com), AB248 (see asherbio.com), MDNA11 (see www.medicenna.com), STK-012 (see www.synthekine.com), and combinations thereof.

In certain embodiments, an arenavirus particle whose genome encodes a tumor antigen, tumor-associated antigen, antigen of a pathogen that causes an infection disease, or antigenic fragment of any of the foregoing as described herein is administered in combination with an immune checkpoint modulator (see above). In certain embodiments, the immune checkpoint modulator is an agonist of the 4-1BB costimulatory pathway (Section 5.8(a)) and/or an agonist of the OX40 co-stimulatory pathway (Section 5.8(b)). The immune checkpoint modulator can be encoded by the arenavirus genome as described herein. In certain embodiments, the immune checkpoint modulator is administered separately. In certain embodiments, one immune checkpoint modulator is encoded by the arenavirus genome and one immune checkpoint modulator is administered separately (and is not encoded by the arenavirus genome). In certain embodiments, a first arenavirus particle comprises a genome that encodes a tumor antigen, tumor associated antigen, antigen of a pathogen that causes an infection disease, or antigenic fragment of any of the foregoing, and a second arenavirus particle comprises a genome that encodes a ligand of 4-1BB. In certain embodiments, a first arenavirus particle comprises a genome that encodes a tumor antigen, tumor associated antigen, antigen of a pathogen that causes an infection disease, or antigenic fragment of any of the foregoing, and a second arenavirus particle comprises a genome that encodes a ligand of OX40.

In certain embodiments, an arenavirus particle whose genome encodes a tumor antigen, tumor-associated antigen, antigen of a pathogen that causes an infection disease, or antigenic fragment of any of the foregoing as described herein is administered in combination with a cytokine (see above). In certain embodiments, the cytokine is IL-12 (Section 5.9). The cytokine can be encoded by the arenavirus genome as described herein. In certain embodiments, the cytokine is administered separately. In certain embodiments, one cytokine is encoded by the arenavirus genome and one cytokine is administered separately (and is not encoded by the arenavirus genome). In certain embodiments, a first arenavirus particle comprises a genome that encodes a tumor antigen, tumor associated antigen, antigen of a pathogen that causes an infection disease, or antigenic fragment of any of the foregoing, and a second arenavirus particle comprises a genome that encodes IL-12. In certain embodiments, the cytokine is directly administered to a subject separately (preferably in the form of a pharmaceutical composition), and is not encoded by any arenavirus genome. In certain embodiments, the composition comprising the cytokine further comprises an antibody that specifically binds to the cytokine. In certain embodiments, the cytokine is IL-2. In certain embodiments, the composition comprising IL-2 further comprises an anti-IL-2 antibody. In certain embodiments, the molar ratio of the IL-2 and anti-IL-2 antibody in the composition is about 2:1. In certain embodiments, the cytokine is a fusion protein comprising IL-2 linked to an immunoglobulin. In certain embodiments, the immunoglobulin is an antibody. In certain embodiments, the immunoglobulin is an anti-IL-2 antibody. In certain embodiments, the anti-IL-2 antibody specifically binds to the IL-2Ra-binding domain of IL-2. In certain embodiments, the cytokine is a fusion protein comprising hIL-2 linked to an antibody specific for the IL-2Ra-binding domain of IL-2. In certain embodiments, the cytokine is ANV419 (see anaveon.com). In certain embodiments, the cytokine is a modified IL-2 that has abrogated binding to CD25. In certain embodiments, the IL-2 is selected from the group consisting of ANV419 (see anaveon.com), XTX202 (see xiliotx.com), AB248 (see asherbio.com), MDNA11 (see www.medicenna.com), STK-012 (see www.synthekine.com), and combinations thereof.

In certain embodiments, one or more arenavirus particles provided herein, or a composition comprising the same, can be administered via intratumoral injection, that is, directly into the tumor. In certain embodiments, such intratumoral injection is administered via multiple injections (e.g., at least 2, 3, 4, 5, 6, 7, 8, 9, 10, 12, 14, 16, 18, 20, 25, 30, 40, 45, or 50 injections). In certain embodiments, said multiple injections administer different arenavirus particles, for example, a first arenavirus particle that does not express a foreign antigen and a second arenavirus particle that expresses a tumor antigen, tumor associated antigen or an antigenic fragment thereof provided herein. In certain embodiments, said multiple injections administer an arenavirus particle and another agent, such as a 4-1BB agonist. In certain embodiments, said multiple injections administer an arenavirus particle and two other agents, such as a 4-1BB agonist and another immune checkpoint modulator.

In certain embodiments, the methods further comprise co-administration of the arenavirus particle provided herein and two other agents, including an agonist of 4-1BB and another immune checkpoint modulator. In certain embodiments, the co-administration of all agents is simultaneous. In another embodiment, the co-administration of all agents is performed separately. In another embodiment, the arenavirus particle is administered prior to administration of the 4-1BB agonist and the other immune checkpoint modulator. In another embodiment, the co-administration occurs in the order of (i) arenavirus particle, (ii) 4-1BB agonist, (iii) the other immune checkpoint modulator. In another embodiment, the co-administration occurs in the order of (i) the other immune checkpoint modulator, (ii) 4-1BB agonist, (iii) arenavirus particle. In certain embodiments, the co-administration occurs in the order of (i) arenavirus particle, (ii) the other immune checkpoint modulator, (iii) 4-1BB agonist. In certain embodiments, the co-administration occurs in the order of (i) 4-1BB agonist, (ii) arenavirus particle, (iii) the other immune checkpoint modulator. In other embodiments, the co-administration occurs in the order of (i) 4-1BB agonist, (ii) immune checkpoint modulator, (iii) arenavirus particle. In another embodiment, the co-administration occurs in the order of (i) immune checkpoint modulator, (ii) arenavirus particle, (iii) 4-1BB agonist. In certain embodiments, the interval between administration of the arenavirus particle, 4-1BB agonist, and the other immune checkpoint modulator is about 1 hour, about 2 hours, about 3 hours, about 4 hours, about 5 hours, about 6 hours, about 7 hours, about 8 hours, about 9 hours, about 10 hours, about 11 hours, or about 12 hours. In certain embodiments, the interval between administration of the arenavirus particle, 4-1BB agonist, and the other immune checkpoint modulator is about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks. In certain embodiments, the interval between administration of the arenavirus particle, 4-1BB agonist, and the other immune checkpoint modulator is about 1 month, about 2 months, about 3 months, about 4 months, about 5 months, or about 6 months. In some embodiments, the method further includes administering at least one additional therapy. In some embodiments, the arenavirus particle, 4-1BB agonist, and the other immune checkpoint modulator are administered via the same route. In another embodiment, the arenavirus particle, 4-1BB agonist, and the other immune checkpoint modulator are each administered via a different route. In another embodiment, the arenavirus particle and 4-1BB agonist are administered via the same route, while the other immune checkpoint modulator is administered via a different route. In another embodiment, the 4-1BB agonist and the other immune checkpoint modulator are administered via the same route, while the arenavirus particle is administered via a different route. In some embodiments, the arenavirus particle and the other immune checkpoint modulator are administered via the same route, while the 4-1BB agonist is administered via a different route.

It shall be understood that the terms “co-administration,” “co-administered,” “administered in combination with” and the like include, but are not limited to, the administration of two or more ingredients at the same time or in the same formulation. Such a term shall be construed to include any means of combination therapy, including the administration of two or more ingredients at different times and/or in different formulations.

In certain embodiments, the methods further comprise co-administration of an arenavirus particle that comprises a genome that encodes a tumor antigen, tumor associated antigen, antigen of a pathogen that causes an infection disease, or antigenic fragment of any of the foregoing and one other agent, including a second arenavirus particle that comprises a genome that encodes a ligand of 4-1BB, or another agonist of the 4-1BB costimulatory pathway. In certain embodiments, the methods further comprise co-administration of an arenavirus particle that comprises a genome that encodes a tumor antigen, tumor associated antigen, antigen of a pathogen that causes an infection disease, or antigenic fragment of any of the foregoing and one other agent, including a second arenavirus particle that comprises a genome that encodes IL-12. In certain embodiments, co-administration of the first and second arenavirus particle is simultaneous. In another embodiment, the co-administration of all agents is performed separately. In certain embodiments, the co-administration occurs in the order of (i) an arenavirus particle that comprises a genome that encodes a tumor antigen, tumor associated antigen, antigen of a pathogen that causes an infection disease, or antigenic fragment of any of the foregoing, (ii) an arenavirus particle that comprises a genome that encodes a ligand of 4-1BB, or another agonist of the 4-1BB costimulatory pathway. In certain embodiments, the co-administration occurs in the order of (i) an arenavirus particle that comprises a genome that encodes a ligand of 4-1BB, or another agonist of the 4-1BB costimulatory pathway, (ii) an arenavirus particle that comprises a genome that encodes a tumor antigen, tumor associated antigen, antigen of a pathogen that causes an infection disease, or antigenic fragment of any of the foregoing. In certain embodiments, the co-administration occurs in the order of (i) an arenavirus particle that comprises a genome that encodes a tumor antigen, tumor associated antigen, antigen of a pathogen that causes an infection disease, or antigenic fragment of any of the foregoing, (ii) an arenavirus particle that comprises a genome that encodes IL-12. In certain embodiments, the co-administration occurs in the order of (i) an arenavirus particle that comprises a genome that encodes IL-12, (ii) an arenavirus particle that comprises a genome that encodes a tumor antigen, tumor associated antigen, antigen of a pathogen that causes an infection disease, or antigenic fragment of any of the foregoing. In certain embodiments, the interval between administration of the first arenavirus particle and the second arenavirus particle or an agonist of the 4-1BB costimulatory pathway is about 1 hour, about 2 hours, about 3 hours, about 4 hours, about 5 hours, about 6 hours, about 7 hours, about 8 hours, about 9 hours, about 10 hours, about 11 hours, or about 12 hours. In certain embodiments, the interval between administration of the arenavirus particle and second arenavirus particle or agonist of the 4-1BB costimulatory pathway is about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks. In certain embodiments, the interval between administration of the arenavirus particle and second arenavirus particle or agonist of the 4-1BB costimulatory pathway is about 1 month, about 2 months, about 3 months, about 4 months, about 5 months, or about 6 months. In some embodiments, the method further includes administering at least one additional therapy. In certain embodiments, the arenavirus particle and second arenavirus particle or agonist of the 4-1BB costimulatory pathway are administered via the same route. In certain embodiments, the arenavirus particle and second arenavirus particle or agonist of the 4-1BB costimulatory pathway are administered via different routes.

Provided herein are methods for treating a solid tumor in a subject comprising injecting an arenavirus particle directly into the tumor wherein the arenavirus particle expresses a tumor antigen or tumor-associated antigen or antigenic fragment thereof. In certain embodiments, injecting comprises multiple administrations of the same arenavirus particle. In certain embodiments, injecting comprises multiple administrations of arenavirus particles derived from the same arenavirus (that is, with the same backbone), but expressing different tumor antigens or tumor-associated antigens or antigenic fragments thereof. In certain embodiments, injecting comprises multiple administrations of arenavirus particles derived from different arenaviruses (that is, with different backbones), but expressing the same tumor antigen or tumor-associated antigen or antigenic fragment thereof. In certain embodiments, injecting comprises multiple administrations of arenavirus particles derived from different arenaviruses (that is, with different backbones), and expressing different tumor antigens or tumor-associated antigens or antigenic fragments thereof.

In other embodiments, provided herein are methods for treating a solid tumor in a subject comprising injecting an arenavirus particle directly into the tumor wherein the arenavirus particle expresses a tumor antigen or tumor-associated antigen or antigenic fragment thereof, and further comprising systemically administering a first arenavirus particle prior to said injecting. In certain embodiments, the arenavirus particle injected directly into the tumor and the systemically administered first arenavirus particle are the same. In certain embodiments, the arenavirus particle injected directly into the tumor and the systemically administered first arenavirus particle are derived from the same arenavirus (that is, with the same backbone), but express different tumor antigens or tumor-associated antigens or antigenic fragments thereof. In certain embodiments, the arenavirus particle injected directly into the tumor and the systemically administered first arenavirus particle are derived from different arenaviruses (that is, with different backbones), but express the same tumor antigen or tumor-associated antigen or antigenic fragment thereof. In certain embodiments, the arenavirus particle injected directly into the tumor and the systemically administered first arenavirus particle are derived from different arenaviruses (that is, with different backbones), and express different tumor antigens or tumor-associated antigens or antigenic fragments thereof. In certain embodiments, systemically administering a first arenavirus particle comprises multiple administrations of the same arenavirus particle. In certain embodiments, systemically administering a first arenavirus particle comprises multiple administrations of arenavirus particles derived from the same arenavirus (that is, with the same backbone), but expressing different tumor antigens or tumor-associated antigens or antigenic fragments thereof. In certain embodiments, systemically administering a first arenavirus particle comprises multiple administrations of arenavirus particles derived from different arenaviruses (that is, with different backbones), but expressing the same tumor antigen or tumor-associated antigen or antigenic fragment thereof. In certain embodiments, systemically administering a first arenavirus particle comprises multiple administrations of arenavirus particles derived from different arenaviruses (that is, with different backbones), and expressing different tumor antigens or tumor-associated antigens or antigenic fragments thereof.

In other embodiments, provided herein are methods for treating a solid tumor in a subject comprising injecting an arenavirus particle directly into the tumor wherein the arenavirus particle expresses a tumor antigen or tumor-associated antigen or antigenic fragment thereof, and further comprising systemically administering a second arenavirus particle after said injecting. In certain embodiments, the arenavirus particle injected directly into the tumor and the systemically administered second arenavirus particle are the same. In certain embodiments, the arenavirus particle injected directly into the tumor and the systemically administered second arenavirus particle are derived from the same arenavirus (that is, with the same backbone), but express different tumor antigens or tumor-associated antigens or antigenic fragments thereof. In certain embodiments, the arenavirus particle injected directly into the tumor and the systemically administered second arenavirus particle are derived from different arenaviruses (that is, with different backbones), but express the same tumor antigen or tumor-associated antigen or antigenic fragment thereof. In certain embodiments, the arenavirus particle injected directly into the tumor and the systemically administered second arenavirus particle are derived from different arenaviruses (that is, with different backbones), and express different tumor antigens or tumor-associated antigens or antigenic fragments thereof. In certain embodiments, systemically administering a second arenavirus particle comprises multiple administrations of the same arenavirus particle. In certain embodiments, systemically administering a second arenavirus particle comprises multiple administrations of arenavirus particles derived from the same arenavirus (that is, with the same backbone), but expressing different tumor antigens or tumor-associated antigens or antigenic fragments thereof. In certain embodiments, systemically administering a second arenavirus particle comprises multiple administrations of arenavirus particles derived from different arenaviruses (that is, with different backbones), but expressing the same tumor antigen or tumor-associated antigen or antigenic fragment thereof. In certain embodiments, systemically administering a second arenavirus particle comprises multiple administrations of arenavirus particles derived from different arenaviruses (that is, with different backbones), and expressing different tumor antigens or tumor-associated antigens or antigenic fragments thereof.

In certain embodiments, provided herein are methods for treating a solid tumor in a subject comprising (a) administering a first arenavirus particle to a subject, wherein the first arenavirus particle does not express a tumor antigen or tumor-associated antigen or antigenic fragment thereof; and (b) administering a second arenavirus particle to a subject, wherein the second arenavirus particle expresses a tumor antigen or tumor-associated antigen or antigenic fragment thereof. In certain embodiments, administering comprises multiple administrations of the same arenavirus particle. In certain embodiments, administering a first arenavirus particle comprises multiple administrations of arenavirus particles derived from different arenaviruses (that is, with different backbones). In certain embodiments, administering a second arenavirus particle comprises multiple administrations of the same arenavirus particle. In certain embodiments, administering a second arenavirus particle comprises multiple administrations of arenavirus particles derived from the same arenavirus (that is, with the same backbone), but expressing different tumor antigens or tumor-associated antigens or antigenic fragments thereof. In certain embodiments, administering a second arenavirus particle comprises multiple administrations of arenavirus particles derived from different arenaviruses (that is, with different backbones), but expressing the same tumor antigen or tumor-associated antigen or antigenic fragment thereof. In certain embodiments, administering a second arenavirus particle comprises multiple administrations of arenavirus particles derived from different arenaviruses (that is, with different backbones), and expressing different tumor antigens or tumor-associated antigens or antigenic fragments thereof.

In another embodiment, provided herein are methods for treating a solid tumor in a subject comprising (a) injecting a first arenavirus particle directly into the tumor, wherein the first arenavirus particle does not express a tumor antigen or tumor-associated antigen or antigenic fragment thereof; and (b) injecting a second arenavirus particle directly into the tumor, wherein the second arenavirus particle expresses a tumor antigen or tumor-associated antigen or antigenic fragment thereof.

In another embodiment, provided herein are methods for treating a solid tumor in a subject comprising (a) intravenously administering a first arenavirus particle to the subject, wherein the first arenavirus particle does not express a tumor antigen or tumor-associated antigen or antigenic fragment thereof, and (b) injecting a second arenavirus particle directly into the tumor, wherein the second arenavirus particle expresses a tumor antigen or tumor-associated antigen or antigenic fragment thereof.

In another embodiment, provided herein are methods for treating a solid tumor in a subject comprising (a) injecting a first arenavirus particle directly into the tumor, wherein the first arenavirus particle does not express a tumor antigen or tumor-associated antigen or antigenic fragment thereof, and (b) intravenously administering a second arenavirus particle to the subject, wherein the second arenavirus particle expresses a tumor antigen or tumor-associated antigen or antigenic fragment thereof.

In certain embodiments, the first arenavirus particle does not express a foreign antigen. In certain embodiments, the first arenavirus particle comprises a nucleotide sequence comprising a deleted or inactivated viral ORF. In certain embodiments, the first arenavirus particle comprises a nucleotide sequence wherein the UTR is directly fused to the IGR. In certain embodiments, the first arenavirus particle comprises a nucleotide sequence comprising an ORF for a marker, such as GFP. In certain embodiments, the first arenavirus particle comprises a nucleotide sequence comprising a heterologous non-coding sequence.

In another embodiment, provided herein are methods for treating a solid tumor in a subject comprising (a) injecting a first arenavirus particle directly into the tumor, wherein the first arenavirus particle does not express a tumor antigen or tumor-associated antigen or antigenic fragment thereof; and (b) administering a second arenavirus particle to the subject, wherein the second arenavirus particle expresses a tumor antigen or tumor-associated antigen or antigenic fragment thereof. In certain embodiments, the first arenavirus particle does not express a foreign antigen. In certain embodiments, the first arenavirus particle comprises a nucleotide sequence comprising a deleted or inactivated viral ORF. In certain embodiments, the first arenavirus particle comprises a nucleotide sequence wherein the UTR is directly fused to the IGR. In certain embodiments, the first arenavirus particle comprises a nucleotide sequence comprising an ORF for a marker, such as GFP. In certain embodiments, the first arenavirus particle comprises a nucleotide sequence comprising a heterologous non-coding sequence. In specific embodiments, the second arenavirus particle is replication-competent. In specific embodiments, the second arenavirus particle is replication-defective. In certain embodiments, the second arenavirus particle is tri-segmented. In specific embodiments, the second arenavirus particle is tri-segmented and replication-competent. In specific embodiments, the second arenavirus particle is tri-segmented and replication-defective.

In another embodiment, provided herein are methods for treating a solid tumor in a subject comprising (a) injecting a first arenavirus particle directly into the tumor, wherein the first arenavirus particle is replication-competent and does not express a tumor antigen or tumor-associated antigen or antigenic fragment thereof, and (b) administering a second arenavirus particle to the subject, wherein the second arenavirus particle expresses a tumor antigen or tumor-associated antigen or antigenic fragment thereof. In certain embodiments, the first arenavirus particle does not express a foreign antigen. In certain embodiments, the first arenavirus particle comprises a nucleotide sequence comprising a deleted or inactivated viral ORF. In certain embodiments, the first arenavirus particle comprises a nucleotide sequence wherein the UTR is directly fused to the IGR. In certain embodiments, the first arenavirus particle comprises a nucleotide sequence comprising an ORF for a marker, such as GFP. In certain embodiments, the first arenavirus particle comprises a nucleotide sequence comprising a heterologous non-coding sequence.

In another embodiment, provided herein are methods for treating a solid tumor in a subject comprising (a) injecting a first arenavirus particle directly into the tumor, wherein the first arenavirus particle is replication-competent and expresses a tumor antigen or tumor-associated antigen or antigenic fragment thereof, and (b) administering a second arenavirus particle to the subject, wherein the second arenavirus particle expresses a tumor antigen or tumor-associated antigen or antigenic fragment thereof. In certain embodiments, the first arenavirus particle is tri-segmented. In specific embodiments, the second arenavirus particle is replication-competent. In specific embodiments, the second arenavirus particle is replication-defective. In certain embodiments, the second arenavirus particle is tri-segmented. In specific embodiments, the second arenavirus particle is tri-segmented and replication-competent. In specific embodiments, the second arenavirus particle is tri-segmented and replication-defective.

In one embodiment, provided herein are methods of treating or preventing a neoplastic disease or an infectious disease in a subject comprising administering to the subject one or more arenavirus particles expressing a tumor antigen, tumor associated antigen, antigen of a pathogen that causes an infectious disease, or an antigenic fragment of any of the foregoing as provided herein or a composition thereof, co-expressing an immune checkpoint modulator or a cytokine, and/or co-administered in combination with an immune checkpoint modulator or a cytokine, and optionally in combination with one or more arenavirus particles that do not express a foreign antigen. In a specific embodiment, a method for treating or preventing a neoplastic disease or an infectious disease described herein comprises administering to a subject in need thereof a therapeutically effective amount of one or more arenavirus particle(s) expressing a tumor antigen, tumor associated antigen, antigen of a pathogen that causes an infectious disease, or an antigenic fragment of any of the foregoing provided herein or a composition thereof, co-expressing an immune checkpoint modulator or a cytokine, and/or co-administered in combination with an immune checkpoint modulator or a cytokine, and optionally in combination with one or more arenavirus particle(s) that do not express a foreign antigen. The subject can be a mammal, such as but not limited to a human, a mouse, a rat, a guinea pig, a domesticated animal, such as, but not limited to, a cow, a horse, a sheep, a pig, a goat, a cat, a dog, a hamster, a donkey. In a specific embodiment, the subject is a human.

In another embodiment, provided herein are methods for inducing an immune response against a tumor cell or infected cell in a subject comprising administering to the subject an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, and optionally in combination with one or more arenavirus particle(s) that do not express a foreign antigen.

In another embodiment, the subjects to whom an arenavirus particle expressing a tumor antigen, tumor associated antigen or an antigenic fragment thereof provided herein, or a composition thereof is administered, wherein the arenavirus particle co-expresses an immune checkpoint modulator or a cytokine, and/or is co-administered in combination with an immune checkpoint modulator or a cytokine, have, are susceptible to, or are at risk for a neoplastic disease.

In another embodiment, the subjects to whom an arenavirus particle expressing a tumor antigen, tumor associated antigen or an antigenic fragment thereof provided herein, or a composition thereof is administered, wherein the arenavirus particle co-expresses an immune checkpoint modulator or a cytokine, and/or is co-administered in combination with an immune checkpoint modulator or a cytokine, have, are susceptible to, or are at risk for development of a neoplastic disease, such as cancer, or exhibit a pre-cancerous tissue lesion. In another specific embodiment, the subjects to whom an arenavirus particle expressing a tumor antigen, tumor associated antigen or an antigenic fragment thereof provided herein, or a composition thereof is administered, wherein the arenavirus particle co-expresses an immune checkpoint modulator or a cytokine, and/or is co-administered in combination with an immune checkpoint modulator or a cytokine, are diagnosed with a neoplastic disease, such as cancer, or exhibit a pre-cancerous tissue lesion.

In another embodiment, the subjects having an infectious disease to whom an arenavirus particle expressing an antigen of a pathogen that causes an infectious disease, or an antigenic fragment thereof provided herein, or a composition thereof is administered, wherein the arenavirus particle co-expresses an immune checkpoint modulator or a cytokine, and/or is co-administered in combination with an immune checkpoint modulator or a cytokine, have, are susceptible to, or are at risk for an infectious disease.

In another embodiment, an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, is administered to a subject of any age group having a neoplastic disease or an infectious disease and suffering from, susceptible to, or at risk for a neoplastic disease or an infectious disease. In a specific embodiment, an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, is administered to a subject having a neoplastic disease or an infectious disease with a compromised immune system, a pregnant subject, a subject undergoing an organ or bone marrow transplant, a subject taking immunosuppressive drugs, a subject undergoing hemodialysis, a subject who has cancer or an infection, or a subject who is suffering from, is susceptible to, or is at risk for a neoplastic disease or an infectious disease. In a more specific embodiment, an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, is administered to a subject who is a child of 0, 1, 2, 3, 4, 5, 6, 7, 8, 9, 10, 11, 12, 13, 14, 15, 16, or 17 years of age suffering from, susceptible to, or at risk for a neoplastic disease or an infectious disease. In yet another specific embodiment, an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, is administered to a subject who is an infant suffering from, susceptible to, or at risk for a neoplastic disease or an infectious disease. In yet another specific embodiment, an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, is administered to a subject who is an infant of 0, 1, 2, 3, 4, 5, 6, 7, 8, 9, 10, 11, or 12 months of age suffering from, susceptible to, or at risk for a neoplastic disease or an infectious disease. In yet another specific embodiment, an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, is administered to an elderly subject who is suffering from, is susceptible to, or is at risk for a neoplastic disease or an infectious disease. In a more specific embodiment, an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, is administered to a subject who is a senior subject of 65, 66, 67, 68, 69, 70, 71, 72, 73, 74, 75, 76, 77, 78, 79, 80, 81, 82, 83, 84, 85, 86, 87, 88, 89, or 90 years of age. Provided herein is a method for preventing a neoplastic disease or an infectious disease in a subject susceptible to, or at risk for a neoplastic disease or an infectious disease.

In another embodiment, an arenavirus particle expressing a tumor antigen, tumor associated antigen or an antigenic fragment thereof provided herein, or a composition thereof, provided herein, co-expressing an immune checkpoint modulator or a cytokine, and/or co-administered in combination with an immune checkpoint modulator or a cytokine, is administered to subjects with a heightened risk of cancer metastasis. In a specific embodiment, an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, is administered to subjects in the neonatal period with a neonatal and therefore immature immune system.

In another embodiment, an arenavirus particle expressing a tumor antigen, tumor associated antigen or an antigenic fragment thereof provided herein, or a composition thereof, co-expressing an immune checkpoint modulator or a cytokine, and/or co-administered in combination with an immune checkpoint modulator or a cytokine, is administered to a subject having grade 0 (i.e., in situ neoplasm), grade 1, grade 2, grade 3 or grade 4 cancer or a subcategory thereof, such as grade 3A, 3B, or 3C, or an equivalent thereof.

In another embodiment, an arenavirus particle expressing a tumor antigen, tumor associated antigen or an antigenic fragment thereof provided herein, or a composition thereof, co-expressing an immune checkpoint modulator or a cytokine, and/or co-administered in combination with an immune checkpoint modulator or a cytokine, is administered to a subject having cancer at a Tumor, Node, Metastasis (TNM) stage of any combination selected from Tumor T1, T2, T3, and T4, and Node NO, N1, N2, or N3, and Metastasis M0 and M1.

Successful treatment of a cancer patient can be assessed as prolongation of expected survival, induction of an anti-tumor immune response, or improvement of a particular characteristic of a cancer. Examples of characteristics of a cancer that might be improved include tumor size (e.g., TO, T is, or T1-4), state of metastasis (e.g., M0, M1), number of observable tumors, node involvement (e.g., NO, N1-4, Nx), grade (i.e., grades 1, 2, 3, or 4), stage (e.g., 0, I, II, III, or IV), presence or concentration of certain markers on the cells or in bodily fluids (e.g., AFP, B2M, beta-HCG, BTA, CA 15-3, CA 27.29, CA 125, CA 72.4, CA 19-9, calcitonin, CEA, chromgrainin A, EGFR, hormone receptors, HER2, HCG, immunoglobulins, NSE, NMP22, PSA, PAP, PSMA, S-100, TA-90, and thyroglobulin), and/or associated pathologies (e.g., ascites or edema) or symptoms (e.g., cachexia, fever, anorexia, or pain). The improvement, if measurable by percent, can be at least 5, 10, 15, 20, 25, 30, 40, 50, 60, 70, 80, or 90% (e.g., survival, or volume or linear dimensions of a tumor).

In another embodiment, an arenavirus particle expressing a tumor antigen, tumor associated antigen or an antigenic fragment thereof provided herein, or a composition thereof, co-expressing an immune checkpoint modulator or a cytokine, and/or co-administered in combination with an immune checkpoint modulator or a cytokine, is administered to a subject having a dormant cancer (e.g., the subject is in remission). Thus, provided herein is a method for preventing reactivation of a cancer. Also provided herein are methods for reducing the frequency of reoccurrence of a cancer.

In another embodiment, an arenavirus particle expressing a tumor antigen, tumor associated antigen or an antigenic fragment thereof provided herein, or a composition thereof, co-expressing an immune checkpoint modulator or a cytokine, and/or co-administered in combination with an immune checkpoint modulator or a cytokine, is administered to a subject having a recurrent a cancer.

In another embodiment, an arenavirus particle expressing a tumor antigen, tumor associated antigen or an antigenic fragment thereof provided herein, or a composition thereof, co-expressing an immune checkpoint modulator or a cytokine, and/or co-administered in combination with an immune checkpoint modulator or a cytokine, is administered, to a subject with a genetic predisposition for a cancer. In another embodiment, an arenavirus particle expressing a tumor antigen, tumor associated antigen or an antigenic fragment thereof provided herein, or a composition thereof, co-expressing an immune checkpoint modulator or a cytokine, and/or co-administered in combination with an immune checkpoint modulator or a cytokine, is administered to a subject with risk factors. Exemplary risk factors include aging, tobacco, sun exposure, radiation exposure, chemical exposure, family history, alcohol, poor diet, lack of physical activity, or being overweight.

In another embodiment, an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, is administered to subjects who suffer from one or more types of cancers or infections. In other embodiments, any type of neoplastic disease, such as cancer, or any type of infectious disease, that is susceptible to treatment with the compositions described herein might be targeted.

In another embodiment, administering an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, to subjects confer cell-mediated immunity (CMI) against a neoplastic cell or tumor, such as a cancer cell or tumor, or against infectious disease. Without being bound by theory, in another embodiment, an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, infects and expresses antigens of interest in antigen presenting cells (APC) of the host (e.g., macrophages) for direct presentation of antigens on Major Histocompatibility Complex (MHC) class I and II. In another embodiment, administering an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, to subjects induces polyfunctional IFN-γ and TNF-α co-producing cancer-specific or pathogen-specific CD4+ and CD8+ T cell responses (both IFN-γ and TNF-α are produced by CD4+ and CD8+ T cells) of high magnitude to treat a neoplastic disease or an infectious disease.

In another embodiment, administering an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, increases or improves one or more clinical outcomes for cancer treatment or treatment of an infectious disease. Non-limiting examples of such outcomes are overall survival, progression-free survival, time to progression, time to treatment failure, event-free survival, time to next treatment, overall response rate and duration of response. The increase or improvement in one or more of the clinical outcomes can be by at least about 10%, at least about 20%, at least about 25%, at least about 30%, at least about 35%, at least about 40%, at least about 50%, at least about 60%, at least about 70%, at least about 80%, at least about 90%, or more, compared to a patient or group of patients having the same neoplastic disease or infectious disease in the absence of such treatment.

Changes in cell-mediated immunity (CMI) response function against a neoplastic cell or tumor, including a cancer cell or tumor, or against an infectious disease, induced by administering an arenavirus particle expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, in subjects can be measured by any assay known to the skilled artisan including, but not limited to flow cytometry (see, e.g., Perfetto S. P. et al., Nat Rev Immun. 2004; 4(8):648-55), lymphocyte proliferation assays (see, e.g., Bonilla F. A. et al., Ann Allergy Asthma Immunol. 2008; 101:101-4; and Hicks M. J. et al., Am J Clin Pathol. 1983; 80:159-63), assays to measure lymphocyte activation including determining changes in surface marker expression following activation of measurement of cytokines of T lymphocytes (see, e.g., Caruso A. et al., Cytometry. 1997; 27:71-6), ELISPOT assays (see, e.g., Czerkinsky C. C. et al., J Immunol Methods. 1983; 65:109-121; and Hutchings P. R., et al., J Immunol Methods. 1989; 120:1-8), or Natural killer cell cytotoxicity assays (see, e.g., Bonilla F. A. et al., Ann Allergy Asthma Immunol. 2005 May; 94(5 Suppl 1):S1-63).

In certain embodiments, the treatments provided herein can further be combined with a chemotherapeutic agent. Chemotherapeutic agents include alkylating agents (e.g., cyclophosphamide), platinum-based therapeutics, antimetabolites, topoisomerase inhibitors, cytotoxic antibiotics, intercalating agents, mitosis inhibitors, taxanes, or combinations of two or more thereof. In certain embodiments, the alkylating agent is a nitrogen mustard, a nitrosourea, an alkyl sulfonate, a non-classical alkylating agent, or a triazene. In certain embodiments, the chemotherapeutic agent comprises one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above. In specific embodiments, the chemotherapeutic agent comprises cyclophosphamide.

In certain embodiments, the treatments for infectious diseases provided herein can further be combined with an antibiotic or an antiviral drug.

In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered in two or more separate injections over a 1-hour period, 2-hour period, 3-hour period, 6-hour period, a 12-hour period, a 24-hour period, or a 48-hour period.

In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered, in two or more separate injections over a 3-day period, a 5-day period, a 1-week period, a 2-week period, a 3-week period, a 4-week period, or a 12-week period.

In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered in two or more separate injections over a 6-month period, a 12-month period, a 24-month period, or a 48-month period.

In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered with a first dose at an elected time, and a second dose at least 2 hours after the first dose. In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered with a first dose at an elected time, a second dose at least 2 hours after the first dose, and a third dose at least 6 hours after the first dose.

In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered with a first dose at an elected date, and a second dose at least 2 days after the first dose. In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered with a first dose at an elected date, a second dose at least 2 days after the first dose, and a third dose at least 6 days after the first dose.

In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered with a first dose at an elected date, and a second dose at least 2 weeks after the first dose. In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered with a first dose at an elected date, a second dose at least 2 weeks after the first dose, and a third dose at least 6 weeks after the first dose.

In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered with a first dose at an elected date, and a second dose at least 2 months after the first dose. In certain embodiments, the one or more arenavirus particles expressing a tumor antigen, tumor associated antigen, antigen of a pathogen that causes an infectious disease, or an antigenic fragment of any of the foregoing provided herein, or a composition thereof, co-expressing an immune checkpoint modulator or a cytokine, and/or co-administered in combination with an immune checkpoint modulator or a cytokine, are administered with a first dose at an elected date, a second dose at least 2 months after the first dose, and a third dose at least 6 months after the first dose.

In certain embodiments, one or more arenavirus particles provided herein, or a composition thereof, are administered via peritumoral injection.

In certain embodiments, one or more arenavirus particles provided herein, or a composition thereof are administered, via intratumoral injection in combination with a second set of one or more arenavirus particles provided herein administered via another method. In certain embodiments, the second set of one or more arenavirus particles provided herein are administered systemically, for example, intravenously. In certain embodiments, one or more arenavirus particles provided herein, or a composition thereof are administered, via intravenous injection in combination with a second set of one or more arenavirus particles provided herein administered via another method. In certain embodiments, the second set of one or more arenavirus particles provided herein are administered systemically.

In embodiments wherein two arenavirus particles are administered in a treatment regime, administration may be at molar ratios ranging from about 1:1 to 1:1000, in particular including: 1:1 ratio, 1:2 ratio, 1:5 ratio, 1:10 ratio, 1:20 ratio, 1:50 ratio, 1:100 ratio, 1:200 ratio, 1:300 ratio, 1:400 ratio, 1:500 ratio, 1:600 ratio, 1:700 ratio, 1:800 ratio, 1:900 ratio, 1:1000 ratio.

In certain embodiments, provided herein is a method of treating a neoplastic disease or an infectious disease wherein a first arenavirus particle is administered first as a “prime”, and a second arenavirus particle is administered as a “boost.” The first and the second arenavirus particles can express the same or different tumor antigens, tumor associated antigens, antigens of a pathogen that causes an infectious disease, or antigenic fragments of any of the foregoing. In a specific embodiment, the first or second arenavirus particle does not express a foreign antigen. Alternatively, or additionally, in some certain embodiments, the “prime” and “boost” administration are performed with an arenavirus particle derived from different arenavirus species. In certain specific embodiments, the “prime” administration is performed with an arenavirus particle derived from LCMV, and the “boost” is performed with an arenavirus particle derived from Pichinde virus. In certain specific embodiments, the “prime” administration is performed with an arenavirus particle derived from Pichinde virus, and the “boost” is performed with an arenavirus particle derived from LCMV.

In certain embodiments, administering a first arenavirus particle expressing a tumor antigen, tumor associated antigen, antigen of a pathogen that causes an infectious disease, or antigenic fragment of any of the foregoing, followed by administering a second arenavirus particle expressing a tumor antigen, tumor associated antigen, antigen of a pathogen that causes an infectious disease, or antigenic fragment of any of the foregoing results in a greater antigen specific CD8+ T cell response than administering a single arenavirus particle expressing a tumor antigen, tumor associated antigen, antigen of a pathogen that causes an infectious disease, or antigenic fragment of any of the foregoing. In certain embodiments, said first or second arenavirus particle does not express a foreign antigen. In certain embodiments, the antigen specific CD8+ T cell count increases by 50%, 100%, 150% or 200% after the second administration compared to the first administration. In certain embodiments, administering a third arenavirus particle expressing a tumor antigen, tumor associated antigen, antigen of a pathogen that causes an infectious disease, or antigenic fragment of any of the foregoing results in a greater antigen specific CD8+ T cell response than administering two consecutive arenavirus particles expressing a tumor antigen, tumor associated antigen, antigen of a pathogen that causes an infectious disease, or antigenic fragment of any of the foregoing. In certain embodiments, the antigen specific CD8+ T cell count increases by about 50%, about 100%, about 150%, about 200% or about 250% after the third administration compared to the first administration.

In certain embodiments, provided herein are methods for treating a neoplastic disease or an infectious disease comprising administering two or more arenavirus particles, wherein the two or more arenavirus particles are homologous, and wherein the time interval between each administration is about 1 week, about 2 weeks, about 3 week, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, about 12 months, about 18 months, or about 24 months.

In certain embodiments, administering a first arenavirus particle expressing a tumor antigen, tumor associated antigen, antigen of a pathogen that causes an infectious disease, or antigenic fragment of any of the foregoing and a second, heterologous, arenavirus particle expressing a tumor antigen, tumor associated antigen, antigen of a pathogen that causes an infectious disease or antigenic fragment of any of the foregoing elicits a greater CD8+ T cell response than administering a first arenavirus particle expressing a tumor antigen, tumor associated antigen, antigen of a pathogen that causes an infectious disease, or antigenic fragment of any of the foregoing and a second, homologous, arenavirus particle expressing a tumor antigen, tumor associated antigen, antigen of a pathogen that causes an infectious disease, or antigenic fragment of any of the foregoing. In certain embodiments, said first or second arenavirus particle does not express a foreign antigen.

It is contemplated that one or more immune checkpoint modulators and/or one or more cytokines can be encoded by any one of the arenavirus particles described herein or be administered in combination with any one of the arenavirus particles described herein.

### 5.10.1 Clinical Outcomes

In certain embodiments, the methods provided herein result in superior clinical outcomes than the current standard of care. Assays/tests provided in Section 5.12 can be used to demonstrate such superior activity.

In certain embodiments, a method provided herein for the treatment of a solid tumor has an abscopal effect. Specifically, if one or more of the active agents provided herein is administered via intratumoral injection into one tumor mass, other tumor masses that were not injected with the active agent(s) also respond to the treatment (e.g., by reduced growth).

In certain embodiments, a method provided herein increases the frequency of T cells within tumors or near cells infected with the pathogen. The frequency of T cells in a tumor, such as a solid tumor, or near cells infected with the pathogen, can be measured using any assay available to the skilled artisan.

In certain embodiments, combining an arenaviral vector encoding a tumor antigen, tumor associated antigen, antigen of a pathogen that causes an infectious disease, or antigenic fragment of any of the foregoing and an agonistic anti-4-1BB antibody, or co-expressing 4-1BBL from the same arenaviral vector or a separate arenaviral vector used in combination with the arenaviral vector encoding a tumor antigen, tumor associated antigen, antigen of a pathogen that causes an infectious disease, or antigenic fragment of any of the foregoing results in superior immunogenicity and efficacy. For example, such superior immunogenicity/efficacy can be demonstrated by showing higher numbers of antigen-specific CD8+ T cells and/or higher expression of CD127 (IL-7 receptor) on antigen-specific CD8+ T cells after immunization.

In certain embodiments, the methods provided herein result in a stronger anti-tumoral or anti-infection effect and survival benefit after administration of an arenavirus vector expressing a tumor antigen, tumor associated antigen, antigen of a pathogen that causes an infectious disease, or antigenic fragment of any of the foregoing and an agonistic anti-4-1BB antibody and/or with co-expression of 4-1BBL from the same arenavirus vector or a separate arenavirus vector used in combination with the arenaviral vector encoding a tumor antigen, tumor associated antigen, antigen of a pathogen that causes an infectious disease, or antigenic fragment of any of the foregoing.

In certain embodiments, the interval between (e.g., intravenous or intratumoral) administration of the arenavirus vector and administration of an immune checkpoint modulator, such as an agonist of the 4-1BB costimulatory pathway, for example, an anti-4-1BB agonistic antibody is at most 5, 4, 3, or 2 days or at most one day, at most 20, 16, 12, 8, 4, 3, or at most 2 hours, or at most 1 hour. In certain embodiments, the administrations of the arenavirus vector and the immune checkpoint modulator, such as the agonist of the 4-1BB costimulatory pathway (e.g., the anti-4-1BB agonistic antibody) are simultaneous.

In certain embodiments, the interval between (e.g., intravenous or intratumoral) administration of the arenavirus vector and administration of a cytokine, such as IL-12 is at most 5, 4, 3, or 2 days or at most one day, at most 20, 16, 12, 8, 4, 3, or at most 2 hours, or at most 1 hour. In certain embodiments, the administrations of the arenavirus vector and the cytokine, such as IL-12 are simultaneous.

Without being bound by theory, intratumoral vector administration can be superior to intravenous administration if the vector expresses or co-expresses an immune checkpoint modulator described herein (e.g., 4-1BBL) or a cytokine described herein. Therefore, in preferred embodiments of treating solid tumors wherein an immune checkpoint modulator (e.g., 4-1BBL) or a cytokine is expressed from an arenavirus particle co-expressing a tumor antigen, tumor associated antigen, or antigenic fragment of any of the foregoing, the arenavirus particle is administered via intratumoral injection. In other embodiments of treating solid tumors wherein an immune checkpoint modulator (e.g., 4-1BBL) or a cytokine is expressed from an arenavirus particle co-expressing a tumor antigen, tumor associated antigen, or antigenic fragment of any of the foregoing, the arenavirus particle is administered via intravenous injection. In preferred embodiments of treating solid tumors wherein an immune checkpoint modulator (e.g., 4-1BBL) or a cytokine is expressed from an arenavirus particle that is co-administered with a different arenavirus particle expressing a tumor antigen, tumor associated antigen, or antigenic fragment of any of the foregoing, one or both of the arenavirus particles are administered via intratumoral injection. In one embodiment of such preferred embodiments, the arenavirus particle encoding the immune checkpoint modulator (e.g., 4-1BBL) or a cytokine is administered via intratumoral injection, and the arenavirus particle encoding the tumor antigen, tumor associated antigen, or antigenic fragment of any of the foregoing, is administered via intravenous injection. In another embodiment of such preferred embodiments, the arenavirus particle encoding the immune checkpoint modulator (e.g., 4-1BBL) or a cytokine is administered via intratumoral injection, and the arenavirus particle encoding the tumor antigen, tumor associated antigen, or antigenic fragment of any of the foregoing, is administered via intratumoral injection. In another embodiment of such preferred embodiments, the arenavirus particle encoding the immune checkpoint modulator (e.g., 4-1BBL) or a cytokine is administered via intravenous injection, and the arenavirus particle encoding the tumor antigen, tumor associated antigen, or antigenic fragment of any of the foregoing, is administered via intratumoral injection. In other embodiments of treating solid tumors wherein an immune checkpoint modulator (e.g., 4-1BBL) or a cytokine is expressed from an arenavirus particle that is co-administered with a different arenavirus particle expressing a tumor antigen, tumor associated antigen, or antigenic fragment of any of the foregoing, both arenavirus particles are administered via intravenous injection.

In certain embodiments wherein an arenavirus particle expressing a tumor antigen, tumor associated antigen, antigen of a pathogen that causes an infectious disease, or antigenic fragment of any of the foregoing is administered in combination with an immune checkpoint modulator described herein (e.g., an agonistic anti-4-1BB antibody) or a cytokine, the arenavirus particle is administered via intravenous injection. In certain embodiments wherein an arenavirus particle expressing a tumor antigen, tumor associated antigen, or antigenic fragment of any of the foregoing is administered in combination with an immune checkpoint modulator described herein (e.g., an agonistic anti-4-1BB antibody) or a cytokine for treating a solid tumor, the arenavirus particle is administered via intratumoral injection. In certain embodiments wherein an arenavirus particle expressing a tumor antigen, tumor associated antigen, antigen of a pathogen that causes an infectious disease, or antigenic fragment of any of the foregoing is administered in combination with an immune checkpoint modulator described herein (e.g., an agonistic anti-4-1BB antibody) or a cytokine and a different arenavirus particle, one or both of the arenavirus particles are administered via intravenous injection. In certain embodiments wherein an arenavirus particle expressing a tumor antigen, tumor associated antigen, or antigenic fragment of any of the foregoing is administered in combination with an immune checkpoint modulator described herein (e.g., an agonistic anti-4-1BB antibody) or a cytokine and a different arenavirus particle for treating a solid tumor, one or both of the arenavirus particles are administered via intratumoral injection.

In certain embodiments of treating a neoplastic disease, the method described herein results in an increase of the concentration of T cells near tumor cells. In specific embodiments, the method results in an increase of the concentration of CD8+ T cells, the concentration of CD4+ T cells, the concentration of tumor antigen specific T cells, the concentration of T cells producing IFN-gamma, and/or the concentration of T cells producing granzyme B, near tumor cells. In a specific embodiment, the method results in an increase of the concentration of CD8+ T cells near tumor cells. In a specific embodiment, the method results in an increase of the concentration of CD4+ T cells near tumor cells. In a specific embodiment, the method results in an increase of the concentration of tumor antigen specific T cells near tumor cells. In a specific embodiment, the method results in an increase of the concentration of T cells producing IFN-gamma near tumor cells. In a specific embodiment, the method results in an increase of the concentration of T cells producing granzyme B near tumor cells.

In certain embodiments of treating a neoplastic disease, the method described herein results in an increase of the ratio of effector T cells/regulatory T cells near tumor cells.

In certain embodiments of treating a neoplastic disease, the neoplastic disease is a solid tumor and the method described herein results in an increase of the concentration of T cells within the solid tumor. In specific embodiments, the method results in an increase of the concentration of CD8+ T cells, the concentration of CD4+ T cells, the concentration of tumor antigen specific T cells, the concentration of T cells producing IFN-gamma, and/or the concentration of T cells producing granzyme B, within the solid tumor. In a specific embodiment, the method results in an increase of the concentration of CD8+ T cells within the solid tumor. In a specific embodiment, the method results in an increase of the concentration of CD4+ T cells within the solid tumor. In a specific embodiment, the method results in an increase of the concentration of tumor antigen specific T cells within the solid tumor. In a specific embodiment, the method results in an increase of the concentration of T cells producing IFN-gamma within the solid tumor. In a specific embodiment, the method results in an increase of the concentration of T cells producing granzyme B within the solid tumor.

In certain embodiments of treating a neoplastic disease, the neoplastic disease is a solid tumor and the method described herein results in an increase of the ratio of effector T cells/regulatory T cells within the solid tumor.

In certain embodiments of treating an infectious disease, the method described herein results in an increase of the concentration of T cells near cells infected with the pathogen. In specific embodiments, the method results in an increase of the concentration of CD8+ T cells, the concentration of CD4+ T cells, the concentration of T cells specific for the antigen of the pathogen, the concentration of T cells producing IFN-gamma, and/or the concentration of T cells producing granzyme B, near cells infected with the pathogen. In a specific embodiment, the method results in an increase of the concentration of CD8+ T cells near cells infected with the pathogen. In a specific embodiment, the method results in an increase of the concentration of CD4+ T cells near cells infected with the pathogen. In a specific embodiment, the method results in an increase of the concentration of T cells specific for the antigen of the pathogen near cells infected with the pathogen. In a specific embodiment, the method results in an increase of the concentration of T cells producing IFN-gamma near cells infected with the pathogen. In a specific embodiment, the method results in an increase of the concentration of T cells producing granzyme B near cells infected with the pathogen.

In certain embodiments of treating an infectious disease, the method described herein results in an increase of the ratio of effector T cells/regulatory T cells near cells infected with the pathogen.

In certain embodiments, the method described herein results in an increase in the survival rate of subjects treated with the method described herein, compared to subjects having the same neoplastic disease or infectious disease in the absence of such treatment.

In certain embodiments, the method described herein has a higher anti-tumor or anti-infection efficacy as compared to administration of a control arenavirus particle expressing the tumor antigen, tumor associated antigen, antigen of a pathogen that causes an infectious disease, or antigenic fragment of any of the foregoing, alone. A control arenavirus particle can be any arenavirus particle deemed suitable by a skilled artisan to serve as a control arenavirus vector for the comparison. Preferably, the control arenavirus particle is derived from the same species as the arenavirus particle with which to be compared. More preferably, the control arenavirus particle has the same backbone as the arenavirus particle with which to be compared. For example, if the method comprises administering (1) an arenavirus particle expressing a tumor antigen, tumor associated antigen, antigen of a pathogen that causes an infectious disease, or antigenic fragment of any of the foregoing, and (2) an immune checkpoint modulator or a cytokine, then the control arenavirus particle preferably is the same arenavirus particle as in (1). If the method comprises administering (1) a first arenavirus particle expressing a tumor antigen, tumor associated antigen, antigen of a pathogen that causes an infectious disease, or antigenic fragment of any of the foregoing, and (2) a second arenavirus particle expressing an immune checkpoint modulator or a cytokine, then the control arenavirus particle preferably is the same as the first arenavirus particle. If the method comprises administering an arenavirus particle expressing (1) a tumor antigen, tumor associated antigen, antigen of a pathogen that causes an infectious disease, or antigenic fragment of any of the foregoing, and (2) an immune checkpoint modulator or a cytokine, then the control arenavirus particle preferably is an arenavirus particle that does not express the immune checkpoint modulator or cytokine but is otherwise the same as the arenavirus particle being administered.

### 5.11 Compositions, Administration, and Dosage

Also provided herein are vaccines, immunogenic compositions (e.g., vaccine formulations), and pharmaceutical compositions comprising an arenavirus particle provided herein, and, in certain embodiments, an immune checkpoint modulator or a cytokine provided herein. In certain embodiments, the vaccines, immunogenic compositions (e.g., vaccine formulations), and pharmaceutical compositions further comprise an antibody that specifically binds to the cytokine. Such vaccines, immunogenic compositions and pharmaceutical compositions can be formulated according to standard procedures in the art.

In another embodiment, provided herein are compositions comprising an infectious arenavirus particle described herein (e.g., a replication-competent arenavirus particle), and, in certain embodiments, an immune checkpoint modulator or a cytokine provided herein. In certain embodiments, the composition further comprises an antibody that specifically binds to the cytokine. Such compositions can be used in methods of treating a neoplastic disease or an infectious disease. In another specific embodiment, the immunogenic compositions provided herein can be used to induce an immune response in a host to whom the composition is administered. The immunogenic compositions described herein can be used as vaccines and can accordingly be formulated as pharmaceutical compositions. In a specific embodiment, the immunogenic compositions described herein are used in the treatment of a neoplastic disease a subject (e.g., human subject). In a specific embodiment, the immunogenic compositions described herein are used in the treatment of an infectious disease a subject (e.g., human subject). In other embodiments, the vaccine, immunogenic composition or pharmaceutical composition are suitable for veterinary and/or human administration.

In certain embodiments, provided herein is a medical system that comprises two or more of the active pharmaceutical ingredients described herein. Such a medical system can further comprise instructions for dosing and administration and/or risk evaluation and mitigation strategies. Such instructions may be in physical form or online.

In certain embodiments, provided herein is a medical kit that comprises two or more of the active pharmaceutical ingredients described herein. Such a medical kit can further comprise instructions for dosing and administration and/or risk evaluation and mitigation strategies. Such instructions may be in physical form as part of the kit or online.

In certain embodiments, provided herein are immunogenic compositions comprising an arenavirus particle (or a combination of different arenavirus particles) as described herein. In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to an infectious arenavirus particle (e.g., a replication-competent arenavirus particle), but when the compound is administered alone does not generate an immune response to the infectious arenavirus particle. In some embodiments, the adjuvant generates an immune response to the infectious arenavirus particle (e.g., a replication-competent arenavirus particle) and does not produce an allergy or other adverse reaction. Adjuvants can enhance an immune response by several mechanisms including, e.g., lymphocyte recruitment, stimulation of B and/or T cells, and stimulation of macrophages. When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)).

The compositions comprise the arenavirus particles described herein alone or together with a pharmaceutically acceptable carrier and/or an immune checkpoint modulator and/or a cytokine. In certain embodiments, the composition further comprises an antibody that specifically binds to the cytokine. Suspensions or dispersions of genetically engineered arenavirus particles, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2-8° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 10³ to about 10¹¹ focus forming units of the genetically engineered arenavirus particles. Unit dose forms for parenteral administration are, for example, ampoules or vials, e.g., vials containing from about 10³ to 10¹⁰ focus forming units or 10⁵ to 10¹⁵ physical particles of genetically engineered arenavirus particles.

In another embodiment, a vaccine or immunogenic composition provided herein is administered to a subject by, including but not limited to, intratumoral injection oral, intradermal, intramuscular, intraperitoneal, intravenous, topical, subcutaneous, percutaneous, intranasal and inhalation routes, and via scarification (scratching through the top layers of skin, e.g., using a bifurcated needle). Specifically, subcutaneous, intramuscular or intravenous routes can be used. For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and a suitable powder base such as lactose or starch.

An arenavirus particle expressing a tumor antigen, tumor associated antigen, antigen of a pathogen that causes an infectious disease, or antigenic fragment of any of the foregoing, as described herein, or a composition thereof, can be administered via any route described herein. In certain embodiments, an arenavirus particle expressing a tumor antigen, tumor associated antigen, antigen of a pathogen that causes an infectious disease, or antigenic fragment of any of the foregoing, as described herein, or a composition thereof, is administered via intravenous injection. In certain embodiments of treating a solid tumor, an arenavirus particle expressing a tumor antigen, tumor associated antigen, or antigenic fragment of any of the foregoing, as described herein, or a composition thereof, is administered via intratumoral injection.

An arenavirus particle expressing an immune checkpoint modulator or a cytokine, as described herein, or a composition thereof, can be administered via any route described herein. In certain embodiments, an arenavirus particle expressing an immune checkpoint modulator or a cytokine, as described herein, or a composition thereof, is administered via intravenous injection. In certain embodiments of treating a solid tumor, an arenavirus particle expressing an immune checkpoint modulator or a cytokine, as described herein, or a composition thereof, is administered via intratumoral injection.

An arenavirus particle expressing both a tumor antigen, tumor associated antigen, antigen of a pathogen that causes an infectious disease, or antigenic fragment of any of the foregoing and an immune checkpoint modulator or a cytokine, as described herein, or a composition thereof, can be administered via any route described herein. In certain embodiments, an arenavirus particle expressing both a tumor antigen, tumor associated antigen, antigen of a pathogen that causes an infectious disease, or antigenic fragment of any of the foregoing and an immune checkpoint modulator or a cytokine, as described herein, or a composition thereof, is administered via intravenous injection. In certain embodiments of treating a solid tumor, an arenavirus particle expressing both a tumor antigen, tumor associated antigen, or antigenic fragment of any of the foregoing and an immune checkpoint modulator or a cytokine, as described herein, or a composition thereof, is administered via intratumoral injection.

An immune checkpoint modulator or a cytokine, not expressed from an arenavirus particle but to be co-administered in combination with an arenavirus particle expressing a tumor antigen, tumor associated antigen, antigen of a pathogen that causes an infectious disease, or antigenic fragment of any of the foregoing, as described herein, or a composition thereof, can be administered via any route described herein. In certain embodiments, an immune checkpoint modulator or a cytokine, not expressed from an arenavirus particle but to be co-administered in combination with an arenavirus particle expressing a tumor antigen, tumor associated antigen, antigen of a pathogen that causes an infectious disease, or antigenic fragment of any of the foregoing, as described herein, or a composition thereof, is administered via intravenous injection. In certain embodiments of treating a solid tumor, an immune checkpoint modulator or a cytokine, not expressed from an arenavirus particle but to be co-administered in combination with an arenavirus particle expressing a tumor antigen, tumor associated antigen, or antigenic fragment of any of the foregoing, as described herein, or a composition thereof, is administered via intratumoral injection. The immune checkpoint modulator or a cytokine, or a composition thereof, can be administered via the same route as the arenavirus particle or a composition thereof. The immune checkpoint modulator or a cytokine, or a composition thereof, can be administered via a different route than the arenavirus particle or a composition thereof. In one embodiment, the immune checkpoint modulator or a cytokine, or a composition thereof, is administered via intratumoral injection, and the arenavirus particle expressing a tumor antigen, tumor associated antigen, or antigenic fragment of any of the foregoing, or a composition thereof, is administered via intravenous injection, for treating a solid tumor. In another embodiment, the immune checkpoint modulator or a cytokine, or a composition thereof, is administered via intratumoral injection, and the arenavirus particle expressing a tumor antigen, tumor associated antigen, or antigenic fragment of any of the foregoing, or a composition thereof, is administered via intratumoral injection, for treating a solid tumor. In another embodiment, the immune checkpoint modulator or a cytokine, or a composition thereof, is administered via intravenous injection, and the arenavirus particle expressing a tumor antigen, tumor associated antigen, or antigenic fragment of any of the foregoing, or a composition thereof, is administered via intratumoral injection, for treating a solid tumor. In another embodiment, the immune checkpoint modulator or a cytokine, or a composition thereof, is administered via intravenous injection, and the arenavirus particle expressing a tumor antigen, tumor associated antigen, antigen of a pathogen that causes an infectious disease, or antigenic fragment of any of the foregoing, or a composition thereof, is administered via intravenous injection.

An immune checkpoint modulator or a cytokine, expressed from an arenavirus particle, which is to be co-administered in combination with an arenavirus particle expressing a tumor antigen, tumor associated antigen, antigen of a pathogen that causes an infectious disease, or antigenic fragment of any of the foregoing, as described herein, or a composition thereof, can be administered via any route described herein. In certain embodiments, an immune checkpoint modulator or a cytokine, expressed from an arenavirus particle, which is to be co-administered in combination with an arenavirus particle expressing a tumor antigen, tumor associated antigen, antigen of a pathogen that causes an infectious disease, or antigenic fragment of any of the foregoing, as described herein, or a composition thereof, is administered via intravenous injection. In certain embodiments of treating a solid tumor, an immune checkpoint modulator or a cytokine, expressed from an arenavirus particle, which is to be co-administered in combination with an arenavirus particle expressing a tumor antigen, tumor associated antigen, or antigenic fragment of any of the foregoing, as described herein, or a composition thereof, is administered via intratumoral injection. The arenavirus particle expressing an immune checkpoint modulator or a cytokine, or a composition thereof, can be administered via the same route as the arenavirus particle expressing a tumor antigen, tumor associated antigen, antigen of a pathogen that causes an infectious disease, or antigenic fragment of any of the foregoing, or a composition thereof. The arenavirus particle expressing an immune checkpoint modulator or a cytokine, or a composition thereof, can be administered via a different route than the arenavirus particle expressing a tumor antigen, tumor associated antigen, antigen of a pathogen that causes an infectious disease, or antigenic fragment of any of the foregoing, or a composition thereof. In one embodiment, the arenavirus particle expressing an immune checkpoint modulator or a cytokine, or a composition thereof, is administered via intratumoral injection, and the arenavirus particle expressing a tumor antigen, tumor associated antigen, or antigenic fragment of any of the foregoing, or a composition thereof, is administered via intravenous injection, for treating a solid tumor. In another embodiment, the arenavirus particle expressing an immune checkpoint modulator or a cytokine, or a composition thereof, is administered via intratumoral injection, and the arenavirus particle expressing a tumor antigen, tumor associated antigen, or antigenic fragment of any of the foregoing, or a composition thereof, is administered via intratumoral injection, for treating a solid tumor. In another embodiment, the arenavirus particle expressing an immune checkpoint modulator or a cytokine, or a composition thereof, is administered via intravenous injection, and the arenavirus particle expressing a tumor antigen, tumor associated antigen, or antigenic fragment of any of the foregoing, or a composition thereof, is administered via intratumoral injection, for treating a solid tumor. In another embodiment, the arenavirus particle expressing the immune checkpoint modulator or a cytokine, or a composition thereof, is administered via intravenous injection, and the arenavirus particle expressing a tumor antigen, tumor associated antigen, antigen of a pathogen that causes an infectious disease, or antigenic fragment of any of the foregoing, or a composition thereof, is administered via intravenous injection.

If a method described herein comprises administering more than two different arenavirus particles described herein (which each can be an arenavirus particle expressing (1) a tumor antigen, tumor associated antigen, antigen of a pathogen that causes an infectious disease, or an antigenic fragment of any of the foregoing, (2) an immune checkpoint modulator, and/or (3) a cytokine) and optionally one or more immune checkpoint modulators and/or one or more cytokines not expressed from the arenavirus particles, any of or all of the different arenavirus particles, immune checkpoint modulators and cytokines can be administered via the same route or via a different route. Any of or all of the different arenavirus particles, immune checkpoint modulators and cytokines can also be administered at the same time or at different times. For example, any of or all of the different arenavirus particles, immune checkpoint modulators and cytokines can also be administered within the same hour, within a 4-hour window, within an 8-hour window, within a 12-hour window, on the same day, within a two-day window, within a three-day window, within a 4-day window, within a 5-day window, within a 6-day window, within one week, within an 8-day window, within a 9-day window, within a 10-day window, within a 11-day window, within a 12-day window, within a 13-day window, or within two weeks. In specific embodiments, any of or all of the different arenavirus particles, immune checkpoint modulators and cytokines are administered on the same day.

The dosage of the active ingredient depends upon the type of vaccination and upon the subject, and their age, weight, individual condition, the individual pharmacokinetic data, and the mode of administration.

In certain embodiments, the compositions can be administered to the patient in a single dosage comprising a therapeutically effective amount of the arenavirus particle and/or a therapeutically effective amount of an immune checkpoint modulator and/or a therapeutically effective amount of a cytokine. In some embodiments, the arenavirus particle can be administered to the patient in a single dose comprising an arenavirus particle and an immune checkpoint modulator, each in a therapeutically effective amount. In some embodiments, the arenavirus particle can be administered to the patient in a single dose comprising an arenavirus particle and a cytokine, each in a therapeutically effective amount.

In certain embodiments, the composition is administered to the patient as a single dose followed by a second dose three to six weeks later. In accordance with these embodiments, the booster inoculations may be administered to the subjects at six to twelve months intervals following the second inoculation. In certain embodiments, the booster inoculations may utilize a different arenavirus particle or composition thereof. In some embodiments, the administration of the same composition as described herein may be repeated and separated by at least 1 day, 2 days, 3 days, 4 days, 5 days, 10 days, 15 days, 30 days, 45 days, 2 months, 75 days, 3 months, or at least 6 months.

In certain embodiments, the vaccine, immunogenic composition, or pharmaceutical composition comprising an arenavirus particle can be used as a live vaccination. Exemplary doses for a live arenavirus particle may vary from 10-100, or more, PFU of live virus per dose. In some embodiments, suitable dosages of an arenavirus particle or the tri-segmented arenavirus particle are 10², 5×10², 10³, 5×10³, 10⁴, 5×10⁴, 10⁵, 5×10⁵, 10⁶, 5×10⁶, 10γ, 5×10⁷, 10, 5×10⁸, 1×10⁹, 5×10⁹, 1×10¹⁰, 5×10¹⁰, 1×10¹¹, 5×10¹¹ or 10¹² pfu, and can be administered to a subject once, twice, three or more times with intervals as often as needed. In another embodiment, a live arenavirus is formulated such that a 0.2-mL dose contains 10⁶0.5-10⁷ 5 fluorescent focal units of live arenavirus particle. In another embodiment, an inactivated vaccine is formulated such that it contains about 15 μg to about 100 μg, about 15 μg to about 75 μg, about 15 μg to about 50 μg, or about 15 μg to about 30 μg of an arenavirus.

In certain embodiments, suitable dosages of an immune checkpoint modulator (e.g., agonist of the 4-1BB costimulatory pathway) are in the range of 0.1-5 mg/kg. In certain embodiments, suitable dosages of an immune checkpoint modulator (e.g., agonist of the 4-1BB costimulatory pathway) is below 0.1 mg/kg. In certain embodiments, suitable dosages of an immune checkpoint modulator (e.g., agonist of the 4-1BB costimulatory pathway) is above 5 mg/kg.

In certain embodiments, suitable dosages of a cytokine (e.g., IL-12) are in the range of 50-500 ng/kg. In specific embodiments, suitable dosages of a cytokine (e.g., IL-12) are in the range of 50-100 ng/kg. In specific embodiments, suitable dosages of a cytokine (e.g., IL-12) are in the range of 100-200 ng/kg. In specific embodiments, suitable dosages of a cytokine (e.g., IL-12) are in the range of 200-300 ng/kg. In specific embodiments, suitable dosages of a cytokine (e.g., IL-12) are in the range of 300-400 ng/kg. In specific embodiments, suitable dosages of a cytokine (e.g., IL-12) are in the range of 400-500 ng/kg.

Also provided are processes and uses of an arenavirus particle and an immune checkpoint modulator or a cytokine for the manufacture of vaccines in the form of pharmaceutical preparations, which comprise the arenavirus particle and the immune checkpoint modulator or the cytokine as an active ingredient. In certain embodiments, the pharmaceutical preparations further comprise an antibody that specifically binds to the cytokine. Still further provided is a combination of an arenavirus particle provided herein and an immune checkpoint modulator or a cytokine provided herein for use in the treatment of a neoplastic disease or an infectious disease described herein. In certain embodiments, the combination further comprises an antibody that specifically binds to the cytokine. In certain embodiments, the combination is in the same pharmaceutical composition. In certain embodiments, the combination is not in the same pharmaceutical composition, such as when the arenavirus particle and the immune checkpoint modulator or cytokine are to be separately administered. The pharmaceutical compositions of the present application are prepared in a manner known per se, for example by means of conventional mixing and/or dispersing processes.

In certain embodiments, the methods and compositions provided herein are used in combination with personalized medicine. Personalized medicine seeks to benefit patients by using information from a patient's unique genetic and/or epigenetic profile to predict a patient's response to different therapies and identify which therapies are more likely to be effective. Techniques that can be used in combination with the methods and compositions provided herein to obtain a patient's unique genetic and/or epigenetic profile include, but are not limited to, genome sequencing, RNA sequencing, gene expression analysis and identification of a tumor antigen (e.g., neoantigen), tumor associated antigen or an antigenic fragment thereof. In certain embodiments, the selection of an arenavirus tumor antigen or tumor associated antigen for use in the methods and compositions provided herein is performed based on the genetic profile of the patient. In certain embodiments, the selection of an arenavirus tumor antigen or tumor associated antigen for use in the methods and compositions provided herein is performed based on the genetic profile of a tumor or tumor cell.

Also provided herein are kits that can be used to perform the methods described herein. In certain embodiments, the kit provided herein can include one or more containers. These containers can hold for storage the compositions (e.g., pharmaceutical, immunogenic or vaccine composition) provided herein. Also included in the kit are instructions for use. These instructions describe, in sufficient detail, a treatment protocol for using the compositions contained therein. For example, the instructions can include dosing and administration instructions as provided herein for the methods of treating a neoplastic disease.

In certain embodiments, a kit provided herein includes containers that each contains the active ingredients for performing the methods described herein. Thus, in certain embodiments, the kit provided herein includes two or more containers and instructions for use, wherein one of the containers comprises an arenavirus particle provided herein and another container that comprises an immune checkpoint modulator or a cytokine provided herein. In certain embodiments, the container comprising the cytokine further comprises an antibody that binds to the cytokine.

### 5.12 Assays to Demonstrate Activity

Described herein are non-limiting exemplary assays that may be used to demonstrate efficacy of a combination therapy method described herein or activity of an ingredient used in the combination therapy. (a) Assay to Measure Number of Antigen-Specific CD8+ T Cells After Therapy

Any assay known to the skilled artisan can be used to measure number of antigen-specific CD8+ T cells after administration of an arenavirus particle expressing a tumor antigen, tumor associated antigen, antigen of a pathogen that causes an infectious disease, or antigenic fragment of any of the foregoing, co-expressing an immune checkpoint modulator or a cytokine, or co-administered in combination with an immune checkpoint modulator or a cytokine. One exemplary assay is described as follows: Spleen cells or blood cell suspensions are stained using either H-2Kb dextramers loaded with GP70 (604-11) peptide (KSPWFTTL) or using H-2Db dextramers loaded with LCMV NP396-404 peptide (FQPQNGQFI) according to the manufacturer's instructions (Immudex). Cells are co-stained with antibodies to identify CD3+CD8+ T cells. Stained cell suspensions are analyzed by multi-color flow cytometry.

(b) Assay to Measure Expression of CD127 on Antigen-Specific CD8+ T Cells after Therapy

Any assay known to the skilled artisan can be used to measure expression of CD127 on antigen-specific CD8+ T cells after administration of an arenavirus particle expressing a tumor antigen, tumor associated antigen, antigen of a pathogen that causes an infectious disease, or antigenic fragment of any of the foregoing, co-expressing an immune checkpoint modulator or a cytokine, or co-administered in combination with an immune checkpoint modulator or a cytokine. One exemplary assay is described as follows: Spleen cells or blood cell suspensions are stained using either H-2Kb dextramers loaded with GP70 (604-11) peptide (KSPWFTTL) or using H-2Db dextramers loaded with LCMV NP396-404 peptide (FQPQNGQFI) according to the manufacturer's instructions (Immudex). Cells are co-stained with antibodies to identify CD3+CD8+ T cells. Anti-CD127 staining is used to determine the relative expression of CD127 on antigen specific CD3+CD8+ T cells. Stained cell suspensions are analyzed by multi-color flow cytometry.

### (c) Assay to Measure Anti-Tumoral or Anti-Infection Effect and Survival Benefit After Therapy

Any assay known to the skilled artisan can be used to measure anti-tumoral or anti-infection effect and survival benefit after administration of an arenavirus particle expressing a tumor antigen, tumor associated antigen, antigen of a pathogen that causes an infectious disease, or antigenic fragment of any of the foregoing, co-expressing an immune checkpoint modulator or a cytokine, or co-administered in combination with an immune checkpoint modulator or a cytokine. One exemplary assay is described as follows: C57BL/6 mice are injected subcutaneously in the flank with 2×10⁵ B16.F10 tumor cells admixed with Cultrex BME Type 3 (see Overwijk and Restifo, 2001, Curr Protoc Immunol Chapter 20: Unit 20.1. Tumor diameter is measured at regular intervals using a caliper 2-3 times a week. Tumor volume is calculated using the formula: V=(length×width²)/2. Mice are sacrificed when tumors reach the humane endpoint of ≤15 mm or require euthanasia due to ulcerations or necrotic tumors. The Kaplan-Meier curve illustrates the survival function. It's a step function illustrating the cumulative survival probability over time.

### (d) Arenavirus Detection Assays

The skilled artesian could detect an arenavirus genomic segment or an arenavirus particle, as described herein using techniques known in the art. For example, RT-PCR can be used with primers that are specific to an arenavirus to detect and quantify an arenavirus genomic segment or a tri-segmented arenavirus particle. Western blot, ELISA, radioimmunoassay, immunoprecipitation, immunocytochemistry, or immunocytochemistry in conjunction with FACS can be used to quantify the gene products of the arenavirus genomic segment or arenavirus particle.

### (e) Assay to Measure Infectivity

Any assay known to the skilled artisan can be used for measuring the infectivity of an arenavirus vector preparation. For example, determination of the virus/vector titer can be done by a “focus forming unit assay” (FFU assay). In brief, complementing cells, e.g., HEK293-TVL cells are plated and inoculated with different dilutions of a virus/vector sample. After an incubation period, to allow cells to form a monolayer and virus to attach to cells, the monolayer is covered with Methylcellulose. When the plates are further incubated, the original infected cells release viral progeny. Due to the Methylcellulose overlay the spread of the new viruses is restricted to neighboring cells. Consequently, each infectious particle produces a circular zone of infected cells called a Focus. Such Foci can be made visible and thus countable using antibodies against LCMV-NP or another protein expressed by the arenavirus particle or the tri-segmented arenavirus particle and a HRP-based color reaction. The titer of a virus/vector can be calculated in focus-forming units per milliliter (FFU/mL). In a similar way, the proportion of tri-segmented, replication competent virus particles can be determined. Instead of complementing cells, non-complementing cell lines are used, e.g. HEK293. This allows only trisegmented virus particles to infect neighboring cells. The titer of the replication competent virus/vector (RCV) can be calculated in focus-forming units per milliliter (RCV FFU/mL).

### (f) Growth of an Arenavirus Particle

Growth of an arenavirus particle described herein can be assessed by any method known in the art or described herein (e.g., cell culture). Viral growth may be determined by inoculating a defined amount/concentration of arenavirus particle described herein into cell cultures (e.g., Vero cells or BHK-21 cells). After incubation of the virus for a specified time, the virus containing supernatant is collected using standard methods and the infectivity can be measured using the assays described herein.

### (g) Serum ELISA

Determination of the humoral immune response upon vaccination of animals (e.g., mice, guinea pigs) can be done by antigen-specific serum ELISAs (enzyme-linked immunosorbent assays). In brief, plates are coated with antigen (e.g., recombinant protein), blocked to avoid unspecific binding of antibodies and incubated with serial dilutions of sera. After incubation, bound serum-antibodies can be detected, e.g., using an enzyme-coupled anti-species (e.g., mouse, guinea pig)-specific antibody (detecting total IgG or IgG subclasses) and subsequent color reaction. Antibody titers can be determined as, e.g., endpoint geometric mean titer.

### (h) Assay to Measure the Neutralizing Activity of Induced Antibodies

Determination of the neutralizing antibodies in sera is performed with the following cell assay using ARPE-19 cells from ATCC and a GFP-tagged virus. In addition supplemental guinea pig serum as a source of exogenous complement is used. The assay is started with seeding of 6.5×10³ cells/well (50 μl/well) in a 384 well plate one or two days before using for neutralization. The neutralization is done in 96-well sterile tissue culture plates without cells for 1 h at 37° C. After the neutralization incubation step the mixture is added to the cells and incubated for additional 4 days for GFP-detection with a plate reader. A positive neutralizing human sera is used as assay positive control on each plate to check the reliability of all results. Titers (EC50) are determined using a 4 parameter logistic curve fitting. As additional testing the wells are checked with a fluorescence microscope.

### (i) Plaque Reduction Assay

In brief, plaque reduction (neutralization) assays for LCMV can be performed by use of a replication-competent or -deficient LCMV that is encoding a reporter gene (e.g., green fluorescent protein), 5% rabbit serum may be used as a source of exogenous complement, and plaques can be enumerated by fluorescence microscopy. Neutralization titers may be defined as the highest dilution of serum that results in a 50%, 75%, 90% or 95% reduction in plaques, compared with that in control (pre-immune) serum samples. qPCR LCMV RNA genomes are isolated using QIAamp Viral RNA mini Kit (QIAGEN), according to the protocol provided by the manufacturer. LCMV RNA genome equivalents are detected by quantitative PCR carried out on an StepOnePlus Real Time PCR System (Applied Biosystems) with SuperScript® III Platinum® One-Step qRT-PCR Kit (Invitrogen) and primers and probes (FAM reporter and NFQ-MGB Quencher) specific for part of the LCMV NP coding region or another genomic stretch of the arenavirus particle or the tri-segmented arenavirus particle. The temperature profile of the reaction may be: 30 min at 60° C., 2 min at 95° C., followed by 45 cycles of 15 s at 95° C., 30 s at 56° C. RNA can be quantified by comparison of the sample results to a standard curve prepared from a log 10 dilution series of a spectrophotometrically quantified, in vitro-transcribed RNA fragment, corresponding to a fragment of the LCMV NP coding sequence or another genomic stretch of the arenavirus particle or the tri-segmented arenavirus particle containing the primer and probe binding sites.

### (j) Western Blotting

Infected cells grown in tissue culture flasks or in suspension are lysed at indicated time points post infection using RIPA buffer (Thermo Scientific) or used directly without cell-lysis. Samples are heated to 99° C. for 10 minutes with reducing agent and NuPage LDS Sample buffer (NOVEX) and chilled to room temperature before loading on 4-12% SDS-gels for electrophoresis. Proteins are blotted onto membranes using Invitrogen's iBlot Gel transfer Device and visualized by Ponceau staining. Finally, the preparations are probed with a primary antibodies directed against proteins of interest and alkaline phosphatase conjugated secondary antibodies followed by staining with 1-Step NBT/BCIP solution (INVITROGEN).

### (k) MHC-Peptide Multimer Staining Assay for Detection of Antigen-Specific CD8+ T-cells

Any assay known to the skilled artisan can be used to test antigen-specific CD8+ T-cell responses. For example, the MHC-peptide tetramer, pentamer, or dextramer staining assay can be used (see, e.g., Altman J. D. et al., Science. 1996; 274:94-96; and Murali-Krishna K. et al., Immunity. 1998; 8:177-187). Briefly, the assay comprises the following steps, a tetramer assay is used to detect the presence of antigen specific T-cells. In order to detect an antigen-specific T-cell, it must bind to both, the peptide and the tetramer of MHC molecules custom made for a defined antigen specificity and MHC haplotype of T-cells (typically fluorescently labeled). The tetramer is then detected by flow cytometry via the fluorescent label.

### (l) ELISPOT Assay for Detection of Antigen-Specific T-Cells

Any assay known to the skilled artisan can be used to test antigen-specific T-cell responses. For example, the ELISPOT assay can be used (see, e.g., Czerkinsky C. C. et al., J Immunol Methods. 1983; 65:109-121; and Hutchings P. R. et al., J Immunol Methods. 1989; 120:1-8). E.g., cytokines such as but not limited to IFN-γ can be measured by the ELISPOT assay. Briefly, the assay comprises the following steps: An immunospot plate is coated with an anti-cytokine antibody. Cells are incubated in the immunospot plate with peptides derived from the antigen of interest. Antigen-specific cells secrete cytokines which bind to the coated antibodies. The cells are then washed off. and a second biotyinlated-anticytokine antibody is added to the plate and visualized with an avidin-HRP system or other appropriate methods.

### (m) Intracellular Cytokine Assay for Detection of Functionality of CD8+ and CD4+ T-Cells

Any assay known to the skilled artisan can be used to test the functionality of CD8+ and CD4+ T cell responses. For example, the intracellular cytokine assay combined with flow cytometry can be used (see, e.g., Suni M. A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: upon activation of cells via specific peptides or protein, an inhibition of protein transport (e.g., brefeldin A) is added to retain the cytokines within the cell. After a defined period of incubation, typically 5 hours, a washing step follows, and antibodies to other cellular markers can be added to the cells. Cells are then fixed and permeabilized. The fluorochrome-conjugated anti-cytokine antibodies are added and the cells can be analyzed by flow cytometry.

### (n) Assay for Confirming Replication-Deficiency of Viral Vectors

Any assay known to the skilled artisan that determines concentration of infectious and replication-competent virus particles can also be used to measure replication-deficient viral particles in a sample. For example, FFU assays with non-complementing cells can be used for this purpose.

Furthermore, plaque-based assays are the standard method used to determine virus concentration in terms of plaque forming units (PFU) in a virus sample. Specifically, a confluent monolayer of non-complementing host cells is infected with the virus at varying dilutions and covered with a semi-solid medium, such as agar to prevent the virus infection from spreading indiscriminately. A viral plaque is formed when a virus successfully infects and replicates itself in a cell within the fixed cell monolayer, and spreads to surrounding cells (see, e.g., Kaufmann, S. H.; Kabelitz, D. (2002). Methods in Microbiology Vol. 32:Immunology of Infection. Academic Press. ISBN 0-12-521532-0). Plaque formation can take 2-14 days, depending on the virus being analyzed. Plaques are generally counted manually and the results, in combination with the dilution factor used to prepare the plate, are used to calculate the number of plaque forming units per sample unit volume (PFU/mL). The PFU/mL result represents the number of infective replication-competent particles within the sample. When C-cells are used, the same assay can be used to titrate replication-deficient arenavirus particles or tri-segmented arenavirus particles.

### (o) Assay for Expression of Viral Antigen

Any assay known to the skilled artisan can be used for measuring expression of viral antigens. For example, FFU assays can be performed. For detection, mono- or polyclonal antibody preparation(s) against the respective viral antigens are used (transgene-specific FFU).

### (p) Animal Models

To investigate recombination and infectivity of an arenavirus particle described herein in vivo animal models can be used. In certain embodiments, the animal models that can be used to investigate recombination and infectivity of a tri-segmented arenavirus particle include mouse, guinea pig, rabbit, and monkeys. In a preferred embodiment, the animal models that can be used to investigate recombination and infectivity of an arenavirus include mouse. In a more specific embodiment, the mice can be used to investigate recombination and infectivity of an arenavirus particle are triple-deficient for type I interferon receptor, type II interferon receptor and recombination activating gene 1 (RAG1).

In certain embodiments, the animal models can be used to determine arenavirus infectivity and transgene stability. In some embodiments, viral RNA can be isolated from the serum of the animal model. Techniques are readily known by those skilled in the art. The viral RNA can be reverse transcribed and the cDNA carrying the arenavirus ORFs can be PCR-amplified with gene-specific primers. Flow cytometry can also be used to investigate arenavirus infectivity and transgene stability.

### (q) Assay to Measure Expression of Granzyme B, Ki67 and BclXL on Antigen-Specific CD8+ T Cells After Therapy

Any assay known to the skilled artisan can be used to measure expression of granzyme B, Ki67 and BclXL on antigen-specific CD8+ T cells after administration of an arenavirus particle expressing a tumor antigen, tumor associated antigen, antigen of a pathogen that causes an infectious disease, or antigenic fragment of any of the foregoing, co-expressing an immune checkpoint modulator or a cytokine, or co-administered in combination with an immune checkpoint modulator or a cytokine. One exemplary assay is described as follows: Spleen, lymph node or tumor cell suspensions are stained using either H-2Kb dextramers loaded with GP70 (604-11) peptide (KSPWFTTL) or using H-2Db dextramers loaded with LCMV NP396-404 peptide (FQPQNGQFI) according to the manufacturer's instructions (Immudex). Cells are co-stained with antibodies to identify CD3+CD8+ T cells. Intracellular staining of granzyme B, Ki67 and BclXL is performed on permeabilized and fixed cells using the Mouse FoxP3 Buffer Set (BD Biosciences), according to manufacturer's instructions. Anti-granzyme B, -Ki67 and -BclXL staining is used to determine the relative expression of granzyme B, Ki67 and BclXL on antigen specific CD3+CD8+ T cells. Granzyme B, Ki67 and BclXL are markers of cellular cytotoxicity, proliferation, and anti-apoptotic pathways, respectively. Stained cell suspensions are analyzed by multi-color flow cytometry.

### 6. SEQUENCES

TABLE 2
Sequences.

| | SEQ | |
|---|---|---|
| Description | ID NO. | Sequence |
| Amino acid sequence | 1 | MEYASDASLDPEAPWPPAPRARACRVLPWALVAGLLLLLLLAA |
| of Human 4-1BBL | | ACAVFLACPWAVSGARASPGSAASPRLREGPELSPDDPAGLLDL |
| (HUMAN Tumor | | RQGMFAQLVAQNVLLIDGPLSWYSDPGLAGVSLTGGLSYKEDTK |
| necrosis factor ligand | | ELVVAKAGVYYVFFQLELRRVVAGEGSGSVSLALHLQPLRSAAG |
| superfamily member 9/ | | AAALALTVDLPPASSEARNSAFGFQGRLLHLSAGQRLGVHLHTE |
| h4-1BBL). | | ARARHAWQLTQGATVLGLFRVTPEIPAGLPSPRSE |
| Reference: Uniprot | | |
| P41273 | | |
| Nucleotide sequence of | 2 | ATGGAGTATGCCAGTGATGCATCCCTGGACCCAGAGGCCCCCT |
| Human 4-1BBL | | GGCCCCCAGCCCCAAGGGCCAGGGCTTGCAGAGTTCTGCCCTG |
| (HUMAN Tumor | | GGCTTTGGTGGCAGGCCTGCTCCTCCTGCTCCTCCTGGCAGCA |
| necrosis factor ligand | | GCCTGTGCTGTGTTCCTGGCCTGCCCCTGGGCAGTCAGTGGGG |
| superfamily member 9/ | | CCAGGGCCAGCCCAGGGTCTGCTGCCAGCCCCAGACTCAGAG |
| h4-1BBL) (codon | | AGGGCCCTGAGTTGAGCCCAGATGACCCTGCAGGCCTGCTGG |
| optimized). | | ACCTGAGACAAGGGATGTTTGCCCAGCTGGTGGCCCAGAATG |
| | | TGCTCCTGATCGATGGCCCCTTGAGCTGGTACAGTGACCCAGG |
| | | CCTGGCTGGGGTCAGCCTGACAGGGGGCCTGAGCTACAAGGA |
| | | GGACACCAAGGAGCTGGTGGTGGCCAAGGCAGGAGTCTACTA |
| | | TGTGTTCTTCCAGCTGGAGCTGAGAAGGGTGGTGGCAGGAGA |
| | | GGGCAGTGGCTCAGTGTCACTGGCCCTCCACCTGCAGCCCCTC |
| | | AGATCAGCTGCTGGGGCTGCGGCCCTGGCCCTCACTGTGGACT |
| | | TGCCCCCTGCCAGCAGTGAGGCCAGAAACAGTGCCTTTGGCTT |
| | | CCAAGGCAGGCTGCTGCACCTGAGTGCCGGGCAGAGACTGGG |
| | | GGTCCACCTCCACACTGAGGCGAGAGCCAGGCATGCCTGGCA |
| | | GCTCACCCAAGGGGCCACTGTCCTGGGCCTGTTCAGAGTCACC |
| | | CCAGAGATCCCTGCTGGCCTCCCCAGCCCCAGGAGTGAGTGA |
| Amino acid sequence | 3 | MYRMQLLSCIALSLALVTNSAPTSSSTKKTQLQLEHLLLDLQMIL |
| of Human Interleukin-2 | | NGINNYKNPKLTRMLTFKFYMPKKATELKHLQCLEEELKPLEEV |
| (hIL-2). | | LNLAQSKNFHLRPRDLISNINVIVLELKGSETTFMCEYADETATIV |
| Reference: Uniprot | | EFLNRWITFCQSIISTLT |
| P60568 | | |
| Nucleotide sequence of | 4 | ATGTACAGAATGCAGCTGCTGAGCTGCATTGCCCTGAGCCTGG |
| Human Interleukin-2 | | CCCTGGTGACCAACAGTGCCCCCACCAGCAGCAGCACCAAGA |
| (hIL-2) (codon | | AGACACAGCTGCAGCTGGAGCACCTGCTGCTGGACCTGCAGA |
| optimized). | | TGATCCTGAATGGCATCAACAACTACAAGAACCCCAAGCTGA |
| | | CCAGAATGCTGACCTTCAAGTTCTACATGCCCAAGAAGGCCAC |
| | | TGAGCTGAAGCACCTGCAGTGCCTGGAGGAGGAGCTGAAGCC |
| | | CCTGGAAGAGGTGCTGAACCTGGCCCAGAGCAAGAACTTCCA |
| | | CCTGAGACCCAGAGACCTGATCAGCAACATCAATGTGATTGTG |
| | | CTGGAGCTGAAGGGCAGTGAGACCACCTTCATGTGTGAGTAT |
| | | GCAGACGAGACAGCCACCATTGTGGAGTTCCTGAACAGATGG |
| | | ATCACCTTCTGCCAGAGCATCATCAGCACCCTGACCTGA |
| Amino acid sequence | 5 | MYRMQLLSCIALSLALVTNSAPTSSSTKKTQLQLEHLLLDLQMIL |
| of Human Interleukin 2- | | NGINNYKNPKLTRMLTFKFYMPKKATELKHLQCLEEELKPLEEV |
| Fusion Protein (hIL2- | | LNLAQSKNFHLRPRDLISNINVIVLELKGSETTFMCEYADETATIV |
| RG). | | EFLNRWITFCQSIISTLTAEAAAKEAAAKEAAAKEAAAKAAAM |
| Reference: Uniprot | | WQLLLPTALLLLVSAGMRTEDLPKAVVFLEPQWYSVLEKDSVTLKCQ |
| O75015; | | GAYSPEDNSTQWFHNESLISSQASSYFIDAATVNDSGEYRCQTNLSTLS |
| FCG3B_HUMAN Low | | DPVQLEVHIGWLLLQAPRWVFKEEDPIHLRCHSWKNTALHKVTYLQ |
| affinity | | NGKDRKYFHHNSDFHIPKATLKDSGSYFCRGLVGSKNVSSETVNITIT |
| immunoglobulin | | QGLAVSTISSFSPPGYQVSFCLVMVLLFAVDTGLYFSVKTNI |
| gamma Fc region | | |
| receptor III-B with | | |
| rigid linker (Chen et al., | | |
| Adv. Drug Deliv. Rev. | | |
| 65, 1357-1369 (2013)) | | |
| Underlined: IL2.. | | |
| Bold: Rigid Linker. | | |
| Italic: CD16b/ | | |
| FCGR3B-GPI anchor. | | |
| Nucleotide sequence of | 6 | ATGTACAGAATGCAGCTGCTGAGCTGCATTGCCCTGAGCCTGG |
| Human Interleukin 2- | | CCCTGGTGACCAACAGTGCCCCCACCAGCAGCAGCACCAAGA |
| Fusion Protein (hIL2- | | AGACCCAGCTGCAGCTGGAGCACCTGCTGCTGGACCTGCAGA |
| RG) (codon optimized). | | TGATCCTGAATGGCATCAACAACTACAAGAACCCCAAGCTGA |
| | | CCAGAATGCTGACGTTCAAGTTCTACATGCCCAAGAAGGCCAC |
| | | AGAGCTGAAGCACCTGCAGTGCCTGGAGGAGGAGCTGAAGCC |
| | | CCTGGAAGAGGTGCTGAACCTGGCCCAGAGCAAGAACTTCCA |
| | | CCTGAGACCCAGAGACCTGATCAGCAACATCAATGTGATTGTG |
| | | CTGGAGCTGAAGGGGAGTGAGACCACCTTCATGTGTGAATAT |
| | | GCAGATGAGACAGCCACCATTGTGGAGTTCCTGAACAGATGG |
| | | ATCACCTTCTGCCAGAGCATCATCAGCACCCTGACAGCTGAGG |
| | | CTGCTGCCAAAGAGGCAGCAGCGAAAGAAGCTGCAGCCAAGG |
| | | AAGCGGCAGCCAAGGCTGCAGCCATGTGGCAGCTCCTGTTGC |
| | | CCACAGCCCTGCTGCTGCTGGTGAGTGCAGGCATGAGAACAG |
| | | AGGACCTGCCCAAGGCAGTGGTGTTCCTGGAGCCCCAGTGGT |
| | | ACAGTGTGCTGGAGAAGGACAGTGTGACCCTGAAGTGCCAAG |
| | | GTGCCTACAGCCCTGAGGACAACAGCACCCAGTGGTTCCACA |
| | | ATGAGAGCCTGATCAGCAGCCAGGCCAGCAGCTACTTCATTG |
| | | ATGCTGCCACCGTGAATGACAGTGGTGAGTACAGATGCCAGA |
| | | CCAACCTGAGCACCCTGAGTGACCCTGTGCAGCTGGAGGTGC |
| | | ACATCGGCTGGCTGCTGCTGCAGGCCCCCAGATGGGTGTTCAA |
| | | GGAGGAGGACCCCATCCACCTGAGATGCCACAGCTGGAAGAA |
| | | CACAGCCCTGCACAAGGTGACCTACCTGCAGAATGGCAAGGA |
| | | CAGAAAGTACTTCCACCACAACAGTGACTTCCACATCCCAAAG |
| | | GCCACCCTGAAGGACAGTGGCAGCTACTTCTGCAGAGGCCTG |
| | | GTGGGCAGCAAGAATGTGAGCAGTGAGACAGTGAACATCACC |
| | | ATCACCCAAGGCCTGGCTGTGAGCACCATCAGCAGCTTCAGCC |
| | | CCCCTGGCTACCAAGTGAGCTTCTGCCTGGTGATGGTGCTGCT |
| | | GTTTGCAGTGGACACAGGCCTGTACTTCAGTGTGAAGACCAAC |
| | | ATCTGA |
| Amino acid sequence | 7 | MFHVSFRYIFGLPPLILVLLPVASSDCDIEGKDGKQYESVLMVSID |
| of Human Interleukin 7 | | QLLDSMKEIGSNCLNNEFNFFKRHICDANKEGMFLFRAARKLRQ |
| (hIL-7). | | FLKMNSTGDFDLHLLKVSEGTTILLNCTGQVKGRKPAALGEAQP |
| Reference: Uniprot | | TKSLEENKSLKEQKKLNDLCFLKRLLQEIKTCWNKILMGTKEH |
| P13232 | | |
| Nucleotide sequence of | 8 | ATGTTCCATGTGAGCTTCAGATACATCTTTGGCCTGCCCCCCCT |
| Human Interleukin 7 | | GATCCTGGTCCTGCTGCCTGTGGCCAGCAGTGACTGTGACATA |
| (hIL-7) (codon | | GAGGGCAAGGATGGCAAGCAGTATGAGAGTGTGCTGATGGTG |
| optimized). | | AGCATTGACCAGCTGCTGGACAGCATGAAGGAGATTGGCAGC |
| | | AACTGCCTGAACAATGAGTTCAACTTCTTCAAGAGACACATCT |
| | | GTGATGCCAACAAGGAGGGCATGTTCCTGTTCAGGGCTGCCA |
| | | GAAAGCTGAGACAGTTCCTGAAGATGAACAGCACTGGGGACT |
| | | TTGACCTGCACCTCCTGAAGGTGAGTGAGGGCACCACCATCCT |
| | | GCTGAACTGCACTGGCCAAGTGAAAGGCAGAAAGCCAGCGGC |
| | | CCTTGGGGAGGCCCAGCCCACCAAGAGCCTGGAGGAGAACAA |
| | | GAGCCTGAAGGAGCAGAAGAAGCTCAATGACCTGTGCTTCCT |
| | | GAAGAGACTGCTCCAAGAGATCAAGACCTGCTGGAACAAGAT |
| | | CCTGATGGGCACCAAGGAGCACTGA |
| Amino acid sequence | 9 | MRPRMKYSNSKISPAKFSSTAGEALVPPCKIRRSQQKTKEFCHVY |
| of Mouse Interleukin- | | CMRLRSGLTIRKETSYFRKEPTKRYSLKSGTKHEENFSAYPRDSR |
| 33 (mIL-33). | | KRSLLGSIQAFAASVDTLSIQGTSLLTQSPASLSTYNDQSVSFVLE |
| Reference: Uniprot | | NGCYVINVDDSGKDQEQDQVLLRYYESPCPASQSGDGVDGKKL |
| Q8BVZ5. | | MVNMSPIKDTDIWLHANDKDYSVELQRGDVSPPEQAFFVLHKKS |
| | | SDFVSFECKNLPGTYIGVKDNQLALVEEKDESCNNIMFKLSKI |
| Nucleotide sequence of | 10 | ATGAGACCCAGAATGAAGTACAGCAACAGCAAGATCAGCCCT |
| Mouse Interleukin-33 | | GCCAAGTTCAGCAGCACGGCAGGGGAGGCCCTGGTGCCCCCC |
| (mIL-33) (codon | | TGCAAGATCAGAAGATCCCAGCAGAAGACCAAGGAGTTCTGC |
| optimized). | | CATGTGTACTGCATGAGACTGAGAAGTGGCCTGACCATCAGA |
| | | AAGGAGACCAGCTACTTCAGAAAGGAGCCCACCAAGAGATAC |
| | | AGCCTGAAGAGTGGCACCAAGCATGAGGAGAACTTCAGTGCC |
| | | TACCCCAGAGACAGCAGAAAGAGAAGCCTGCTGGGCAGCATC |
| | | CAAGCCTTTGCTGCCAGTGTGGACACCCTGAGCATCCAAGGCA |
| | | CCAGCCTGCTGACCCAGTCCCCAGCCAGCCTGAGCACCTACAA |
| | | TGACCAGAGTGTGAGCTTTGTGCTGGAGAATGGCTGCTATGTC |
| | | ATCAATGTGGATGACAGTGGCAAGGACCAAGAGCAAGACCAA |
| | | GTGCTGCTGAGATACTATGAGAGCCCCTGCCCCGCCTCCCAGA |
| | | GTGGTGATGGTGTGGATGGCAAGAAGCTCATGGTGAACATGA |
| | | GCCCCATCAAGGACACAGACATCTGGCTGCATGCCAATGACA |
| | | AGGACTACAGTGTGGAGCTGCAGAGAGGAGATGTGAGCCCCC |
| | | CAGAGCAAGCCTTCTTTGTGCTGCACAAGAAGAGCAGTGACTT |
| | | TGTGAGCTTTGAGTGCAAGAACCTGCCAGGCACCTACATTGGT |
| | | GTGAAGGACAACCAGCTGGCCCTGGTGGAGGAGAAGGATGAG |
| | | AGCTGCAACAACATCATGTTCAAGCTGAGCAAGATCTGA |
| Amino acid sequence | 11 | MCPQKLTISWFAIVLLVSPLMAMWELEKDVYVVEVDWTPDAPG |
| of Mouse IL-12p70 | | ETVNLTCDTPEEDDITWTSDQRHGVIGSGKTLTITVKEFLDAGQY |
| heterodimer (mIL12). | | TCHKGGETLSHSHLLLHKKENGIWSTEILKNFKNKTFLKCEAPNY |
| Reference: bioactive | | SGRFTCSWLVQRNMDLKFNIKSSSSSPDSRAVTCGMASLSAEKV |
| mouse IL-12p70 | | TLDQRDYEKYSVSCQEDVTCPTAEETLPIELALEARQQNKYENYS |
| heterodimer, fusion | | TSFFIRDIIKPDPPKNLQMKPLKNSQVEVSWEYPDSWSTPHSYFSL |
| protein of p40, GS | | KFFVRIQRKKEKMKETEEGCNQKGAFLVEKTSTEVQCKGGNVC |
| linker and p35 (without | | VQAQDRYYNSSCSKWACVPCRVRSGGGGSGGGGSGGGGSRVI |
| signal sequence) IL- | | PVSGPARCLSQSRNLLKTTDDMVKTAREKLKHYSCTAEDIDHEDITR |
| 12.p40.L.Ap35 (see | | DQTSTLKTCLPLELHKNESCLATRETSSTTRGSCLPPQKTSLMMTLCL |
| Lieschke et al., Nat | | GSIYEDLKMYQTEFQAINAALQNHNHQQIILDKGMLVAIDELMQSLN |
| Biotechnol 1997; | | HNGETLRQKPPVGEADPYRVKMKLCILLHAFSTRVVTINRVMGYLSSA |
| 15:35-40). | | |
| Underlined: mIl12 p40. | | |
| Bold: GS linker V1. | | |
| Italic: mll12 p35. | | |
| Nucleotide sequence of | 12 | ATGTGCCCCCAGAAGCTCACCATCAGCTGGTTTGCCATTGTGC |
| Mouse IL-12p70 | | TGCTGGTCAGCCCCCTGATGGCTATGTGGGAACTGGAGAAGG |
| heterodimer (mIL12) | | ATGTGTATGTGGTGGAGGTGGATTGGACCCCAGATGCCCCAG |
| (codon optimized). | | GGGAGACTGTCAACCTGACTTGTGACACCCCTGAGGAAGATG |
| | | ACATCACCTGGACCTCAGACCAGAGGCATGGAGTGATTGGCA |
| | | GTGGCAAGACTCTCACCATCACAGTCAAGGAGTTTCTGGATGC |
| | | AGGCCAGTACACTTGTCACAAAGGGGGGGAGACTCTGAGCCA |
| | | CAGCCACCTCCTCCTCCACAAGAAGGAGAATGGCATCTGGTCC |
| | | ACTGAGATCCTGAAGAACTTCAAGAACAAGACCTTTCTGAAGT |
| | | GTGAGGCCCCCAATTACAGTGGCAGGTTCACTTGCAGCTGGCT |
| | | GGTCCAGAGAAACATGGACCTCAAGTTCAACATCAAGTCCAG |
| | | CAGCAGCAGCCCTGACAGCAGAGCAGTGACTTGTGGCATGGC |
| | | CTCTCTCTCTGCAGAGAAGGTCACCCTGGATCAGAGGGACTAT |
| | | GAGAAGTACTCTGTGAGCTGCCAAGAGGATGTCACATGCCCC |
| | | ACTGCTGAGGAAACTCTGCCCATAGAGCTGGCCCTTGAGGCA |
| | | AGGCAGCAGAACAAGTATGAGAATTACAGCACCAGTTTCTTC |
| | | ATCAGGGACATCATCAAGCCAGATCCTCCCAAGAATCTGCAG |
| | | ATGAAGCCCCTGAAGAACAGCCAGGTGGAAGTCAGCTGGGAG |
| | | TACCCAGACAGCTGGAGCACCCCCCATTCCTACTTCTCCCTCA |
| | | AGTTCTTTGTCAGAATCCAGAGGAAGAAGGAGAAGATGAAGG |
| | | AGACAGAGGAGGGCTGCAATCAGAAGGGAGCCTTCCTGGTTG |
| | | AAAAGACCAGCACTGAAGTCCAGTGCAAGGGGGGCAATGTCT |
| | | GTGTTCAAGCCCAAGACAGGTACTACAACTCCAGTTGCAGCA |
| | | AGTGGGCTTGTGTCCCTTGCAGAGTCAGGAGTGGAGGAGGAG |
| | | GCAGTGGAGGAGGAGGCAGTGGTGGGGGAGGCAGCAGAGTC |
| | | ATTCCAGTCAGTGGCCCAGCCAGGTGTCTTTCCCAGTCCAGGA |
| | | ACCTCCTCAAGACAACAGATGACATGGTCAAGACTGCTAGGG |
| | | AGAAACTGAAGCACTACAGCTGCACAGCTGAAGACATTGACC |
| | | ATGAGGACATCACCAGGGACCAGACCAGCACTCTGAAGACTT |
| | | GCCTGCCTCTGGAACTCCACAAGAATGAGAGTTGTCTGGCCAC |
| | | AAGAGAGACCAGCAGCACAACCAGAGGCAGCTGCCTCCCCCC |
| | | CCAGAAAACCAGCCTCATGATGACCCTCTGCCTGGGCAGCATC |
| | | TATGAGGACCTGAAGATGTATCAGACTGAGTTCCAAGCCATCA |
| | | ATGCTGCTCTGCAGAATCACAACCATCAACAGATCATCCTAGA |
| | | CAAGGGCATGCTGGTGGCTATTGATGAGCTCATGCAGTCCCTC |
| | | AATCACAATGGGGAGACCCTGAGGCAGAAGCCCCCAGTTGGT |
| | | GAGGCTGACCCCTACAGAGTCAAGATGAAGCTGTGCATTCTGC |
| | | TCCATGCCTTCTCAACCAGGGTGGTCACCATCAACAGGGTCAT |
| | | GGGCTACCTGAGCAGTGCCTGA |
| Amino acid sequence | 13 | MIETYSQPSPRSVATGLPASMKIFMYLLTVFLITQMIGSVLFAVYL |
| of Mouse CD40L | | HRRLDKVEEEVNLHEDFVFIKKLKRCNKGEGSLSLLNCEEMRRQ |
| (mCD40L). | | FEDLVKDITLNKEEKKENSFEMQRGDEDPQIAAHVVSEANSNAA |
| Reference: | | SVLQWAKKGYYTMKSNLVMLENGKQLTVKREGLYYVYTQVTF |
| NM_011616.2; Uniprot | | CSNREPSSQRPFIVGLWLKPSSGSERILLKAANTHSSSQLCEQQSV |
| P27548-1 | | HLGGVFELQAGASVFVNVTEASQVIHRVGFSSFGLLKL |
| Nucleotide sequence of | 14 | ATGATTGAAACATACAGCCAGCCTTCCCCCAGATCAGTTGCCA |
| Mouse CD40L | | CTGGACTTCCAGCAAGCATGAAGATTTTCATGTATTTGCTCAC |
| (mCD40L) (codon | | TGTCTTCCTCATCACACAAATGATTGGATCTGTACTTTTTGCTG |
| optimized). | | TGTATCTTCACAGAAGATTGGACAAAGTTGAAGAGGAAGTGA |
| | | ACCTTCATGAAGATTTTGTCTTCATCAAAAAGCTCAAGAGATG |
| | | CAACAAAGGAGAAGGATCTTTGTCTTTGCTGAACTGTGAGGA |
| | | GATGAGGAGGCAATTTGAAGATCTTGTGAAGGACATCACTTTG |
| | | AACAAAGAAGAGAAAAAAGAAAACAGCTTTGAAATGCAAAG |
| | | AGGAGATGAGGATCCCCAAATTGCAGCACATGTTGTCAGTGA |
| | | AGCCAACAGCAATGCAGCATCAGTTCTTCAGTGGGCCAAGAA |
| | | GGGATATTACACAATGAAAAGCAACTTGGTGATGCTTGAAAA |
| | | TGGGAAACAACTGACAGTCAAAAGAGAAGGACTCTATTATGT |
| | | TTACACTCAAGTCACCTTCTGTTCAAACAGGGAGCCTTCCAGT |
| | | CAAAGACCATTCATTGTTGGCCTCTGGCTCAAGCCAAGCAGTG |
| | | GATCAGAGAGAATCTTGCTCAAGGCAGCAAACACCCACAGTT |
| | | CCTCCCAACTTTGTGAGCAGCAGTCAGTTCATTTGGGGGGTGT |
| | | CTTTGAATTGCAAGCTGGTGCATCTGTTTTTGTCAATGTGACTG |
| | | AAGCAAGCCAAGTGATCCACAGAGTTGGCTTCTCATCTTTTGG |
| | | CTTGCTCAAACTCTGA |
| Amino acid sequence | 15 | MDQHTLDVEDTADARHPAGTSCPSDAALLRDTGLLADAALLSD |
| of Mouse 4-1BBL | | TVRPTNAALPTDAAYPAVNVRDREAAWPPALNFCSRHPKLYGL |
| (m4-1BBL). | | VALVLLLLIAACVPIFTRTEPRPALTITTSPNLGTRENNADQVTPVS |
| Reference: | | HIGCPNTTQQGSPVFAKLLAKNQASLCNTTLNWHSQDGAGSSYL |
| NM_009404.3; Uniprot | | SQGLRYEEDKKELVVDSPGLYYVFLELKLSPTFTNTGHKVQGWV |
| P41274-1 | | SLVLQAKPQVDDFDNLALTVELFPCSMENKLVDRSWSQLLLLKA |
| | | GHRLSVGLRAYLHGAQDAYRDWELSYPNTTSFGLFLVKPDNPW |
| | | E |
| Nucleotide sequence of | 16 | ATGGACCAGCACACCCTTGATGTGGAGGACACAGCTGATGCC |
| Mouse 4-1BBL (m4- | | AGACACCCTGCTGGCACTTCCTGCCCTTCAGATGCTGCCCTCC |
| 1BBL) (codon | | TCAGAGACACTGGGCTCCTGGCAGATGCTGCTCTCCTCTCAGA |
| optimized). | | CACTGTGAGACCCACAAATGCTGCACTCCCCACTGATGCTGCC |
| | | TACCCTGCTGTCAATGTGAGGGACAGAGAGGCAGCCTGGCCC |
| | | CCTGCCCTGAACTTCTGTTCCAGACACCCCAAGCTCTATGGCC |
| | | TGGTTGCCTTGGTTTTGCTGCTGCTGATAGCAGCCTGTGTTCCT |
| | | ATCTTCACCAGAACTGAGCCAAGGCCTGCCCTCACCATCACCA |
| | | CCTCACCCAACCTGGGAACCAGAGAGAACAATGCAGACCAGG |
| | | TCACCCCTGTTTCTCACATTGGCTGCCCCAACACCACACAACA |
| | | GGGCTCTCCTGTGTTTGCCAAGCTCCTTGCTAAAAACCAAGCC |
| | | TCCTTGTGCAACACAACCCTGAACTGGCACAGCCAGGATGGG |
| | | GCTGGGAGCTCATACCTCTCTCAAGGGCTGAGGTATGAGGAA |
| | | GACAAGAAGGAGTTGGTGGTTGACAGTCCTGGTCTCTACTATG |
| | | TGTTCTTGGAGCTGAAGCTCAGTCCCACATTCACCAACACAGG |
| | | ACACAAGGTGCAGGGCTGGGTCTCTCTGGTCTTGCAAGCAAA |
| | | GCCTCAGGTTGATGACTTTGACAACTTGGCCCTGACAGTGGAA |
| | | CTGTTCCCTTGCTCCATGGAGAACAAGTTGGTGGACAGGTCCT |
| | | GGAGCCAACTGTTGCTCCTGAAGGCTGGCCACAGGCTGAGTGT |
| | | GGGTCTGAGGGCTTATCTGCATGGAGCCCAGGATGCCTACAG |
| | | AGACTGGGAGCTGTCCTATCCCAACACCACCAGCTTTGGGCTC |
| | | TTTCTTGTGAAACCAGACAACCCCTGGGAATGA |
| Amino acid sequence | 17 | MER VQPLEENVGNAARPRFERNKLLLVASVIQGLGLLLCFTYICL |
| of Human Tumor | | HFSALQVSHRYPRIQSIKVQFTEYKKEKGFILTSQKEDEIMKVQN |
| necrosis factor ligand | | NSVIINCDGFYLISLKGYFSQEVNISLHYQKDEEPLFQLKKVRSVN |
| superfamily member 4 | | SLMVASLTYKDKVYLNVTTDNTSLDDFHVNGGELILIHQNPGEF |
| (hOX40L). | | CVL |
| Reference: Uniprot | | |
| P23510 | | |
| Nucleotide sequence of | 18 | ATGGAGAGAGTGCAGCCCCTGGAGGAGAATGTGGGCAATGCA |
| Human Tumor necrosis | | GCCAGACCCAGATTTGAGAGAAACAAGCTGCTGCTGGTGGCC |
| factor ligand | | AGTGTGATCCAAGGCCTGGGCCTGCTGCTGTGCTTCACCTACA |
| superfamily member 4 | | TCTGCCTGCACTTCAGTGCCCTGCAAGTGAGCCACAGATACCC |
| (hOX40L) (codon | | CAGAATCCAGAGCATCAAGGTGCAGTTCACTGAGTACAAGAA |
| optimized). | | GGAGAAGGGCTTCATCCTGACCTCCCAGAAGGAGGATGAGAT |
| | | CATGAAGGTGCAGAACAACAGTGTGATCATCAACTGTGATGG |
| | | CTTCTACCTGATCAGCCTGAAGGGCTACTTCAGCCAAGAGGTG |
| | | AACATCAGCCTGCACTACCAGAAGGATGAGGAGCCCCTGTTC |
| | | CAGCTGAAGAAGGTGAGAAGTGTGAACAGCCTGATGGTGGCC |
| | | AGCCTGACCTACAAGGACAAGGTGTACCTGAATGTGACCACA |
| | | GACAACACCAGCCTGGATGACTTCCATGTGAATGGTGGTGAG |
| | | CTGATCCTGATCCACCAGAACCCTGGCGAGTTCTGTGTGCTGT |
| | | GA |
| Amino acid sequence | 19 | METDTLLLWVLLLWVPGSTGNWVNVISDLKKIEDLIQSMHIDAT |
| of ALT-803 | | LYTESDVHPSCKVTAMKCELLELQVISLESGDASIHDTVENLIILA |
| (IL15/IL15Ra). | | NDSLSSNGNVTESGCKECEELEEKNIKEFLQSFVHIVQMFINTSM |
| Reference: U.S. patent | | DRLTSSFLLLIVPAYVLSITCPPPMSVEHADIWVKSYSLYSRER |
| application Publication | | YICNSGFKRKAGTSSLTECVLNKATNVAHWTTPSLKCIREPKS |
| No. US20170088597. | | CDKTHTCPPCPAPELLGGPSVFLFPPKPKDTLMISRTPEVTCVVVDV |
| Underlined: IL15- | | SHEDPEVKFNWYVDGVEVHNAKTKPREEQYNSTYRVVSVLTVLHQD |
| N72D. | | WLNGKEYKCKVSNKALPAPIEKTISKAKGQPREPQVYTLPPSRDELTK |
| Bold: IL15RaSu. | | NQVSLTCLVKGFYPSDIAVEWESNGQPENNYKTTPPVLDSDGSFFLYS |
| Italic: IgG1 CH2-CH3 | | KLTVDKSRWQQGNVFSCSVMHEALHNHYTQKSLSLSPGK |
| (Fc Domain). | | |
| Nucleotide sequence of | 20 | ATGGAGACGGACACCCTCCTGCTGTGGGTGCTGTTGCTGTGGG |
| ALT-803 | | TGCCAGGCAGCACAGGCAACTGGGTGAATGTGATCAGTGACC |
| (IL15/IL15Ra) (codon | | TGAAGAAGATCGAGGACCTGATCCAGAGCATGCACATTGATG |
| optimized). | | CCACCCTGTACACAGAGAGTGATGTGCACCCCAGCTGCAAGG |
| | | TGACAGCCATGAAGTGTGAGCTGCTGGAGCTCCAAGTGATCA |
| | | GCCTGGAGAGTGGTGATGCCAGCATCCATGACACAGTGGAGA |
| | | ACCTGATCATCCTGGCCAATGACAGCCTGAGCAGCAATGGCA |
| | | ATGTGACTGAGAGTGGCTGCAAGGAGTGTGAGGAGCTGGAGG |
| | | AGAAGAACATCAAGGAGTTCCTGCAGAGCTTTGTGCACATTGT |
| | | GCAGATGTTCATCAACACCAGCATGGACAGACTGACCAGCAG |
| | | CTTCCTGCTGCTGATTGTGCCTGCCTATGTGCTGAGCATCACCT |
| | | GCCCCCCCCCCATGAGTGTGGAGCATGCAGACATCTGGGTGA |
| | | AGAGCTACAGCCTGTACAGCAGAGAGAGATACATCTGCAACA |
| | | GTGGCTTCAAGAGAAAGGCAGGCACCAGCAGCCTGACAGAGT |
| | | GTGTGCTGAACAAGGCCACCAATGTGGCCCACTGGACCACCC |
| | | CCAGCCTGAAGTGCATCAGAGAGCCGAAGAGCTGTGACAAGA |
| | | CCCACACCTGCCCACCCTGCCCAGCCCCAGAACTCCTGGGAGG |
| | | CCCCAGTGTGTTCCTGTTCCCCCCCAAGCCCAAGGACACCCTG |
| | | ATGATCAGCAGAACCCCAGAAGTGACCTGTGTGGTGGTGGAT |
| | | GTGAGCCATGAGGACCCAGAGGTGAAGTTCAACTGGTATGTG |
| | | GACGGGGTGGAGGTGCACAATGCCAAGACCAAGCCCAGAGAG |
| | | GAGCAGTACAACAGCACCTACAGAGTGGTGAGTGTGCTGACA |
| | | GTGCTGCACCAAGACTGGCTGAATGGCAAGGAGTACAAGTGC |
| | | AAGGTGAGCAACAAGGCCCTGCCAGCCCCCATCGAGAAGACC |
| | | ATCAGCAAGGCCAAGGGGCAGCCCAGAGAGCCCCAAGTGTAC |
| | | ACCCTGCCCCCCAGCAGGGATGAGCTGACCAAGAACCAAGTG |
| | | AGCCTGACCTGCCTGGTGAAGGGCTTCTACCCCAGTGACATTG |
| | | CAGTGGAGTGGGAGAGCAATGGGCAGCCAGAGAACAACTACA |
| | | AGACCACCCCCCCTGTGCTGGACAGTGATGGCAGCTTCTTCCT |
| | | GTACAGCAAGCTGACAGTGGACAAGAGCAGATGGCAGCAAGG |
| | | CAATGTGTTCAGCTGCAGTGTGATGCATGAGGCCCTGCACAAC |
| | | CACTACACCCAGAAGAGCCTGAGCCTGAGCCCTGGCAAGTGA |

### 7. EQUIVALENTS

All patents and publications mentioned in this specification are incorporated herein by reference in their entireties. From the foregoing description, it will be apparent that variations and modifications can be made to the invention described herein to adopt it to various uses and conditions. Such embodiments are also within the scope of the following claims.

### 8. EXAMPLES

Certain embodiments provided herein are illustrated by the following non-limiting examples, which demonstrate that the immunogenicity and anti-tumoral efficacy of arenaviral vector treatment can be enhanced by concurrent stimulation of tumor necrosis factor receptor (TNFR) signaling.

### 8.1 Example 1

This example shows that combination treatment using an arenaviral vector encoding a tumor antigen (e.g., GP70) and antibodies targeting members of the tumor necrosis factor receptor (TNFR) family resulted in superior immunogenicity in mice. Similar effects were also shown to be achieved by arenaviral vectors co-expressing a TNFR ligand.

To analyze the ability of an LCMV-based vector construct encoding GP70 to induce an antigen-specific immune response in C57BL/6 mice, intravenous immunization was performed with the indicated vector constructs at 1×10⁵ RCV FFU/dose (see Table 3 Study Layout). A vector encoding gp70 alone (i.e., artLCMV-GP70) was administered to animals in group 2. An artLCMV vector expressing GP70 on the NP-S-segment and 4-1BB ligand (4-1BBL) on the GP-S-segment (i.e., artLCMV-GP70/4-1BBL) was administered to animals in group 3. Animals of groups 4 and 5 were treated with artLCMV-GP70, co-administered with 100 μg of an anti-CD40 antibody (group 4) or 350 μg of anti-4-1BB antibody (group 5). Buffer only was injected to control mice (group 1).

On day 7 post immunization, freshly isolated splenocytes were stained with antibodies and MHC I dextramer and analyzed by flow cytometry.

TABLE 3
Study Layout.

| | Vaccination | | | | | | | | | |
|---|---|---|---|---|---|---|---|---|---|---|
| | | Dose | | | | | | | | |
| | | [RCV | | | Agonistic antibody | | | | | Mouse |
| Group | Vector prime | FFU] | Day | Route | Ab | Dose | Day | Route | Analysis | number |
| 1 | Buffer | n.a. | Day | i.v. | | | | | spleen | 5 |
| 2 | artLCMV-GP70 | 1 × 10⁵ | 0 | 200 μl | | | | | day 7 | 5 |
| 3 | artLCMV-GP70/4-1BBL | 1 × 10⁵ | | | | | | | | 5 |
| 4 | artLCMV-GP70 + αCD40 | 1 × 10⁵ | | | αCD40 | 100 μg | Day | i.v. | | 5 |
| 5 | artLCMV-GP70 + α4-1BB | 1 × 10⁵ | | | α4-1BB | 350 μg | 0 | 50 μl | | 5 |

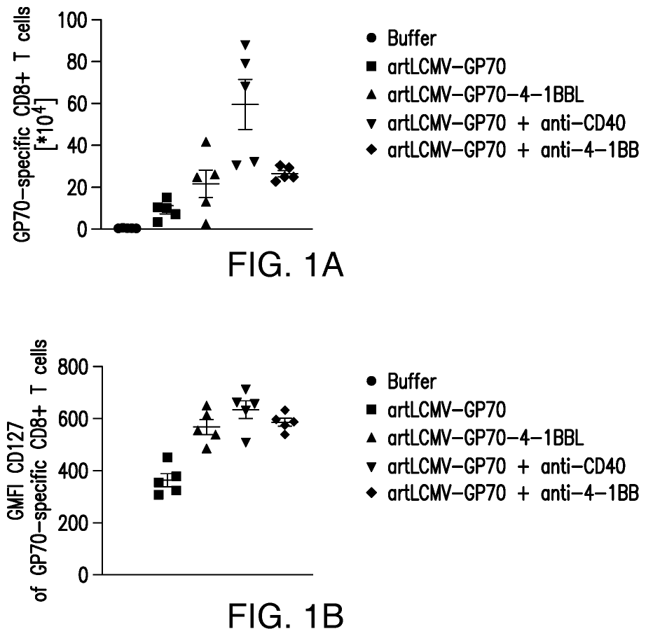

GP70-specific CD8 T cell responses were detected in all vector-treated test groups as compared to the buffer control (group 1) (FIG. 1A). Higher numbers of antigen-specific CD8 T cells were observed after immunization with a vector co-expressing 4-1BBL (group 3) as well as after immunization with artLCMV-GP70 in combination with an anti-4-1BB antibody (group 5), indicating that 4-1BBL encoded by the viral vector recapitulated the effect of an anti-4-1BB antibody co-administered with the vector. Particularly high numbers of GP70-specific CD8+ T cells were induced in mice treated with artLCMV-GP70 in combination with an anti-CD40 antibody (group 4). Interestingly, the expression of CD127 (interleukin-7 receptor) was increased on GP70-specific CD8 T cells induced after immunization with artLCMV-GP70 vector co-expressing 4-1BBL (group 3) as well as after immunization with artLCMV-GP70 vector in combination with either anti-α4-1BB (group 5) or anti-CD40 antibody (group 4) (FIG. 1i). Enhanced CD127 (IL-7R) expression results in increased response to survival signals (i.e., IL-7) and identifies memory precursor effector cells (MPECs) that display increased ability to form long-lived memory cells.

Respective data demonstrate an increase of GP70-specific CD8+ T cell numbers and increased expression of IL-7R (CD127) by 4-1BB and CD40 agonists administered in combination with GP70-encoding arenaviral vectors.

### 8.2 Example 2

This example shows that combination of an arenaviral vector encoding gp70 (artLCMV-GP70) and anti-4-1BB resulted in superior efficacy in the B16F10 tumor model.

To investigate the anti-tumor efficacy of LCMV-vectored GP70, B16F10 tumor-bearing C57BL/6 mice were immunized intravenously on day 8 after tumor challenge with the indicated vector constructs at 1×10⁵ RCV FFU/dose (see Table 4 Study Layout). Animals in groups 4 and 5 were treated simultaneously with the indicated vector construct in combination with either 100 μg of an anti-CD40 antibody (group 4) or 350 μg of anti-4-1BB antibody (group 5)

TABLE 4
Study Layout.

| | B16F10 | | Vaccination | | | | Agonistic antibody | | | | Mouse |
|---|---|---|---|---|---|---|---|---|---|---|---|
| Group | Dose | Day | Vector prime | Dose | Day | Route | Ab | Dose | Day | Route | number |
| 1 | 2 × 10⁵ | Day 0 | Buffer | n.a. | Day 8 | i.v. | | | Day 8 | i.v. | 7 |
| 2 | cells | | artLCMV-GP70 | 1 × 10⁵ | | 200 μl | | | | 200 μl | 7 |
| 3 | s.c. | | artLCMV-GP70/4-1BBL | 1 × 10⁵ | | | | | | | 7 |
| 4 | | | artLCMV-GP70 + αCD40 | 1 × 10⁵ | | | αCD40 | 100 μg | | | 7 |
| 5 | | | artLCMV-GP70 + α4-1BB | 1 × 10⁵ | | | α4-1BB | 350 μg | | | 7 |

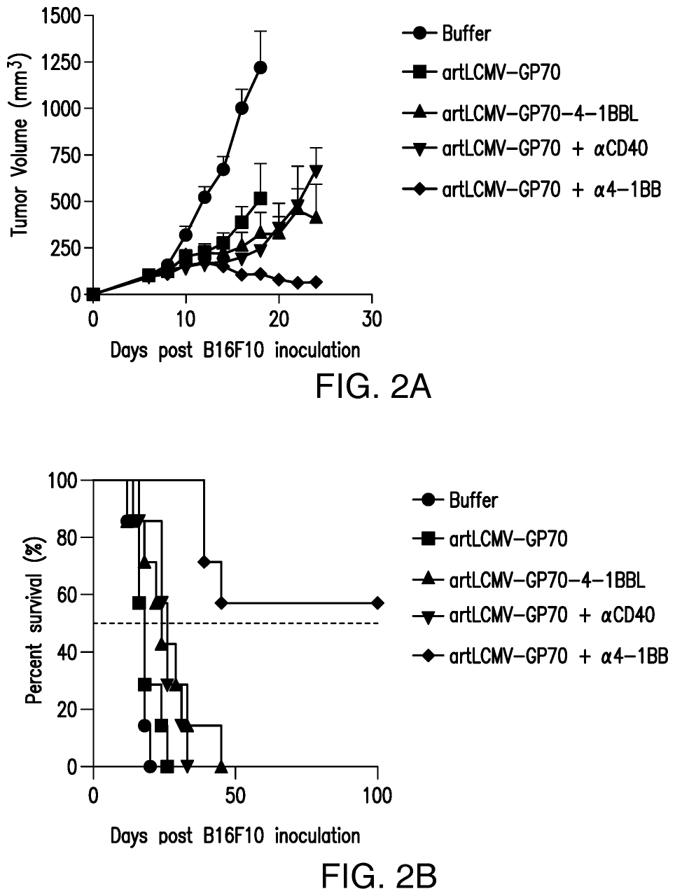

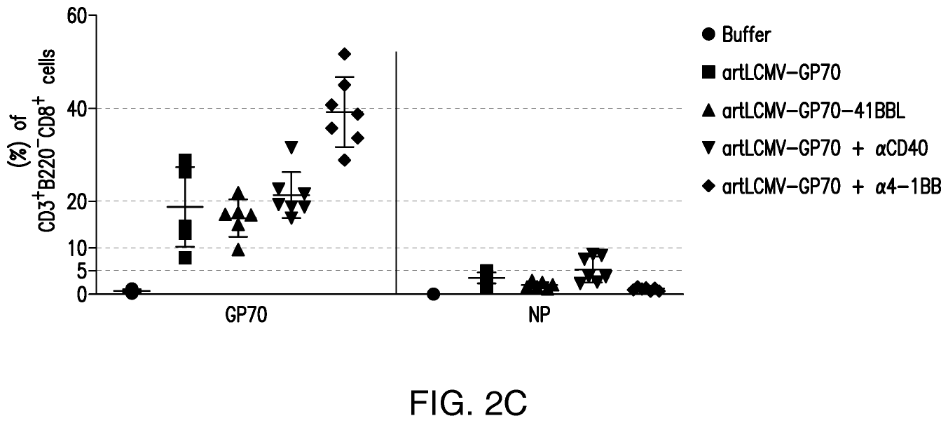

As shown in FIG. 2A, tumor growth was delayed in all vector-treated animals as compared to buffer-treated control mice (group 1). The anti-tumoral effect induced by the artLCMV-GP70 vector alone was increased when mice were treated with a vector co-expressing 4-1BBL (group 3) as well as in mice treated with LCMV-vectored GP70 in combination with an anti-CD40 antibody (group 4). The most pronounced anti-tumor effect was observed in animals treated with LCMV-vectored GP70 in combination with an anti-4-1BB antibody (group 5).

The observed control of tumor growth in mice of group 5 also translated into strongly increased survival times and survival rates (FIG. 2B). In group 5 four out of seven test animals were completely cured after treatment with artLCMV-GP70 in combination with 350 μg anti-4-1BB antibody. Three of the surviving mice were further protected against subsequent challenge with B16F10 tumor cells.

Importantly, the combination of artLCMV-GP70 and anti-4-1BB antibody resulted in a selective increase of tumor-antigen (i.e., GP70)-specific but not backbone (LCMV NP)-specific CD8 T cell responses (FIG. 2C).

### 8.3 Example 3

This example shows that the synergistic effect of co-administering anti-4-1BB with artLCMV-gp70 was observed over a wide dose range

It was investigated whether administration of an agonistic anti-4-1BB antibody alone also resulted in an anti-tumor effect in the B16F10 tumor model. B16F10 tumor-bearing C57BL/6 mice were treated intravenously 6 days after tumor challenge with 350 μg (group 4) or 100 μg (group 7) of an anti-4-1BB antibody only. Tumor development and survival of test animals in groups 4 and 7 were subsequently monitored and compared to that of control mice treated with formulation buffer only (group 1), mice immunized with artLCMV-GP70 vector alone (group 2), artLCMV-GP70 in combination with 350 μg (group 5) or 100 μg (group 6) of anti-4-1BB antibody, or artLCMV-GP70 co-expressing 4-1BBL (group 3), see Table 5 Study Layout.

TABLE 5
Study Layout.

| | B16F10 | | Vaccination | | | | | Agonistic antibody | | | | | Mouse |
|---|---|---|---|---|---|---|---|---|---|---|---|---|---|
| Group | Dose | Day | Vector | Dose | Day | Volume | Route | Ab | Dose | Day | Volume | Route | number |
| 1 | 2 × 10⁵ | Day 0 | Buffer | n.a. | Day 6 | 200 μl | i.v. | Buffer | n.a. | Day 6 | 50 μl | i.v | 7 |
| 2 | cells | | artLCMV-GP70 | 1 × 10⁵ | | 200 μl | | Buffer | n.a. | | 50 μl | i.v | 7 |
| 3 | s.c. | | artLCMV-GP70-4-1BBL | 1 × 10⁵ | | 200 μl | | Buffer | n.a. | | 50 μl | i.v. | 7 |
| 4 | | | α4-1BB | n.a. | | 200 μl | | α 4-1BB | 100 μg | | 50 μl | i.v | 7 |
| 5 | | | artLCMV-GP70 + α4-1BB | 1 × 10⁵ | | 200 μl | | α 4-1BB | 100 μg | | 50 μl | i.v. | 7 |
| 6 | | | α4-1BB | n.a. | | 200 μl | | α 4-1BB | 350 μg | | 50 μl | i.v | 7 |
| 7 | | | artLCMV-GP70 + α4-1BB | 1 × 10⁵ | | 200 μl | | α 4-1BB | 350 μg | | 50 μl | i.v. | 7 |

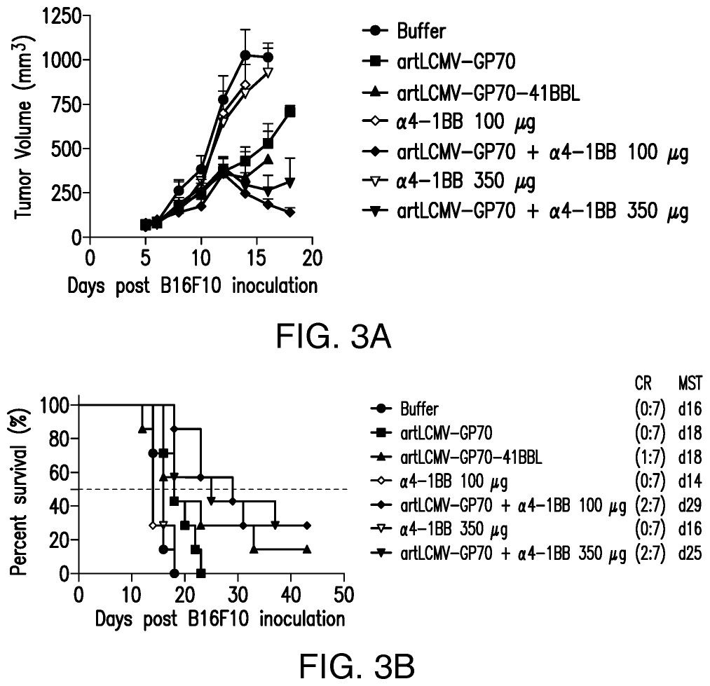

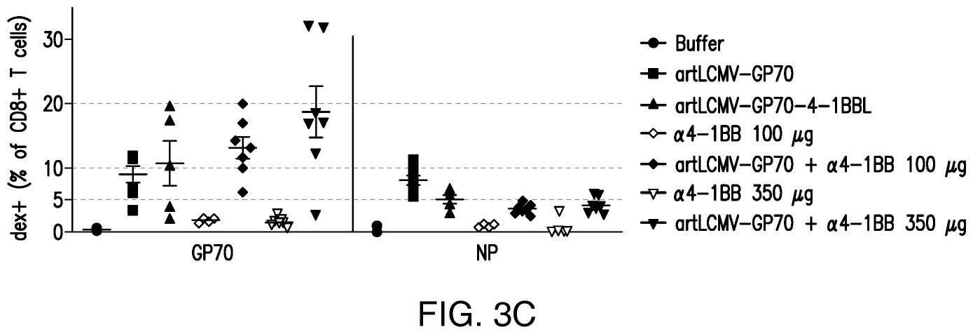

As demonstrated in FIG. 3A, treatment with agonistic 4-1BB antibody alone (groups 4 and 6) had no effect on tumor growth compared to buffer control (group 1), independent of the antibody dose. In contrast, tumor growth was delayed in all vector-treated animals (groups 2, 3,5 and 7) as compared to buffer-treated control mice (group 1) or animals treated with anti-4-1BB alone (groups 4 and 6). Unexpectedly, despite the lack of anti-tumor efficacy conveyed by agonistic 4-1BB antibody alone in the B16F10 model, the anti-tumoral effect induced by artLCMV-GP70 vector alone was significantly increased when mice were treated with LCMV-vectored GP70 in combination with anti-4-1BB antibody at a dose of 100 μg (group 5) or 350 μg (group 7).

The anti-tumor efficacy demonstrated by reduced tumor growth in FIG. 3A, also resulted in a survival benefit as shown in FIG. 3B. Whereas there were no tumor-free, long-term surviving mice in experimental groups 1, 2, 4 and 6, treated with either buffer, vector alone or 4-1BB antibody alone, two out of seven test animals could be cured completely after treatment with artLCMV-GP70 in combination with 100 μg or 350 μg anti-4-1BB antibody. Co-expression of 4-1BBL by the artLCMV-GP70 vector also had a beneficial effect resulting in one surviving mouse in this test group.

As observed before, the combination of artLCMV-GP70 and anti-4-1BB antibody resulted in a selective increase of tumor-antigen (i.e., GP70)-specific but not backbone (LCMV NP)-specific CD8 T cell responses (FIG. 3C).

### 8.4 Example 4

This example shows that the strongest anti-tumor effect was induced by simultaneous treatment of artLCMV-GP70 vector and anti-4-1BB antibody

It was investigated whether the synergistic effect observed upon combined administration of artLCMV-GP70 vector and agonistic 4-1BB antibody depends on the interval between administration of the vector and the antibody, respectively.

B16F10 tumor-bearing C57BL/6 mice in experimental groups 2 to 7 were treated intravenously with artLCMV-GP70 vector at 1×10⁵ RCV FFU/dose on day 7 after tumor challenge. Animals in groups 3 to 6 received one additional dose of anti-4-1BB antibody (100 μg) on day 7 (group 3), day 10 (group 4), day 13 (group 5), day 19 (group 6). Mice in group 7 were treated with 100 μg of anti-4-1BB antibody on days 7, 10, 13 and 19, see Table 6 Study Layout.

TABLE 6
Study Layout.

| | B16F110 | | Vaccination | | | | | Agonistic antibody | | | | | Mouse |
|---|---|---|---|---|---|---|---|---|---|---|---|---|---|
| Group | Dose | Day | Vector | Dose | Day | Volume | Route | Ab | Dose | Day | Volume | Route | number |
| 1 | 2 × 10⁵ | Day 0 | Buffer | n.a. | Day 7 | 100 μl | i.v. | Buffer | n.a. | Day 7 | 50 μl | i.p. | 7 |
| 2 | cells | | artLCMV-GP70 | 1 × 10⁵ | | 100 μl | | Buffer | n.a. | Day 7 | 50 μl | | 7 |
| 3 | s.c. | | artLCMV-GP70 | 1 × 10⁵ | | 100 μl | | α 4-1BB | 100 μg | Day 7 | 50 μl | | 7 |
| 4 | | | artLCMV-GP70 | 1 × 10⁵ | | 100 μl | | α 4-1BB | 100 μg | Days 10 | 50 μl | | 7 |
| 5 | | | artLCMV-GP70 | 1 × 10⁵ | | 100 μl | | α 4-1BB | 100 μg | Days 13 | 50 μl | | 7 |
| 6 | | | artLCMV-GP70 | 1 × 10⁵ | | 100 μl | | α 4-1BB | 100 μg | Days 19 | 50 μl | | 7 |
| 7 | | | artLCMV-GP70 | 1 × 10⁵ | | 100 μl | | α 4-1BB | 100 μg | Days 7, | 50 μl | | 7 |
| | | | | | | | | | | 10, 13, 19 | | | |

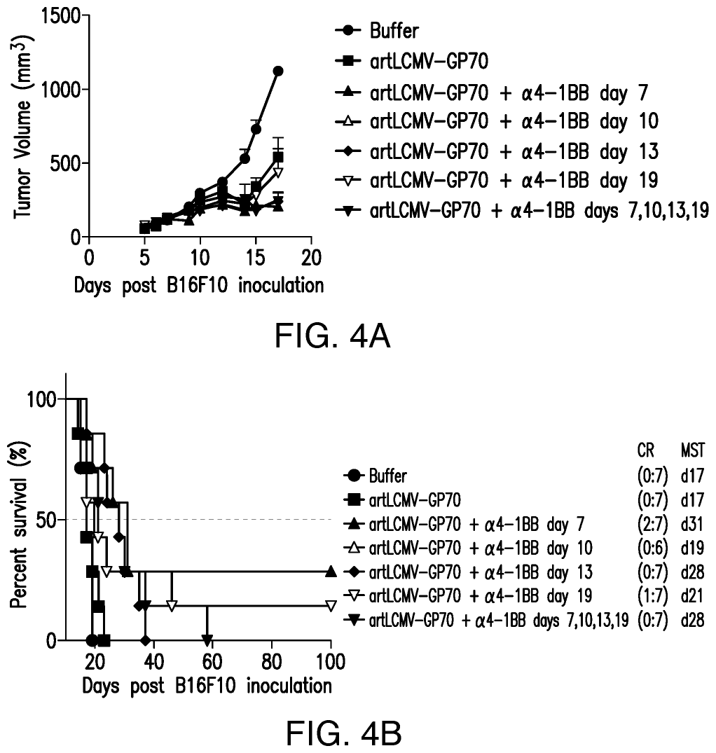

As demonstrated in FIG. 4A, tumor growth was delayed in a test groups treated with artLCMV-GP70 vector (groups 2 to 7) as compared to buffer-treated control mice (group 1). Combination treatment with anti-4-1BB antibody further enhanced the anti-tumor efficacy, with the most pronounced effects observed when the vector and the anti-4-1BB antibody were administered on the same day or within short time intervals. The observed synergistic effect of the combination treatment decreased at longer intervals between administration of the vector and the anti-4-1BB antibody (e.g., group 6). Interestingly, multiple administrations of anti-4-1BB antibody did not further enhance the anti-tumor effect of the vector/anti-4-1BB combination.

In accordance with improved anti-tumor efficacy, the beneficial effect on median survival times (MST) decreased with increasing intervals between vector and anti-4-1BB antibody administration. The strongest effect was observed in mice of group 3, treated with artLCMV-GP70 in combination with 100 μg anti-4-1BB antibody on the same day (FIG. 4B).

### 8.5 Example 5

This example shows the anti-tumor efficacy of artLCMV-GP70 vector in combination with anti-4-1BB and/or anti-OX40

It was tested whether combination with agonists targeting additional members of the tumor necrosis factor receptor (TNFR) superfamily could further enhance the anti-tumor efficacy of the artLCMV vector treatment.

B16F10 tumor-bearing C57BL/6 mice were either treated intravenously with artLCMV-GP70 vector alone (group 2), with artLCMV-GP70 vector in combination with 100 μg of anti-4-1BB antibody (group 4), with artLCMV-GP70 vector in combination with 100 μg of anti-OX40 antibody (group 5), or with artLCMV-GP70 vector in combination with 100 μg of anti-4-1BB as well as 100 μg of anti-OX40 antibody (group 6). Formulation buffer and IgG1 isotype control antibody were administered to control animals in group 1. Mice in group 3 were solely treated with 100 μg of anti-4-1BB as well as 100 μg of anti-OX40 antibody, however, did not receive artLCMV-GP70 vector. artLCMV-GP70 vector was administered at 1×10⁵ RCV FFU/dose on day 8 after tumor challenge (groups 2, 4, 5, 6). Agonistic antibodies or IgG1 isotype control antibody was administered on day 10 after tumor challenge. The Study Layout is shown in Table 7.

TABLE 7
Study Layout.

| | B16.F10 | | Vaccination | | | | | Agonistic antibody | | | | | Mouse |
|---|---|---|---|---|---|---|---|---|---|---|---|---|---|
| Group | Dose | Day | Vector | Dose | Day | Volume | Route | Ab | Dose | Day | Volume | Route | number |
| 1 | 2 × 10⁵ | Day 0 | Buffer | n.a. | Day 8 | 100 μl | i.v. | IgG1 | 100 μg | day 10 | 100 μl | i.p. | 6 |
| 2 | cells | | artLCMV-GP70 | 1 × 10⁵ | | 100 μl | | IgG1 | 100 μg | | 100 μl | | 7 |
| 3 | s.c. | | Buffer | n.a. | | 100 μl | | α 4-1BB + | 100 μg | | 100 μl | | 6 |
| | | | | | | | | α OX40 | each | | | | |
| 4 | | | artLCMV-GP70 | n.a. | | 100 μl | | α 4-1BB | 100 μg | | 100 μl | | 7 |
| 5 | | | artLCMV-GP70 | n.a. | | 100 μl | | α OX40 | 100 μg | | 100 μl | | 7 |
| 6 | | | artLCMV-GP70 | 1 × 10⁵ | | 100 μl | | α 4-1BB + | 100 μg | | 100 μl | | 7 |
| | | | | | | | | α OX40 | each | | | | |

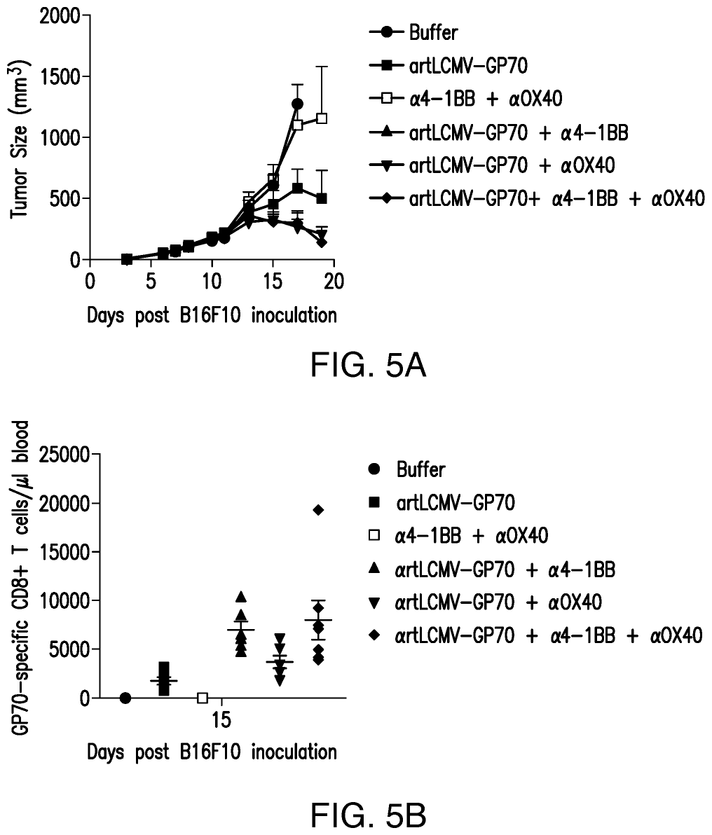

As demonstrated in FIG. 5A, tumor growth was delayed in all test groups treated with artLCMV-GP70 vector (groups 2, 4, 5, 6), however, no anti-tumor effect was observed in mice after administration of a combination of the agonistic antibodies anti-4-1BB and anti-OX40 (group 3). Surprisingly, despite the lack of anti-tumor efficacy conveyed by agonistic 4-1BB and anti-OX40 antibodies alone in the B16F10 model, the anti-tumoral effect induced by artLCMV-GP70 vector alone was significantly increased when mice were treated with LCMV-vectored GP70 in combination with either anti-4-1BB antibody (group 4), anti-OX40 antibody (group 5), or a combination of both anti-4-1BB and anti-OX40 antibodies (group 6).

As shown in FIG. 5B, GP70-specific CD8 T cell responses were detected in all artLCMV-GP70 vector-treated test groups (groups 2, 4, 5, 6) after prime immunization as compared to the buffer control (group 1) or animals treated with agonistic antibodies only (group 3). Interestingly, higher numbers of antigen-specific CD8 T cells were observed after immunization with artLCMV-GP70 in combination with an agonistic antibody (groups 4 to 6). However, highest immunogenicity was observed in mice after treatment with artLCMV-GP70 in combination with anti-4-1BB antibody (groups 4 and 6).

### 8.6 Example 6

This example shows the anti-tumor efficacy of intratumoral administration of artLCMV vectors expressing interleukin 12.

To investigate the anti-tumor efficacy of intratumorally administered LCMV vector constructs, B16F10 tumor-bearing C57BL/6 mice were immunized intratumorally on day 7 after tumor challenge with the indicated vector constructs at 1x 105 RCV FFU/dose (see Table 8 Study Layout). Animals in groups 2 and 3 were treated with artLCMV vectors encoding either an irrelevant protein (i.e., GFP, group 2) or a relevant tumor antigen GP70 (group 3). Mice in groups 4 and 5 were injected intratumorally with artLCMV vectors co-expressing the cytokine interleukin 12 (IL-12) in combination with GFP (group 4) or GP70 (group 5). Buffer-treated animals in group 1 served as control.

TABLE 8
Study Layout.

| | | | Vaccination | | | | |
|---|---|---|---|---|---|---|---|
| | B16.F10 | | | Dose | | | Mouse |
| Group | Dose | Day | Vector | (RCV FFU) | Route | Volume | number |
| 1 | 2 × 10⁵ | Day 0 | Buffer | n.a. | i.t. | 50 μl | 7 |
| 2 | cells | | artLCMV-GFP | 1 × 10⁵ | i.t. | 50 μl | 7 |
| 3 | s.c. | | artLCMV-GP70 | 1 × 10⁵ | i.t. | 50 μl | 7 |
| 4 | | | artLCMV-GFP-IL12 | 1 × 10⁵ | i.t. | 50 μl | 7 |
| 5 | | | artLCMV-GP70-IL12 | 1 × 10⁵ | i.t. | 50 μl | 7 |

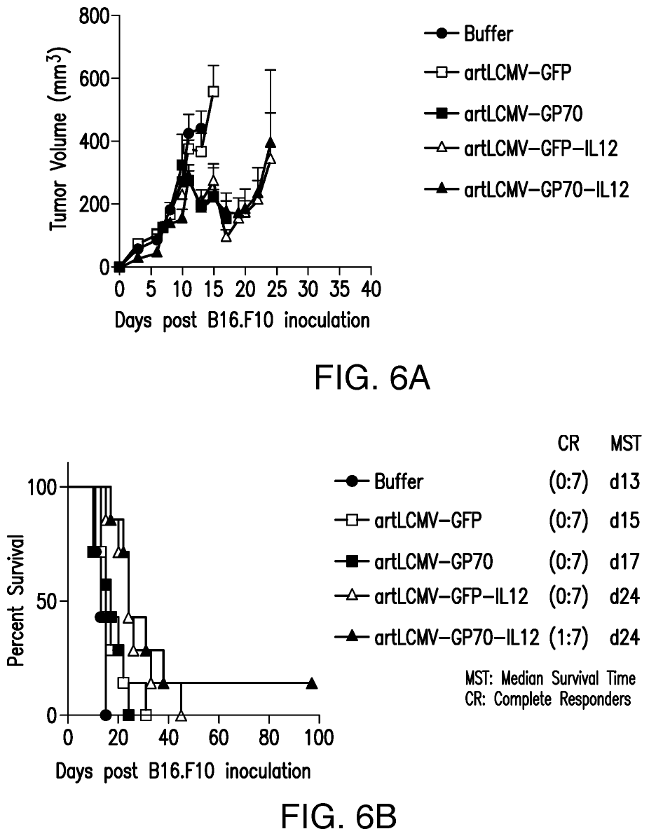

As demonstrated in FIG. 6A, tumor growth was delayed in test groups treated with artLCMV-GP70 vectors (groups 3 and 5), with a more significant reduction observed after administration of the artLCMV-GP70-IL12 vector co-expressing GP70 and IL-12 (group 5). Surprisingly, tumor growth was also considerably inhibited in mice of group 4, treated intratumorally with a vector co-encoding the irrelevant GFP protein in combination with IL-12. Unexpectedly, the inhibitory effect of the artLCMV vectors co-expressing IL-12 with either GP70 (group 5) or GFP (group 4) were comparable.

The anti-tumor efficacy demonstrated by reduced tumor growth in FIG. 6A, also resulted in a survival benefit as shown in FIG. 6B. Whereas there was an increase in the median survival time in animals of group 3, treated intratumorally with the artLCMV-GP70 vector, one long-term surviving mouse was only observed in experimental group 5, treated with an artLCMV vector co-expressing IL-12 with GP70.

### 8.7 Example 7

This example shows the anti-tumor efficacy of intratumoral administration of artLCMV vectors encoding 4-1BBL

To investigate the anti-tumor efficacy of intratumorally administered LCMV vector constructs, B16F10 tumor-bearing C57BL/6 mice were immunized intratumorally on day 7 after tumor challenge with the indicated vector constructs at 1x 105 RCV FFU/dose (see Table 9 Study Layout). Animals in groups 2 and 3 were treated with artLCMV vectors encoding either the tumor antigen GP70 (group 2) or GP70 and 4-1BBL (group 3). Buffer-treated animals in group 1 served as control.

TABLE 9
Study Layout.

| | | | Vaccination | | | | |
|---|---|---|---|---|---|---|---|
| | B16.F10 | | | Dose | | | Mouse |
| Group | Dose | Day | Vector | (RCV FFU) | Route | Volume | number |
| 1 | 2 × 10⁵ | Day 0 | Buffer | n.a. | i.t. | 50 μl | 7 |
| 2 | cells | | artLCMV-GP70 | 1 × 10⁵ | i.t. | 50 μl | 7 |
| 3 | s.c. | | artLCMV-GP70-4-1BBL | 1 × 10⁵ | i.t. | 50 μl | 7 |

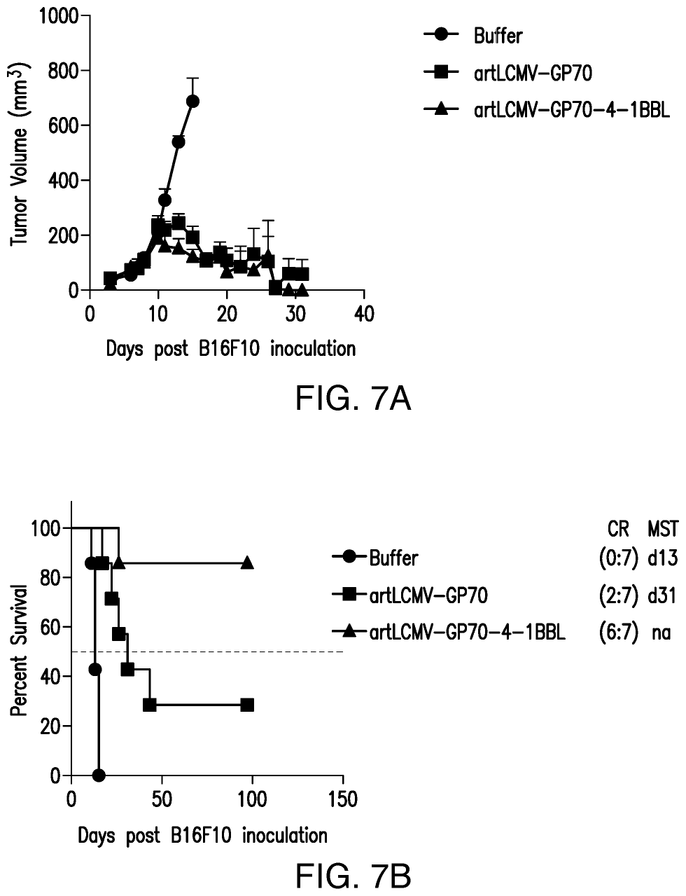

As demonstrated in FIG. 7A, tumor growth was delayed in test groups treated with artLCMV-GP70 vectors (groups 2 and 3). The anti-tumor efficacy demonstrated by reduced tumor growth in FIG. 7A, also resulted in a survival benefit as shown in FIG. 7B. The increase in the median survival time and number of tumor-free long-term surviving animals of group 2, treated intratumorally with the artLCMV-GP70 vector, were further enhanced in group 3 by intratumoral treatment of mice with artLCMV-GP70-4-1BBL, where 6 of 7 mice eliminated the tumor and survived. All tumor-free mice were completely protected against a re-challenge with the same tumor cell line, demonstrating long-term memory.

### 8.8 Example 8

This example shows the anti-tumor efficacy of intravenous and intratumoral administration of artLCMV vectors encoding 4-1BBL and a tumor self-antigen.

To investigate whether the enhanced anti-tumor effect of 4-1BBL encoding vectors is also applicable to vectors encoding another tumor antigen instead of GP70, B16F10 tumor-bearing C57BL/6 mice were immunized intravenously or intratumorally on day 7 after tumor challenge with the indicated vector constructs at 1x 105 RCV FFU/dose (see Table 10 Study Layout).

TABLE 10
Study Layout.

| | | | Vaccination | | | | |
|---|---|---|---|---|---|---|---|
| | B16.F10 | | | Dose | | | Mouse |
| Group | Dose | Day | Vector prime | [RCV FFU] | Route | Volume | number |
| 1 | 2 × 10⁵ | Day 0 | Formulation buffer | n.a. | i.v. | 100 μl | 8 |
| 2 | cells | | artLCMV-Trp2 | 1 × 10⁵ | i.v. | 100 μl | 8 |
| 3 | s.c. | | artLCMV-Trp2-41BBL | 1 × 10⁵ | i.v. | 100 μl | 8 |
| 4 | | | artLCMV-Trp2 | 1 × 10⁵ | i.t. | 100 μl | 8 |
| 5 | | | artLCMV-Trp2-41BBL | 1 × 10⁵ | i.t. | 100 μl | 8 |

Animals in groups 2 and 3 were treated intravenously with artLCMV vectors encoding either the tumor self-antigen TRP2 (group 2) or TRP2 and 4-1BBL (group 3). Animals in groups 4 and 5 were treated intratumorally with artLCMV vectors encoding either the tumor self-antigen TRP2 (group 4) or TRP2 and 4-1BBL (group 5). Buffer-treated animals in group 1 served as control.

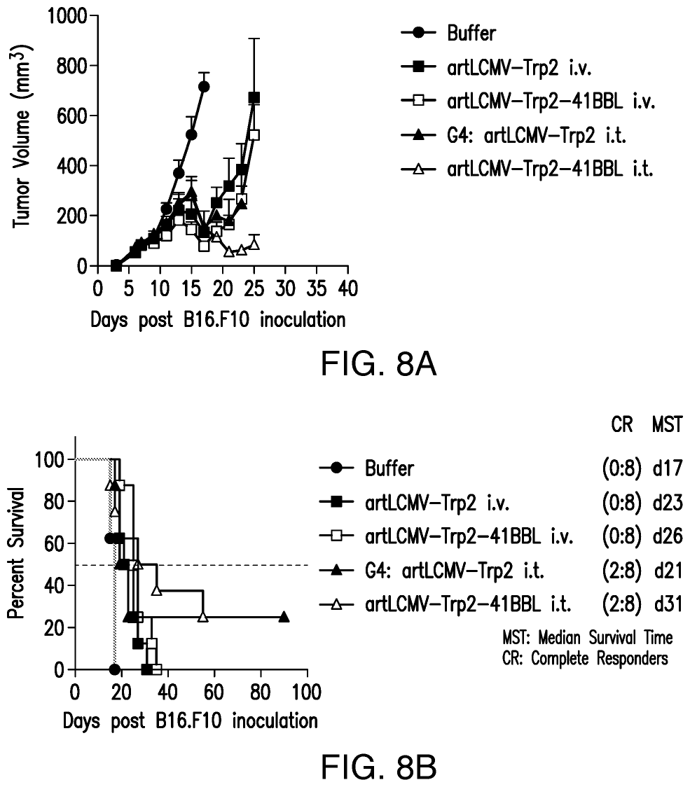

As demonstrated in FIG. 8A, tumor growth was delayed in test groups treated with artLCMV-TRP2 vectors irrespective of the administration route (groups 2-5). The tumor growth was most strongly inhibited in animals treated intratumorally with artLCMV-TRP2-4-1BBL (group 5). The anti-tumor efficacy demonstrated by reduced tumor growth in FIG. 8A, also resulted in a survival benefit as shown in FIG. 8B. Intravenous and intratumoral treatment with artLCMV-TRP2 and artLCMV-TRP2-4-1BBL led to increased median survival times compared to buffer treatment. The longest median survival time was achieved by intratumoral treatment with artLCMV-TRP2-4-1BBL. Intratumoral treatment with artLCMV-TRP2 and artLCMV-TRP2-4-1BBL resulted in 25% tumor-free mice.

### 8.9 Example 9

This example shows that combination of an arenaviral vector encoding E7E6 (artLCMV-E7E6 and artPICV-E7E6) and anti-NKG2A resulted in superior efficacy in the TC-1 tumor model.

To investigate the anti-tumor efficacy of LCMV- and PICV-vectored E7E6, TC-1 tumor-bearing C57BL/6 mice were immunized intravenously on day 11 after tumor challenge with the indicated vector constructs at 1×10⁵ RCV FFU/dose (see Table 11 Study Layout). Animals in groups 4 and 6 were treated with the indicated vector construct in combination with 200 μg of an anti-NKG2A antibody. The antibody was administered on days 11, 15 and 18 after tumor challenge.

TABLE 11
Study Layout.

| | | | | | | | | | | | | | C57BL/ |
|---|---|---|---|---|---|---|---|---|---|---|---|---|---|
| | TC1 | | Vaccination | | | | | Antibody administration | | | | | 6J mice |
| Group | Dose | Day | Vector prime | Dose | Day | Volume | Route | Ab | Dose | Day | Volume | Route | number |
| 1 | 1 × 10⁶ | Day 0 | Formulation | n.a. | Day 11 | 100 μl | i.v. | isotype control | 200 μg | days 11, | 100 μl | i.p. | 10 |
| | cells | | buffer | | | | | | | 15, 18 | | | |
| 2 | | | Formulation | n.a. | | | | αNKG2A | 200 μg | | | | 10 |
| | | | buffer | | | | | | | | | | |
| 3 | | | artLCMV-E7E6 | 1 × 10⁵ | | | | isotype control | 200 μg | | | | 10 |
| 4 | | | artLCMV-E7E6 | 1 × 10⁵ | | | | αNKG2A | 200 μg | | | | 10 |
| 5 | | | artPICV-E7E6 | 1 × 10⁵ | | | | isotype control | 200 μg | | | | 10 |
| 6 | | | artPICV-E7E6 | 1 × 10⁵ | | | | αNKG2A | 200 μg | | | | 10 |

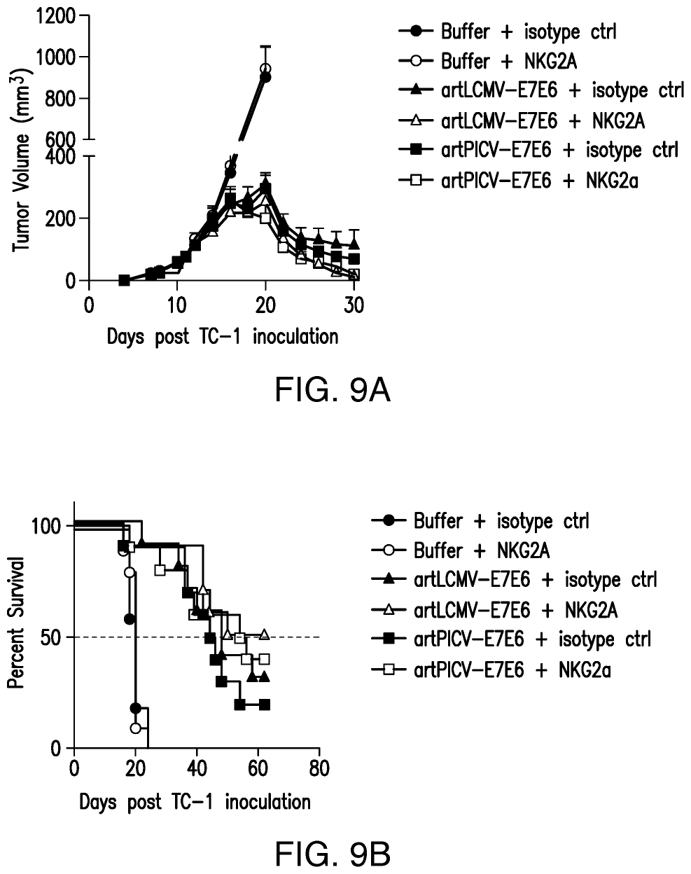

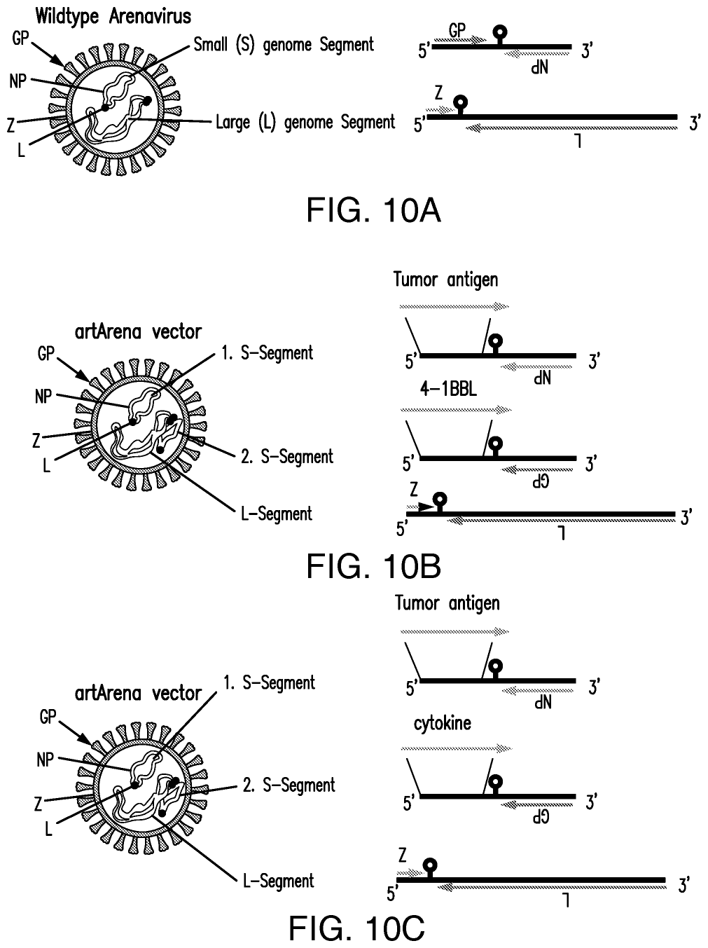

As shown in FIG. 9A, tumor growth was delayed in all vector-treated animals as compared to buffer-treated control mice (group 1) and mice treated with anti-NKG2A antibody alone (group 2). The anti-tumoral effect induced by the artLCMV-E7E6 and artPICV-E7E6 vectors alone was increased when mice were treated in combination with an anti-NKG2A antibody (group 4 and 6, respectively). The most pronounced anti-tumor effect was observed in animals treated with LCMV-vectored E7E6 in combination with an anti-NKG2A antibody (group 4).

The observed control of tumor growth in mice of groups 4 and 6 also translated into strongly increased survival times and survival rates (FIG. 9B). In group 4 four out of ten test animals were completely cured after treatment with artLCMV-E7E6 in combination with anti-NKG2A antibody. In group 6 two out of ten test animals were completely cured after treatment with artPICV-E7E6 in combination with anti-NKG2A antibody.

### 8.10 Example 10

The present disclosure discovered that 4-1BB agonists could help to overcome intratumoral immune suppression by maintaining or even enhancing the functionality of vector-induced T cell responses.

This example shows that combination of arenaviral vectors encoding the self-antigens TRP2 (artLCMV-TRP2) and anti-4-1BB resulted in superior efficacy in the B16F10 tumor model.

To investigate the anti-tumor efficacy of LCMV-vectored TRP2, B16F10 tumor-bearing C57BL/6 mice in groups 3 and 4 were immunized intravenously on day 7 after tumor challenge with the indicated vector constructs at 1×10⁵ RCV FFU/dose (see Table 12 for Study Layout). Animals in group 4 were treated simultaneously with the indicated vector construct in combination with 100 μg of anti-4-1BB antibody.

TABLE 12
Study Layout.

| | B16F10 | | Vaccination | | | | Agonistic antibody | | | | Mouse |
|---|---|---|---|---|---|---|---|---|---|---|---|
| Group | Dose | Day | Vector prime | Dose | Day | Route | Ab | Dose | Day | Route | number |
| 1 | 2 × 10⁵ | Day 0 | Buffer | n.a. | Day 7 | i.v. | | | Day 7 | i.p. | 8 |
| 2 | cells | | Buffer + α4-1BB | n.a. | | | α4-1BB | 100 μg | | | 8 |
| 3 | s.c. | | artLCMV-TRP2 | 1 × 10⁵ | | | | | | | 8 |
| 4 | | | artLCMV-TRP2 + α4-1BB | 1 × 10⁵ | | | α4-1BB | 100 μg | | | 8 |

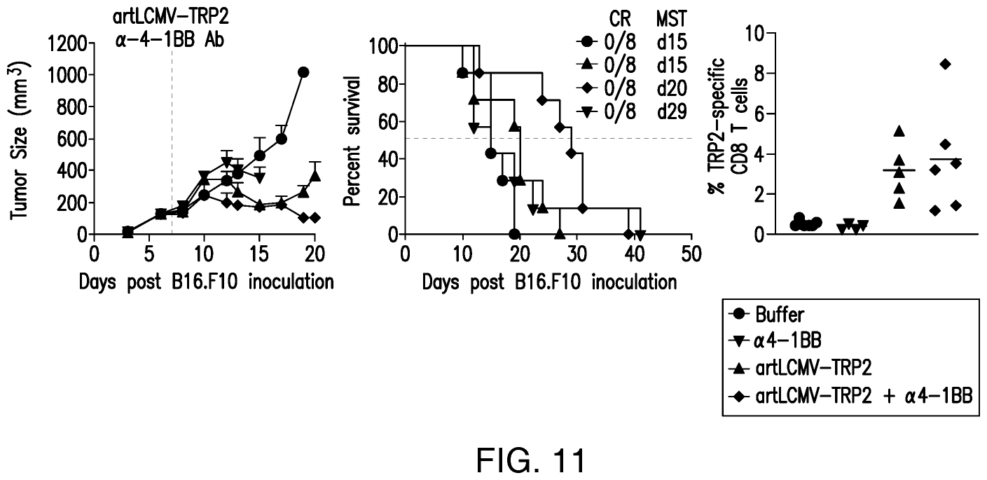

As shown in FIG. 11 (left panel), tumor growth was delayed in all vector-treated animals as compared to buffer-treated control mice (group 1). The anti-tumoral effect induced by the artLCMV-TRP2 vector alone (group 3) was increased when mice were co-treated with an anti-4-1BB antibody (group 4). The observed control of tumor growth in mice of group 4 also translated into strongly increased survival times (FIG. 11, middle panel).

The combination of artLCMV-TRP2 and anti-4-1BB antibody did not result in higher frequencies of TRP2-specific CD8 T cells in the blood on day 14 after immunization compared to vector treatment alone (FIG. 11, right panel). Anti-4-1BB treatment without vector immunization (group 2) did neither affect tumor growth, nor survival times, nor did it induce TRP2-specific CD8 T cell responses.

### 8.11 Example 11

This example shows that combination of an arenaviral vector encoding GP70 (artLCMV-GP70) and anti-4-1BB resulted in higher numbers of GP70 specific CD8 T cells in tumor and tumor draining lymph nodes in the B16F10 tumor model.

To investigate the tumor specific CD8 T cell response in B16F10 tumor-bearing C57BL/6 mice, spleen, tumor and tumor draining lymph nodes were analyzed by flow cytometry on days 8 and 12 after treatment with artLCMV-GP70 (group 2) or a combination of artLCMV-GP70 and 100 μg of anti-4-1BB (group 3). Control mice were treated with buffer (see Table 13 Study Layout).

TABLE 13
Study Layout.

| | B16F10 | | Vaccination | | | | Agonistic antibody | | | | Mouse |
|---|---|---|---|---|---|---|---|---|---|---|---|
| Group | Dose | Day | Vector prime | Dose | Day | Route | Ab | Dose | Day | Route | number |
| 1 | 2 × 10⁵ | Day 0 | Buffer | n.a. | Day 8 | i.v. | | | Day 8 | i.p. | 10 |
| 2 | cells | | artLCMV-GP70 + | 1 × 10⁵ | | | Isotype | 100 μg | | | 10 |
| | s.c. | | isotype ctrl | | | | ctrl | | | | |
| 3 | | | artLCMV-GP70 + | 1 × 10⁵ | | | α4-1BB | 100 μg | | | 10 |
| | | | α4-1BB | | | | | | | | |

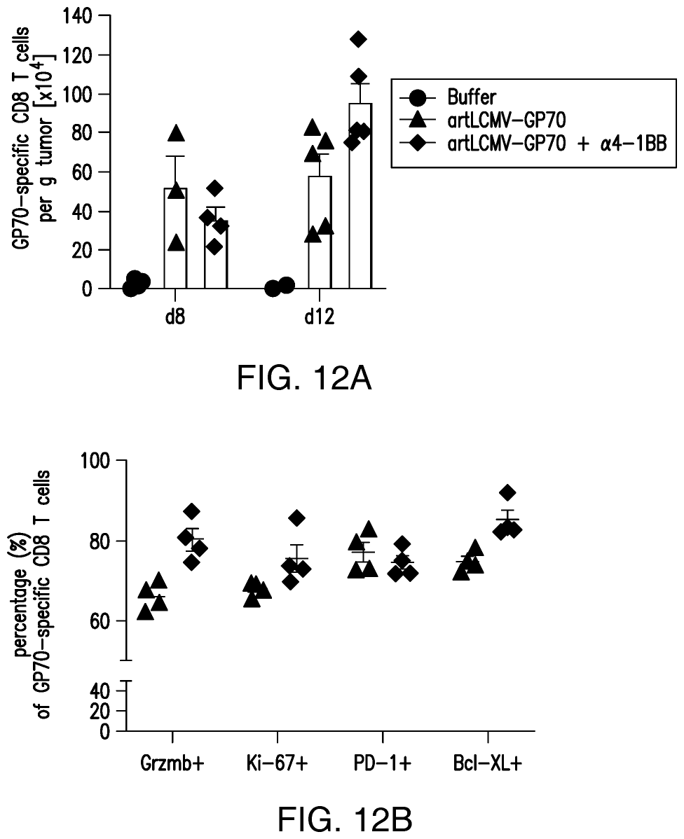

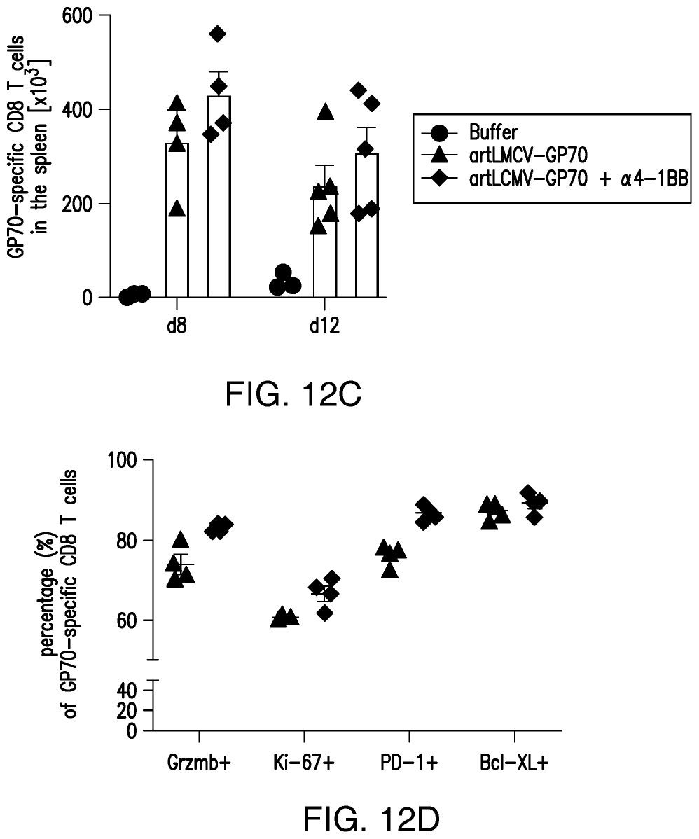

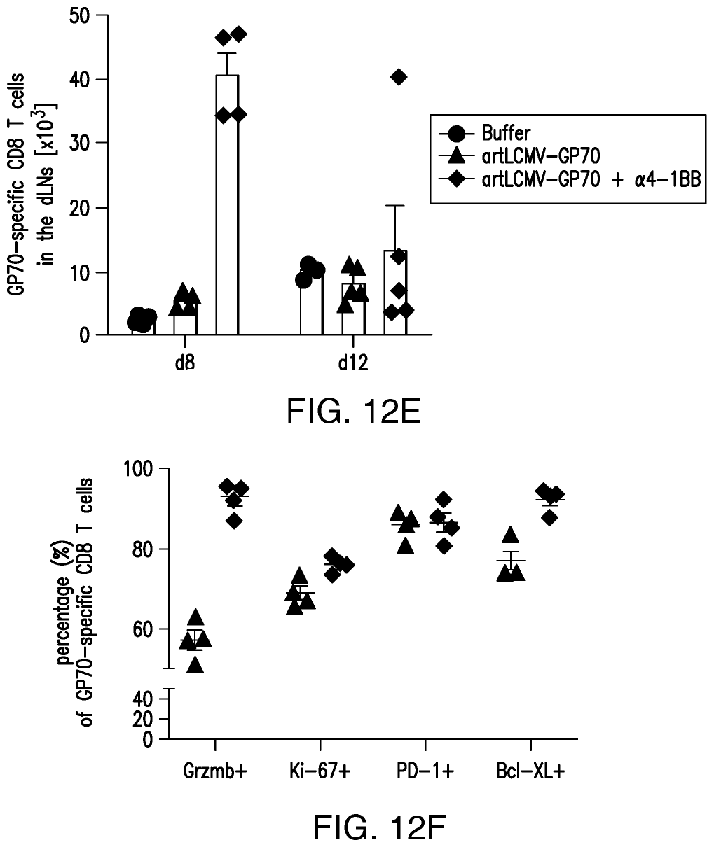

Co-treatment of vector and antibody (group 4) led to higher GP70 specific CD8 T cell numbers on day 12 in the tumor (FIG. 12A) and on day 8 in tumor draining lymph nodes (FIG. 12E) compared to vector treatment alone. T cell numbers in the spleen were not different between groups 2 and 3 (FIG. 12C). Vector plus anti-4-1BB combination treatment increased the percentage of granzyme B, Ki67, BclXL but not PD-1 expressing GP70 specific CD8 T cells in the tumor (FIG. 12B). In the spleen, more GP70 specific CD8 T cells expressing granzyme B, Ki67, PD-1 but not BclXL were detected in group 3 compared to group 2 (FIG. 12D). More GP70 specific CD8 T cells expressed granzyme B, Ki67, BclXL but not PD-1 when mice were treated with the combination therapy compared to vector treatment alone (FIG. 12F). These data show that anti-4-1BB had its strongest effect on vector induced tumor-antigen specific CD8 T cells in tumor draining lymph nodes.

### 8.12 Example 12

This example shows the anti-tumor efficacy of intravenous and intratumoral administration of artLCMV vectors encoding either GP70 or TRP2 tumor antigens together with 4-1BBL in the immunogenic MC38 model or in the non-immunogenic, ‘cold’ B16.F10 model.

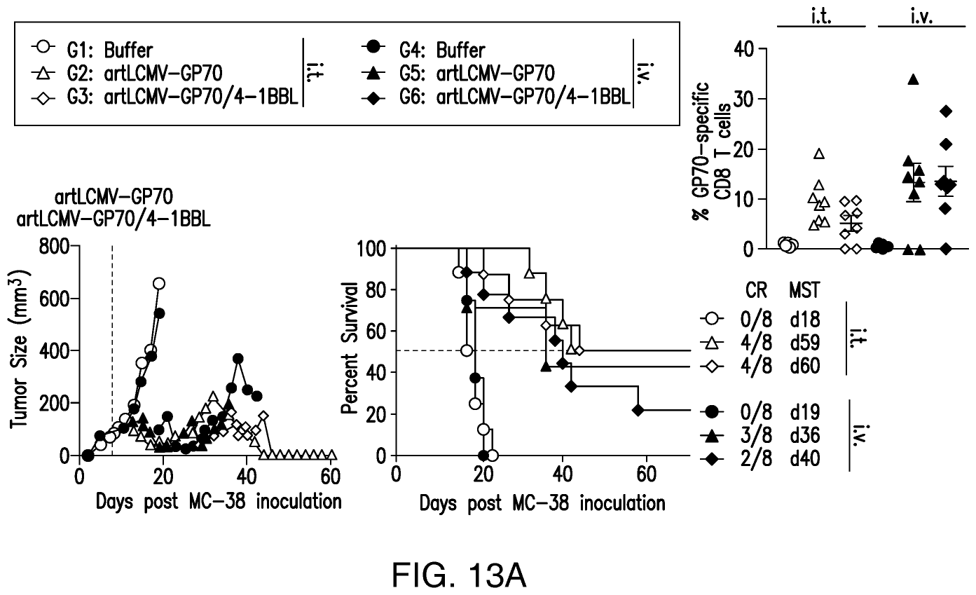

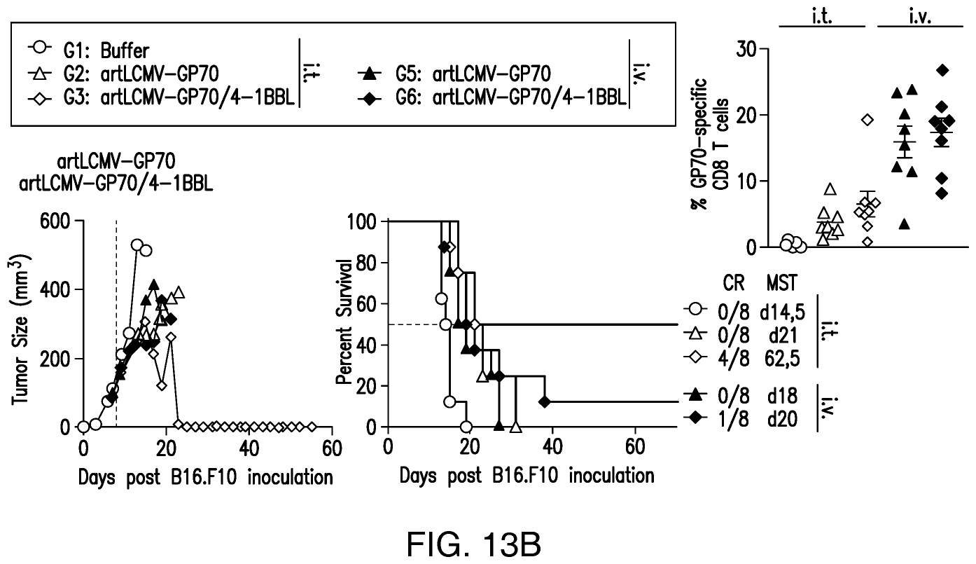

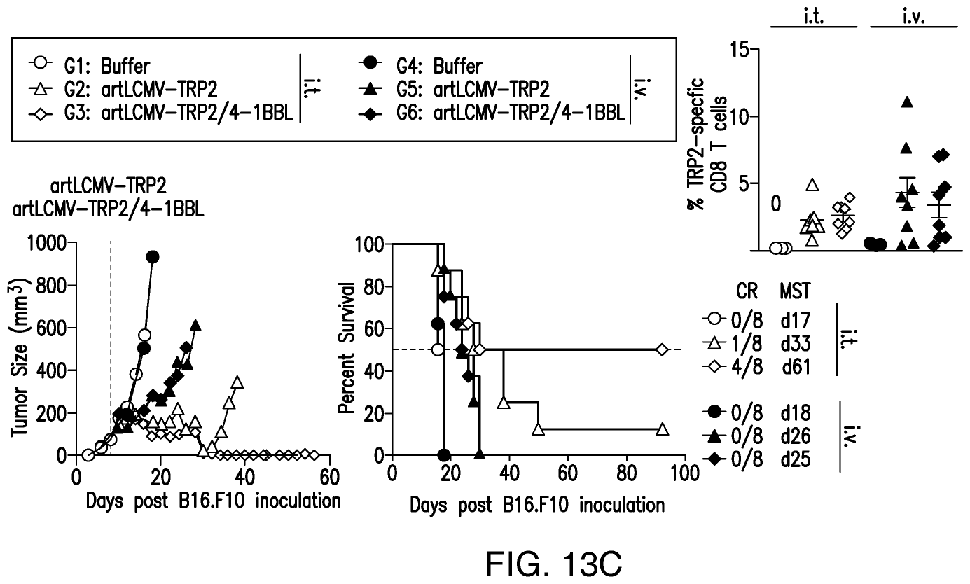

To investigate the anti-tumor efficacy of intravenous and intratumorally administered LCMV vector constructs in the immunogenic MC38 model (FIG. 13A), MC38 tumor-bearing C57BL/6 mice were immunized intratumorally (groups 2 and 3) or intravenously (groups 5 and 6) on day 8 after tumor challenge with the indicated vector constructs at 1x 105 RCV FFU/dose (see Table 14 Study Layout). Animals in groups 2, 3, 5 and 6 were treated with artLCMV vectors encoding the tumor antigen GP70 (groups 2 and 5) or GP70 and 4-1BBL (groups 3 and 6). Buffer-treated animals in groups 1 and 4 served as control.

TABLE 14
Study Layout.

| | | | Vaccination | | | | |
|---|---|---|---|---|---|---|---|
| | MC-38 | | | Dose | | | Mouse |
| Group | Dose | Day | Vector prime | [RCV FFU] | Day | Route | number |
| 1 | 1 × 10⁶ | Day 0 | Formulation buffer | n.a. | Day 8 | i.t. | 8 |
| 2 | cells | | artLCMV-GP70 | 1 × 10⁵ | | i.t. | 8 |
| 3 | s.c. | | artLCMV-GP70/41BBL | 1 × 10⁵ | | i.t. | 8 |
| 4 | | | Formulation buffer | n.a. | | i.v. | 8 |
| 5 | | | artLCMV-GP70 | 1 × 10⁵ | | i.v. | 8 |
| 6 | | | artLCMV-GP70/41BBL | 1 × 10⁵ | | i.v. | 8 |

As demonstrated in FIG. 13A (left panel), tumor growth was delayed in test groups treated with artLCMV-GP70 vectors irrespective of the administration route (groups 2, 3, 5 and 6). The tumor growth was most strongly inhibited in animals treated intratumorally with artLCMV-GP70 (group 2) and artLCMV-GP70-4-1BBL (group 3). The anti-tumor efficacy demonstrated by reduced tumor growth in FIG. 13A, also resulted in a survival benefit as shown in FIG. 13A (middle panel). Intravenous and intratumoral treatment with artLCMV-GP70 and artLCMV-GP70-4-1BBL led to increased median survival times compared to buffer treatment. The longest median survival time was achieved by intratumoral treatment with artLCMV-GP70 (59 days) and artLCMV-GP70-4-1BBL (60 days). Intratumoral treatment with artLCMV-GP70 and artLCMV-GP70-4-1BBL resulted in 5000 tumor-free mice.

To investigate the anti-tumor efficacy of intravenous and intratumorally administered LCMV vector constructs in the non-immunogenic, ‘cold’ B16.F10 model (FIGS. 13B and 13C), B16.F10 tumor-bearing C57BL/6 mice were immunized intratumorally (groups 2 and 3) or intravenously (groups 5 and 6) on day 8 after tumor challenge with the indicated vector constructs at 1×10⁵ RCV FFU/dose (see Table 15 Study Layout). Buffer-treated animals in group 1 served as control.

TABLE 15
Study Layout.

| | | | Vaccination | | | | |
|---|---|---|---|---|---|---|---|
| | B16.F10 | | | Dose | | | Mouse |
| Group | Dose | Day | Vector prime | [RCV FFU] | Day | Route | number |
| 1 | 2 × 10⁵ | Day 0 | Formulation buffer | n.a | Day 8 | i.t. | 8 |
| 2 | cells | | artLCMV-GP70 | 1 × 10⁵ | | i.t. | 8 |
| 3 | s.c. | | artLCMV-GP70/41BBL | 1 × 10⁵ | | i.t. | 8 |
| 4 | | | artLCMV-GP70 | 1 × 10⁵ | | i.v. | 8 |
| 5 | | | artLCMV-GP70/41BBL | 1 × 10⁵ | | i.v | 8 |
| 1 | 2 × 10⁵ | Day 0 | Formulation buffer | n.a. | Day 8 | i.t. | 8 |
| 2 | cells | | artLCMV-TRP2 | 1 × 10⁵ | | i.t. | 8 |
| 3 | s.c. | | artLCMV-TRP2/41BBL | 1 × 10⁵ | | i.t. | 8 |
| 4 | | | Formulation buffer | n.a. | | i.v. | 8 |
| 5 | | | artLCMV-TRP2 | 1 × 10⁵ | | i.v. | 8 |
| 6 | | | artLCMV-TRP2/41BBL | 1 × 10⁵ | | i.v | 8 |

As demonstrated in FIG. 13B (left panel), tumor growth was delayed in test groups treated with artLCMV-GP70 vectors irrespective of the administration route (groups 2, 3, 5 and 6). The tumor growth was most strongly inhibited in animals treated intratumorally with artLCMV-GP70/4-1BBL (group 3). The anti-tumor efficacy demonstrated by reduced tumor growth in FIG. 13B (left panel), also resulted in a survival benefit as shown in FIG. 13B (middle panel). Intravenous and intratumoral treatment with artLCMV-GP70 and artLCMV-GP70/4-1BBL led to increased median survival times compared to buffer treatment. Intratumoral treatment with artLCMV-GP70/4-1BBL resulted in 50% tumor-free mice, compared to 1/8 tumor free mice after intravenous treatment with the same vector. Neither intravenous nor intratumoral treatment with artLCMV-GP70 resulted in complete tumor clearance. GP70 specific CD8 T cell frequency was higher after intravenous as compared to intratumoral treatment and slightly enhanced by 4-1BBL co-expression (FIG. 13B, right panel). Similar results were seen after treatment with vectors encoding the melanoma self-antigen TRP2, artLCMV-TRP2 and artLCMV-TRP2/4-1BBL. The most pronounced tumor growth control (FIG. 13C, left panel) and the highest number of complete responders (FIG. 13C, middle panel) were observed after intratumoral treatment with artLCMV-TRP2/4-1BBL. Peripheral TRP2 specific CD8 T cell frequencies were higher after intravenous as compared to intratumoral treatment (FIG. 13C, right panel).

### 8.13 Example 13

This example shows the anti-tumor efficacy of intravenous and intratumoral administration of artLCMV vectors expressing interleukin 12.

To investigate the anti-tumor efficacy of intravenously and intratumorally administered LCMV vector constructs, B16F10 tumor-bearing C57BL/6 mice were treated intravenously or intratumorally on day 7 after tumor challenge with the indicated vector constructs at 1×10⁵ RCV FFU/dose (see Table 16 Study Layout). Animals in groups 2 and 3 were treated with artLCMV vectors encoding the cytokine interleukin 12 (IL12) in combination with either a relevant tumor antigen GP70 (groups 2 and 4) or an irrelevant protein (i.e., GFP, groups 3 and 5). Mice in groups 2 and 3 were injected intravenously and groups 4 and 5 were injected intratumorally with artLCMV vectors. Buffer-treated animals in group 1 served as control.

TABLE 16
Study Layout.

| | | | Vaccination | | | | |
|---|---|---|---|---|---|---|---|
| | B16.F10 | | | Dose | | | Mouse |
| Group | Dose | Day | Vector prime | [RCV FFU] | Day | Route | number |
| 1 | 2 × 10⁵ | Day 0 | Formulation buffer | n.a. | Day 8 | i.v. | 7 |
| 2 | cells | | artLCMV-GP70-IL12 | 1 × 10⁵ | | i.v. | 7 |
| 3 | s.c. | | artLCMV-GFP-IL12 | 1 × 10⁵ | | i.v. | 7 |
| 4 | | | artLCMV-GP70-IL12 | 1 × 10⁵ | | i.t. | 7 |
| 5 | | | artLCMV-GFP-IL12 | 1 × 10⁵ | | i.t. | 7 |

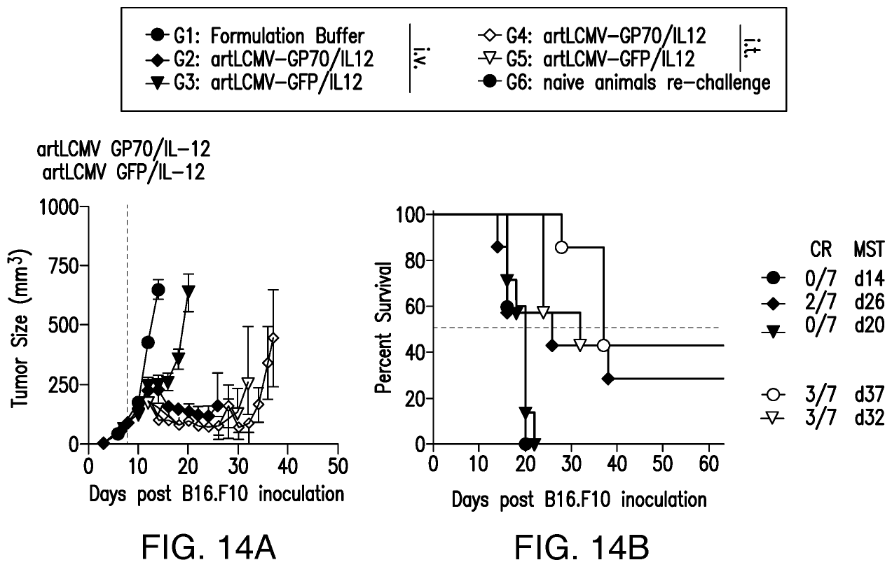

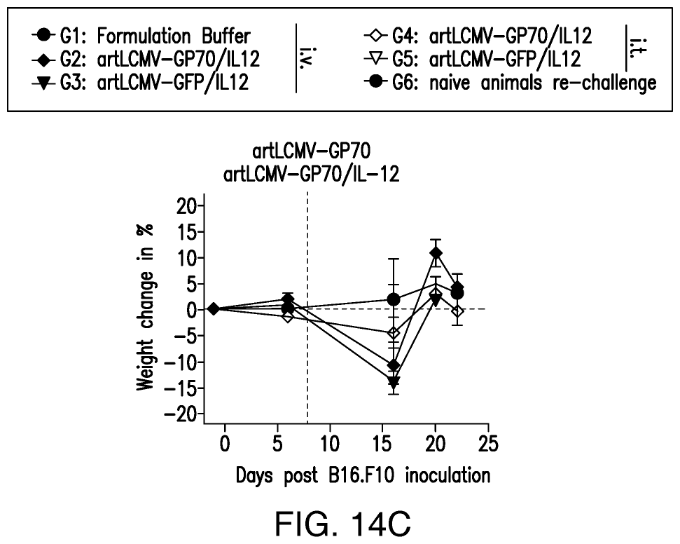

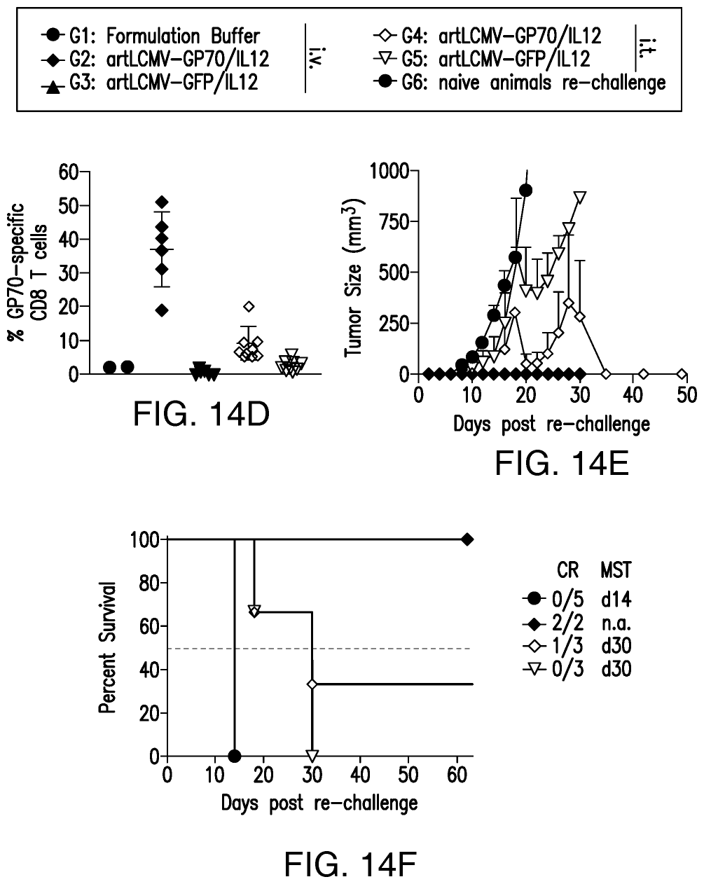

As demonstrated in FIG. 14A, tumor growth was slightly delayed in group 3 treated with artLCMV-GFP-IL12 vectors but not resulting in complete tumor clearance (FIG. 14B). Test groups treated intravenously with artLCMV-GP70-IL12 (group 2) or intratumorally with artLCMV-GP70-IL12 (group 4) and surprisingly with artLCMV-GFP-IL12 (group 5) had a more significant tumor size reduction (FIG. 14A) and prolonged median survival times (FIG. 14B). Unexpectedly, the inhibitory effect on tumor growth and the number of complete responders (3/7) observed after intratumoral treatment with artLCMV-GP70-IL12 (group 4) and vectors co-encoding the irrelevant GFP protein in combination with IL12 (group 5) were comparable. Body weight loss was higher on day 8 after intravenous as compared to intratumoral treatment (FIG. 14C). Intravenous treatment with artLCMV-GP70-IL12 resulted in the highest GP70 specific CD8 T cell frequencies in blood seven days after vector administration (FIG. 14D). This data shows that a single dose of artLCMV-GP70-IL12, without the administration of heterologous prime or boost was able to induce up to 50% GP70 specific CD8 T cells in blood. Surprisingly, intratumoral but not intravenous treatment with vectors encoding the irrelevant GFP protein and IL12 resulted in significant GP70 specific CD8 T cell frequencies in blood, which is a sign of epitope spreading.

To assess the function of memory CD8 T cells, tumor free mice were re-challenged with tumor cells on study day 107. Mice from group 2 treated intravenously with artLCMV-GP70-IL12 were fully protected against re-challenge and remained tumor-free (FIGS. 14E and F). Tumor-free animals from group 5 treated intratumorally with artLCMV-GFP-IL12 developed tumors with delayed kinetics as compared to naïve animals after re-challenge but could not eliminate the tumor. One out of three (1/3) mice that were tumor-free after intratumoral treatment with artLCMV-GP70-IL12 survived the tumor re-challenge.

### 8.14 Example 14

This example shows the anti-tumor efficacy of different doses of intravenously administered artLCMV vectors encoding the tumor antigen GP70 and interleukin 12. To investigate the anti-tumor efficacy of different doses of intravenously administered LCMV vector constructs, B16F10 tumor-bearing C57BL/6 mice were treated intravenously on day 7 after tumor challenge with the indicated vector constructs (see Table 17 Study Layout). Animals in group 1 were treated with artLCMV vectors encoding the tumor antigen GP70 at 1x 105 RCV FFU. Groups 3, 4 and 4 were treated with artLCMV vectors encoding the tumor antigen GP70 and IL12 at 1×10³, 1×10⁴, and 1×10⁵ RCV FFU, respectively.

TABLE 17
Study Layout.

| | | | Vaccination | | | | |
|---|---|---|---|---|---|---|---|
| | B16.F10 | | | Dose | | | Mouse |
| Group | Dose | Day | Vector prime | [RCV FFU] | Day | Route | number |
| 1 | 2 × 10⁵ | Day 0 | Formulation buffer | n.a. | Day 7 | i.v. | 5 |
| 2 | cells | | artLCMV-GP70 | 1 × 10⁵ | | i.v. | 5 |
| 3 | s.c. | | artLCMV-GP70- IL12 | 1 × 10³ | | i.v. | 5 |
| 4 | | | artLCMV-GP70- IL12 | 1 × 10⁴ | | i.v. | 5 |
| 5 | | | artLCMV-GP70- IL12 | 1 × 10⁵ | | i.v. | 5 |

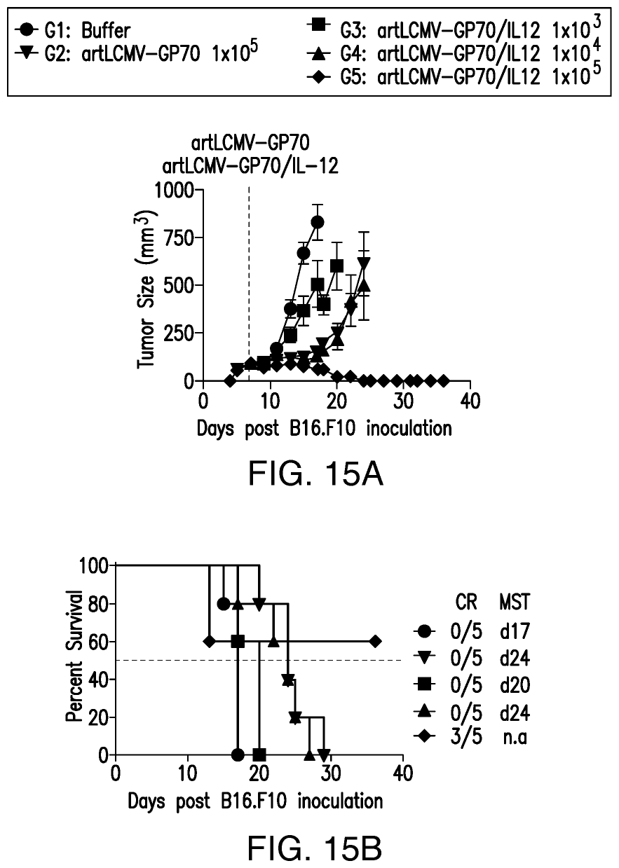

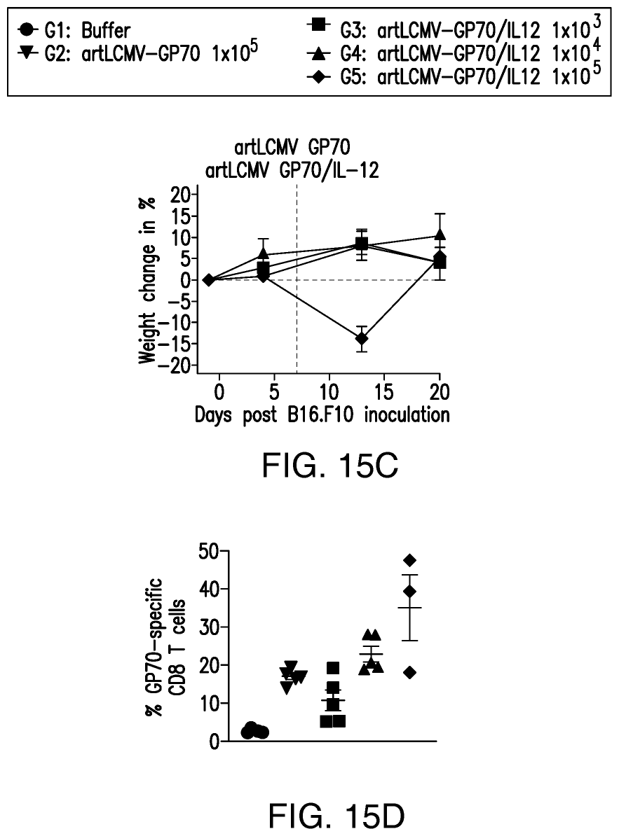

Treatment with artLCMV-GP70 resulted in tumor growth delay (FIG. 15A), longer median survival times as compared to buffer treated control mice but no complete responders (FIG. 15B). The lowest dose of artLCMV-GP70-IL12 delayed tumor growth and prolonged survival times as compared to buffer treatment but less than artLCMV-GP70 treatment. Surprisingly, at a tenfold lower dose than artLCMV-GP70, artLCMV-GP70-IL12 resulted in comparable tumor growth reduction and longer median survival times. Administration of 1×10⁵ RCV FFU artLCMV-GP70-IL12 had the strongest effect on tumor growth and resulted in 3/5 complete responders. This dose led to temporary weight loss six days after vector administration (FIG. 15C). The frequency of GP70 specific CD8 T cells in the blood after artLCMV-GP70-IL12 treatment was dose dependent (FIG. 15D). Surprisingly, 1x 104 RCV FFU of artLCMV-GP70-IL12 was more potent in inducing a GP70 specific CD8 T cell responses than 1×10⁵ RCV FFU of artLCMV-GP70.

### 8.15 Example 15

This example shows that the combination of artLCMV-E7E6 vectors and IL-2 immune complexes resulted in a complete tumor clearance in the TC-1 tumor model.

To investigate the potential synergism of arenaviral therapy and IL-2 complexes, C57BL/6 mice were subcutaneously injected with TC-1 cells and immunized intravenously with artLCMV vectors encoding E7E6 (groups 3 and 4) on day 10 after tumor cell injection. Animals in groups 2 and 4 received intraperitoneal injections of IL-2 complexes (recombinant human IL-2 mixed with anti-huIL-2 antibodies in a 2:1 molar ratio) 2 to 3 days apart starting 4 days after vector administration (5 treatments in total). Study design is shown in Table 18.

TABLE 18
Study design

| | | | | | | | | | | | | | C57BL/6J |
|---|---|---|---|---|---|---|---|---|---|---|---|---|---|
| | TC1 | | Vaccination | | | | | Antibody administration | | | | | mice |
| Group | Dose | Day | Vector prime | Dose | Day | Volume | Route | Ab | Dose | Day | Volume | Route | number |
| 1 | 1 × 10⁶ | Day 0 | Formulation | n.a. | Day 10 | 100 μl | i.v. | PBS | n.a. | days 14, | 100 μl | i.p. | 8 |
| | cells | | buffer | | | | | | | 16, 18, | | | |
| 2 | | | Formulation | n.a. | | | | IL-2 | 15000 U* | 21 and | | | 8 |
| | | | buffer | | | | | complex | | 23 | | | |
| 3 | | | artLCMV-E7E6 | 1 × 10⁵ | | | | PBS | n.a. | | | | 8 |
| 4 | | | artLCMV-E7E6 | 1 × 10⁵ | | | | IL-2 | 15000 U* | | | | 8 |
| | | | | | | | | complex | | | | | |

*15000 U human IL-2 mixed in a 2:1 molar ratio with anti-huIL-2 antibodies (MAB602)

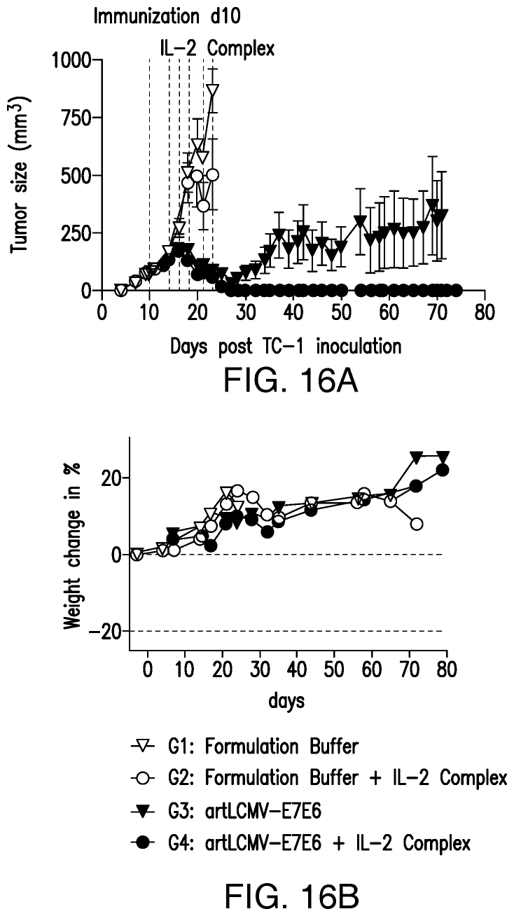

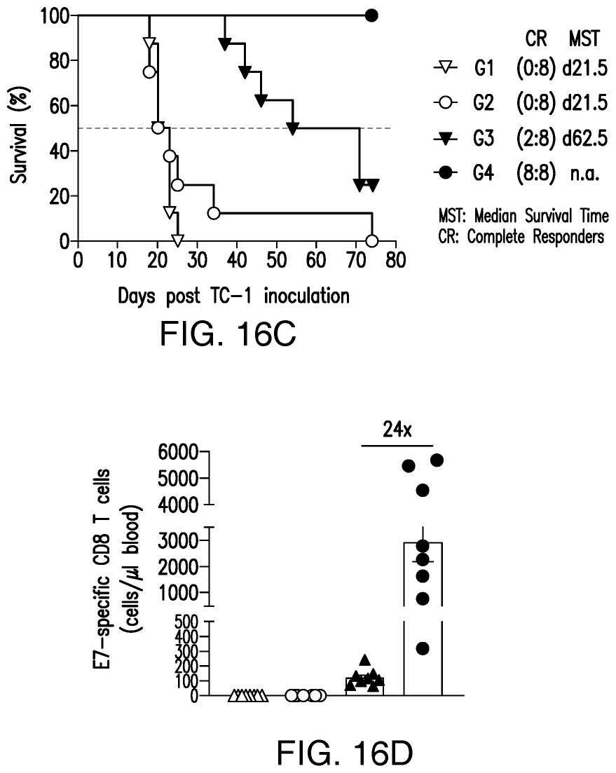

Tumor growth (FIG. 16A) and survival (FIG. 16C) were monitored and the frequency of antigen-specific CD8 T cells was analyzed in blood 8 days (after two rounds of IL-2 complex treatment), 15 days (2 days after all treatments were completed) and 22 days after vector administration (9 days after completed IL-2 complex treatment) (FIG. 16D).

Treatment with artLCMV encoding tumor-specific E7E6 antigen (artLCMV-E7E6) resulted in reduced tumor growth and significantly increased survival times of the animals. In two of the eight animals (25%), the tumor was completely cleared. Strikingly, the combination of artLCMV E7E6 vectors with IL-2 complex treatment resulted in complete tumor clearance in all treated animals and consequently a survival rate of 100% despite no effect of IL-2 complex alone on tumor growth and survival (FIGS. 16A and 16C). This high tumor efficacy also corresponded with large numbers of antigen-specific T cells, which were sustained in blood during and after completion of the IL-2 treatment cycle (FIG. 16D). Animals which had received combination therapy showed an up to 32-fold increase in antigen-specific CD8+ T cells compared to animals which were treated with artLCMV-E7E6 vector alone (FIG. 16D). It is also noteworthy that IL-2 complex treatment was well tolerated by the animals and did not result in any adverse events such as body weight loss (FIG. 16B).

What is claimed is:

1. A method for treating or preventing a neoplastic disease or an infectious disease in a subject in need thereof, wherein the method comprises administering to the subject (i) an arenavirus particle, and (ii) an immune checkpoint modulator and/or a cytokine, optionally wherein the cytokine is IL-12; wherein
a. the arenavirus particle comprises an arenavirus genome comprising a heterologous ORF encoding an antigen or an antigenic fragment thereof, and
b. (i) at least one arenavirus open reading frame (ORF) of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

2. The method of claim 1, wherein the method further comprises administering to the subject an antibody that specifically binds to the cytokine, wherein the antibody is administered with the cytokine in the same composition, optionally wherein the cytokine is IL-2 and the antibody is an anti-IL-2 antibody.

3. The method of claim 1 or 2, wherein the cytokine is selected from the group consisting of an IL-2-immunoglobulin fusion protein, a modified IL-2 molecule having abrogated binding to CD25, ANV419, XTX202, AB248, MDNA11, STK-012, and combinations thereof.

4. The method of any one of claims 1-3, wherein the arenavirus particle is tri-segmented and replication-competent and comprises one L segment and two S segments, wherein one of the two S segments is selected from the group consisting of:
a. an S segment, wherein the ORF encoding the NP is under control of an arenavirus genomic 5′ UTR;
b. an S segment, wherein the ORF encoding the Z protein is under control of an arenavirus genomic 5′ UTR;
c. an S segment, wherein the ORF encoding the L protein is under control of an arenavirus genomic 5′ UTR;
d. an S segment, wherein the ORF encoding the GP is under control of an arenavirus genomic 3′ UTR;
e. an S segment, wherein the ORF encoding the L protein is under control of an arenavirus genomic 3′ UTR; and
f. an S segment, wherein the ORF encoding the Z protein is under control of an arenavirus genomic 3′ UTR.

5. A method for treating or preventing a neoplastic disease or an infectious disease in a subject in need thereof, wherein the method comprises administering to the subject an arenavirus particle, wherein
a. the arenavirus particle comprises an arenavirus genome comprising:
i. a first heterologous ORF encoding an antigen; and
ii. a second heterologous ORF encoding an immune checkpoint modulator or a cytokine, optionally wherein the cytokine is IL-12; and
b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

6. The method of claim 5, wherein the arenavirus particle is tri-segmented and replication-competent and comprises one L segment and two S segments, wherein one of the two S segments is selected from the group consisting of:
a. an S segment, wherein the ORF encoding the NP is under control of an arenavirus genomic 5′ UTR;
b. an S segment, wherein the ORF encoding the Z protein is under control of an arenavirus genomic 5′ UTR;
c. an S segment, wherein the ORF encoding the L protein is under control of an arenavirus genomic 5′ UTR;
d. an S segment, wherein the ORF encoding the GP is under control of an arenavirus genomic 3′ UTR;
e. an S segment, wherein the ORF encoding the L protein is under control of an arenavirus genomic 3′ UTR; and
f. an S segment, wherein the ORF encoding the Z protein is under control of an arenavirus genomic 3′ UTR.

7. The method of claim 6, wherein (i) a first S segment is engineered to carry an arenaviral ORF encoding GP in a position under control of an arenavirus genomic 3′ UTR and the first heterologous ORF in a position under control of an arenavirus genomic 5′ UTR, and a second S segment is engineered to carry an arenaviral ORF encoding NP in a position under control of an arenavirus genomic 3′ UTR and the second heterologous ORF in a position under control of an arenavirus genomic 5′ UTR; or (ii) a first S segment is engineered to carry an arenaviral ORF encoding NP in a position under control of an arenavirus genomic 3′ UTR and the first heterologous ORF in a position under control of an arenavirus genomic 5′ UTR, and a second S segment is engineered to carry an arenaviral ORF encoding GP in a position under control of an arenavirus genomic 3′ UTR and the second heterologous ORF in a position under control of an arenavirus genomic 5′ UTR; or (iii) an S segment is engineered to carry both the first and the second heterologous ORFs.

8. The method of any one of claims 1-7, wherein the arenavirus particle is administered via intravenous injection or via intratumoral injection.

9. The method of any one of claims 1-8, wherein the at least one arenavirus ORF encodes the glycoprotein (“GP”), the nucleoprotein (“NP”), the matrix protein Z (“Z protein”) or the RNA dependent RNA polymerase L (“L protein”) of the arenavirus particle.

10. The method of any one of claims 1-3, 5, 8, and 9, wherein the at least one arenavirus ORF is either functionally inactivated or deleted and wherein the arenavirus particle has the ability to amplify and express its genetic information in cells infected with the arenavirus particle but is unable to produce further infectious progeny particles in normal, non-complementing cells.

11. The method of any one of claims 1-10, wherein the arenavirus particle is derived from lymphocytic choriomeningitis virus (LCMV) or Pichinde virus.

12. A method for treating or preventing a neoplastic disease or an infectious disease in a subject in need thereof, wherein the method comprises administering to the subject a first and a second arenavirus particles, wherein
(a) the first arenavirus particle comprises a first arenavirus genome comprising: a first heterologous ORF encoding an antigen; and (i) at least one first arenavirus ORF of the first arenavirus genome is either functionally inactivated or deleted, or (ii) at least one first arenavirus ORF is located in a position other than the wild-type position of said at least one first arenavirus ORF, or (iii) a fragment of at least one first arenavirus ORF is located in a position other than the wild-type position of said fragment of the at least one first arenavirus ORF; and
(b) the second arenavirus particle comprises a second arenavirus genome comprising: a second heterologous ORF encoding an immune checkpoint modulator or a cytokine, optionally wherein the cytokine is IL-12; and (i) at least one second arenavirus ORF of the second arenavirus genome is either functionally inactivated or deleted, or (ii) at least one second arenavirus ORF is located in a position other than the wild-type position of said at least one second arenavirus ORF, or (iii) a fragment of at least one second arenavirus ORF is located in a position other than the wild-type position of said fragment of the at least one second arenavirus ORF.

13. The method of claim 12, wherein the first arenavirus particle, the second arenavirus particle, or both the first and second arenavirus particles are tri-segmented and replication-competent and comprise one L segment and two S segments, wherein one of the two S segments is selected from the group consisting of:
a. an S segment, wherein the ORF encoding the NP is under control of an arenavirus genomic 5′ UTR;
b. an S segment, wherein the ORF encoding the Z protein is under control of an arenavirus genomic 5′ UTR;
c. an S segment, wherein the ORF encoding the L protein is under control of an arenavirus genomic 5′ UTR;
d. an S segment, wherein the ORF encoding the GP is under control of an arenavirus genomic 3′ UTR;
e. an S segment, wherein the ORF encoding the L protein is under control of an arenavirus genomic 3′ UTR; and
f. an S segment, wherein the ORF encoding the Z protein is under control of an arenavirus genomic 3′ UTR.

14. The method of claim 12 or 13, wherein the first and/or the second arenavirus particles are administered via intravenous injection or via intratumoral injection.

15. The method of any one of claims 12-14, wherein the at least one first arenavirus ORF encodes the glycoprotein (“GP”), the nucleoprotein (“NP”), the matrix protein Z (“Z protein”) or the RNA dependent RNA polymerase L (“L protein”) of the first arenavirus particle; and/or the at least one second arenavirus ORF encodes the glycoprotein (“GP”), the nucleoprotein (“NP”), the matrix protein Z (“Z protein”) or the RNA dependent RNA polymerase L (“L protein”) of the second arenavirus particle.

16. The method of any one of claims 12, 14, and 15, wherein the at least one first arenavirus ORF is either functionally inactivated or deleted and wherein the first arenavirus particle has the ability to amplify and express its genetic information in cells infected with the first arenavirus particle but is unable to produce further infectious progeny particles in normal, non-complementing cells; and/or wherein the at least one second arenavirus ORF is either functionally inactivated or deleted and wherein the second arenavirus particle has the ability to amplify and express its genetic information in cells infected with the second arenavirus particle but is unable to produce further infectious progeny particles in normal, non-complementing cells.

17. The method of any one of claims 12-16, wherein the first arenavirus particle, the second arenavirus particle, or both the first and second arenavirus particles are derived from lymphocytic choriomeningitis virus (LCMV) or Pichinde virus.

18. A method for treating or preventing a neoplastic disease or preventing or treating an infectious disease in a subject in need thereof, wherein the method comprises administering to the subject an arenavirus particle; wherein
a. the arenavirus particle comprises an arenavirus genome comprising a heterologous ORF encoding (i) a cytokine, optionally the cytokine is IL-12, or (ii) an immune checkpoint modulator, optionally the immune checkpoint modulator is a ligand of 4-1BB; and
b. (i) at least one arenavirus ORF of the arenavirus genome is either functionally inactivated or deleted, or (ii) at least one arenavirus ORF is located in a position other than the wild-type position of said at least one arenavirus ORF, or (iii) a fragment of at least one arenavirus ORF is located in a position other than the wild-type position of said fragment of the at least one arenavirus ORF.

19. The method of any one of claims 1-18, further comprising administering an additional immune checkpoint modulator that is different from the immune checkpoint modulator.

20. The method of any one of claims 1-19, wherein the immune checkpoint modulator of any one of claims 1-18 and/or the additional immune checkpoint modulator of claim 19 is an agonist of 4-1BB costimulatory pathway, an agonist of OX40 costimulatory pathway, an antagonist of NKG2A coinhibitory pathway, or a combination thereof;
optionally wherein
(i) the agonist of 4-1BB costimulatory pathway is an agonistic antibody of 4-1BB or 4-1BBL, optionally wherein the agonist of the 4-1BB costimulatory pathway is selected from the group consisting of utomilumab (PF-05082566), INBRX-105, ABL503, ATOR-1017, FS222, RG7827 (FAP 4-1BBL FP), RG6076 (CD19-4-1BBL), urelumab (BMS-663513), CHU CD137 agonist switch antibody, AGEN-2373, CTX-471, FS-120, LVGN-6051, MCLA-145, AMG-506, PRS-343, STA-551, ADG-106, DSP-107, DuoBody-CD40x4-1BB (BNT-312, GEN1042), DuoBody-PD-L1x 4-1BB (GEN-1046, BNT-311), ALG.APV-527, CB307, ABP-300, NM21-1480, EU101, RO7227166, ABL111, HERA-4-1BBL, SL-279137 (PD-1-Fc-4-1BBL), and combinations thereof;
(ii) the agonist of OX40 costimulatory pathway is an agonistic antibody of OX40, optionally wherein the agonist of the OX40 costimulatory pathway is selected from the group consisting of INBRX-106, PF-04518600, BMS-986178, BGB-A445, MEDI0562, MOXR-0916 (pogalizumab, RG 7888), anti-FAP/anti-OX40 bispecific agonistic antibody, anti-FAP/OX40L agonist fusion protein, INCAGN01949, MEDI6469, GSK3174998, HERA-OX40L, SL-279252 (PD1-Fc-OX40L), and combinations thereof, and/or
(iii) the antagonist of the NKG2A coinhibitory pathway is an antagonistic antibody of NKG2A.

21. The method of any one of claims 1-17, 19, and 20, wherein the method is for treating or preventing a neoplastic disease, and the antigen is a tumor antigen, tumor associated antigen, or antigenic fragment thereof.

22. The method of claim 21, wherein the neoplastic disease is a solid tumor and wherein the method results in an increase of the concentration of T cells within the solid tumor, optionally wherein the method results in an increased concentration of CD8+ T cells, an increased concentration of CD4+ T cells, an increased concentration of tumor antigen specific T cells, an increased concentration of T cells producing IFN-gamma, an increased concentration of T cells producing granzyme B, and/or an increased ratio of effector T cells/regulatory T cells within the solid tumor.

23. The method claim 21 or 22, wherein the method has a higher anti-tumor efficacy as compared to administration of a control arenavirus particle expressing the tumor antigen, tumor associated antigen, or antigenic fragment thereof, alone; and/or the method results in an increase in the survival rate of subjects treated with the method, compared to subjects having the same neoplastic disease in the absence of such treatment.

24. The method of any one of claims 1-17, 19, and 20, wherein the method is for preventing or treating an infectious disease, and the antigen is an antigen of a pathogen that causes the infectious disease, or antigenic fragment thereof.

25. The method of claim 24, wherein the method results in an increase of the concentration of T cells near cells infected with the pathogen, optionally wherein the method results in an increased concentration of CD8+ T cells, an increased concentration of CD4+ T cells, an increased concentration of tumor antigen specific T cells, an increased concentration of T cells producing IFN-gamma, an increased concentration of T cells producing granzyme B, and/or an increased ratio of effector T cells/regulatory T cells, near cells infected with the pathogen.

26. The method of claim 24 or 25, wherein the method has a higher anti-infection efficacy as compared to administration of a control arenavirus particle expressing the antigen of a pathogen that causes the infectious disease, or antigenic fragment thereof, alone; and/or the method results in an increase in the survival rate of subjects treated with the method, compared to subjects having the same infectious disease in the absence of such treatment.
